SEQUENCE LISTING (532 sequences)

SEQ ID NO: 1 - protein, 11 aa, synthetic construct
  REGION 1..11: note = Description of Artificial Sequence: Synthetic peptide
CTTDHRRRGLW

SEQ ID NO: 2 - protein, 12 aa, synthetic construct
  REGION 1..12: note = Description of Artificial Sequence: Synthetic peptide
CTRRNPGGMDVW

SEQ ID NO: 3 - protein, 16 aa, synthetic construct
  REGION 1..16: note = Description of Artificial Sequence: Synthetic peptide
CARLRVGRDYKGMDVR

SEQ ID NO: 4 - protein, 16 aa, synthetic construct
  REGION 1..16: note = Description of Artificial Sequence: Synthetic peptide
CVTYTYCGGDCYSEDW

SEQ ID NO: 5 - protein, 15 aa, synthetic construct
  REGION 1..15: note = Description of Artificial Sequence: Synthetic peptide
CAKVRRGYSYGTDYW

SEQ ID NO: 6 - protein, 10 aa, synthetic construct [sequence omitted]

SEQ ID NO: 7 - protein, 16 aa, synthetic construct
  REGION 1..16: note = Description of Artificial Sequence: Synthetic peptide
CALARSYDYYYGVDVW

SEQ ID NO: 8 - protein, 10 aa, synthetic construct [sequence omitted]

SEQ ID NO: 9 - protein, 12 aa, synthetic construct
  REGION 1..12: note = Description of Artificial Sequence: Synthetic peptide
CSRDYGYYFDFW

SEQ ID NO: 10 - protein, 13 aa, synthetic construct
  REGION 1..13: note = Description of Artificial Sequence: Synthetic peptide
CATRSVLTGSPIW

SEQ ID NO: 11 - protein, 19 aa, synthetic construct
  REGION 1..19: note = Description of Artificial Sequence: Synthetic peptide
CAREAVAGTHPQAGDFDLW

SEQ ID NO: 12 - protein, 22 aa, synthetic construct
  REGION 1..22: note = Description of Artificial Sequence: Synthetic peptide
CVRGTLYHYTSGSYYSDAFDIW

SEQ ID NO: 13 - protein, 22 aa, synthetic construct
  REGION 1..22: note = Description of Artificial Sequence: Synthetic peptide
CARGTLYHYTSGSYYSDAFDIW

SEQ ID NO: 14 - protein, 18 aa, synthetic construct
  REGION 1..18: note = Description of Artificial Sequence: Synthetic peptide
CAKAGSYDGSGYNYFDSW

SEQ ID NO: 15 - protein, 19 aa, synthetic construct
  REGION 1..19: note = Description of Artificial Sequence: Synthetic peptide
CVREGSLTGPGWENWFDPW

SEQ ID NO: 16 - protein, 13 aa, synthetic construct
  REGION 1..13: note = Description of Artificial Sequence: Synthetic peptide
CARGLRLGELKQW

SEQ ID NO: 17 - protein, 9 aa, synthetic construct
  REGION 1..9: note = Description of Artificial Sequence: Synthetic peptide
FTFSSYAMS

SEQ ID NO: 18 - protein, 9 aa, synthetic construct
  REGION 1..9: note = Description of Artificial Sequence: Synthetic peptide
FTLSDSTVH

SEQ ID NO: 19 - protein, 9 aa, synthetic construct
  REGION 1..9: note = Description of Artificial Sequence: Synthetic peptide
FTFTNAWMG

SEQ ID NO: 20 - protein, 9 aa, synthetic construct
  REGION 1..9: note = Description of Artificial Sequence: Synthetic peptide
FAFSSYAMS

SEQ ID NO: 21 - protein, 9 aa, synthetic construct
  REGION 1..9: note = Description of Artificial Sequence: Synthetic peptide
FTFSGYGMH

SEQ ID NO: 22 - protein, 9 aa, synthetic construct
  REGION 1..9: note = Description of Artificial Sequence: Synthetic peptide
FAFSSYAMN

SEQ ID NO: 23 - protein, 9 aa, synthetic construct
  REGION 1..9: note = Description of Artificial Sequence: Synthetic peptide
GTFSSDAIT

SEQ ID NO: 24 - protein, 10 aa, synthetic construct
  REGION 1..10: note = Description of Artificial Sequence: Synthetic peptide
GSINIGNWYN

SEQ ID NO: 25 - protein, 9 aa, synthetic construct
  REGION 1..9: note = Description of Artificial Sequence: Synthetic peptide
HTFSDYYMH

SEQ ID NO: 26 - protein, 9 aa, synthetic construct
  REGION 1..9: note = Description of Artificial Sequence: Synthetic peptide
YTFTSYHMH

SEQ ID NO: 27 - protein, 9 aa, synthetic construct
  REGION 1..9: note = Description of Artificial Sequence: Synthetic peptide
YTFTDHYIH

SEQ ID NO: 28 - protein, 9 aa, synthetic construct
  REGION 1..9: note = Description of Artificial Sequence: Synthetic peptide
FTFSNFDMH

SEQ ID NO: 29 - protein, 9 aa, synthetic construct
  REGION 1..9: note = Description of Artificial Sequence: Synthetic peptide
FTFSTFDMH

SEQ ID NO: 30 - protein, 9 aa, synthetic construct
  REGION 1..9: note = Description of Artificial Sequence: Synthetic peptide
FTFDDFAMH

SEQ ID NO: 31 - protein, 9 aa, synthetic construct
  REGION 1..9: note = Description of Artificial Sequence: Synthetic peptide
FIVSDYGMH

SEQ ID NO: 32 - protein, 9 aa, synthetic construct
  REGION 1..9: note = Description of Artificial Sequence: Synthetic peptide
GSFRDYAVS

SEQ ID NO: 33 - protein, 13 aa, synthetic construct
  REGION 1..13: note = Description of Artificial Sequence: Synthetic peptide
SAISGSGGSTYYA

SEQ ID NO: 34 - protein, 15 aa, synthetic construct
  REGION 1..15: note = Description of Artificial Sequence: Synthetic peptide
AYIRSKVNSYATGYA

SEQ ID NO: 35 - protein, 15 aa, synthetic construct
  REGION 1..15: note = Description of Artificial Sequence: Synthetic peptide
ARIKSKSDGGKTDYA

SEQ ID NO: 36 - protein, 13 aa, synthetic construct
  REGION 1..13: note = Description of Artificial Sequence: Synthetic peptide
ATISYDGSNKYYA

SEQ ID NO: 37 - protein, 12 aa, synthetic construct
  REGION 1..12: note = Description of Artificial Sequence: Synthetic peptide
STVSGSGSTYYA

SEQ ID NO: 38 - protein, 13 aa, synthetic construct
  REGION 1..13: note = Description of Artificial Sequence: Synthetic peptide
GWINPNSGGTNYA

SEQ ID NO: 39 - protein, 12 aa, synthetic construct
  REGION 1..12: note = Description of Artificial Sequence: Synthetic peptide
GEVYHSGSTNYN

SEQ ID NO: 40 - protein, 13 aa, synthetic construct
  REGION 1..13: note = Description of Artificial Sequence: Synthetic peptide
GRINPDSGATDYA

SEQ ID NO: 41 - protein, 12 aa, synthetic construct
  REGION 1..12: note = Description of Artificial Sequence: Synthetic peptide
SGLDHSGGAHYA

SEQ ID NO: 42 - protein, 12 aa, synthetic construct
  REGION 1..12: note = Description of Artificial Sequence: Synthetic peptide
STFDHSGGAHYA

SEQ ID NO: 43 - protein, 13 aa, synthetic construct
  REGION 1..13: note = Description of Artificial Sequence: Synthetic peptide
SSISWNSVNIDYA

SEQ ID NO: 44 - protein, 13 aa, synthetic construct
  REGION 1..13: note = Description of Artificial Sequence: Synthetic peptide
AVMSYDGSQKFYS

SEQ ID NO: 45 - protein, 13 aa, synthetic construct
  REGION 1..13: note = Description of Artificial Sequence: Synthetic peptide
GGIIPMFGRGEYT

SEQ ID NO: 46 - protein, 11 aa, synthetic construct
  REGION 1..11: note = Description of Artificial Sequence: Synthetic peptide
QASQDISNYLN

SEQ ID NO: 47 - protein, 13 aa, synthetic construct
  REGION 1..13: note = Description of Artificial Sequence: Synthetic peptide
SGSSSNIGSNYVY

SEQ ID NO: 48 - protein, 11 aa, synthetic construct
  REGION 1..11: note = Description of Artificial Sequence: Synthetic peptide
RASQNINNYLN

SEQ ID NO: 49 - protein, 11 aa, synthetic construct
  REGION 1..11: note = Description of Artificial Sequence: Synthetic peptide
RASQGIRNDLG

SEQ ID NO: 50 - protein, 11 aa, synthetic construct
  REGION 1..11: note = Description of Artificial Sequence: Synthetic peptide
RASRSIRNDLA

SEQ ID NO: 51 - protein, 11 aa, synthetic construct
  REGION 1..11: note = Description of Artificial Sequence: Synthetic peptide
RASQSISSWLA

SEQ ID NO: 52 - protein, 11 aa, synthetic construct
  REGION 1..11: note = Description of Artificial Sequence: Synthetic peptide
RASQDVSSWLA

SEQ ID NO: 53 - protein, 11 aa, synthetic construct
  REGION 1..11: note = Description of Artificial Sequence: Synthetic peptide
GGDNIGDKRVH

SEQ ID NO: 54 - protein, 11 aa, synthetic construct
  REGION 1..11: note = Description of Artificial Sequence: Synthetic peptide
RASQDIRNDLG

SEQ ID NO: 55 - protein, 11 aa, synthetic construct
  REGION 1..11: note = Description of Artificial Sequence: Synthetic peptide
RASQSISSYLN

SEQ ID NO: 56 - protein, 11 aa, synthetic construct
  REGION 1..11: note = Description of Artificial Sequence: Synthetic peptide
GGDRLHFKTVD

SEQ ID NO: 57 - protein, 17 aa, synthetic construct
  REGION 1..17: note = Description of Artificial Sequence: Synthetic peptide
KSSQSVLDSSKNKNYLA

SEQ ID NO: 58 - protein, 11 aa, synthetic construct
  REGION 1..11: note = Description of Artificial Sequence: Synthetic peptide
RASQSINNWLA

SEQ ID NO: 59 - protein, 11 aa, synthetic construct
  REGION 1..11: note = Description of Artificial Sequence: Synthetic peptide
GGNNIGSKSVH

SEQ ID NO: 60 - protein, 14 aa, synthetic construct
  REGION 1..14: note = Description of Artificial Sequence: Synthetic peptide
TGSSSNIGAGFDVH

SEQ ID NO: 61 - protein, 11 aa, synthetic construct
  REGION 1..11: note = Description of Artificial Sequence: Synthetic peptide
RASQSISTYLN

SEQ ID NO: 62 - protein, 11 aa, synthetic construct
  REGION 1..11: note = Description of Artificial Sequence: Synthetic peptide
RASQSISSFLN

SEQ ID NO: 63 - protein, 11 aa, synthetic construct
  REGION 1..11: note = Description of Artificial Sequence: Synthetic peptide
RASQGISDNLN

SEQ ID NO: 64 - protein, 11 aa, synthetic construct
  REGION 1..11: note = Description of Artificial Sequence: Synthetic peptide
RASETISIYLN

SEQ ID NO: 65 - protein, 11 aa, synthetic construct
  REGION 1..11: note = Description of Artificial Sequence: Synthetic peptide
RASQGISSWLA

SEQ ID NO: 66 - protein, 13 aa, synthetic construct
  REGION 1..13: note = Description of Artificial Sequence: Synthetic peptide
SGGSSNIGDYDVS

SEQ ID NO: 67 - protein, 11 aa, synthetic construct
  REGION 1..11: note = Description of Artificial Sequence: Synthetic peptide
RASQGVSSWLA

SEQ ID NO: 68 - protein, 7 aa, synthetic construct
  REGION 1..7: note = Description of Artificial Sequence: Synthetic peptide
GASNLHS

SEQ ID NO: 69 - protein, 7 aa, synthetic construct
  REGION 1..7: note = Description of Artificial Sequence: Synthetic peptide
RNNQRPS

SEQ ID NO: 70 - protein, 7 aa, synthetic construct
  REGION 1..7: note = Description of Artificial Sequence: Synthetic peptide
DASNLET

SEQ ID NO: 71 - protein, 7 aa, synthetic construct
  REGION 1..7: note = Description of Artificial Sequence: Synthetic peptide
AASTLQS

SEQ ID NO: 72 - protein, 7 aa, synthetic construct
  REGION 1..7: note = Description of Artificial Sequence: Synthetic peptide
KASSLES

SEQ ID NO: 73 - protein, 7 aa, synthetic construct
  REGION 1..7: note = Description of Artificial Sequence: Synthetic peptide
DDADRPT

SEQ ID NO: 74 - protein, 7 aa, synthetic construct
  REGION 1..7: note = Description of Artificial Sequence: Synthetic peptide
AASSLQS

SEQ ID NO: 75 - protein, 7 aa, synthetic construct
  REGION 1..7: note = Description of Artificial Sequence: Synthetic peptide
GDSDRPS

SEQ ID NO: 76 - protein, 7 aa, synthetic construct
  REGION 1..7: note = Description of Artificial Sequence: Synthetic peptide
WASIRES

SEQ ID NO: 77 - protein, 7 aa, synthetic construct
  REGION 1..7: note = Description of Artificial Sequence: Synthetic peptide
KASTLES

SEQ ID NO: 78 - protein, 7 aa, synthetic construct
  REGION 1..7: note = Description of Artificial Sequence: Synthetic peptide
DDTNRPS

SEQ ID NO: 79 - protein, 7 aa, synthetic construct
  REGION 1..7: note = Description of Artificial Sequence: Synthetic peptide
GNSNRPS

SEQ ID NO: 80 - protein, 7 aa, synthetic construct
  REGION 1..7: note = Description of Artificial Sequence: Synthetic peptide
AASSLES

SEQ ID NO: 81 - protein, 7 aa, synthetic construct
  REGION 1..7: note = Description of Artificial Sequence: Synthetic peptide
AASNLQM

SEQ ID NO: 82 - protein, 7 aa, synthetic construct
  REGION 1..7: note = Description of Artificial Sequence: Synthetic peptide
GASTLQS

SEQ ID NO: 83 - protein, 7 aa, synthetic construct
  REGION 1..7: note = Description of Artificial Sequence: Synthetic peptide
ATSRLQS

SEQ ID NO: 84 - protein, 11 aa, synthetic construct [sequence omitted]

SEQ ID NO: 85 - protein, 13 aa, synthetic construct
  REGION 1..13: note = Description of Artificial Sequence: Synthetic peptide
CAAWDDRLAGYVF

SEQ ID NO: 86 - protein, 11 aa, synthetic construct [sequence omitted]

SEQ ID NO: 87 - protein, 11 aa, synthetic construct
  REGION 1..11: note = Description of Artificial Sequence: Synthetic peptide
CQQANSYPVTF

SEQ ID NO: 88 - protein, 11 aa, synthetic construct
  REGION 1..11: note = Description of Artificial Sequence: Synthetic peptide
CQRSYTTPPTF

SEQ ID NO: 89 - protein, 11 aa, synthetic construct
  REGION 1..11: note = Description of Artificial Sequence: Synthetic peptide
CQQAKSFPVSF

SEQ ID NO: 90 - protein, 11 aa, synthetic construct
  REGION 1..11: note = Description of Artificial Sequence: Synthetic peptide
CQQHYTTPPTF

SEQ ID NO: 91 - protein, 13 aa, synthetic construct
  REGION 1..13: note = Description of Artificial Sequence: Synthetic peptide
CQVYDRASNHVVF

SEQ ID NO: 92 - protein, 11 aa, synthetic construct [sequence omitted]

SEQ ID NO: 93 - protein, 13 aa, synthetic construct
  REGION 1..13: note = Description of Artificial Sequence: Synthetic peptide
CQVWDHNGDGRVF

SEQ ID NO: 94 - protein, 11 aa, synthetic construct [sequence omitted]

SEQ ID NO: 95 - protein, 11 aa, synthetic construct
  REGION 1..11: note = Description of Artificial Sequence: Synthetic peptide
CQQYNNHSRTF

SEQ ID NO: 96 - protein, 13 aa, synthetic construct
  REGION 1..13: note = Description of Artificial Sequence: Synthetic peptide
CQLWDSSGEHWVF

SEQ ID NO: 97 - protein, 13 aa, synthetic construct
  REGION 1..13: note = Description of Artificial Sequence: Synthetic peptide
CRSYDSSLSGSVF

SEQ ID NO: 98 - protein, 10 aa, synthetic construct [sequence omitted]

SEQ ID NO: 99 - protein, 11 aa, synthetic construct [sequence omitted]

SEQ ID NO: 100 - protein, 11 aa, synthetic construct [sequence omitted]

SEQ ID NO: 101 - protein, 11 aa, synthetic construct [sequence omitted]

SEQ ID NO: 102 - protein, 11 aa, synthetic construct [sequence omitted]

SEQ ID NO: 103 - protein, 11 aa, synthetic construct [sequence omitted]

SEQ ID NO: 104 - protein, 11 aa, synthetic construct [sequence omitted]

SEQ ID NO: 105 - protein, 10 aa, synthetic construct [sequence omitted]

SEQ ID NO: 106 - protein, 11 aa, synthetic construct [sequence omitted]

SEQ ID NO: 107 - protein, 13 aa, synthetic construct [sequence omitted]

SEQ ID NO: 108 - protein, 11 aa, synthetic construct
  REGION 1..11: note = Description of Artificial Sequence: Synthetic peptide
CQQSYFTPVTF

SEQ ID NO: 109 - protein, 26 aa, synthetic construct
  REGION 1..26: note = Description of Artificial Sequence: Synthetic peptide
QVTLKESGGGVVQPGRSLRLSCAASG

SEQ ID NO: 110 - protein, 26 aa, synthetic construct
  REGION 1..26: note = Description of Artificial Sequence: Synthetic peptide
EVQLVESGGGLVQPGGSLRLSCAASG

SEQ ID NO: 111 - protein, 26 aa, synthetic construct
  REGION 1..26: note = Description of Artificial Sequence: Synthetic peptide
EVQLVESGGGVVQPGRSLRLSCAASG

SEQ ID NO: 112 - protein, 26 aa, synthetic construct
  REGION 1..26: note = Description of Artificial Sequence: Synthetic peptide
EVQLVESGGDLVQPGGSLRLSCAASG

SEQ ID NO: 113 - protein, 26 aa, synthetic construct
  REGION 1..26: note = Description of Artificial Sequence: Synthetic peptide
QVTLKESGGGLVQPGGSLRLSCAASG

SEQ ID NO: 114 - protein, 26 aa, synthetic construct
  REGION 1..26: note = Description of Artificial Sequence: Synthetic peptide
QVTLKESGGGLVKPGGSLRLSCAASG

SEQ ID NO: 115 - protein, 26 aa, synthetic construct
  REGION 1..26: note = Description of Artificial Sequence: Synthetic peptide
  VARIANT 1: note = MOD_RES - Any amino acid
XVQLQESGGGVVQPGRSLRLSCAGSG

SEQ ID NO: 116 - protein, 26 aa, synthetic construct
  REGION 1..26: note = Description of Artificial Sequence: Synthetic peptide
  VARIANT 3: note = MOD_RES - Any amino acid
QVXLQESGGGLVQPGGSLRLSCAASG

SEQ ID NO: 117 - protein, 26 aa, synthetic construct
  REGION 1..26: note = Description of Artificial Sequence: Synthetic peptide
HVTLKESGGGVVQPGRSLRLSCAASG

SEQ ID NO: 118 - protein, 26 aa, synthetic construct
  REGION 1..26: note = Description of Artificial Sequence: Synthetic peptide
EVQLVESGAEVKKPGASVKVSCKASG

SEQ ID NO: 119 - protein, 26 aa, synthetic construct
  REGION 1..26: note = Description of Artificial Sequence: Synthetic peptide
QVQLVESGPGLVAPSGTLSLTCTVSG

SEQ ID NO: 120 - protein, 26 aa, synthetic construct
  REGION 1..26: note = Description of Artificial Sequence: Synthetic peptide
QVQLQQSGAEVKKPGASVKVSCKASG

SEQ ID NO: 121 - protein, 26 aa, synthetic construct
  REGION 1..26: note = Description of Artificial Sequence: Synthetic peptide
QVQLVQSGAEVKEPGASVKVSCKASG

SEQ ID NO: 122 - protein, 26 aa, synthetic construct
  REGION 1..26: note = Description of Artificial Sequence: Synthetic peptide
QVQLVQSGAEVKKPGASMKVSCKASG

SEQ ID NO: 123 - protein, 26 aa, synthetic construct
  REGION 1..26: note = Description of Artificial Sequence: Synthetic peptide
QVQLQESGGGLVQPGGSLRLSCAASG

SEQ ID NO: 124 - protein, 26 aa, synthetic construct
  REGION 1..26: note = Description of Artificial Sequence: Synthetic peptide
QVQLQQSGGGLVQPGGSLRLSCAASG

SEQ ID NO: 125 - protein, 26 aa, synthetic construct
  REGION 1..26: note = Description of Artificial Sequence: Synthetic peptide
EVQLVESGGGFVQPGGSLRLSCAASG

SEQ ID NO: 126 - protein, 26 aa, synthetic construct
  REGION 1..26: note = Description of Artificial Sequence: Synthetic peptide
QVQLVQSGGGLVQPGGSLRLSCAASG

SEQ ID NO: 127 - protein, 26 aa, synthetic construct
  REGION 1..26: note = Description of Artificial Sequence: Synthetic peptide
QVQLVQSGGGVVQPGSSLRLSCEASG

SEQ ID NO: 128 - protein, 26 aa, synthetic construct
  REGION 1..26: note = Description of Artificial Sequence: Synthetic peptide
  VARIANT 6: note = MOD_RES - Any amino acid
EVQLLXSAAEVKRPGSSVRVSCKVSG

SEQ ID NO: 129 - protein, 13 aa, synthetic construct
  REGION 1..13: note = Description of Artificial Sequence: Synthetic peptide
  VARIANT 4: note = MOD_RES - Any amino acid
WARXAPGKGLEWV

SEQ ID NO: 130 - protein, 13 aa, synthetic construct
  REGION 1..13: note = Description of Artificial Sequence: Synthetic peptide
WVRQASGKGLERV

SEQ ID NO: 131 - protein, 13 aa, synthetic construct
  REGION 1..13: note = Description of Artificial Sequence: Synthetic peptide
WVRQAPGKGLEWV

SEQ ID NO: 132 - protein, 13 aa, synthetic construct
  REGION 1..13: note = Description of Artificial Sequence: Synthetic peptide
WVRQVPGTGLEWV

SEQ ID NO: 133 - protein, 13 aa, synthetic construct
  REGION 1..13: note = Description of Artificial Sequence: Synthetic peptide
WVRQAPGQGLEWM

SEQ ID NO: 134 - protein, 13 aa, synthetic construct
  REGION 1..13: note = Description of Artificial Sequence: Synthetic peptide
WVRQSPGKGLEWI

SEQ ID NO: 135 - protein, 13 aa, synthetic construct
  REGION 1..13: note = Description of Artificial Sequence: Synthetic peptide
WVRQSPGKGLEWV

SEQ ID NO: 136 - protein, 13 aa, synthetic construct
  REGION 1..13: note = Description of Artificial Sequence: Synthetic peptide
WVRQAPGKSLEWV

SEQ ID NO: 137 - protein, 13 aa, synthetic construct
  REGION 1..13: note = Description of Artificial Sequence: Synthetic peptide
WVRQAPGQGPEWM

SEQ ID NO: 138 - protein, 34 aa, synthetic construct
  REGION 1..34: note = Description of Artificial Sequence: Synthetic polypeptide
DSVRGRFTISRDNSKNTLYLQMNSLRAEDTAVYY

SEQ ID NO: 139 - protein, 34 aa, synthetic construct
  REGION 1..34: note = Description of Artificial Sequence: Synthetic polypeptide
ASVKGRFTISRDDSKNTAYLQMNSLKIEDTAVYY

SEQ ID NO: 140 - protein, 34 aa, synthetic construct
  REGION 1..34: note = Description of Artificial Sequence: Synthetic polypeptide
DSVKGRFTISRDNSKNMLYLQMNSLRAEDTALYY

SEQ ID NO: 141 - protein, 34 aa, synthetic construct
  REGION 1..34: note = Description of Artificial Sequence: Synthetic polypeptide
APVKGRFTISRDDSQNTLYLQMNSLRTEDSAVYY

SEQ ID NO: 142 - protein, 34 aa, synthetic construct
  REGION 1..34: note = Description of Artificial Sequence: Synthetic polypeptide
DSVKGRFTISRDNSKNTLYLQMNSLRAEDTAVYY

SEQ ID NO: 143 - protein, 34 aa, synthetic construct
  REGION 1..34: note = Description of Artificial Sequence: Synthetic polypeptide
DSVKGRFTISRDNSKNTLSLQMNGLRAEDTAVYY

SEQ ID NO: 144 - protein, 34 aa, synthetic construct
  REGION 1..34: note = Description of Artificial Sequence: Synthetic polypeptide
QKFQGRVTMTRDTSTSTVYMELSSLRSEDTAVYY

SEQ ID NO: 145 - protein, 34 aa, synthetic construct
  REGION 1..34: note = Description of Artificial Sequence: Synthetic polypeptide
PSLESRVTISLDTSKNQFSLKLTSATAADTAVYY

SEQ ID NO: 146 - protein, 34 aa, synthetic construct
  REGION 1..34: note = Description of Artificial Sequence: Synthetic polypeptide
QHFQGRVTMTRDTSTSTVYMELSSLRSEDTAVYY

SEQ ID NO: 147 - protein, 34 aa, synthetic construct
  REGION 1..34: note = Description of Artificial Sequence: Synthetic polypeptide
GSVKGRFTISREDAKNSLDLQMNNLRVDDTAVYF

SEQ ID NO: 148 - protein, 34 aa, synthetic construct
  REGION 1..34: note = Description of Artificial Sequence: Synthetic polypeptide
DSVKGRFTTSRDNAKNSLYLQMNSLRAEDTALYY

SEQ ID NO: 149 - protein, 34 aa, synthetic construct
  REGION 1..34: note = Description of Artificial Sequence: Synthetic polypeptide
ASVKGRFTISREAGKHSLDLQMNSLRVDDTAVYF

SEQ ID NO: 150 - protein, 34 aa, synthetic construct
  REGION 1..34: note = Description of Artificial Sequence: Synthetic polypeptide
DSVKGRFTISRDNAKSSLFLQMSSLRVEDTALYY

SEQ ID NO: 151 - protein, 34 aa, synthetic construct
  REGION 1..34: note = Description of Artificial Sequence: Synthetic polypeptide
GSVKGRFTISRDISNNTLFLQMNDLRPEDTAVYH

SEQ ID NO: 152 - protein, 34 aa, synthetic construct
  REGION 1..34: note = Description of Artificial Sequence: Synthetic polypeptide
AKFQDRVTISADDSTKTVFVDLISLASDDTAVYY

SEQ ID NO: 153 - protein, 10 aa, synthetic construct
  REGION 1..10: note = Description of Artificial Sequence: Synthetic peptide
GRGTLVTVSS

SEQ ID NO: 154 - protein, 10 aa, synthetic construct
  REGION 1..10: note = Description of Artificial Sequence: Synthetic peptide
GQGTTVTVSS

SEQ ID NO: 155 - protein, 10 aa, synthetic construct
  REGION 1..10: note = Description of Artificial Sequence: Synthetic peptide
GQGTLVTVSS

SEQ ID NO: 156 - protein, 10 aa, synthetic construct
  REGION 1..10: note = Description of Artificial Sequence: Synthetic peptide
GRGTTVTVSS

SEQ ID NO: 157 - protein, 23 aa, synthetic construct
  REGION 1..23: note = Description of Artificial Sequence: Synthetic peptide
DVVMTQSPSSLSASVGDRVTITC

SEQ ID NO: 158 - protein, 23 aa, synthetic construct
  REGION 1..23: note = Description of Artificial Sequence: Synthetic peptide
SNFMLTQPPSASGTPGQRVTISC

SEQ ID NO: 159 - protein, 23 aa, synthetic construct
  REGION 1..23: note = Description of Artificial Sequence: Synthetic peptide
DVVMTQSPSSLSASVGDSVTITC

SEQ ID NO: 160 - protein, 23 aa, synthetic construct
  REGION 1..23: note = Description of Artificial Sequence: Synthetic peptide
DIVMTQSPSTLSASVGDRVTITC

SEQ ID NO: 161 - protein, 23 aa, synthetic construct
  REGION 1..23: note = Description of Artificial Sequence: Synthetic peptide
EIVLTQSPSTLSASVGDRVTITC

SEQ ID NO: 162 - protein, 23 aa, synthetic construct
  REGION 1..23: note = Description of Artificial Sequence: Synthetic peptide
EIVLTQSPSSLSASVGDRVTITC

SEQ ID NO: 163 - protein, 23 aa, synthetic construct
  REGION 1..23: note = Description of Artificial Sequence: Synthetic peptide
SSYELTQPPSVSVAPGQTARISC

SEQ ID NO: 164 - protein, 23 aa, synthetic construct
  REGION 1..23: note = Description of Artificial Sequence: Synthetic peptide
DIQMTQSPSSLSASVGDRVTITC

SEQ ID NO: 165 - protein, 23 aa, synthetic construct
  REGION 1..23: note = Description of Artificial Sequence: Synthetic peptide
SQSVVTQEPSLSVAPGKTAMITC

SEQ ID NO: 166 - protein, 23 aa, synthetic construct
  REGION 1..23: note = Description of Artificial Sequence: Synthetic peptide
ETTLTQSPDSLAVSLGERATINC

SEQ ID NO: 167 - protein, 23 aa, synthetic construct
  REGION 1..23: note = Description of Artificial Sequence: Synthetic peptide
DIVMTQSPSTLSASVGDRVIITC

SEQ ID NO: 168 - protein, 23 aa, synthetic construct
  REGION 1..23: note = Description of Artificial Sequence: Synthetic peptide
SQPGLTQPPSVSMAPGQTASITC

SEQ ID NO: 169 - protein, 23 aa, synthetic construct
  REGION 1..23: note = Description of Artificial Sequence: Synthetic peptide
SSYELTQPPSVSGAPGQRVTISC

SEQ ID NO: 170 - protein, 23 aa, synthetic construct
  REGION 1..23: note = Description of Artificial Sequence: Synthetic peptide
DIQMTQSPPSLSASVGDRVTITC

SEQ ID NO: 171 - protein, 23 aa, synthetic construct
  REGION 1..23: note = Description of Artificial Sequence: Synthetic peptide
DIVMTQSPSSLSASVGDRVTITC

SEQ ID NO: 172 - protein, 23 aa, synthetic construct
  REGION 1..23: note = Description of Artificial Sequence: Synthetic peptide
DVVMTQSPSSVSASVGDRVTITC

SEQ ID NO: 173 - protein, 23 aa, synthetic construct
  REGION 1..23: note = Description of Artificial Sequence: Synthetic peptide
DVVMTQSPSSLFASVGDRVTITC

SEQ ID NO: 174 - protein, 23 aa, synthetic construct
  REGION 1..23: note = Description of Artificial Sequence: Synthetic peptide
EIVLTQSPSSVSASVGDRVTITC

SEQ ID NO: 175 - protein, 23 aa, synthetic construct
  REGION 1..23: note = Description of Artificial Sequence: Synthetic peptide
ETTLTQSPSSVSASVGDRVTITC

SEQ ID NO: 176 - protein, 23 aa, synthetic construct
  REGION 1..23: note = Description of Artificial Sequence: Synthetic peptide
SNFMLTQPPSASGAPGHSVTISC

SEQ ID NO: 177 - protein, 23 aa, synthetic construct
  REGION 1..23: note = Description of Artificial Sequence: Synthetic peptide
EIVLTQSPSSLSASAGDRVTITC

SEQ ID NO: 178 - protein, 15 aa, synthetic construct
  REGION 1..15: note = Description of Artificial Sequence: Synthetic peptide
WYQQKPGKAPKLLIY

SEQ ID NO: 179 - protein, 15 aa, synthetic construct
  REGION 1..15: note = Description of Artificial Sequence: Synthetic peptide
WYHQLPGTAPKLLIQ

SEQ ID NO: 180 - protein, 15 aa, synthetic construct
  REGION 1..15: note = Description of Artificial Sequence: Synthetic peptide
WYQQRPGKTPNLLIY

SEQ ID NO: 181 - protein, 15 aa, synthetic construct
  REGION 1..15: note = Description of Artificial Sequence: Synthetic peptide
WYHQRPGQAPVLVVY

SEQ ID NO: 182 - protein, 15 aa, synthetic construct
  REGION 1..15: note = Description of Artificial Sequence: Synthetic peptide
WFRQRPGQAPVLVVY

SEQ ID NO: 183 - protein, 15 aa, synthetic construct
  REGION 1..15: note = Description of Artificial Sequence: Synthetic peptide
WYQQKPGQPPKLLIY

SEQ ID NO: 184 - protein, 15 aa, synthetic construct
  REGION 1..15: note = Description of Artificial Sequence: Synthetic peptide
WYQQKSGKAPKLLIY

SEQ ID NO: 185 - protein, 15 aa, synthetic construct
  REGION 1..15: note = Description of Artificial Sequence: Synthetic peptide
WFQQKSGQAPALVVY

SEQ ID NO: 186 - protein, 15 aa, synthetic construct
  REGION 1..15: note = Description of Artificial Sequence: Synthetic peptide
WYQQLPGTAPKLLIY

SEQ ID NO: 187 - protein, 15 aa, synthetic construct
  REGION 1..15: note = Description of Artificial Sequence: Synthetic peptide
WYQQKPGEAPKFLIY

SEQ ID NO: 188 - protein, 15 aa, synthetic construct
  REGION 1..15: note = Description of Artificial Sequence: Synthetic peptide
WYQQKPGKAPKLLIN

SEQ ID NO: 189 - protein, 15 aa, synthetic construct
  REGION 1..15: note = Description of Artificial Sequence: Synthetic peptide
WYQQLPGTAPKVLIY

SEQ ID NO: 190 - protein, 15 aa, synthetic construct
  REGION 1..15: note = Description of Artificial Sequence: Synthetic peptide
WYQQKPGKAPRLLIY

SEQ ID NO: 191 - protein, 31 aa, synthetic construct
  REGION 1..31: note = Description of Artificial Sequence: Synthetic polypeptide
GVPSRFSGSGSGTDFTLTISSLHPEDFATYY

SEQ ID NO: 192 - protein, 31 aa, synthetic construct
  REGION 1..31: note = Description of Artificial Sequence: Synthetic polypeptide
GVPERFSGSKSGTSASLAISGLRSEDEADYF

SEQ ID NO: 193 - protein, 31 aa, synthetic construct
  REGION 1..31: note = Description of Artificial Sequence: Synthetic polypeptide
GVPSRFSGSGSGTDFTLTISSLQPEDFATYY

SEQ ID NO: 194 - protein, 31 aa, synthetic construct
  REGION 1..31: note = Description of Artificial Sequence: Synthetic polypeptide
GIPERFSGSNSGNTATLTISRVEAGDEADYY

SEQ ID NO: 195 - protein, 31 aa, synthetic construct
  REGION 1..31: note = Description of Artificial Sequence: Synthetic polypeptide
GVPSRFSGSGSGTDFTLTIGSLQPEDFATYY

SEQ ID NO: 196 - protein, 31 aa, synthetic construct
  REGION 1..31: note = Description of Artificial Sequence: Synthetic polypeptide
GIPERISGSKSGNTATLTISRVEDGDFADYY

SEQ ID NO: 197 - protein, 31 aa, synthetic construct
  REGION 1..31: note = Description of Artificial Sequence: Synthetic polypeptide
GVPDRFSGSGSGTDFSLTISSLQAEDVAVYY

SEQ ID NO: 198 - protein, 31 aa, synthetic construct
  REGION 1..31: note = Description of Artificial Sequence: Synthetic polypeptide
GVPSRFSGSGSGTEFTLTISSLQPDDIATYY

SEQ ID NO: 199 - protein, 31 aa, synthetic construct
  REGION 1..31: note = Description of Artificial Sequence: Synthetic polypeptide
GIPERFSGSNSGNTATLTISRVEAGDEADYF

SEQ ID NO: 200 - protein, 31 aa, synthetic construct
  REGION 1..31: note = Description of Artificial Sequence: Synthetic polypeptide
GVPDRFSGSKSGTSASLAITGLQAEDEADYY

SEQ ID NO: 201 - protein, 31 aa, synthetic construct
  REGION 1..31: note = Description of Artificial Sequence: Synthetic polypeptide
GVPSRFSGGGSGTDFTLTISSLQPEDFATYY

SEQ ID NO: 202 - protein, 31 aa, synthetic construct
  REGION 1..31: note = Description of Artificial Sequence: Synthetic polypeptide
GVPSRFSGRGSGTDFTLTISSLQPEDFTSYY

SEQ ID NO: 203 - protein, 31 aa, synthetic construct
  REGION 1..31: note = Description of Artificial Sequence: Synthetic polypeptide
GVPSRLSGSGSGTDFTLTISSLQPEDFATYY

SEQ ID NO: 204 - protein, 31 aa, synthetic construct
  REGION 1..31: note = Description of Artificial Sequence: Synthetic polypeptide
GVPSRFSGSGSGTDFTLTISSQQPEDFATYY

SEQ ID NO: 205 - protein, 31 aa, synthetic construct
  REGION 1..31: note = Description of Artificial Sequence: Synthetic polypeptide
GVPSRFSGSGSGTDFTLTISSPQPEDFATYY

SEQ ID NO: 206 - protein, 31 aa, synthetic construct
  REGION 1..31: note = Description of Artificial Sequence: Synthetic polypeptide
GVPSRFSGSGSGTDFTLTISSLQPEDSATYY

SEQ ID NO: 207 - protein, 31 aa, synthetic construct
  REGION 1..31: note = Description of Artificial Sequence: Synthetic polypeptide
GVPSRFSGSGSVADFTLTISSLQPEDFATYY

SEQ ID NO: 208 - protein, 31 aa, synthetic construct
  REGION 1..31: note = Description of Artificial Sequence: Synthetic polypeptide
GVPSRFSGSGSGTEFTLTISSLQPDDFATYY

SEQ ID NO: 209 - protein, 31 aa, synthetic construct
  REGION 1..31: note = Description of Artificial Sequence: Synthetic polypeptide
GVPDRFSASKSGTSASLAISGLRSEDEADYY

SEQ ID NO: 210 - protein, 10 aa, synthetic construct
  REGION 1..10: note = Description of Artificial Sequence: Synthetic peptide
GGGTKLEIKR

SEQ ID NO: 211 - protein, 10 aa, synthetic construct
  REGION 1..10: note = Description of Artificial Sequence: Synthetic peptide
GTGTKLTVLG

SEQ ID NO: 212 - protein, 10 aa, synthetic construct
  REGION 1..10: note = Description of Artificial Sequence: Synthetic peptide
GGGTKVEIKR

SEQ ID NO: 213 - protein, 10 aa, synthetic construct
  REGION 1..10: note = Description of Artificial Sequence: Synthetic peptide
GQGTKVEIKR

SEQ ID NO: 214 - protein, 10 aa, synthetic construct
  REGION 1..10: note = Description of Artificial Sequence: Synthetic peptide
GQGTKLEIKR

SEQ ID NO: 215 - protein, 10 aa, synthetic construct
  REGION 1..10: note = Description of Artificial Sequence: Synthetic peptide
GQGTKVDIKR

SEQ ID NO: 216 - protein, 10 aa, synthetic construct
  REGION 1..10: note = Description of Artificial Sequence: Synthetic peptide
GGGTKLTVLR

SEQ ID NO: 217 - protein, 10 aa, synthetic construct
  REGION 1..10: note = Description of Artificial Sequence: Synthetic peptide
GGGTKLTVKR

SEQ ID NO: 218 - protein, 10 aa, synthetic construct
  REGION 1..10: note = Description of Artificial Sequence: Synthetic peptide
GGGTKVDIKR

SEQ ID NO: 219 - protein, 10 aa, synthetic construct
  REGION 1..10: note = Description of Artificial Sequence: Synthetic peptide
GTGTKLTVKR

SEQ ID NO: 220 - protein, 10 aa, synthetic construct
  REGION 1..10: note = Description of Artificial Sequence: Synthetic peptide
GTGTKLTVLR

SEQ ID NO: 221 - protein, 10 aa, synthetic construct
  REGION 1..10: note = Description of Artificial Sequence: Synthetic peptide
GRGTKVEIKR

SEQ ID NO: 222 - protein, 10 aa, synthetic construct
  REGION 1..10: note = Description of Artificial Sequence: Synthetic peptide
GGGTNLEIKR

SEQ ID NO: 223 - protein, 10 aa, synthetic construct
  REGION 1..10: note = Description of Artificial Sequence: Synthetic peptide
GQGTRLEIKR

SEQ ID NO: 224 - protein, 116 aa, synthetic construct
  REGION 1..116: note = Description of Artificial Sequence: Synthetic polypeptide
  VARIANT 39: note = MOD_RES - Any amino acid
QVTLKESGGGVVQPGRSLRLSCAASGFTFSSYAMSWARXAPGKGLEWVSAISGSGGSTYYADSVRGRFTISRDNSKNTLYLQMNSLRAEDTAVYYCTTDHRRRGLWGRGTLVTVSS

SEQ ID NO: 225 - protein, 119 aa, synthetic construct
  REGION 1..119: note = Description of Artificial Sequence: Synthetic polypeptide
EVQLVESGGGLVQPGGSLRLSCAASGFTLSDSTVHWVRQASGKGLERVAYIRSKVNSYATGYAASVKGRFTISRDDSKNTAYLQMNSLKIEDTAVYYCTRRNPGGMDVWGQGTTVTVSS

SEQ ID NO: 226 - protein, 121 aa, synthetic construct
  REGION 1..121: note = Description of Artificial Sequence: Synthetic polypeptide
EVQLVESGGGVVQPGRSLRLSCAASGFTFSSYAMSWVRQAPGKGLEWVSAISGSGGSTYYADSVKGRFTISRDNSKNMLYLQMNSLRAEDTALYYCARLRVGRDYKGMDVRGQGTTVTVSS

SEQ ID NO: 227 - protein, 119 aa, synthetic construct
  REGION 1..119: note = Description of Artificial Sequence: Synthetic polypeptide
EVQLVESGGDLVQPGGSLRLSCAASGFTLSDSTVHWVRQASGKGLERVAYIRSKVNSYATGYAASVKGRFTISRDDSKNTAYLQMNSLKIEDTAVYYCTRRNPGGMDVWGQGTLVTVSS

SEQ ID NO: 228 - protein, 119 aa, synthetic construct
  REGION 1..119: note = Description of Artificial Sequence: Synthetic polypeptide
QVTLKESGGGLVQPGGSLRLSCAASGFTLSDSTVHWVRQASGKGLERVAYIRSKVNSYATGYAASVKGRFTISRDDSKNTAYLQMNSLKIEDTAVYYCTRRNPGGMDVWGQGTLVTVSS

SEQ ID NO: 229 - protein, 123 aa, synthetic construct
  REGION 1..123: note = Description of Artificial Sequence: Synthetic polypeptide
QVTLKESGGGLVKPGGSLRLSCAASGFTFTNAWMGWVRQVPGTGLEWVARIKSKSDGGKTDYAAPVKGRFTISRDDSQNTLYLQMNSLRTEDSAVYYCVTYTYCGGDCYSEDWGQGTLVTVSS

SEQ ID NO: 230 - protein, 120 aa, synthetic construct
  REGION 1..120: note = Description of Artificial Sequence: Synthetic polypeptide
  VARIANT 1: note = MOD_RES - Any amino acid
XVQLQESGGGVVQPGRSLRLSCAGSGFTFSSYAMSWVRQAPGKGLEWVSAISGSGGSTYYADSVKGRFTISRDNSKNTLYLQMNSLRAEDTAVYYCAKVRRGYSYGTDYWGQGTLVTVSS

SEQ ID NO: 231 - protein, 115 aa, synthetic construct
  REGION 1..115: note = Description of Artificial Sequence: Synthetic polypeptide
  VARIANT 3: note = MOD_RES - Any amino acid
QVXLQESGGGLVQPGGSLRLSCAASGFAFSSYAMSWVRQAPGKGLEWVSAISGSGGSTYYADSVKGRFTISRDNSKNTLYLQMNSLRAEDTAVYYCATSRTFDYWGQGTLVTVSS

SEQ ID NO: 232 - protein, 121 aa, synthetic construct
  REGION 1..121: note = Description of Artificial Sequence: Synthetic polypeptide
HVTLKESGGGVVQPGRSLRLSCAASGFTFSGYGMHWVRQAPGKGLEWVATISYDGSNKYYADSVKGRFTISRDNSKNTLYLQMNSLRAEDTAVYYCALARSYDYYYGVDVWGRGTTVTVSS

SEQ ID NO: 233 - protein, 114 aa, synthetic construct
  REGION 1..114: note = Description of Artificial Sequence: Synthetic polypeptide
EVQLVESGGGLVQPGGSLRLSCAASGFAFSSYAMNWVRQAPGKGLEWVSTVSGSGSTYYADSVKGRFTISRDNSKNTLSLQMNGLRAEDTAVYYCARGRRFDPWGQGTLVTVSS

SEQ ID NO: 234 - protein, 117 aa, synthetic construct
  REGION 1..117: note = Description of Artificial Sequence: Synthetic polypeptide
EVQLVESGAEVKKPGASVKVSCKASGGTFSSDAITWVRQAPGQGLEWMGWINPNSGGTNYAQKFQGRVTMTRDTSTSTVYMELSSLRSEDTAVYYCSRDYGYYFDFWGQGTLVTVSS

SEQ ID NO: 235 - protein, 118 aa, synthetic construct
  REGION 1..118: note = Description of Artificial Sequence: Synthetic polypeptide
QVQLVESGPGLVAPSGTLSLTCTVSGGSINIGNWYNWVRQSPGKGLEWIGEVYHSGSTNYNPSLESRVTISLDTSKNQFSLKLTSATAADTAVYYCATRSVLTGSPIWGQGTTVTVSS

SEQ ID NO: 236 - protein, 124 aa, synthetic construct
  REGION 1..124: note = Description of Artificial Sequence: Synthetic polypeptide
  VARIANT 32: note = MOD_RES - Any amino acid
QVQLQQSGAEVKKPGASVKVSCKASGHTFSDXYMHWVRQAPGQGLEWMGRINPDSGATDYAQHFQGRVTMTRDTSTSTVYMELSSLRSEDTAVYYCAREAVAGTHPQAGDFDLWGRGTLVTVSS

SEQ ID NO: 237 - protein, 124 aa, synthetic construct
  REGION 1..124: note = Description of Artificial Sequence: Synthetic polypeptide
QVQLVQSGAEVKEPGASVKVSCKASGYTFTSYHMHWVRQAPGQGLEWMGRINPDSGATDYAQHFQGRVTMTRDTSTSTVYMELSSLRSEDTAVYYCAREAVAGTHPQAGDFDLWGRGTTVTVSS

SEQ ID NO: 238 - protein, 124 aa, synthetic construct
  REGION 1..124: note = Description of Artificial Sequence: Synthetic polypeptide
QVQLVQSGAEVKKPGASMKVSCKASGYTFTDHYIHWVRQAPGQGLEWMGRINPDSGATDYAQHFQGRVTMTRDTSTSTVYMELSSLRSEDTAVYYCAREAVAGTHPQAGDFDLWGRGTTVTVSS

SEQ ID NO: 239 - protein, 126 aa, synthetic construct
  REGION 1..126: note = Description of Artificial Sequence: Synthetic polypeptide
QVQLQESGGGLVQPGGSLRLSCAASGFTFSNFDMHWVRQSPGKGLEWVSGLDHSGGAHYAGSVKGRFTISREDAKNSLDLQMNNLRVDDTAVYFCVRGTLYHYTSGSYYSDAFDIWGQGTLVTVSS

SEQ ID NO: 240 - protein, 126 aa, synthetic construct
  REGION 1..126: note = Description of Artificial Sequence: Synthetic polypeptide
QVQLQQSGGGLVQPGGSLRLSCAASGFTFSNFDMHWVRQSPGKGLEWVSGLDHSGGAHYAGSVKGRFTISREDAKNSLDLQMNNLRVDDTAVYFCVRGTLYHYTSGSYYSDAFDIWGQGTTVTVSS

SEQ ID NO: 241 - protein, 121 aa, synthetic construct
  REGION 1..121: note = Description of Artificial Sequence: Synthetic polypeptide
EVQLVESGGGLVQPGGSLRLSCAASGFTFSSYAMSWVRQAPGKGLEWVSAISGSGGSTYYADSVKGRFTTSRDNAKNSLYLQMNSLRAEDTALYYCARLRVGRDYKGMDVRGQGTTVTVSS

SEQ ID NO: 242 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGLDHSGGAHYAGSVKGRFTISREDAKNSLDLQMNNLRVDDTAVYFCVRGTLYHYTSGSYYSDAFDIWGQGTLVTVSS

SEQ ID NO: 243 - protein, 126 aa, synthetic construct
  REGION 1..126: note = Description of Artificial Sequence: Synthetic polypeptide
QVTLKESGGGLVQPGGSLRLSCAASGFTFSNFDMHWVRQSPGKGLEWVSGLDHSGGAHYAGSVKGRFTISREDAKNSLDLQMNNLRVDDTAVYFCVRGTLYHYTSGSYYSDAFDIWGQGTLVTVSS

SEQ ID NO: 244 - protein, 126 aa, synthetic construct
  REGION 1..126: note = Description of Artificial Sequence: Synthetic polypeptide
QVQLVQSGGGLVQPGGSLRLSCAASGFTFSNFDMHWVRQSPGKGLEWVSGLDHSGGAHYAGSVKGRFTISREDAKNSLDLQMNNLRVDDTAVYFCVRGTLYHYTSGSYYSDAFDIWGQGTLVTVSS

SEQ ID NO: 245 - protein, 126 aa, synthetic construct
  REGION 1..126: note = Description of Artificial Sequence: Synthetic polypeptide
EVQLVESGGDLVQPGGSLRLSCAASGFTFSTFDMHWVRQSPGKGLEWVSTFDHSGGAHYAASVKGRFTISREAGKHSLDLQMNSLRVDDTAVYFCARGTLYHYTSGSYYSDAFDIWGQGTLVTVSS

SEQ ID NO: 246 - protein, 123 aa, synthetic construct
  REGION 1..123: note = Description of Artificial Sequence: Synthetic polypeptide
EVQLVESGGGLVQPGGSLRLSCAASGFTFDDFAMHWVRQAPGKSLEWVSSISWNSVNIDYADSVKGRFTISRDNAKSSLFLQMSSLRVEDTALYYCAKAGSYDGSGYNYFDSWGQGTLVTVSS

SEQ ID NO: 247 - protein, 124 aa, synthetic construct
  REGION 1..124: note = Description of Artificial Sequence: Synthetic polypeptide
QVQLVQSGGGVVQPGSSLRLSCEASGFIVSDYGMHWVRQAPGKGLEWVAVMSYDGSQKFYSGSVKGRFTISRDISNNTLFLQMNDLRPEDTAVYHCVREGSLTGPGWENWFDPWGQGTLVTVSS

SEQ ID NO: 248 - protein, 118 aa, synthetic construct
  REGION 1..118: note = Description of Artificial Sequence: Synthetic polypeptide
  VARIANT 6: note = MOD_RES - Any amino acid
EVQLLXSAAEVKRPGSSVRVSCKVSGGSFRDYAVSWVRQAPGQGPEWMGGIIPMFGRGEYTAKFQDRVTISADDSTKTVFVDLISLASDDTAVYYCARGLRLGELKQWGQGTLVTVSS

SEQ ID NO: 249 - protein, 108 aa, synthetic construct
  REGION 1..108: note = Description of Artificial Sequence: Synthetic polypeptide
DVVMTQSPSSLSASVGDRVTITCQASQDISNYLNWYQQKPGKAPKLLIYGASNLHSGVPSRFSGSGSGTDFTLTISSLHPEDFATYYCQQSYSTPLTFGGGTKLEIKR

SEQ ID NO: 250 - protein, 112 aa, synthetic construct
  REGION 1..112: note = Description of Artificial Sequence: Synthetic polypeptide
SNFMLTQPPSASGTPGQRVTISCSGSSSNIGSNYVYWYHQLPGTAPKLLIQRNNQRPSGVPERFSGSKSGTSASLAISGLRSEDEADYFCAAWDDRLAGYVFGTGTKLTVLG

SEQ ID NO: 251 - protein, 108 aa, synthetic construct
  REGION 1..108: note = Description of Artificial Sequence: Synthetic polypeptide
DVVMTQSPSSLSASVGDSVTITCRASQNINNYLNWYQQRPGKTPNLLIYDASNLETGVPSRFSGSGSGTDFTLTISSLQPEDFATYYCQQSHITPLTFGGGTKVEIKR

SEQ ID NO: 252 - protein, 108 aa, synthetic construct
  REGION 1..108: note = Description of Artificial Sequence: Synthetic polypeptide
DIVMTQSPSTLSASVGDRVTITCRASQGIRNDLGWYQQKPGKAPKLLIYAASTLQSGVPSRFSGSGSGTDFTLTISSLQPEDFATYYCQQANSYPVTFGGGTKVEIKR

SEQ ID NO: 253 - protein, 108 aa, synthetic construct
  REGION 1..108: note = Description of Artificial Sequence: Synthetic polypeptide
DVVMTQSPSSLSASVGDRVTITCRASRSIRNDLAWYQQKPGKAPKLLIYDASNLETGVPSRFSGSGSGTDFTLTISSLQPEDFATYYCQRSYTTPPTFGQGTKVEIKR

SEQ ID NO: 254 - protein, 108 aa, synthetic construct
  REGION 1..108: note = Description of Artificial Sequence: Synthetic polypeptide
EIVLTQSPSTLSASVGDRVTITCRASQSISSWLAWYQQKPGKAPKLLIYKASSLESGVPSRFSGSGSGTDFTLTISSLQPEDFATYYCQQAKSFPVSFGQGTKLEIKR

SEQ ID NO: 255 - protein, 108 aa, synthetic construct
  REGION 1..108: note = Description of Artificial Sequence: Synthetic polypeptide
EIVLTQSPSSLSASVGDRVTITCRASQDVSSWLAWYQQKPGKAPKLLIYAASTLQSGVPSRFSGSGSGTDFTLTISSLQPEDFATYYCQQHYTTPPTFGQGTKVDIKR

SEQ ID NO: 256 - protein, 110 aa, synthetic construct
  REGION 1..110: note = Description of Artificial Sequence: Synthetic polypeptide
SSYELTQPPSVSVAPGQTARISCGGDNIGDKRVHWYHQRPGQAPVLVVYDDADRPTGIPERFSGSNSGNTATLTISRVEAGDEADYYCQVYDRASNHVVFGGGTKLTVLR

SEQ ID NO: 257 - protein, 110 aa, synthetic construct
  REGION 1..110: note = Description of Artificial Sequence: Synthetic polypeptide
SSYELTQPPSVSVAPGQTARISCGGDNIGDKRVHWYHQRPGQAPVLVVYDDADRPTGIPERFSGSNSGNTATLTISRVEAGDEADYYCQVYDRASNHVVFGGGTKLTVKR

SEQ ID NO: 258 - protein, 108 aa, synthetic construct
  REGION 1..108: note = Description of Artificial Sequence: Synthetic polypeptide
EIVLTQSPSSLSASVGDRVTITCRASQDIRNDLGWYQQKPGKAPKLLIYAASTLQSGVPSRFSGSGSGTDFTLTIGSLQPEDFATYYCQQANSFPLTFGGGTKVDIKR

SEQ ID NO: 259 - protein, 108 aa, synthetic construct
  REGION 1..108: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 260 - protein, 110 aa, synthetic construct
  REGION 1..110: note = Description of Artificial Sequence: Synthetic polypeptide
SQSVVTQEPSLSVAPGKTAMITCGGDRLHFKTVDWFRQRPGQAPVLVVYGDSDRPSGIPERISGSKSGNTATLTISRVEDGDFADYYCQVWDHNGDGRVFGTGTKLTVLR

SEQ ID NO: 261 - protein, 110 aa, synthetic construct
  REGION 1..110: note = Description of Artificial Sequence: Synthetic polypeptide
SQSVVTQEPSLSVAPGKTAMITCGGDRLHFKTVDWFRQRPGQAPVLVVYGDSDRPSGIPERISGSKSGNTATLTISRVEDGDFADYYCQVWDHNGDGRVFGTGTKLTVKR

SEQ ID NO: 262 - protein, 114 aa, synthetic construct
  REGION 1..114: note = Description of Artificial Sequence: Synthetic polypeptide
ETTLTQSPDSLAVSLGERATINCKSSQSVLDSSKNKNYLAWYQQKPGQPPKLLIYWASIRESGVPDRFSGSGSGTDFSLTISSLQAEDVAVYYCQQYYGTPYTFGQGTKLEIKR

SEQ ID NO: 263 - protein, 108 aa, synthetic construct
  REGION 1..108: note = Description of Artificial Sequence: Synthetic polypeptide
DIVMTQSPSTLSASVGDRVIITCRASQSINNWLAWYQQKSGKAPKLLIYKASTLESGVPSRFSGSGSGTEFTLTISSLQPDDIATYYCQQYNNHSRTFGRGTKVEIKR

SEQ ID NO: 264 - protein, 110 aa, synthetic construct
  REGION 1..110: note = Description of Artificial Sequence: Synthetic polypeptide
SQPGLTQPPSVSMAPGQTASITCGGNNIGSKSVHWFQQKSGQAPALVVYDDTNRPSGIPERFSGSNSGNTATLTISRVEAGDEADYFCQLWDSSGEHWVFGGGTKLTVLR

SEQ ID NO: 265 - protein, 110 aa, synthetic construct
  REGION 1..110: note = Description of Artificial Sequence: Synthetic polypeptide
SQPGLTQPPSVSMAPGQTASITCGGNNIGSKSVHWFQQKSGQAPALVVYDDTNRPSGIPERFSGSNSGNTATLTISRVEAGDEADYFCQLWDSSGEHWVFGGGTKLTVKR

SEQ ID NO: 266 - protein, 113 aa, synthetic construct
  REGION 1..113: note = Description of Artificial Sequence: Synthetic polypeptide
SSYELTQPPSVSGAPGQRVTISCTGSSSNIGAGFDVHWYQQLPGTAPKLLIYGNSNRPSGVPDRFSGSKSGTSASLAITGLQAEDEADYYCRSYDSSLSGSVFGGGTKLTVLR

SEQ ID NO: 267 - protein, 113 aa, synthetic construct
  REGION 1..113: note = Description of Artificial Sequence: Synthetic polypeptide
SSYELTQPPSVSGAPGQRVTISCTGSSSNIGAGFDVHWYQQLPGTAPKLLIYGNSNRPSGVPDRFSGSKSGTSASLAITGLQAEDEADYYCRSYDSSLSGSVFGGGTKLTVKR

SEQ ID NO: 268 - protein, 108 aa, synthetic construct
  REGION 1..108: note = Description of Artificial Sequence: Synthetic polypeptide
DIQMTQSPPSLSASVGDRVTITCRASQSISSYLNWYQQKPGKAPKLLIYAASSLQSGVPSRFSGGGSGTDFTLTISSLQPEDFATYYCQQSYSTPLTFGGGTKVEIKR

SEQ ID NO: 269 - protein, 107 aa, synthetic construct
  REGION 1..107: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTHTFGQGTKVEIKR

SEQ ID NO: 270 - protein, 108 aa, synthetic construct
  REGION 1..108: note = Description of Artificial Sequence: Synthetic polypeptide
DIQMTQSPSSLSASVGDRVTITCRASQSISTYLNWYQQKPGKAPKLLIYAASSLQSGVPSRFSGSGSGTDFTLTISSLQPEDFATYYCQQSYSTPYTFGGGTNLEIKR

SEQ ID NO: 271 - protein, 108 aa, synthetic construct
  REGION 1..108: note = Description of Artificial Sequence: Synthetic polypeptide
EIVLTQSPSSLSASVGDRVTITCRASQSISSFLNWYQQKPGKAPKLLIYAASSLQSGVPSRFSGRGSGTDFTLTISSLQPEDFTSYYCQQSYSPPLTFGGGTKVDIKR

SEQ ID NO: 272 - protein, 108 aa, synthetic construct
  REGION 1..108: note = Description of Artificial Sequence: Synthetic polypeptide
DIVMTQSPSSLSASVGDRVTITCRASQGISDNLNWYQQKPGKAPKLLIYAASTLQSGVPSRLSGSGSGTDFTLTISSLQPEDFATYYCQQANSFPLTFGGGTKVEIKR

SEQ ID NO: 273 - protein, 108 aa, synthetic construct
  REGION 1..108: note = Description of Artificial Sequence: Synthetic polypeptide
EIVLTQSPSSLSASVGDRVTITCQASQDISNYLNWYQQKPGKAPKLLIYAASSLESGVPSRFSGSGSGTDFTLTISSQQPEDFATYYCQQANSFPYTFGQGTKVEIKR

SEQ ID NO: 274 - protein, 108 aa, synthetic construct
  REGION 1..108: note = Description of Artificial Sequence: Synthetic polypeptide
EIVLTQSPSSLSASVGDRVTITCRASETISIYLNWYQQKPGEAPKFLIYAASSLQSGVPSRFSGSGSGTDFTLTISSPQPEDFATYYCQQSYSTPWTFGQGTKVDIKR

SEQ ID NO: 275 - protein, 108 aa, synthetic construct
  REGION 1..108: note = Description of Artificial Sequence: Synthetic polypeptide
DVVMTQSPSSVSASVGDRVTITCRASQGISSWLAWYQQKPGKAPKLLIYAASNLQMGVPSRFSGSGSGTDFTLTISSLQPEDSATYYCQQSYTTPLTFGGGTKVEIKR

SEQ ID NO: 276 - protein, 108 aa, synthetic construct
  REGION 1..108: note = Description of Artificial Sequence: Synthetic polypeptide
DVVMTQSPSSLFASVGDRVTITCRASQDIRNDLGWYQQKPGKAPKLLIYGASTLQSGVPSRFSGSGSVADFTLTISSLQPEDFATYYCQQSYITPLTFGGGTKLEIKR

SEQ ID NO: 277 - protein, 107 aa, synthetic construct
  REGION 1..107: note = Description of Artificial Sequence: Synthetic polypeptide
DIVMTQSPSTLSASVGDRVTITCRASQSISSWLAWYQQKPGKAPKLLIYAASSLQSGVPSRFSGSGSGTDFTLTISSLQPEDFATYYCQQSYSTITFGQGTKVDIKR

SEQ ID NO: 278 - protein, 108 aa, synthetic construct
  REGION 1..108: note = Description of Artificial Sequence: Synthetic polypeptide
EIVLTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKLEIKR

SEQ ID NO: 279 - protein, 108 aa, synthetic construct
  REGION 1..108: note = Description of Artificial Sequence: Synthetic polypeptide
ETTLTQSPSSVSASVGDRVTITCRASQSISSWLAWYQQKPGKAPKLLINKASSLESGVPSRFSGSGSGTEFTLTISSLQPDDFATYYCQQYHSYPLTFGGGTKVDIKR

SEQ ID NO: 280 - protein, 112 aa, synthetic construct
  REGION 1..112: note = Description of Artificial Sequence: Synthetic polypeptide
SNFMLTQPPSASGAPGHSVTISCSGGSSNIGDYDVSWYQQLPGTAPKVLIYRNNQRPSGVPDRFSASKSGTSASLAISGLRSEDEADYYCAAWDDSLNGRVFGTGTKLTVLG

SEQ ID NO: 281 - protein, 108 aa, synthetic construct
  REGION 1..108: note = Description of Artificial Sequence: Synthetic polypeptide
EIVLTQSPSSLSASAGDRVTITCRASQGVSSWLAWYQQKPGKAPRLLIYATSRLQSGVPSRFSGSGSGTDFTLTISSLQPEDFATYYCQQSYFTPVTFGQGTRLEIKR

SEQ ID NO: 282 - protein, 114 aa, synthetic construct; identical to SEQ ID NO: 262
  REGION 1..114: note = Description of Artificial Sequence: Synthetic polypeptide

SEQ ID NO: 283 - protein, 15 aa, synthetic construct
  REGION 1..15: note = Description of Artificial Sequence: Synthetic peptide
GGGGSGGGGSGGGGS

SEQ ID NO: 284 - protein, 60 aa, Dendroaspis sp.
QPLRKLCILHRNPGRCYQKIPAFYYNQKKKQCEGFTWSGCGGNSNRFKTIEECRRTCIRK

SEQ ID NO: 285 - protein, 76 aa, Bungarus sp.
YTLLCHTTSTSPISTVTCPSGENLCYTKMWCDAFCSSRGKVIELGCVATCPQPKPYEEVTCCSTDKCNPHPKQRPG

SEQ ID NO: 286 - protein, 72 aa, Dendroaspis sp.
RTCNKTFSDQSKICPPGENICYTKTWCDAFCSQRGKRVELGCAATCPKVKAGVEIKCCSTDNCNKFQFGKPR

SEQ ID NO: 287 - protein, 71 aa, Naja sp.
IRCFITPDVTSQACPDGHVCYTKMWCDNFCGMRGKRVDLGCAATCPKVKPGVNIKCCSRDNCNPFPTRKRS

SEQ ID NO: 288 - protein, 71 aa, Oxyuranus sp.
RRCFTTPSVRSERCPPGQEVCYTKTWTDGHGGSRGKRVDLGCAATCPTPKKKDIKIICCSTDNCNTFPKWP

SEQ ID NO: 289 - protein, 73 aa, Notechis sp.
LICYMGPKTPRTCPRGQNLCYTKTWCDAFCSSRGKVVELGCAATCPIAKSYEDVTCCSTDNCNPFPVRPRPHP

SEQ ID NO: 290 - protein, 82 aa, Pseudonaja textilis
RTCFITPDVKSKPCPPGQEVCYTKTWCDGFCGIRGKRVDLGCAATCPTPKKTGIDIICCSTDDCNTFPLRPRGRLSSIKDHP

SEQ ID NO: 291 - protein, 68 aa, Pseudonaja textilis
LICYLDFSVPHTCAPGEKLCYTRTWNDGRGTRIERGCAATCPIPKKPEIHVTCCSTDRCNPHPKQKPH

SEQ ID NO: 292 - protein, 60 aa, Notechis sp.
MTCCNQQSSQPKTTTTCAESSCYKKTWRDHRGTITERGCGCPNVKPGVQINCCKTDECNN

SEQ ID NO: 293 - protein, 62 aa, Oxyuranus sp.
MTCYNQQSSEAKTTTTCSGGVSSCYKKTWSDGRGTIIERGCGCPSVKKGIERICCRTDKCNN

SEQ ID NO: 294 - protein, 60 aa, Dendroaspis sp.
RICYNHQSTTRATTKSCEENSCYKKYWRDHRGTIIERGCGCPKVKPGVGIHCCQSDKCNY

SEQ ID NO: 295 - protein, 61 aa, Naja sp.
MICHNQQSSQRPTIKTCPGETNCYKKRWRDHRGTIIERGCGCPSVKKGVGIYCCKTDKCNR

SEQ ID NO: 296 - protein, 121 aa, Agkistrodon sp.
SVLELGKMILQETGKNAITSYGSYGCNCGWGHRGQPKDATDRCCFVHKCCYKKLTDCNHKTDRYSYSWKNKAIICEEKNPCLKEMCECDKAVAICLRENLDTYNKKYKAYFKLKCKKPDTC

SEQ ID NO: 297 - protein, 121 aa, Crotalus sp.
SLVELGKMILQETGKNPITSYGIYGCNCGVGSRHKPKDGTDRCCFVHKCCYKKLTDCDPKMDGYTYSFKDKTIICDVNNPCLKEMCECDKAVAICLRENLDTYNKKYKIYPKFLCKKPDTC

SEQ ID NO: 298 - protein, 122 aa, Echis sp.
SVVELGKMIIQETGKSPFPSYTSYGCFCGGGERGPPLDATDRCCLAHSCCYDTLPDCSPKTDRYKYKRENGEIICENSTSCKKRICECDKAVAVCLRKNLNTYNKKYTYYPNFWCKGDIEKC

SEQ ID NO: 299 - protein, 123 aa, Agkistrodon sp.
NLFQFEKLIKKMTGKSGMLWYSAYGCYCGWGGQGRPKDATDRCCFVHDCCYGKVTGCNPKMDIYTYSVDNGNIVCGGTNPCKKQICECDRAAAICFRDNLKTYDSKTYWKYPKKNCKEESEPC

SEQ ID NO: 300 - protein, 122 aa, Crotalus sp.
SLVQFETLIMKIAGRSGLLWYSAYGCYCGWGGHGLPQDATDRCCFVHDCCYGKATDCNPKTVSYTYSEENGEIICGGDDPCGTQICECDKAAAICFRDNIPSYDNKYWLFPPKNCREEPEPC

SEQ ID NO: 301 - protein, 123 aa, Echis sp.
NLYQFGKMIKNKTGKPAMFSYSAYGCYCGWGGQGKPQDPSDRCCFLHDCCYTRVNNCSPKMTLYSYRFENGDIICGDNDPCRKAVCECDREAAICLGENVNTYDEKYRFYSSSYCTEEESEKC

SEQ ID NO: 302 - protein, 118 aa, Bungarus sp.
NLYQLMNMIQCANTRTWPSYTNYGCYCGKGGSGTPVDDLDRCCYTHDHCYNDAKNIDGCNPVTKTYSYTCTEPTITCNDSKDKCARFVCDCDRTAAICFAKAPYNTSNVMIRSTNSCQ

SEQ ID NO: 303 - protein, 119 aa, Oxyuranus sp.
NLLQFGFMIRCANRRSRPVWHYMDYGCYCGKGGSGTPVDDLDRCCQVHDECYGEAVRRFGCAPYWTLYSWKCYGKAPTCNTKTRCQRFVCRCDAKAAECFARSPYQNSNWNINTKARCR

SEQ ID NO: 304 - protein, 118 aa, Oxyuranus sp.
NLVQFGKMIECAIRNRRPALDFMNYGCYCGKGGSGTPVDDLDRCCQVHDECYAEAEKHGCYPSLTTYTWECRQVGPYCNSKTQCEVFVCACDFAAAKCFAQEDYNPAHSNINTGERCK

SEQ ID NO: 305 - protein, 122 aa, Daboia sp.
NFFQFAEMIVKMTGKEAVHSYAIYGCYCGWGGQGKPQDATDRCCFVHDCCYGTVNDCNPKMATYSYSFENGDIVCGDNNLCLKTVCECDRAAAICLGQNVNTYDKNYENYAISHCTEESEQC

SEQ ID NO: 306 - protein, 119 aa, Naja sp.
NLYQFKNMIKCTVPSRSWWDFADYGCYCGRGGSGTPVDDLDRCCQVHDNCYNEAEKISGCWPYFKTYSYECSQGTLTCKGDNNACAASVCDCDRLAAICFAGAPYNDNNYNIDLKARCQ

SEQ ID NO: 307 - protein, 119 aa, Notechis sp.
NLVQFSYLIQCANHGKRPTWHYMDYGCYCGAGGSGTPVDELDRCCKIHDDCYDEAGKKGCFPKMSAYDYYCGENGPYCRNIKKKCLRFVCDCDVEAAFCFAKAPYNNANWNIDTKKRCQ

SEQ ID NO: 308 - protein, 118 aa, unidentified
  REGION 1..118: note = Description of Unknown: pla2-kingbrown-ACII4-3V9M sequence
NLIQFGNMIQCANKGSRPSLDYADYGCYCGWGGSGTPVDELDRCCQVHDNCYEQAGKKGCFPKLTLYSWKCTGNVPTCNSKPGCKSFVCACDAAAAKCFAKAPYKKENYNIDTKKRCK

SEQ ID NO: 309 - protein, 118 aa, Bungarus sp.
NLQQFKNMIQCAGTRTWTAYINYGCYCGKGGSGTPVDKLDRCCYTHDHCYNQADSIPGCNPNIKTYSYTCTQPNITCTRTADACAKFLCDCDRTAAICFASAPYNINNIMISASNSCQ

SEQ ID NO: 310 - protein, 123 aa, Deinagkistrodon sp.
SLIQFETLIMKVVKKSGMFWYSAYGCYCGWGGHGRPQDATDRCCFVHDCCYGKVTGCDPKMDSYTYSEENGDIVCGGDDPCKREICECDRVAADCFRDNLDTYNSDTYWRYPRQDCEESPEPC

SEQ ID NO: 311 - protein, 124 aa, Agkistrodon sp.
SLVQFETLIMKVAKKSGMQWYSNYGCYCGWGGQGRPQDATDRCCFVHDCCYGKVTGCDPKMDVYSFSEENGDIVCGGDDPCKKEICECDRAAAICFRDNLNTYNDKKYWAFGAKNCPQEESEPC

SEQ ID NO: 312 - protein, 120 aa, Echis sp.
NLYQFGRMIWNRTGKLPILSYGSYGCYCGWGGQGPPKDATDRCCLVHDCCYTRVGDCSPKMTLYSYRFENGDIICDNKDPCKRAVCECDREAAICLGENVNTYDKKYKSYEDCTEEVQEC

SEQ ID NO: 313 - protein, 135 aa, Agkistrodon sp.
NNCPHDWLPMNGLCYKIFDELKAWEDAERFCRKYKPGCHLASFHTYGESLEIAEYISDYHKGQAEVWIGLWDKKKDFSWEWTDRSCTDYLTWDKNQPDVYQNKEFCVELVSLTGYRLWNDQVCESKNAFLCQCKF

SEQ ID NO: 314 - protein, 135 aa, Crotalus sp.
NNCPLDWLPMNGLCYKIFNQLKTWEDAEMFCRKYKPGCHLASFHRYGESLEIAEYISDYHKGQENVWIGLRDKKKDFSWEWTDRSCTDYLTWDKNQPDHYQNKEFCVELVSLTGYRLWNDQVCESKDAFLCQCKF

SEQ ID NO: 315 - protein, 124 aa, Echis sp.
VCCPLGWSGYDQNCYKAFEELMNWADAEKFCTQQHKGSHLVSLHNIAEADFVVKKIVSVLKDGVIWMGLNDVWNECNWGWTDGAQLDYKAWNVESNCFIFKTAENHWSRTDCSGTHSFVCKSPA

SEQ ID NO: 316 - protein, 136 aa, Oxyuranus sp.
SCCCPQDWLPKNGFCYKVFNDHKNWNDAEMFCRKFKPGCHLASIHSNADSADLAEYVSDYLKSDGNVWIGLNDPRKQRTWVWSDRSPTNYYSWNRGEPNNSKNNEYCVHLWALSGYLKWNDTPCKALYSFICQCRF

SEQ ID NO: 317 - protein, 62 aa, Oxyuranus sp.
RPKFCHLPPKPGPCRAAIPRFYYNPHSKQCEKFIYGGCHGNANSFKTPDECNYTCLGVSLPK

SEQ ID NO: 318 - protein, 57 aa, Naja sp.
RPRFCELPAETGLCKARIRSFHYNRAAQQCLEFIYGGCGGNANRFKTIDECHRTCVG

SEQ ID NO: 319 - protein, 59 aa, Dendroaspis sp.
LQHRTFCKLPAEPGPCKASIPAFYYNWAAKKCQLFHYGGCKGNANRFSTIEKCRHACVG

SEQ ID NO: 320 - protein, 221 aa, Agkistrodon sp.
SVDFDSESPRKPEIQNQIVDLHNSLRRSVNPTASNMLKMEWYPEAAANAERWAYRCIESHSPRNSRVLGGIKCGENIYMSSIPIKWTEIIHAWHGENKNFKYGIGADPPNAVIGHFTQIVWYKSYLVGCAAAYCPSSEYSYFYVCQYCPAGNIIGKIATPYKSGPPCGDCPSACVNGLCTNPCTKEDKYTNCKSLVQQYGCQDKQMQSECSAICFCQNKII

SEQ ID NO: 321 - protein, 221 aa, Crotalus sp.
SVDFDSESPRKPEIQNKIVDLHNFLRRSVNPTASNMLKMEWYPEAAANAERWAYRCIESHSPRDSRVLGGIKCGENIYMSPVPIKWTEIIHAWHGENKNFKYGIGAVPPNAVTGHFSQVVWYKSYRIGCAAAYCPSSKYSYFYVCQYCPAGNIIGKTATPYKSGPPCGDCPSACDNGLCTNPCTKEDKYTNCKSLVQQAGCQDKQMQSDCPAICFCQNKII

SEQ ID NO: 322 - protein, 219 aa, Oxyuranus sp.
TVDFASESSNKKDYRKEIVDKHNDLRRSVKPTARNMLQMKWNSRAAQNAKRWANRCTFAHSPPYTRTVGKLRCGENIFMSSQPFAWSGVVQAWYDEVKKFVYGIGAKPPSSVIGHYTQVVWYKSHLLGCASAKCSSTKYLYVCQYCPAGNIIGSIATPYKSGPPCGDCPSACDNGLCTNPCKHNNDFSNCKALAKKSKCQTEWIKSKCPATCFCRTEII

SEQ ID NO: 323 - protein, 122 aa, Echis sp.
SIVELGKMIIQETGKSPFPSYTSYGCFCGGGERGPPLDATDRCCLAHSCCYDTLPDCSPKTDRYKYKRENGEIICENSTSCKKRICECDKAMAVCLRKNLNTYNKKYTYYPNFWCKGDIEKC

SEQ ID NO: 324 - protein, 120 aa, Bungarus sp.
NLINFMNMIRYTIPCEKTWGEYTNYGCYCGAGGSGTPIDALDRCCYVHDNCYGDAEKIHKCSPKTQSYSYKLTRRTIICYGAAGTCARIVCDCDRTAALCFGDSEYIEGHKRIDTKRYCQ

SEQ ID NO: 325 - protein, 127 aa, Oxyuranus sp.
SLLNFANLIECANHGTRSALAYADYGCYCGKGGRGTPLDDLDRCCHVHDDCYGEAEKLPACNYLMSSPYFNSYSYKCNEGKVTCTDDNDECKAFICNCDRTAAICFAGATYNDENFMISKKRNDICQ

SEQ ID NO: 326 - protein, 6 aa, synthetic construct
  REGION 1..6: note = Description of Artificial Sequence: Synthetic 6xHis tag
HHHHHH

SEQ ID NO: 327 - protein, 22 aa, synthetic construct
  REGION 1..22: note = Description of Artificial Sequence: Synthetic peptide
CVRGTLYHYTSGSYCSDAFDIW

SEQ ID NO: 328 - protein, 22 aa, synthetic construct
  REGION 1..22: note = Description of Artificial Sequence: Synthetic peptide
CVRGTLYHYTSGSYYSDAFDIW

SEQ ID NO: 329 - protein, 22 aa, synthetic construct
  REGION 1..22: note = Description of Artificial Sequence: Synthetic peptide
CARGTLYHYTSGSYYSDAFDIW

SEQ ID NO: 330 - protein, 22 aa, synthetic construct
  REGION 1..22: note = Description of Artificial Sequence: Synthetic peptide
CVRGTLYHYTSGSYYSDAFDIW

SEQ ID NO: 331 - protein, 22 aa, synthetic construct
  REGION 1..22: note = Description of Artificial Sequence: Synthetic peptide
CVRGTLYHYTSGSYYSDAFDIW

SEQ ID NO: 332 - protein, 22 aa, synthetic construct
  REGION 1..22: note = Description of Artificial Sequence: Synthetic peptide
CVRGTLYHYTSGSYYSDAFDIW

SEQ ID NO: 333 - protein, 22 aa, synthetic construct
  REGION 1..22: note = Description of Artificial Sequence: Synthetic peptide
CVRGTLYHYTSGSYYSDAFDIW

SEQ ID NO: 334 - protein, 22 aa, synthetic construct
  REGION 1..22: note = Description of Artificial Sequence: Synthetic peptide
CVRGTLYHYTSGSYYSDAFDIW

SEQ ID NO: 335 - protein, 22 aa, synthetic construct
  REGION 1..22: note = Description of Artificial Sequence: Synthetic peptide
CVRGTLYHYTSGSYYSDAFDIW

SEQ ID NO: 336 - protein, 22 aa, synthetic construct
  REGION 1..22: note = Description of Artificial Sequence: Synthetic peptide
CVRGTLYHYTSGSYYSDAFDIW

SEQ ID NO: 337 - protein, 22 aa, synthetic construct
  REGION 1..22: note = Description of Artificial Sequence: Synthetic peptide
CVRGTLYHYTSGSYYSDAFDIW

SEQ ID NO: 338 - protein, 22 aa, synthetic construct
  REGION 1..22: note = Description of Artificial Sequence: Synthetic peptide
CVRGTLYHYTSGSYYSDAFDIW

SEQ ID NO: 339 - protein, 22 aa, synthetic construct
  REGION 1..22: note = Description of Artificial Sequence: Synthetic peptide
CVRGTLYHYTSGSYYSDAFDIW

SEQ ID NO: 340 - protein, 22 aa, synthetic construct
  REGION 1..22: note = Description of Artificial Sequence: Synthetic peptide
CVRGTLYHYTSGSYYSDAFDIW

SEQ ID NO: 341 - protein, 22 aa, synthetic construct
  REGION 1..22: note = Description of Artificial Sequence: Synthetic peptide
CVRGTLYHYTSGSYYSDAFDIW

SEQ ID NO: 342 - protein, 22 aa, synthetic construct
  REGION 1..22: note = Description of Artificial Sequence: Synthetic peptide
CVRGTLYHYTSGSYYSDAFDIW

SEQ ID NO: 343 - protein, 22 aa, synthetic construct
  REGION 1..22: note = Description of Artificial Sequence: Synthetic peptide
CVRGTLYHYTSGSYYSDAFDIW

SEQ ID NO: 344 - protein, 22 aa, synthetic construct
  REGION 1..22: note = Description of Artificial Sequence: Synthetic peptide
CVRGTLYHYTSGSYYSDAFDIW

SEQ ID NO: 345 - protein, 22 aa, synthetic construct
  REGION 1..22: note = Description of Artificial Sequence: Synthetic peptide
CVRGTLYHYTSGSYYSDAFDIW

SEQ ID NO: 346 - protein, 22 aa, synthetic construct
  REGION 1..22: note = Description of Artificial Sequence: Synthetic peptide
CVRGTLYHYTSGSYYSDAFDIW

SEQ ID NO: 347 - protein, 22 aa, synthetic construct
  REGION 1..22: note = Description of Artificial Sequence: Synthetic peptide
CVRGTLYHYTSGSYYSDAFDIW

SEQ ID NO: 348 - protein, 22 aa, synthetic construct
  REGION 1..22: note = Description of Artificial Sequence: Synthetic peptide
CVRGTLYHYTSGSYYSDAFDIW

SEQ ID NO: 349 - protein, 9 aa, synthetic construct
  REGION 1..9: note = Description of Artificial Sequence: Synthetic peptide
FTFSNFDMH

SEQ ID NO: 350 - protein, 9 aa, synthetic construct
  REGION 1..9: note = Description of Artificial Sequence: Synthetic peptide
FTFSNFDMH

SEQ ID NO: 351 - protein, 9 aa, synthetic construct
  REGION 1..9: note = Description of Artificial Sequence: Synthetic peptide
FTFSTFDMH

SEQ ID NO: 352 - protein, 9 aa, synthetic construct
  REGION 1..9: note = Description of Artificial Sequence: Synthetic peptide
FTFSNFDMH

SEQ ID NO: 353 - protein, 9 aa, synthetic construct
  REGION 1..9: note = Description of Artificial Sequence: Synthetic peptide
FTFSNFDMH

SEQ ID NO: 354 - protein, 9 aa, synthetic construct
  REGION 1..9: note = Description of Artificial Sequence: Synthetic peptide
FTFSNFDMH

SEQ ID NO: 355 - protein, 9 aa, synthetic construct
  REGION 1..9: note = Description of Artificial Sequence: Synthetic peptide
FTLSNFDMH

SEQ ID NO: 356 - protein, 9 aa, synthetic construct
  REGION 1..9: note = Description of Artificial Sequence: Synthetic peptide
FTFSNFDMH

SEQ ID NO: 357 - protein, 9 aa, synthetic construct
  REGION 1..9: note = Description of Artificial Sequence: Synthetic peptide
FTFRNFDMH

SEQ ID NO: 358 - protein, 9 aa, synthetic construct
  REGION 1..9: note = Description of Artificial Sequence: Synthetic peptide
FTFSNFDMH

SEQ ID NO: 359 - protein, 9 aa, synthetic construct
  REGION 1..9: note = Description of Artificial Sequence: Synthetic peptide
FTFSNFDMH

SEQ ID NO: 360 - protein, 9 aa, synthetic construct
  REGION 1..9: note = Description of Artificial Sequence: Synthetic peptide
FTFSNYDMH

SEQ ID NO: 361 - protein, 9 aa, synthetic construct
  REGION 1..9: note = Description of Artificial Sequence: Synthetic peptide
FTFSNFDMH

SEQ ID NO: 362 - protein, 9 aa, synthetic construct
  REGION 1..9: note = Description of Artificial Sequence: Synthetic peptide
FTFSNLDMH

SEQ ID NO: 363 - protein, 9 aa, synthetic construct
  REGION 1..9: note = Description of Artificial Sequence: Synthetic peptide
FTFSNFDMH

SEQ ID NO: 364 - protein, 9 aa, synthetic construct
  REGION 1..9: note = Description of Artificial Sequence: Synthetic peptide
FTFSNFDMH

SEQ ID NO: 365 - protein, 9 aa, synthetic construct
  REGION 1..9: note = Description of Artificial Sequence: Synthetic peptide
FTFSNFDMH

SEQ ID NO: 366 - protein, 9 aa, synthetic construct
  REGION 1..9: note = Description of Artificial Sequence: Synthetic peptide
FTFSNFDMH

SEQ ID NO: 367 - protein, 9 aa, synthetic construct
  REGION 1..9: note = Description of Artificial Sequence: Synthetic peptide
FTFSNFDMH

SEQ ID NO: 368 - protein, 9 aa, synthetic construct
  REGION 1..9: note = Description of Artificial Sequence: Synthetic peptide
FTFGNFDMH

SEQ ID NO: 369 - protein, 9 aa, synthetic construct
  REGION 1..9: note = Description of Artificial Sequence: Synthetic peptide
FSFSNFDIH

SEQ ID NO: 370 - protein, 9 aa, synthetic construct
  REGION 1..9: note = Description of Artificial Sequence: Synthetic peptide
FNFSNFDMH

SEQ ID NO: 371 - protein, 12 aa, synthetic construct
  REGION 1..12: note = Description of Artificial Sequence: Synthetic peptide
SGLDHSGGAHYA

SEQ ID NO: 372 - protein, 12 aa, synthetic construct
  REGION 1..12: note = Description of Artificial Sequence: Synthetic peptide
SGLDHSGGAHYA

SEQ ID NO: 373 - protein, 12 aa, synthetic construct
  REGION 1..12: note = Description of Artificial Sequence: Synthetic peptide
STFDHSGGAHYA

SEQ ID NO: 374 - protein, 12 aa, synthetic construct
  REGION 1..12: note = Description of Artificial Sequence: Synthetic peptide
SGVDHSGGAHYA

SEQ ID NO: 375 - protein, 12 aa, synthetic construct
  REGION 1..12: note = Description of Artificial Sequence: Synthetic peptide
PGLDHSGGAHYA

SEQ ID NO: 376 - protein, 12 aa, synthetic construct
  REGION 1..12: note = Description of Artificial Sequence: Synthetic peptide
SGLDHNGGAHYA

SEQ ID NO: 377 - protein, 12 aa, synthetic construct
  REGION 1..12: note = Description of Artificial Sequence: Synthetic peptide
SGLDHSGGAHYA

SEQ ID NO: 378 - protein, 12 aa, synthetic construct
  REGION 1..12: note = Description of Artificial Sequence: Synthetic peptide
SGLDHRGGAHYA

SEQ ID NO: 379 - protein, 12 aa, synthetic construct
  REGION 1..12: note = Description of Artificial Sequence: Synthetic peptide
SVLDYSGGEHYA

SEQ ID NO: 380 - protein, 12 aa, synthetic construct
  REGION 1..12: note = Description of Artificial Sequence: Synthetic peptide
SGLDLSGGAHYA

SEQ ID NO: 381 - protein, 12 aa, synthetic construct
  REGION 1..12: note = Description of Artificial Sequence: Synthetic peptide
SGLAHSGGAHYA

SEQ ID NO: 382 - protein, 12 aa, synthetic construct
  REGION 1..12: note = Description of Artificial Sequence: Synthetic peptide
SGLDHSGGAHYA

SEQ ID NO: 383 - protein, 12 aa, synthetic construct
  REGION 1..12: note = Description of Artificial Sequence: Synthetic peptide
SGLDHSVGAHYA

SEQ ID NO: 384 - protein, 12 aa, synthetic construct
  REGION 1..12: note = Description of Artificial Sequence: Synthetic peptide
SGLDHSGGAHYA

SEQ ID NO: 385 - protein, 12 aa, synthetic construct
  REGION 1..12: note = Description of Artificial Sequence: Synthetic peptide
HGLDHSGGAHYA

SEQ ID NO: 386 - protein, 12 aa, synthetic construct
  REGION 1..12: note = Description of Artificial Sequence: Synthetic peptide
SGLDHSGGAQYA

SEQ ID NO: 387 - protein, 12 aa, synthetic construct
  REGION 1..12: note = Description of Artificial Sequence: Synthetic peptide
SGLDHSGGAHHA

SEQ ID NO: 388 - protein, 12 aa, synthetic construct
  REGION 1..12: note = Description of Artificial Sequence: Synthetic peptide
SGIDHSGGAHYA

SEQ ID NO: 389 - protein, 12 aa, synthetic construct
  REGION 1..12: note = Description of Artificial Sequence: Synthetic peptide
SGLDHSGGAHDA

SEQ ID NO: 390 - protein, 12 aa, synthetic construct
  REGION 1..12: note = Description of Artificial Sequence: Synthetic peptide
SGLDHTGGAHSE

SEQ ID NO: 391 - protein, 12 aa, synthetic construct
  REGION 1..12: note = Description of Artificial Sequence: Synthetic peptide
SGLGLCFGAHYA

SEQ ID NO: 392 - protein, 12 aa, synthetic construct
  REGION 1..12: note = Description of Artificial Sequence: Synthetic peptide
SGLDHSGGAHYA

SEQ ID NO: 393 - protein, 11 aa, synthetic construct
  REGION 1..11: note = Description of Artificial Sequence: Synthetic peptide
RASETISIYLN

SEQ ID NO: 394 - protein, 11 aa, synthetic construct
  REGION 1..11: note = Description of Artificial Sequence: Synthetic peptide
RASQGISSWLA

SEQ ID NO: 395 - protein, 11 aa, synthetic construct
  REGION 1..11: note = Description of Artificial Sequence: Synthetic peptide
QASQDISNYLN

SEQ ID NO: 396 - protein, 11 aa, synthetic construct
  REGION 1..11: note = Description of Artificial Sequence: Synthetic peptide
RASQSISSYLN

SEQ ID NO: 397 - protein, 11 aa, synthetic construct
  REGION 1..11: note = Description of Artificial Sequence: Synthetic peptide
RASQGISSWLA

SEQ ID NO: 398 - protein, 11 aa, synthetic construct
  REGION 1..11: note = Description of Artificial Sequence: Synthetic peptide
RASQSISSYLN

SEQ ID NO: 399 - protein, 11 aa, synthetic construct
  REGION 1..11: note = Description of Artificial Sequence: Synthetic peptide
RASQSISSFLN

SEQ ID NO: 400 - protein, 11 aa, synthetic construct
  REGION 1..11: note = Description of Artificial Sequence: Synthetic peptide
RASQGISDNLN

SEQ ID NO: 401 - protein, 11 aa, synthetic construct
  REGION 1..11: note = Description of Artificial Sequence: Synthetic peptide
RASQDIRNDLG

SEQ ID NO: 402 - protein, 11 aa, synthetic construct
  REGION 1..11: note = Description of Artificial Sequence: Synthetic peptide
RASQSISSWLA

SEQ ID NO: 403 - protein, 11 aa, synthetic construct
  REGION 1..11: note = Description of Artificial Sequence: Synthetic peptide
RASQSISSYLN

SEQ ID NO: 404 - protein, 11 aa, synthetic construct
  REGION 1..11: note = Description of Artificial Sequence: Synthetic peptide
RASQGIRNDLN

SEQ ID NO: 405 - protein, 7 aa, synthetic construct
  REGION 1..7: note = Description of Artificial Sequence: Synthetic peptide
AASSLQS

SEQ ID NO: 406 - protein, 7 aa, synthetic construct
  REGION 1..7: note = Description of Artificial Sequence: Synthetic peptide
AASSLQS

SEQ ID NO: 407 - protein, 7 aa, synthetic construct
  REGION 1..7: note = Description of Artificial Sequence: Synthetic peptide
AASSLES

SEQ ID NO: 408 - protein, 7 aa, synthetic construct
  REGION 1..7: note = Description of Artificial Sequence: Synthetic peptide
AASSLQS

SEQ ID NO: 409 - protein, 7 aa, synthetic construct
  REGION 1..7: note = Description of Artificial Sequence: Synthetic peptide
AASNLQM

SEQ ID NO: 410 - protein, 7 aa, synthetic construct
  REGION 1..7: note = Description of Artificial Sequence: Synthetic peptide
AASSLQS

SEQ ID NO: 411 - protein, 7 aa, synthetic construct
  REGION 1..7: note = Description of Artificial Sequence: Synthetic peptide
AASSLQS

SEQ ID NO: 412 - protein, 7 aa, synthetic construct
  REGION 1..7: note = Description of Artificial Sequence: Synthetic peptide
AASTLQS

SEQ ID NO: 413 - protein, 7 aa, synthetic construct
  REGION 1..7: note = Description of Artificial Sequence: Synthetic peptide
GASTLQS

SEQ ID NO: 414 - protein, 7 aa, synthetic construct
  REGION 1..7: note = Description of Artificial Sequence: Synthetic peptide
AASSLQS

SEQ ID NO: 415 - protein, 7 aa, synthetic construct
  REGION 1..7: note = Description of Artificial Sequence: Synthetic peptide
AASTLQS

SEQ ID NO: 416 - protein, 7 aa, synthetic construct
  REGION 1..7: note = Description of Artificial Sequence: Synthetic peptide
AASNSLS

SEQ ID NO: 417 - protein, 11 aa, synthetic construct [sequence omitted]

SEQ ID NO: 418 - protein, 11 aa, synthetic construct [sequence omitted]

SEQ ID NO: 419 - protein, 11 aa, synthetic construct [sequence omitted]

SEQ ID NO: 420 - protein, 11 aa, synthetic construct [sequence omitted]

SEQ ID NO: 421 - protein, 11 aa, synthetic construct [sequence omitted]

SEQ ID NO: 422 - protein, 10 aa, synthetic construct [sequence omitted]

SEQ ID NO: 423 - protein, 11 aa, synthetic construct [sequence omitted]

SEQ ID NO: 424 - protein, 11 aa, synthetic construct [sequence omitted]

SEQ ID NO: 425 - protein, 11 aa, synthetic construct [sequence omitted]

SEQ ID NO: 426 - protein, 10 aa, synthetic construct [sequence omitted]

SEQ ID NO: 427 - protein, 11 aa, synthetic construct [sequence omitted]

SEQ ID NO: 428 - protein, 11 aa, synthetic construct [sequence omitted]

SEQ ID NO: 429 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGLDHSGGAHYAGSVKGRFTISREDAKNSLDLQMNNLRVDDTAVYFCVRGTLYHYTSGSYCSDAFDIWGQGTLVTVSS

SEQ ID NO: 430 - protein, 126 aa, synthetic construct; identical to SEQ ID NO: 242
  REGION 1..126: note = Description of Artificial Sequence: Synthetic polypeptide

SEQ ID NO: 431 - protein, 126 aa, synthetic construct
  REGION 1..126: note = Description of Artificial Sequence: Synthetic polypeptide
EVQLVESGGGFVQPGGSLRLSCAASGFTFSTFDMHWVRQSPGKGLEWVSTFDHSGGAHYAGSVKGRFTISREDAKNSLDLQMNNLRVDDTAVYFCARGTLYHYTSGSYYSDAFDIWGQGTLVTVSS

SEQ ID NO: 432 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGVDHSGGAHYAGSVKGRFTISREDAKNSLDLQMNNLRVDDTAVYFCVRGTLYHYTSGSYYSDAFDIWGQGTLVTVSS

SEQ ID NO: 433 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVPGLDHSGGAHYAGSVKGRFTISREDAKNSLDLQMNNLRVDDTAVYFCVRGTLYHYTSGSYYSDAFDIWGQGTLVTVSS

SEQ ID NO: 434 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGLDHNGGAHYAGSVKGRFTISREDAKNSLDLQMNNLRVDDTAVYFCVRGTLYHYTSGSYYSDAFDIWGQGTLVTVSS

SEQ ID NO: 435 - protein, 126 aa, synthetic construct
  REGION 1..126: note = Description of Artificial Sequence: Synthetic polypeptide
EVQLVESGGGFVQPGGSLRLSCAASGFTLSNFDMHWVRQSPGKGLEWVSGLDHSGGAHYAGSVKGRFTISREDAKNSLDLQMNNLRVDDTAVYFCVRGTLYHYTSGSYYSDAFDIWGQGTLVTVSS

SEQ ID NO: 436 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGLDHRGGAHYAGSVKGRFTISREDAKNSLDLQMNNLRVDDTAVYFCVRGTLYHYTSGSYYSDAFDIWGQGTLVTVSS

SEQ ID NO: 437 - protein, 126 aa, synthetic construct
  REGION 1..126: note = Description of Artificial Sequence: Synthetic polypeptide
EVQLVESGGGFVQPGGSLRLSCAASGFTFRNFDMHWVRQSPGKGLEWVSVLDYSGGEHYAGSVKGRFTISREDAKNSLDLQMNNLRVDDTAVYFCVRGTLYHYTSGSYYSDAFDIWGQGTLVTVSS

SEQ ID NO: 438 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGLDLSGGAHYAGSVKGRFTISREDAKNSLDLQMNNLRVDDTAVYFCVRGTLYHYTSGSYYSDAFDIWGQGTLVTVSS

SEQ ID NO: 439 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGLAHSGGAHYAGSVKGRFTISREDAKNSLDLQMNNLRVDDTAVYFCVRGTLYHYTSGSYYSDAFDIWGQGTLVTVSS

SEQ ID NO: 440 - protein, 126 aa, synthetic construct
  REGION 1..126: note = Description of Artificial Sequence: Synthetic polypeptide
EVQLVESGGGFVQPGGSLRLSCAASGFTFSNYDMHWVRQSPGKGLEWVSGLDHSGGAHYAGSVKGRFTISREDAKNSLDLQMNNLRVDDTAVYFCVRGTLYHYTSGSYYSDAFDIWGQGTLVTVSS

SEQ ID NO: 441 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGLDHSVGAHYAGSVKGRFTISREDAKNSLDLQMNNLRVDDTAVYFCVRGTLYHYTSGSYYSDAFDIWGQGTLVTVSS

SEQ ID NO: 442 - protein, 126 aa, synthetic construct
  REGION 1..126: note = Description of Artificial Sequence: Synthetic polypeptide
EVQLVESGGGFVQPGGSLRLSCAASGFTFSNLDMHWVRQSPGKGLEWVSGLDHSGGAHYAGSVKGRFTISREDAKNSLDLQMNNLRVDDTAVYFCVRGTLYHYTSGSYYSDAFDIWGQGTLVTVSS

SEQ ID NO: 443 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVHGLDHSGGAHYAGSVKGRFTISREDAKNSLDLQMNNLRVDDTAVYFCVRGTLYHYTSGSYYSDAFDIWGQGTLVTVSS

SEQ ID NO: 444 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGLDHSGGAQYAGSVKGRFTISREDAKNSLDLQMNNLRVDDTAVYFCVRGTLYHYTSGSYYSDAFDIWGQGTLVTVSS

SEQ ID NO: 445 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGLDHSGGAHHAGSVKGRFTISREDAKNSLDLQMNNLRVDDTAVYFCVRGTLYHYTSGSYYSDAFDIWGQGTLVTVSS

SEQ ID NO: 446 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGIDHSGGAHYAGSVKGRFTISREDAKNSLDLQMNNLRVDDTAVYFCVRGTLYHYTSGSYYSDAFDIWGQGTLVTVSS

SEQ ID NO: 447 - protein, 126 aa, synthetic construct
  REGION 1..126: note = Description of Artificial Sequence: Synthetic polypeptide
EVQLVESGGGFVQPGGSLRLSCAASGFTFSNFDMHWVRQSPGKGLEWVSGLDHSGGAHDAGSVKGRFTISREDAKNSLDLQMNNLRVDDTAVYFCVRGTLYHYTSGSYYSDAFDIWGQGTLVTVSS

SEQ ID NO: 448 - protein, 126 aa, synthetic construct
  REGION 1..126: note = Description of Artificial Sequence: Synthetic polypeptide
EVQLVESGGGFVQPGGSLRLSCAASGFTFGNFDMHWVRQSPGKGLEWVSGLDHTGGAHSEGSVKGRFTISREDAKNSLDLQMNNLRVDDTAVYFCVRGTLYHYTSGSYYSDAFDIWGQGTLVTVSS

SEQ ID NO: 449 - protein, 126 aa, synthetic construct; identical to SEQ ID NO: 429
  REGION 1..126: note = Description of Artificial Sequence: Synthetic polypeptide

SEQ ID NO: 450-499 - skipped (50 entries)

SEQ ID NO: 500 - protein, 126 aa, synthetic construct; identical to SEQ ID NO: 242
  REGION 1..126: note = Description of Artificial Sequence: Synthetic polypeptide

SEQ ID NO: 501 - protein, 108 aa, synthetic construct
  REGION 1..108: note = Description of Artificial Sequence: Synthetic polypeptide
EIVLTQSPSSLSASVGDRVTITCRASETISIYLNWYQQKPGKAPKLLIYAASSLQSGVPSRFSGSGSGTDFTLTISSQQPEDFATYYCQQSYSTPWTFGQGTKVEIKR

SEQ ID NO: 502 - protein, 108 aa, synthetic construct
  REGION 1..108: note = Description of Artificial Sequence: Synthetic polypeptide
EIVLTQSPSSLSASVGDRVTITCRASQGISSWLAWYQQKPGKAPKLLIYAASSLQSGVPSRFSGSGSGTDFTLTISSQQPEDFATYYCQQANSFPLTFGQGTKVEIKR

SEQ ID NO: 503 - protein, 108 aa, synthetic construct; identical to SEQ ID NO: 273
  REGION 1..108: note = Description of Artificial Sequence: Synthetic polypeptide

SEQ ID NO: 504 - protein, 108 aa, synthetic construct
  REGION 1..108: note = Description of Artificial Sequence: Synthetic polypeptide
EIVLTQSPSSLSASVGDRVTITCRASQSISSYLNWYQQKPGKAPKLLIYAASSLQSGVPSRFSGSGSGTDFTLTISSQQPEDFATYYCQQSYSTPLTFGQGTKVEIKR

SEQ ID NO: 505 - protein, 108 aa, synthetic construct
  REGION 1..108: note = Description of Artificial Sequence: Synthetic polypeptide
EIVLTQSPSSLSASVGDRVTITCRASQGISSWLAWYQQKPGKAPKLLIYAASNLQMGVPSRFSGSGSGTDFTLTISSQQPEDFATYYCQQSYTTPLTFGQGTKVEIKR

SEQ ID NO: 506 - protein, 107 aa, synthetic construct
  REGION 1..107: note = Description of Artificial Sequence: Synthetic polypeptide
EIVLTQSPSSLSASVGDRVTITCRASQSISSYLNWYQQKPGKAPKLLIYAASSLQSGVPSRFSGSGSGTDFTLTISSQQPEDFATYYCQQSYSTHTFGQGTKVEIKR

SEQ ID NO: 507 - protein, 108 aa, synthetic construct
  REGION 1..108: note = Description of Artificial Sequence: Synthetic polypeptide
EIVLTQSPSSLSASVGDRVTITCRASQSISSFLNWYQQKPGKAPKLLIYAASSLQSGVPSRFSGSGSGTDFTLTISSQQPEDFATYYCQQSYSPPLTFGQGTKVEIKR

SEQ ID NO: 508 - protein, 108 aa, synthetic construct
  REGION 1..108: note = Description of Artificial Sequence: Synthetic polypeptide
EIVLTQSPSSLSASVGDRVTITCRASQGISDNLNWYQQKPGKAPKLLIYAASTLQSGVPSRFSGSGSGTDFTLTISSQQPEDFATYYCQQANSFPLTFGQGTKVEIKR

SEQ ID NO: 509 - protein, 108 aa, synthetic construct
  REGION 1..108: note = Description of Artificial Sequence: Synthetic polypeptide
EIVLTQSPSSLSASVGDRVTITCRASQDIRNDLGWYQQKPGKAPKLLIYGASTLQSGVPSRFSGSGSGTDFTLTISSQQPEDFATYYCQQSYITPLTFGQGTKVEIKR

SEQ ID NO: 510 - protein, 107 aa, synthetic construct
  REGION 1..107: note = Description of Artificial Sequence: Synthetic polypeptide
EIVLTQSPSSLSASVGDRVTITCRASQSISSWLAWYQQKPGKAPKLLIYAASSLQSGVPSRFSGSGSGTDFTLTISSQQPEDFATYYCQQSYSTITFGQGTKVEIKR

SEQ ID NO: 511 - protein, 108 aa, synthetic construct
  REGION 1..108: note = Description of Artificial Sequence: Synthetic polypeptide
EIVLTQSPSSLSASVGDRVTITCRASQSISSYLNWYQQKPGKAPKLLIYAASTLQSGVPSRFSGSGSGTDFTLTISSQQPEDFATYYCQQANSFPPTFGQGTKVEIKR

SEQ ID NO: 512 - protein, 108 aa, synthetic construct
  REGION 1..108: note = Description of Artificial Sequence: Synthetic polypeptide
EIVLTQSPSSLSASVGDRVTITCRASQGIRNDLNWYQQKPGKAPKLLIYAASNSLSGVPSRFSGSGSGTDFTLTISSQQPEDFATYYCQQANSFPLTFGQGTKVEIKR

SEQ ID NO: 513 - protein, 126 aa, synthetic construct
  REGION 1..126: note = Description of Artificial Sequence: Synthetic polypeptide
EVQLVESGGGLVQPGGSLRLSCAASGFTFSNFDMHWVRQATGKGLEWVSGLDHSGGAHYAGSVKGRFTISRENAKNSLYLQMNSLRAGDTAVYYCVRGTLYHYTSGSYYSDAFDIWGQGTLVTVSS

SEQ ID NO: 514 - protein, 126 aa, synthetic construct; identical to SEQ ID NO: 242
  REGION 1..126: note = Description of Artificial Sequence: Synthetic polypeptide

SEQ ID NO: 515 - protein, 126 aa, synthetic construct; identical to SEQ ID NO: 513
  REGION 1..126: note = Description of Artificial Sequence: Synthetic polypeptide

SEQ ID NO: 516 - protein, 126 aa, synthetic construct; identical to SEQ ID NO: 242
  REGION 1..126: note = Description of Artificial Sequence: Synthetic polypeptide

SEQ ID NO: 517 - protein, 126 aa, synthetic construct; identical to SEQ ID NO: 242
  REGION 1..126: note = Description of Artificial Sequence: Synthetic polypeptide

SEQ ID NO: 518 - protein, 107 aa, synthetic construct
  REGION 1..107: note = Description of Artificial Sequence: Synthetic polypeptide
EIVLTQSPSSLSASVGDRVTITCQASQDISNYLNWYQQKPGKAPKLLIYAASSLESGVPSRFSGSGSGTDFTLTISSQQPEDFATYYCQQANSFPYTFGQGTKVEIK

SEQ ID NO: 519 - protein, 107 aa, synthetic construct
  REGION 1..107: note = Description of Artificial Sequence: Synthetic polypeptide
DIQMTQSPSSLSASVGDRVTITCQASQDISNYLNWYQQKPGKAPKLLIYAASSLESGVPSRFSGSGSGTDFTLTISSLQPEDFATYYCQQANSFPYTFGQGTKVEIK

SEQ ID NO: 520 - protein, 107 aa, synthetic construct; identical to SEQ ID NO: 519
  REGION 1..107: note = Description of Artificial Sequence: Synthetic polypeptide

SEQ ID NO: 521 - protein, 108 aa, synthetic construct
  REGION 1..108: note = Description of Artificial Sequence: Synthetic polypeptide
EIVLTQSPSSLSASVGDRVTITCQASQDISNYLNWYQQKPGKAPKLLIYAASSLESGVPSRFSGSGSGTDFTLTISSQQPEDFATYYCQQAQSFPYTFGQGTKVEIKR

SEQ ID NO: 522 - protein, 108 aa, synthetic construct
  REGION 1..108: note = Description of Artificial Sequence: Synthetic polypeptide
EIVLTQSPSSLSASVGDRVTITCQASQDISNYLNWYQQKPGKAPKLLIYAASSLESGVPSRFSGSGSGTDFTLTISSQQPEDFATYYCQQANTFPYTFGQGTKVEIKR

SEQ ID NO: 523 - protein, 122 aa, synthetic construct
  REGION 1..122: note = Description of Artificial Sequence: Synthetic polypeptide
QVQLQESGPGLVKPSGTLSLTCAVSGDSFSSDGYSWSWIRQAPGKGLEWIGEIYHIGSTNYNPSLKSRVSMSVDKSKNQFSLRLTSVTAADTAVYYCAKHDYGKLWGGLDVWGQGTLVTVSS

SEQ ID NO: 524 - protein, 108 aa, synthetic construct
  REGION 1..108: note = Description of Artificial Sequence: Synthetic polypeptide
QPGLTQPPSVSVAPGKTAMITCGGNNIGTKSVHWYQQKPGQAPVLVVFDDSDRPSGIPERFSGSNSGNTATLAITGLQAEDQADYYCQSYDSSLSGVIFGGGTKLTVL

SEQ ID NO: 525-526 - skipped (2 entries)

SEQ ID NO: 527 - protein, 22 aa, synthetic construct
  REGION 1..22: note = Description of Artificial Sequence: Synthetic peptide
  VARIANT 2: note = MOD_RES - V or A
  VARIANT 15: note = MOD_RES - Y or C
CXRGTLYHYTSGSYXSDAFDIW

SEQ ID NO: 528 - protein, 11 aa, synthetic construct
  REGION 1..11: note = Description of Artificial Sequence: Synthetic peptide
  VARIANT 1: note = MOD_RES - Q or R
  VARIANT 4: note = MOD_RES - Q or E
  VARIANT 5: note = MOD_RES - D, G, T, or S
  VARIANT 7: note = MOD_RES - R or S
  VARIANT 8: note = MOD_RES - S, D, N, or I
  VARIANT 9: note = MOD_RES - N, F, Y, W, or D
  VARIANT 11: note = MOD_RES - N, G, or A
XASXXIXXXLX

SEQ ID NO: 529 - skipped

SEQ ID NO: 530 - protein, 10 aa, synthetic construct
  REGION 1..10: note = Description of Artificial Sequence: Synthetic peptide
  VARIANT 8: note = MOD_RES - I or H
CQQSYSTXTF

SEQ ID NO: 531 - protein, 11 aa, synthetic construct
  REGION 1..11: note = Description of Artificial Sequence: Synthetic peptide
  VARIANT 4: note = MOD_RES - A or S
  VARIANT 5: note = MOD_RES - N or Y
  VARIANT 6: note = MOD_RES - I, T, or S
  VARIANT 7: note = MOD_RES - P, F, or T
  VARIANT 9: note = MOD_RES - P, Y, L, or W
CQQXXXXPXTF

SEQ ID NO: 532 - protein, 10 aa, synthetic construct
HHHHHHHHHH

(19) United States
(12) Patent Application Publication
(10) Pub. No.: US 2024/0294618 A1
(43) Pub. Date: Sep. 5, 2024

(54) ANTI-VENOM ANTIBODIES AND USES THEREOF

(71) Applicant: Centivax, Inc., South San Francisco, CA (US)

(72) Inventors: Jacob E. Glanville, San Francisco, CA (US); Timothy Paul Friede, Green Bay, WI (US); David Tsao, Sunnyvale, CA (US); Sindy Andrea Liao Chan, San Jose, CA (US); I-Chieh Wang, San Bruno, CA (US)

(21) Appl. No.: 18/481,129

(22) Filed: Oct. 4, 2023

Related U.S. Application Data
(63) Continuation of application No. PCT/US2022/024109, filed on Apr. 8, 2022.
(60) Provisional application No. 63/172,782, filed on Apr. 9, 2021.

(51) Int. Cl. C07K 16/18; A61K 31/404; A61K 39/00; A61P 39/02; G01N 33/68

(52) U.S. Cl. CPC C07K 16/18; A61K 31/404; A61P 39/02; G01N 33/6857; A61K 2039/505; C07K 2317/565; C07K 2317/76; G01N 2333/4616; G01N 2333/4646

(57) ABSTRACT

This disclosure provides antibodies and antigen-binding fragments that can be administered to a subject that has been bitten by a venomous snake. Antibodies and antigen-binding fragments herein can be capable of treating or curing the subject and may provide protection against snake venom for up to several weeks. A combination or population of antibodies and antigen-binding fragments can be administered to the subject where the type of snake is not known or where a subject has been bitten by more than one species of snake. This disclosure further provides methods for identification of such broadly-neutralizing antibodies.

### CROSS-REFERENCE

This application is a continuation application of International Patent Application No. PCT/US2022/024109, filed Apr. 8, 2022, which claims the benefit of U.S. Provisional Application No. 63/172,782, filed Apr. 9, 2021, each of which application is incorporated herein by reference in its entirety.

### STATEMENT AS TO FEDERALLY SPONSORED RESEARCH

This invention was made with government support under Grant Number 1R43AI147898-01 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Dec. 21, 2023, is named 60333-712_301_SL.xml and is 634,355 bytes in size.

### SUMMARY

This application discloses broadly-neutralizing, fully human universal antivenom antibodies and cocktails of antibodies that exhibit(s) vastly superior properties than those currently available. Reference to an antibody herein may be interchanged with an antigen-binding fragment. Disclosed are an antivenom antibody and an antibody cocktail capable of neutralizing venom from multiple snake species with reduced potential for negative side effects. Also disclosed are populations antibodies, where the population contains multiple copies of a single antibody, which are capable of broadly neutralizing snake venom from more than one species of snake, or broadly neutralizing snake venom from more than one genus of snake. This disclosure further provides methods for identification of such broadly-neutralizing antibodies.

Such broadly neutralizing monoclonal antibodies, effective against entire classes of venoms across multiple snake species, make possible the prospect of a single universal antivenom against all snake species. Broadly neutralizing antibodies (bnAbs) are required for antibody-based universal antivenom because there are over 550 venomous snake species, and each venom consists of 10-70 unique toxic proteins. Thus, without anti-venom bnAbs, it would be an unrealistically ambitious task to generate 5,500-38,500 unique antibodies dedicated to each of the toxins from every species, let alone combine them such that they individually were at a sufficient concentration to provide any meaningful protection. However, with antivenom bnAbs, it is possible to have individual antibodies that neutralize shared common variant toxin homologs found across all snake venom from all species. As snake venom toxins belong to 10 distinct toxin classes, and of those only the “big four” (long-neurotoxin, PLA2, SVSP, SVMP) drive human morbidity and mortality from snakebite, in principle, a universal antivenom could be comprised of a cocktail of just four broadly neutralizing antivenom monoclonal antibodies.

Our studies recovered Centi-D9, a broadly neutralizing, ultra-high affinity antibody against long neurotoxin that was able to neutralize and provide in vivo protection against long-neurotoxins from cobras, taipans, and mambas. Crystal structures of Centi-D9 in complex with the long-neurotoxin of cobra, taipan, krait, and mamba were obtained and revealed a common mechanism of broad neutralization of these evolutionarily related but diverse toxin family members. Critically, the inventors demonstrated that Centi-D9, as a monoclonal, could independently provide mice complete in vivo protection from whole venom live challenge for black mamba and multiple cobra species, as well as partial protection in taipan. The results of these studies demonstrate that a universal antivenom is possible.

In 2021, snakebite envenoming, with its hemotoxic or neurotoxic effects, continues to be ranked on the World Health Organization's list of neglected tropical diseases, killing between 81,000-138,000 people annually and leaving up to 300,000 as amputees or permanently disabled each year (2018 reported numbers). Snake envenomation affects every continent except Antarctica, with the highest rates of envenomation occurring in Asia, Africa, and South America, disproportionally affecting rural populations (i.e., fieldworkers) and children. Snake envenomation affects the United States as well, with 7,000-8,000 cases annually, including an average of 50 from imported exotic snake species; 59.3% of these envenomations result in moderate to major outcomes and an average of 3 deaths. Although a global issue, major pharmaceutical manufacturers do not invest in improving envenomation treatments and have even abandoned the market in recent years due to breadth of species, causing the market to be fractured.

In contrast to existing approaches, the strategy proposed by the instant inventors is to isolate and characterize broadly neutralizing, fully human, antivenom antibodies that are stable and ready for field-use with no need for refrigeration. Due to low immunogenicity of fully human IgG, this approach will greatly reduce the potential for adverse effects, such as anaphylactic shock, pervasive in the existing therapies of animal-derived antivenom. Importantly, the therapy targets multiple snake species regionally, removing the need for the snakebite victim to identify the envenoming species. These molecules can be optimized to have better safety profiles and potentially higher efficacy. They are also be designed to be thermostable, enabling field-use for rapid treatment in the critical minutes post-snake-bite. With more lenient storage requirements and longer-shelf life, this therapy makes treatment available at a more competitive price point, greatly expanding the types of health centers that can store and access it, and enabling access to a more global, diverse population.

The technology described in this application utilizes a novel, diverse immune library harvested from a middle-aged male who has been administering dose-escalating self-immunizations (over 700 boosts and 200 live bites) from the world's most venomous snakes, including mambas, taipans, kraits, vipers, rattlers, and cobras, over the last two decades. Venoms from multiple snake species were panned against our library over several rounds in order to identify cross-reactive clones. Preliminary studies revealed 282 unique clones with VH CDR3 enrichment between different venoms, indicating possible cross-reactive binders in the output pools. This is highly significant as cross-reactive antibodies are vital in developing a broadly neutralizing antivenom. With the discovery of Centi-D9 targeting the long alpha-neurotoxin, the inventors have already demonstrated successful completion of one therapeutic or prophylactic antibody that can protect against whole venom in multiple types of elapid (e.g., taipan, mamba, cobra).

Development of a broad-spectrum vaccine has been limited by both the many diverse species and the diversity of toxins in the venom of each species. For over a century, snakebite envenomation treatment has been horse or sheep serum therapy with animal-derived antivenom preparations containing either immunoglobulin G (IgG) or derivative antigen-binding fragments (Fabs), typically from a single venom. More recently, some sheep or horse derived serum products have been created with some breadth of species coverage (CroFab for American pit vipers, EchiTAb for West African saw-scaled or carpet viper). While effective at reducing mortality and permanent disability in many cases, heterologous antivenom serotherapies present several challenges; depending on the antivenom, 6-59% of patients experience early-onset adverse reactions to a non-self reaction to the non-human horse or sheep plasma derived antivenoms including early adverse anaphylactic reactions (up to 24 h). Antivenoms are composed of ˜70% Fabs or Igs that are not directed against venom components but antigens the immunized animal has encountered. This reduces their potency and requires larger infusions of drug, typically with an intravenous infusion (IV) at a hospital setting that may not be local to the site of envenomation, and results in significant access problems and mortality prior to arriving at hospital or inability to treat by the time the patient has arrived at the hospital.

Polyclonal Fab-based formulations can lead to treatments with shortened half-lives and inconsistent batch quality in comparison to monoclonal IgGs. While digestion into Fab fragments reduces serum sickness by removal of the non-human Fc region, these treatments shorten the half-life from weeks to hours, requiring 8-10 doses of antivenom. Yet, patients cannot receive any additional future administrations, as they will have a severe reaction mounted against these antibodies. High concentrations of IgG can cause adverse reactions, including inflammation and serum sickness 1-2 weeks following therapy. Finally, antivenom developed for a single species requires the correct identification of the specific snake that bit the victim, which can be extremely difficult for victims and healthcare workers not well-versed in snake phenotypes and requires that the clinic has stocked the correct antivenom of many possible productions for different envenomations.

The antivenom compositions described herein are broadly protective for major deadly snake families. In addition, treatment does not require positive identification of the envenoming species and can be administered rapidly following snakebite, or prophylactically to those at high risk of snake bite. This will reduce costs by not having to stock multiple antivenoms, and by the extended shelf life of Centivax's lyophilized formulations. Snake envenomation is a high unmet need that has traditionally been under-invested in, especially by large pharmaceutical companies, and is akin to the niche market that is rare disease. Yet, a key difference is that snake envenomation is not rare, with 5 million envenomations, 300,000 permanent disabilities and 125,000 deaths annually. As the market is currently fractured amongst the multiple venoms for snake species, a single agent has the potential to merge and increase existing markets for anti-venom. Major end-users and customers will be militaries of 25 top nations, where deployed service members can carry our anti-venom as an effective prophylactic or acute treatment option while in austere environments. Additional use includes stocking many of the 28,600 hospitals globally, and with national subsidies aiding distribution in heavily impacted nations.

Non-human immunotherapy. For over a century, snakebite envenoming treatment has been horse or sheep serum therapy with animal-derived antivenom preparations containing derivative antigen-binding fragments (Fabs), usually from a single venom. More recently, some sheep or horse derived serum products have been created with some breadth of species coverage. CroFab (sheep-derived antivenom, polyvalent and lyophilized) and AnaVip (equine-derived Fab₂, lyophilized powder for injection) are both specific to North American snakes: rattlesnakes, cottonmouths, and copperheads. EchiTAb-G is sheep antisera effective against only the African West African Saw-Scaled Viper or Carpet Viper, recommended for use in sub-Saharan Africa. While effective at reducing mortality and permanent disability in many cases, heterologous antivenom serotherapies present several challenges; depending on the antivenom, 6-59% of patients experience early-onset adverse reactions due to a non-self reaction to the foreign horse or sheep plasma derived antivenoms. These adverse reactions can include early adverse anaphylactic reactions (up to 24 h).

Traditional antivenoms require IV administration. Antivenoms are composed of ˜70% Fabs or Igs that are not directed against venom components but other antigens. This has multiple drawbacks, since it reduces their potency and requires a larger infusion of the drug. The drug is often delivered IV, not at the site of envenomation, leading to increased mortality due to the time delay in administration. IV drugs greatly limit access to treatment, due to time delays, healthcare personnel required, and site of care. Polyclonal Fab based formulations can lead to treatments with shortened half-lives and inconsistent batch quality in comparison to monoclonal IgGs. While digestion into Fab fragments reduces serum sickness by removal of the non-human Fc region, these treatments shorten the half-life from weeks to hours, requiring 8-10 doses of antivenom. Furthermore, additional administration of these antibodies is prevented due to anticipated serum toxicity. High concentrations of non-human IgG antivenom can cause inflammation and serum sickness 1-2 weeks following therapy.

To date, antivenoms composed of monoclonal IgGs have not been employed in the clinical setting. Other antivenom-based companies, such as Venomyx, have demonstrated some cross-reactivity in preliminary studies but still derive their molecules from immunized camelids, requiring expensive and time-consuming humanizations. Moreover, their single-domain nature can result in a reduced half-life in comparison to fully human IgGs. If not properly neutralized with a single dose, repeated injections are necessary due to their small size that allows for renal elimination.

In contrast, the technology described in this application utilizes a novel, diverse immune library harvested from a middle-aged male who has been administering dose-escalating self-immunizations (over 700 boosts and 200 live bites) from the world's most venomous snakes including: mambas, taipans, kraits, vipers, rattlesnakes, and cobras, over the last 17 years.

The library construction utilized a unique Next Generation Sequencing (NGS) technology to amplify antibody variable domains from blood draws before and 7 days post-final venom immunization of the male subject, followed by deep sequencing and antibody phage display, enabling downstream venom binders to be traced back to the source blood draw. Antibody candidates against venoms from black mamba, Western diamondback rattlesnake, and coastal taipan were isolated during initial library panning.

Thermostabilized antibodies. Standard antivenom requires continuous refrigeration at 2-8° C. to preserve stability. This poses a significant challenge to rural communities, often in arid regions with endemic venomous species, that may not have the infrastructure to properly store antivenom. Current research is investigating the stability of antivenoms at room temperature. This application will develop thermostabilized antibodies with more lenient storage requirements, longer-shelf life, and is anticipated to be available at a more competitive price point, greatly expanding the types of health centers that can store and access it. Antivenom cross reactivity is a complex biochemical challenge, despite all medically relevant snakes belonging to 3 families: Atractaspidae, Viperidae, and Elapidae, with the overwhelming majority belonging to Viperidae or Elapidae. Within these groups, toxin proteomes of their venoms fall into 8-11 subfamilies, 4 of which are the dominant components across all venoms. Some existing antivenoms perform well in specific snake clades despite 8 million years of separation while other, more closely-related species, show poor neutralization for the same antivenom because of the sufficiency of a single mutation in an amino acid of a toxin molecule to interfere with antibody recognition.

In the hands of the instant inventors, preliminary deep sequencing panning analysis of our human subject's antibodies demonstrated VH CDR3 domain enrichment across multiple species, indicating potential cross-reactive candidates to multiple snake venom species. It is reasonable to assume our subject has developed antibodies capable of binding conserved epitopes across related peptides between snake species as he has immunized with a variety of snakes from both Viperidae and Elapidae families for almost two decades. This application utilizes high-throughput surface plasmon resonance (SPR), full-spectrum fluorescence and light scattering, and in vivo functional assays, to isolate and characterize broadly neutralizing fully human antivenom antibodies to develop a broad antivenom.

Broadly neutralizing antibodies (bnAbs). There are 550+ venomous snake species, and each venom consists of 10-70 unique toxic proteins. To develop a single universal anti-venom, broadly neutralizing monoclonal antibodies will need to be effective against entire classes of venoms across multiple snake species. Without anti-venom bnAbs, it would be an unrealistically ambitious task to generate 5,500-38,500 unique antibodies dedicated to each of the toxins from every species, let alone combine them such that they individually were at a sufficient concentration to provide any meaningful protection. However, with antivenom bnAbs, it is possible to have individual antibodies that neutralize shared common variant toxin homologs found across all snake venom from all species. As snake venom toxins belong to 10 distinct toxin classes, and of those, only the “big four” (long-neurotoxin, PLA2, SVSP, SVMP) drive human morbidity and mortality from snakebite, a universal antivenom can be comprised of a cocktail of just four broadly neutralizing antivenom monoclonal antibodies (FIG. 11).

Broadly neutralizing antibodies will overcome antivenom therapy limitations. 1) Broadly neutralizing antibodies can recognize whole families of snake toxins enabling a single antivenom product for each lethal snake family. This is of dramatic benefit to avoid the need for snake identification, risk of incorrect antivenom assignment, and will ultimately consolidate the economics of the market by producing a single product for all global regions. 2) Fully human antibodies can avoid serum toxicity by reducing the generation of adverse reactions, as human monoclonals are not rejected as immunogenic by the immune system of the recipient. These molecules could be easily optimized to have better safety profiles and potentially higher efficacy. Monoclonal IgGs have positive effects against snake toxins in in vivo lethality studies, neutralization testing, and in neutralizing abilities against multiple specific toxins responsible for myonecrosis and proteolytic effects.) Recombinant antivenom can be pure 100% antivenom-specific product, thus far more potent, providing the ability to reduce the volume and be delivered outside of hospital setting in dual chamber syringes.) Due to an improved toxicity profile compared to sheep IgG, fully human IgGs can be offered as prophylaxis with a 3-6 week window of protection, which is appropriate for individuals in high-risk exposure regions and/or occupations. Recombinant antibodies and antibody fragments of human origin are attractive as an alternative to antivenom production.

Fully human antivenom antibodies. This application describes the development of the next generation of safer and more effective antivenom biotherapeutics by isolating and characterizing broadly neutralizing fully human anti-venom antibody cocktails to treat snakebite envenomation. Monoclonal fully human-derived IgG greatly reduce the potential for adverse effects pervasive in animal-derived antivenom and allow for greater optimization that is expected to be more cost-effective than traditionally produced antivenoms.

Provided herein are universal anti-venom compositions that comprise a population of anti-venom antibodies or antigen-binding fragments. The population of anti-venom antibodies or antigen-binding fragments can comprise one or one or more antibodies or antigen-binding fragments. Alternatively, the population of anti-venom antibodies or antigen-binding fragments can comprise two antibodies or antigen-binding fragments, three antibodies or antigen-binding fragments, four antibodies or antigen-binding fragments, five antibodies or antigen-binding fragments, six antibodies or antigen-binding fragments, or more.

The one or more antibodies or antigen-binding fragments can comprise an IgG, an IgM, an IgE, an IgA, or an IgD, is derived therefrom, or a combination thereof. The one or more antibodies or antigen-binding fragments can comprise a monoclonal antibody, a grafted antibody, a chimeric antibody, a human antibody, or a humanized antibody. The antigen-binding fragment can comprise a Fab, a Fab′, a F(ab′)₂, a variable fragment (Fv), a triabody, a tetrabody, a minibody, a bispecific F(ab′)₂, a trispecific F(ab′)₂, a diabody, a bispecific diabody, a single chain variable fragment (scFv), a scFv-Fc, a Fab-Fc, a VHH, or a bispecific scFv.

The one or more antibodies or antigen-binding fragments comprise a VH CDR1, a VH CDR2, a VH CDR3, a VL CDR1, a VL CDR2, and a VL CDR3. The one or more antibodies or antigen-binding fragments can comprise a VH CDR1 having an amino acid sequence that is at least about 80%, 85%, 90%, 91%, 92%, 93%, 94%95%, 96%, 97%, 98%, 99%, or 100% identical to any one of the sequences of Table 2. Alternatively, or in addition, the one or more antibodies or antigen-binding fragments comprises a VH CDR2 having an amino acid sequence that is at least about 80%, 85%, 90%, 91%, 92%, 93%, 94%95%, 96%, 97%, 98%, 99%, or 100% identical to any one of the sequences of Table 3. Alternatively, or in addition, the one or more antibodies or antigen-binding fragments comprises a VH CDR3 having an amino acid sequence that is at least about 80%, 85%, 90%, 91%, 92%, 93%, 94% 95%, 96%, 97%, 98%, 99%, or 100% identical to any one of the sequences of Table 1. Alternatively, or in addition, the one or more antibodies or antigen-binding fragments comprises a VL CDR1 having an amino acid sequence that is at least about 80%, 85%, 90%, 91%, 92%, 93%, 94%95%, 96%, 97%, 98%, 99%, or 100% identical to any one of the sequences of Table 4. Alternatively, or in addition, the one or more antibodies or antigen-binding fragments comprises a VL CDR2 having an amino acid sequence that is at least about 80%, 85%, 90%, 91%, 92%, 93%, 94%95%, 96%, 97%, 98%, 99%, or 100% identical to any one of the sequences of Table 5. Alternatively, or in addition, the one or more antibodies or antigen-binding fragments comprises a VL CDR3 having an amino acid sequence that is at least about 80%, 85%, 90%, 91%, 92%, 93%, 94%95%, 96%, 97%, 98%, 99%, or 100% identical to any one of the sequences of Table 6.

Alternatively, or in addition, the one or more antibodies or antigen-binding fragments comprises a FW-H1 having an amino acid sequence that is at least about 80%, 85%, 90%, 91%, 92%, 93%, 94% 95%, 96%, 97%, 98%, 99%, or 100% identical to any one of the sequences of Table 7. Alternatively, or in addition, the one or more antibodies or antigen-binding fragments comprises a FW-H2 having an amino acid sequence that is at least about 80%, 85%, 90%, 91%, 92%, 93%, 94%95%, 96%, 97%, 98%, 99%, or 100% identical to any one of the sequences of Table 8. Alternatively, or in addition, the one or more antibodies or antigen-binding fragments comprises a FW-H3 having an amino acid sequence that is at least about 80%, 85%, 90%, 91%, 92%, 93%, 94%95%, 96%, 97%, 98%, 99%, or 100% identical to any one of the sequences of Table 9. Alternatively, or in addition, the one or more antibodies or antigen-binding fragments comprises a FW-H4 having an amino acid sequence that is at least about 80%, 85%, 90%, 91%, 92%, 93%, 94%95%, 96%, 97%, 98%, 99%, or 100% identical to any one of the sequences of Table 10.

Alternatively, or in addition, the one or more antibodies or antigen-binding fragments comprises a FW-L1 having an amino acid sequence that is at least about 80%, 85%, 90%, 91%, 92%, 93%, 94% 95%, 96%, 97%, 98%, 99%, or 100% identical to any one of the sequences of Table 11. Alternatively, or in addition, the one or more antibodies or antigen-binding fragments comprises a FW-L2 having an amino acid sequence that is at least about 80%, 85%, 90%, 91%, 92%, 93%, 94%95%, 96%, 97%, 98%, 99%, or 100% identical to any one of the sequences of Table 12. Alternatively, or in addition, the one or more antibodies or antigen-binding fragments comprises a FW-L3 having an amino acid sequence that is at least about 80%, 85%, 90%, 91%, 92%, 93%, 94%95%, 96%, 97%, 98%, 99%, or 100% identical to any one of the sequences of Table 13. Alternatively, or in addition, the one or more antibodies or antigen-binding fragments comprises a FW-L4 having an amino acid sequence that is at least about 80%, 85%, 90%, 91%, 92%, 93%, 94%95%, 96%, 97%, 98%, 99%, or 100% identical to any one of the sequences of Table 14.

Alternatively, or in addition, the one or more antibodies or antigen-binding fragments comprises a VH having an amino acid sequence that is at least about 80%, 85%, 90%, 91%, 92%, 93%, 94%95%, 96%, 97%, 98%, 99%, or 100% identical to any one of the sequences of Table 15. Alternatively, or in addition, the one or more antibodies or antigen-binding fragments comprises a VL having an amino acid sequence that is at least about 80%, 85%, 90%, 91%, 92%, 93%, 94%95%, 96%, 97%, 98%, 99%, or 100% identical to any one of the sequences of Table 16.

In another aspect, provided herein is method of treating a subject who suffers from an envenomation, comprising administering to the subject a composition comprising an effective amount of a universal anti-venom that comprises one or more antibodies or antigen-binding fragments. In some instances, the envenomation occurs from one or more snake(s).

The method can comprise administering to the subject one or more antibodies or antigen-binding fragments. Alternatively, the method can comprise administering to the subject two antibodies or antigen-binding fragments, three antibodies or antigen-binding fragments, four antibodies or antigen-binding fragments, five antibodies or antigen-binding fragments, six antibodies or antigen-binding fragments, seven antibodies or antigen-binding fragments, eight antibodies or antigen-binding fragments, nine antibodies or antigen-binding fragments, ten antibodies or antigen-binding fragments, or more. The one or more antibodies or antigen-binding fragments to be administered to the subject can comprise an IgG, an IgM, an IgE, an IgA, or an IgD, is derived therefrom, or a combination thereof. The one or more antibodies or antigen-binding fragments to be administered to the subject can comprise a monoclonal antibody, a grafted antibody, a chimeric antibody, a human antibody, or a humanized antibody. The antigen-binding fragment to be administered to the subject can comprise a Fab, a Fab′, a F(ab′)₂, a variable fragment (Fv), a triabody, a tetrabody, a minibody, a bispecific F(ab′)₂, a trispecific F(ab′)₂, a diabody, a bispecific diabody, a single chain variable fragment (scFv), a scFv-Fc, a Fab-Fc, a VHH, a bispecific scFv, or a combination thereof.

In any of such methods, the subject can be further administered one or more additional therapies or drugs to the subject. An additional therapy or drug can comprise, for example, an NSAID. Alternatively, or in addition, an additional therapy or drug can comprise, for example, a PLA2 inhibitor. PLA2 inhibitors include, but are not limited to, comprises varespladib, methylvarespladib, or a combination thereof. Administration can be via any suitable means such as, for example, one or more injections.

In any of such methods, the one or more antibodies or antigen-binding fragments to be administered to the subject can comprise a VH CDR3 having an amino acid sequence that is at least about 80%, 85%, 90%, 91%, 92%, 93%, 94%95%, 96%, 97%, 98%, 99%, or 100% identical to any one of the sequences of Table 1. Alternatively, or in addition, the one or more antibodies or antigen-binding fragments to be administered to the subject comprises a VH CDR1 having an amino acid sequence that is at least about 80%, 85%, 90%, 91%, 92%, 93%, 94%95%, 96%, 97%, 98%, 99%, or 100% identical to any one of the sequences of Table 2. Alternatively, or in addition, the one or more antibodies or antigen-binding fragments to be administered to the subject comprises a VH CDR2 having an amino acid sequence that is at least about 80%, 85%, 90%, 91%, 92%, 93%, 94%95%, 96%, 97%, 98%, 99%, or 100% identical to any one of the sequences of Table 3.

Alternatively, or in addition, the one or more antibodies or antigen-binding fragments to be administered to the subject comprises a VL CDR1 having an amino acid sequence that is at least about 80%, 85%, 90%, 91%, 92%, 93%, 94%95%, 96%, 97%, 98%, 99%, or 100% identical to any one of the sequences of Table 4. Alternatively, or in addition, the one or more antibodies or antigen-binding fragments to be administered to the subject comprises a VL CDR2 having an amino acid sequence that is at least about 80%, 85%, 90%, 91%, 92%, 93%, 94%95%, 96%, 97%, 98%, 99%, or 100% identical to any one of the sequences of Table 5. Alternatively, or in addition, the one or more antibodies or antigen-binding fragments to be administered to the subject comprises a VL CDR3 having an amino acid sequence that is at least about 80%, 85%, 90%, 91%, 92%, 93%, 94%95%, 96%, 97%, 98%, 99%, or 100% identical to any one of the sequences of Table 6.

Alternatively, or in addition, the one or more antibodies or antigen-binding fragments to be administered to the subject comprises a FW-H1 having an amino acid sequence that is at least about 80%, 85%, 90%, 91%, 92%, 93%, 94%95%, 96%, 97%, 98%, 99%, or 100% identical to any one of the sequences of Table 7. Alternatively, or in addition, the one or more antibodies or antigen-binding fragments to be administered to the subject comprises a FW-H2 having an amino acid sequence that is at least about 80%, 85%, 90%, 91%, 92%, 93%, 94%95%, 96%, 97%, 98%, 99%, or 100% identical to any one of the sequences of Table 8. Alternatively, or in addition, the one or more antibodies or antigen-binding fragments to be administered to the subject comprises a FW-H3 having an amino acid sequence that is at least about 80%, 85%, 90%, 91%, 92%, 93%, 94%95%, 96%, 97%, 98%, 99%, or 100% identical to any one of the sequences of Table 9. Alternatively, or in addition, the one or more antibodies or antigen-binding fragments to be administered to the subject comprises a FW-H4 having an amino acid sequence that is at least about 80%, 85%, 90%, 91%, 92%, 93%, 94%95%, 96%, 97%, 98%, 99%, or 100% identical to any one of the sequences of Table 10.

Alternatively, or in addition, the one or more antibodies or antigen-binding fragments to be administered to the subject comprises a FW-L1 having an amino acid sequence that is at least about 80%, 85%, 90%, 91%, 92%, 93%, 94%95%, 96%, 97%, 98%, 99%, or 100% identical to any one of the sequences of Table 11. Alternatively, or in addition, the one or more antibodies or antigen-binding fragments to be administered to the subject comprises a FW-L2 having an amino acid sequence that is at least about 80%, 85%, 90%, 91%, 92%, 93%, 94%95%, 96%, 97%, 98%, 99%, or 100% identical to any one of the sequences of Table 12. Alternatively, or in addition, the one or more antibodies or antigen-binding fragments to be administered to the subject comprises a FW-L3 having an amino acid sequence that is at least about 80%, 85%, 90%, 91%, 92%, 93%, 94%95%, 96%, 97%, 98%, 99%, or 100% identical to any one of the sequences of Table 13. Alternatively, or in addition, the one or more antibodies or antigen-binding fragments to be administered to the subject comprises a FW-L4 having an amino acid sequence that is at least about 80%, 85%, 90%, 91%, 92%, 93%, 94%95%, 96%, 97%, 98%, 99%, or 100% identical to any one of the sequences of Table 14.

Alternatively, or in addition, the one or more antibodies or antigen-binding fragments to be administered to the subject comprises a VH having an amino acid sequence that is at least about 80%, 85%, 90%, 91%, 92%, 93%, 94%95%, 96%, 97%, 98%, 99%, or 100% identical to any one of the sequences of Table 15. Alternatively, or in addition, the one or more antibodies or antigen-binding fragments to be administered to the subject comprises a VL having an amino acid sequence that is at least about 80%, 85%, 90%, 91%, 92%, 93%, 94%95%, 96%, 97%, 98%, 99%, or 100% identical to any one of the sequences of Table 16.

In any of such instances of the universal anti-venom compositions or methods, the one or more antibodies or antigen-binding fragments, bind to one or more toxins from one or more snakes selected from the group consisting of Boiga irregularis (Brown Tree Snake), Boiga cyanea (Green Cat Snake), Boiga dendrophila (Mangrove Snake), Dispholidus typus (Boomslang), Salvadora grahamiae (Mountain Patchnose Snake), Spalerosophis diadema (Diadem Snake), Tantilla nigriceps (Plains Blackhead Snake), Thelotornis capensis (Southern Twig Snake), Thelotornis kirtlandii (Northern Twig Snake), Toxicodryas blandingii (Blandings Tree Snake), Trimorphodon lambda (Sonoran Lyre Snake), Amphiesma stolatum (Buff Striped Keelback), Natrix tessellate (Dice Snake), Rhabdophis subminiatus (Red Necked Keelback), Rhabdophis tigrinus (Tiger Keelback), Thamnophis elegans (Western Terrestrial Garter Snake), Thamnophis sirtalis (Common Garter Snake), Ahaetulla nasuta (Long Nosed Whip Snake), Atractaspis bibronii (Bibron's Burrowing Asp), Atractaspis dahomeyensis (Dahomey Burrowing Asp), Atractaspis engaddensis (Palestinian Mole Viper), Atractaspis microlepidota (Small Scaled Burrowing Asp), Malpolon monspessulanus (Montpellier Snake), Acanthophis antarcticus (Common Death Adder), Aipysurus laevis (Olive Brown Sea Snake), Aipysurus duboisii (Dubois' Sea Snake), Austrelaps superbus (Lowland Copperhead), Cryptophis nigrescens (Small Eyed Snake), Demansia olivacea (Olive Whip Snake), Emydocephalus annulatus (Annulated Turtle Headed Sea Snake), Furina tristis (Stephen's Banded Snake), Hydrophis melanocephalus (Black Headed Slender Necked Sea Snake), Hydrophis curtus (Short Sea Snake), Hydrophis gracilis (Slender Sea Snake), Hydrophis elegans (Elegant Sea Snake), Hydrophis jerdonii (Cone Nosed Sea Snake), Hydrophis klossi (Selangor Sea Snake), Hydrophis peronii (Horned Sea Snake), Hydrophis belcheri (Belcher's Sea Snake), Hydrophis stricticollis (Bengal Sea Snake), Hydrophis major (Olive Headed Sea Snake), Hydrophis stokesii (Large Headed Sea Snake), Hydrophis melanosoma (Black Banded Robust Sea Snake), Hydrophis hardwickii (Spine Bellied Sea Snake), Hydrophis cyanocinctus (Annulated Sea Snake), Hydrophis spiralis (Narrow Banded Sea Snake), Hydrophis nigrocinctus (Black Banded Sea Snake), Hydrophis platurus (Yellowbelly Sea Snake), Hydrophis ornatus (Ornate Reef Sea Snake), Hydrophis viperinus (Viperine Sea Snake), Hydrophis schistosus (Beaked Sea Snake), Notechis scutatus (Mainland Tiger Snake), Oxyuranus scutellatus (Coastal Taipan), Oxyuranus temporalis (Central Ranges Taipan), Pseudechis australis (Mulga Snake), Pseudechis butleri (Butler's Black Snake), Pseudechis colletti (Collett's Black Snake), Pseudechis guttatus (Blue Bellied Black Snake), Pseudechis papuanus (Papuan Black Snake), Pseudechis porphyriacus (Red Bellied Black Snake), Pseudonaja affinis (Dugite), Pseudonaja guttata (Speckled Brown Snake), Pseudonaja inframacula (Peninsula Brown Snake), Pseudonaja nuchalis (Western Brown Snake), Pseudonaja textilis (Eastern Brown Snake), Tropidechis carinatus (Clarence River Snake), Aspidelaps lubricus (Cape Coral Snake), Aspidelaps scutatus (Shield Nose Snake), Bungarus fasciatus (Banded Krait), Bungarus caeruleus (Indian Krait), Bungarus candidus (Blue Krait), Bungarus flaviceps (Red Headed Krait), Bungarus multicinctus (Many Banded Krait), Dendroaspis viridis (Western Green Mamba), Dendroaspis angusticeps (Eastern Green Mamba), Dendroaspis jamesoni (Jameson's Mamba), Dendroaspis polylepis (Black Mamba), Elapsoidea sundevallii (Sundevall's African Garter Snake), Hemachatus haemachatus (Rinkhals), Laticauda colubrina (Common Yellow Lipped Sea Krait), Laticauda laticaudata (Blue Lipped Sea Krait), Laticauda semifasciata (Black Banded Sea Krait), Micrurus obscurus (Bolivian Coral Snake), Micrurus frontalis (Southern Coral Snake), Micrurus alleni (Allen's Coral Snake), Micrurus altirostris (Uruguayan Coral Snake), Micrurus clarki (Clark's Coral Snake), Micrurus corallinus (Painted Coral Snake), Micrurus distans (West Mexican Coral Snake), Micrurus dumerilii (Dumeril's Coral Snake), Micrurus fulvius (Eastern Coral Snake), Micrurus hemprichii (Hemprich's Coral Snake), Micrurus ibiboboca (Caatinga Coral Snake), Micrurus lemniscatus (South American Coral Snake), Micrurus mipartitus (Redtail Coral Snake), Micrurus mosquitensis (Costa Rican Coral Snake), Micrurus multifasciatus (Many Banded Coral Snake), Micrurus nigrocinctus (Central American Coral Snake), Micrurus pyrrhocryptus (Argentinean Coral Snake), Micrurus spixii (Amazon Coral Snake), Micrurus surinamensis (Aquatic Coral Snake), Micrurus tener (Texas Coral Snake), Micrurus tschudii (Desert Coral Snake), Naja siamensis (Indochinese Spitting Cobra), Naja annulata (Ringed Water Cobra), Naja annulifera (Banded Cobra), Naja ashei (Giant Spitting Cobra), Naja atra (Chinese Cobra), Naja christyi (Congo Water Cobra), Naja haje (Egyptian Cobra), Naja kaouthia (Monocled Cobra), Naja katiensis (Mali Cobra), Naja melanoleuca (Forest Cobra), Naja mossambica (Mozambique Spitting Cobra), Naja (Spectacled Cobra), Naja nigricollis (Black Necked Spitting Cobra), Naja nivea (Cape Cobra), Naja nubiae (Nubian Spitting Cobra), Naja oxiana (Caspian Cobra), Naja pallida (Red Spitting Cobra), Naja philippinensis (Northern Philippine Cobra), Naja samarensis (Samar Cobra), Naja sputatrix (Javan Spitting Cobra), Naja sumatrana (Sumatran Spitting Cobra), Ophiophagus hannah (King Cobra), Walterinnesia aegyptia (Western Black Desert Cobra), Homalopsis buccata (Linne's Water Snake), Myrrophis chinensis (Chinese Mud Snake), Subsessor bocourti (Bocourt's Water Snake), Azemiops fede (Fea's Viper), Agkistrodon bilineatus (Mexican Cantil), Agkistrodon contortrix (Copperhead), Agkistrodon piscivorus (Cottonmouth), Agkistrodon taylori (Castellana), Agkistrodon laticinctus (Broad Banded Copperhead), Atropoides picadoi (Picado's Jumping Pitviper), Bothriechis lateralis (Side Striped Palm Viper), Bothriechis nigroviridis (Black Speckled Palm Pitviper), Bothriechis schlegelii (Eyelash Palm Pitviper), Bothrops diporus (Chaco Lancehead), Bothrops erythromelas (Caatinga Lancehead), Bothrops insularis (Golden Lancehead Viper), Bothrops jararaca (Jararaca), Bothrops neuwiedi (Neuwied's Lancehead), Bothrops pauloensis (Black Faced Lancehead), Bothrops asper (Terciopelo), Bothrops atrox Common Lancehead), Bothrops ayerbei Ayerbe's Lancehead), Bothrops caribbaeus Saint Lucia Lancehead), Bothrops jararacussu Jararacussu), Bothrops lanceolatus (Martinique Lancehead), Bothrops leucurus (Whitetail Lancehead), Bothrops moojeni (Brazilian Lancehead), Bothrops alternatus (Urutú), Bothrops cotiara (Cotiara), Bothrops fonsecai (Fonseca's Lancehead), Bothrops itapetiningae (São Paulo Lancehead), Bothrops taeniatus (Speckled Forest Pitviper), Bothrops mattogrossensis (Mato Grosso Lanzenotter), Calloselasma rhodostoma (Malayan Pitviper), Cerrophidion godmani (Godman's Montane Pitviper), Cerrophidion sasai (Costa Rica Montane Pitviper), Crotalus viridis (Prairie Rattlesnake), Crotalus atrox (Western Diamondback Rattlesnake), Crotalus adamanteus (Eastern Diamondback Rattlesnake), Crotalus basiliscus (Mexican West Coast Rattlesnake), Crotalus catalinensis (Santa Catalina Island Rattlesnake), Crotalus cerastes (Sidewinder Rattlesnake), Crotalus Cerberus (Arizona Black Rattlesnake), Crotalus durissus (South American Rattlesnake), Crotalus enyo (Baja California Rattlesnake), Crotalus horridus (Timber Rattlesnake), Crotalus lepidus (Mottled Rock Rattlesnake), Crotalus mitchellii (San Lucan Speckled Rattlesnake), Crotalus molossus (Northern Black Tailed Rattlesnake), Crotalus oreganus (Northern Pacific Rattlesnake), Crotalus pricei (Western Twin Spotted Rattlesnake), Crotalus pusillus (Tancitaran Dusky Rattlesnake), Crotalus ravus (Mexican Pygmy Rattlesnake), Crotalus ruber (Red Diamond Rattlesnake), Crotalus scutulatus (Mohave Rattlesnake), Crotalus simus (Central American Rattlesnake), Crotalus tigris (Tiger Rattlesnake), Crotalus totonacus (Totonacan Rattlesnake), Crotalus tzabcan (Yucatán Neotropical Rattlesnake), Crotalus willardi (Arizona Ridgenose Rattlesnake), Crotalus Pyrrhus (Southwestern Speckled Rattlesnake), Crotalus vegrandis (Uracoan Rattlesnake), Deinagkistrodon acutus (Chinese Sharp Nosed Pitviper), Gloydius intermedius (Intermediate Mamushi), Gloydius blomhoffii (Mamushi), Gloydius brevicaudus (Short Tailed Mamushi), Gloydius halys (Siberian Pitviper), Gloydius shedaoensis (Shedao Island Pitviper), Gloydius ussuriensis (Ussuri Mamushi), Hypnale (Hump Nosed Pitviper), Lachesis melanocephala (Black Headed Bushmaster), Lachesis muta (Atlantic Bushmaster), Ovophis okinavensis (Hime Habu), Porthidium nasutum (Rainforest Hog Nosed Pitviper), Porthidium ophryomegas (Slender Hognosed Pitviper), Protobothrops elegans (Sakishima Habu), Protobothrops flavoviridis (Okinawa Habu), Protobothrops mangshanensis (Mangshan Pitviper), Protobothrops mucrosquamatus (Taiwan Habu), Protobothrops tokarensis (Tokara Island Pit Viper), Sistrurus catenatus (Massassauga), Sistrurus miliarius (Pygmy Rattlesnake), Trimeresurus stejnegeri (Chinese Green Tree Viper), Trimeresurus albolabris (White Lipped Pitviper), Trimeresurus erythrurus (Red Tailed Bamboo Pitviper), Trimeresurus gramineus (Bamboo Pitviper), Trimeresurus labialis (Nicobar Bamboo Pitviper), Trimeresurus macrops (Large Eyed Pitviper), Trimeresurus malabaricus (Malabar Pitviper), Trimeresurus popeiorum (Pope's Bamboo Pitviper), Trimeresurus purpureomaculatus (Mangrove Pitviper), Trimeresurus sumatranus (Sumatran Pitviper), Tropidolaemus wagleri (Wagler's Pitviper), Metlapilcoatlus mexicanus (Mexican Jumping Pitviper), Metlapilcoatlus nummifer (Central American Jumping Pitviper), Atheris squamigera (African Bush Viper), Bitis arietans (Puff Adder), Bitis Atropos (Cape Mountain Adder), Bitis caudalis (Horned Puff Adder), Bitis cornuta (Western Many Horned Adder), Bitis gabonica (Central African Gaboon Viper), Bitis nasicornis (Rhinoceros Viper), Bitis parviocula (Ethiopian Mountain Adder), Bitis rhinoceros (West African Gaboon Viper), Causus rhombeatus (Common Night Adder), Cerastes (Horned Viper), Cerastes gasperettii (Arabian Horned Viper), Cerastes vipera (Sahara Sand Viper), Daboia palaestinae (Palestine Viper), Daboia russelii (Russell's Viper), Daboia siamensis (Eastern Russell's Viper), Daboia mauritanica (Moorish Viper), Echis ocellatus (West African Carpet Viper), Echis carinatus (Saw Scaled Viper), Echis coloratus (Painted Saw Scaled Viper), Echis pyramidum (Egyptian Saw Scaled Viper), Macrovipera schweizeri (Milos Viper), Macrovipera lebetinus (Levantine Viper), Montivipera bornmuelleri (Lebanon Viper), Montivipera latifii (Latifi's Viper), Montivipera raddei (Armenian Mountain Viper), Montivipera xanthine (Ottoman Viper), Pseudocerastes fieldi (Field's Horned Viper), Pseudocerastes persicus (Persian Horned Viper), Vipera ammodytes (European Nose Horned Viper), Vipera aspis (Aspic Viper), Vipera berus (Common European Adder), Vipera latastei (Lataste's Viper), Vipera lotievi (Caucasian Meadow Viper), Vipera seoanei (Baskian Viper), Vipera ursinii (Ursini's Viper), Diadophis punctatus (Ringneck Snake), Hydrodynastes gigas (False Water Cobra), Hypsiglena torquata (Night Snake), Hypsiglena jani (San Luis Potosi Night Snake), Leptodeira ashmeadii, Philodryas olfersii (Lichtenstein's Green Racer), and a combination thereof. In one instance, the envenomation is from saw-scaled viper, water moccasin, lancehead, rattlesnake, Russell's viper, puff adder, or a combination thereof. In another instance, the envenomation is from saw-scaled viper, water moccasin, lancehead, rattlesnake, Russell's viper, and puff adder. In another instance, the envenomation is from krait (Bungarus caeruleus), black mamba (Dendroaspis polylepsis), coastal taipan (Oxyuranus scutatellus), cape cobra (Naja nivea), or a combination thereof. In another instance, the envenomation is from krait (Bungarus caeruleus), black mamba (Dendroaspis polylepsis), coastal taipan (Oxyuranus scutatellus), and cape cobra (Naja nivea).

Provided herein is an antibody or antigen-binding fragment that is a broadly neutralizing antibody against a three-fingered toxin (3FTx). In one embodiment, the 3FTx is a long neurotoxin. The antibody or antigen-binding fragment can neutralize alpha-bungarotoxin (krait), alpha-elapitoxin (mamba), pseudonajatoxin (brown snake), alpha-cobra toxin (cobra), toxin B (king cobra), or a combination thereof. In one instance, the antibody or antigen-binding fragment broadly neutralizes toxin from 2, 3, 4, 5, or more of the long neurotoxins. In a preferred embodiment, the antibody or antigen-binding fragment neutralizes the effect of the long neurotoxin. In a preferred embodiment, the antibody or antigen-binding fragment binds one or more three-fingered toxins in such a manner that prevents binding of the toxin to human nicotinic acetylcholine receptors (nAChRs). In some embodiments, the antibody or antigen-binding fragment binds at least 30%, 40%, 50%, 60%, 70%, 80%, or 90% of the toxin accessible surface area that is buried upon the toxin binding nAChR. The antibody or antigen-binding fragment can be, for example, SNEURO_P01_D09. In one instance, SNEURO_P01_D09 comprises a VH CDR1 of SEQ ID NO: 28, a VH CDR2 of SEQ ID NO: 41, a VH CDR3 of SEQ ID NO: 12, a VL CDR1 of SEQ ID NO: 46, a VL CDR2 of SEQ ID NO: 80, and a VL CDR3 of SEQ ID NO: 101. In another instance, SNEURO_P01_D09 comprises a VH having an amino acid sequence of SEQ ID NO: 242 and a VL having an amino acid sequence of SEQ ID NO: 273.

Provided herein is a method of identifying a broadly-neutralizing antibody or antigen-binding fragment that selectively binds to 2 or more snake venom toxins belonging to a family of homologous antigens, the method comprising (a) immunizing a subject with two or more homologous antigens; and (b) conducting an iterative selection process to specifically identify cross-reactive antibodies or antigen-binding fragments from the B cell repertoire of the subject, wherein the iterative selection process down-selects possible candidates using 2 or more homologous antigens. A subject can be, for example, a mammal (e.g., a human). In some embodiments, the family of homologous antigens comprises three-finger toxins (3FTXs) including, but not limited to, long neurotoxins. The iterative selection process can comprise immunoprecipitation of antibodies from serum obtained from the subject, fluorescence-activated cell sorting (FACS) of B cells obtained from the subject, panning of a library derived from B cell RNA obtained from the subject displayed in phage or yeast, or any combination thereof.

In some instances, the subject has been immunized one or more time(s) with multiple homologous antigens from the same family. In some instances, the subject has been immunized with 2, 3, 4, 5, 6, 7, 8, 9, 10, 11, 12, 13, 14, 15, 16, 17, 18, 19, 20 or more different snake venom toxins. In one, embodiment, the method further comprises screening identified antibodies or antigen-binding fragments for binding against multiple homologous antigens. Screening can be any suitable assay including, but not limited to, an affinity assay, a kinetic assay, or a combination thereof. In some instances, the affinity assay or a kinetic assay comprise an enzyme-linked immunosorbant assay (ELISA), an Octet HTX assay, a Biacore assay, or a combination thereof.

Provided herein is an antibody or antigen-binding fragment herein that selectively binds to one or more snake toxins, comprising a VH CDR1, a VH CDR2, a VH CDR3, a VL CDR1, a VL CDR2, and a VL CDR3, wherein: (i) the VH CDR1 comprises the amino acid sequence FX₂X₃X₄X₅X₆DX₈H, wherein X₂ is selected from N, T, and S; X₃ is selected from F, L; X₄ is selected from R, G, and S; X₅ is selected from N and T; X₆ is selected from Y, F, L; and X₈ is selected from M, I; (ii) the VH CDR2 comprises the amino acid sequence X₁X₂X₃X₄X₅X₆X₇GX₉X₁₀X₁₁X₁₂, wherein X₁ is selected from P, S, and H; X₂ is selected from V, G, and T; X₃ is selected from V, F, L, and I; X₄ is selected from D, G, and A; X₅ is selected from Y, L, and H; X₆ is selected from N, C, R, and T, and S; X₇ is selected from V, F, and G; X₉ is selected from A, E; X₁₀ is selected from Q, H; and X₁₁ is selected from S, D, Y, and H; and X₁₂ is selected from A and E; (iii) the VH CDR3 comprises the amino acid sequence CX₂RGTLYHYTSGSYX₁₅SDAFDIW, wherein X₂ is selected from V and A; and X₁₅ is selected from Y and C (SEQ ID NO: 527); (iv) the VL CDR1 comprises the amino acid sequence X₁ASX₄X₅IX₇X₈X₉LX₁₁, wherein X₁ is selected from Q and R; X₄ is selected from Q and E; X₅ is selected from D, G, T, and S; X₇ is selected from R and S; X₈ is selected from S, D, N, and I; X₉ is selected from N, F, Y, W, and D; and X₁₁ is selected from N, G, and A (SEQ ID NO: 528); (v) the VL CDR2 comprises the amino acid sequence X₁ASX₄X₅X₆X₇, wherein X₁ is selected from G and A; X₄ is selected from N, T, and S; X₅ is selected from L and S; X₆ is selected from Q, L, and E; and X₇ is selected from M and S; and (vi) the VL CDR3 comprises the amino acid sequence CQQSYSTX₈TF, wherein X₈ is selected from I and H (SEQ ID NO: 530); or the VL CDR3 comprises the amino acid sequence CQQX₄X₅X₆X₇PX₉TF, wherein X₄ is selected from A and S; X₅ is selected from N and Y; X₆ is selected from I, T, and S; X₇ is selected from P, F, and T; and X₉ is selected from P, Y, L, and W (SEQ ID NO: 531).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DISCLOSURE OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A provides a pictorial illustration of clone SNEURO_P01_D09 light chain (D09LC) and SNEURO_P01_D09 heavy chain (D09HC) bound to Toxin B.

FIG. 1B provides a pictorial illustration of an α-Bungarotoxin/nAChR Complex (PDB:4UY2) where α9 ECD of nicotinic Acetylcholine Receptor (nAChR) interacts with α-Bungarotoxin at Loop C.

FIB. 1C provides a pictorial illustration of the footprint overlap of Toxin B with D09 (dotted line) or nAChR (solid line).

FIG. 2 is a graph illustrating SNEURO_P01_D09 binding to Mamba-AVI-HIS at 0.8, 4, 20, 100, or 500 nM using Octect HTX BL1.

FIG. 3 is a graph illustrating SNEURO_P01_D09 binding to Taipan-AVI-HIS at 0.8, 4, 20, 100, or 500 nM using Octect HTX BL1.

FIG. 4 is a graph illustrating SNEURO_P01_D09 binding to Cobra-AVI-HIS at 0.8, 4, 20, 100, or 500 nM using Octect HTX BL1.

FIG. 5 is a graph illustrating SNEURO_P01_D09 binding at 25° C. to Mamba-AVI-HIS at 0.8, 4, 20, or 100 nM. KD was determined to be <74 pM.

FIG. 6 is a graph illustrating SNEURO_P01_D09 binding at 25° C. to Taipan-AVI-HIS at 0.8, 4, 20, or 100 nM. KD was determined to be 490 pM.

FIG. 7 is a graph illustrating SNEURO_P01_D09 binding at 25° C. to Cobra-AVI-HIS at 0.8, 4, or 20 nM. KD was determined to be <37 pM.

FIG. 8 is a graph illustrating SNEURO_P01_D09 binding at 37° C. to Mamba-AVI-HIS at 0.8, 4, 20, or 100 nM. KD was determined to be <57 pM.

FIG. 9 is a graph illustrating SNEURO_P01_D09 binding at 37° C. to Taipan-AVI-HIS at 0.8, 4, or 20 nM. KD was determined to be 960 pM.

FIG. 10 is a graph illustrating SNEURO_P01_D09 binding at 37° C. to Cobra-AVI-HIS at 0.8, 4, or 20 nM. KD was determined to be <37 pM.

FIG. 11 illustrates generation of a universal antivenom from toxins derived from just four snake venoms.

FIG. 12. illustrates development progress of broad-spectrum monoclonal antibody candidates against alpha-neurotoxin, dendrotoxin, and PLA2.

FIGS. 13A-F provide structures of Neurotoxin B/Centi-LNX-D9 Fab Complex at 2.1 Å Reveals Mechanism of Neutralization. X-ray crystals of Centi-LNX-D9 were obtained against coastal taipan (FIG. 13A), cape cobra (FIG. 13B), mamba (FIG. 13C), and krait (FIG. 13D). Structures reveal mechanism of broad neutralization through molecular mimicry of native ligand epitope by the VH CDR3 and supporting residues (FIG. 13E). High resolution epitope mapping (FIG. 13F) provides a basis for the broad neutralizing capacity of the Centi-LNX-D09 antibody observed from kinetics and in vivo protection.

FIGS. 14A-B illustrate the results of recombinant toxin challenge in mice. FIG. 14A provides data demonstrating that Centi-D09 (D9 in the figure) provided complete broadly neutralizing protection against recombinant long neurotoxin from black mamba, cape cobra, coastal taipan, and common krait, over two hours. Note: lines for D9-treated animals all overlap at 100% survival. FIG. 14B provides data demonstrating continued complete protection against black mamba, coastal taipan, and cape cobra over twenty-five hours.

FIG. 15 illustrates venom survival proportions with bnAb Centi-D09. Following the LD100's obtained for multiple venoms in mice, live challenge studies demonstrated that Centi-D09 (Centi-D09 in figure) provided complete broadly neutralizing protection against whole venom from monocled cobra (Naja kaouthia), black mamba (Dendroaspis polylepsis), and cape cobra (Naja nivea). Note: lines for D9-treated animals all overlap at 100% survival.

FIG. 16 illustrates the ability of Centi-D09 to bind and neutralize 3FTX snake venom toxins from a diversity of species (phylogeny shown on the left panel). Centi-D09 bound 3FTX long neurotoxins from all species tested (right panel, left column). Centi-D09 protected against in vivo challenge with isolated long neurotoxin from a variety of species (right panel, middle column). Finally, Centi-D09 protected against whole venom challenge in vivo from a diversity of species; and in multiple cases where its protection was incomplete, its effect was shown to be synergistic with PLA2 inhibitor varespladib (right panel, right column).

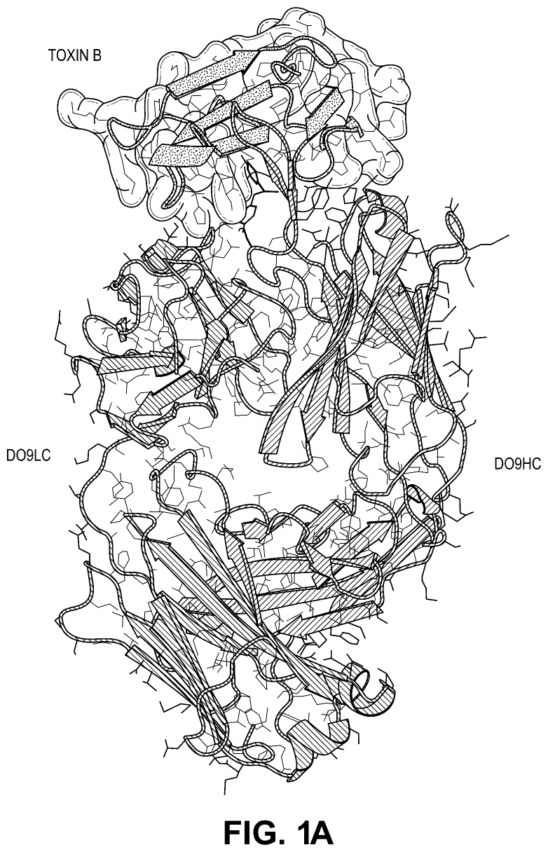

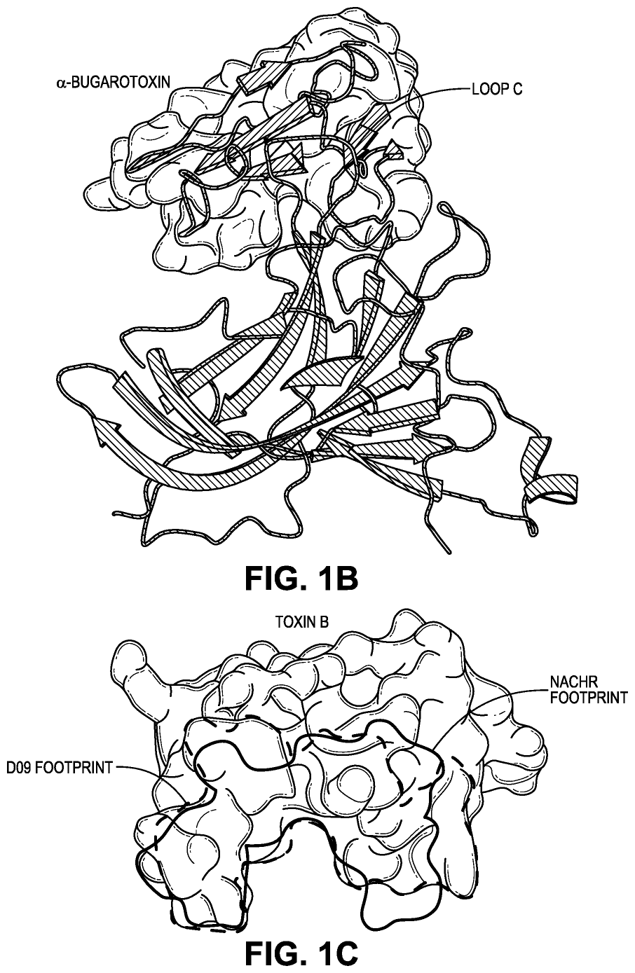

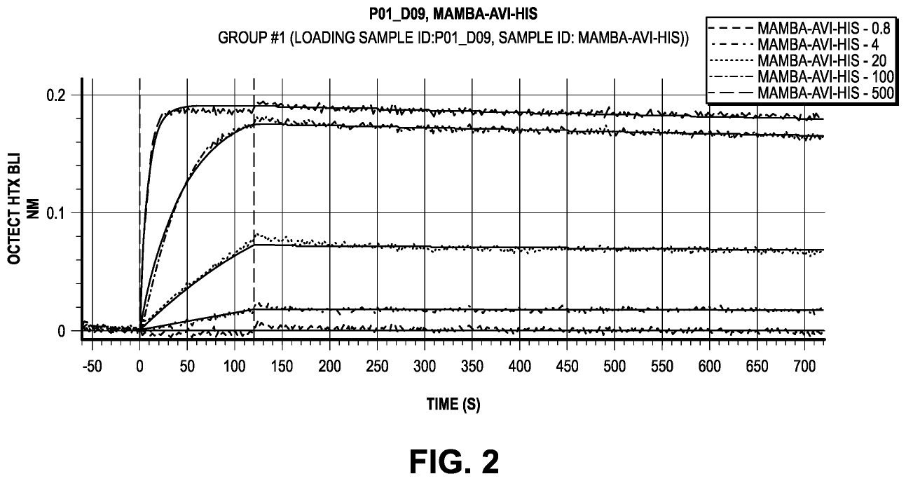

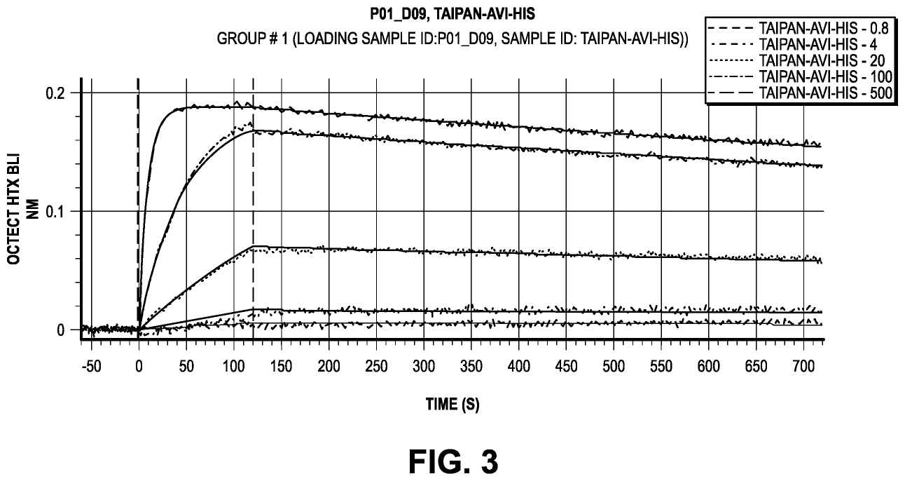

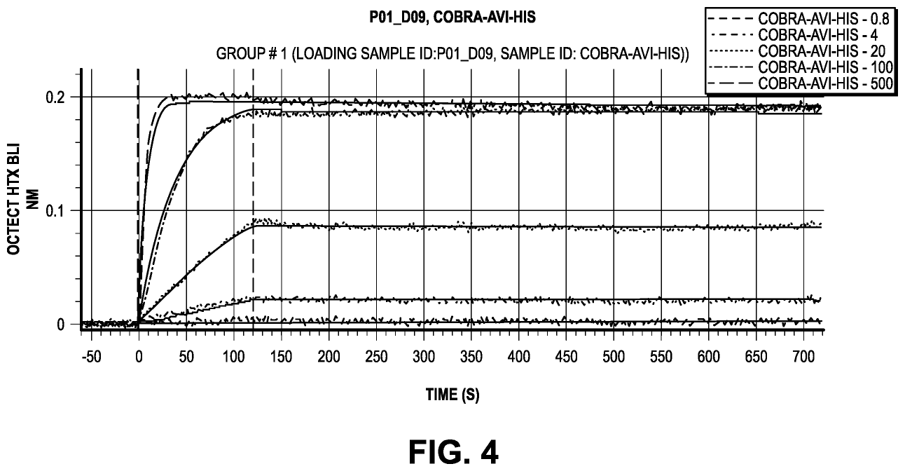

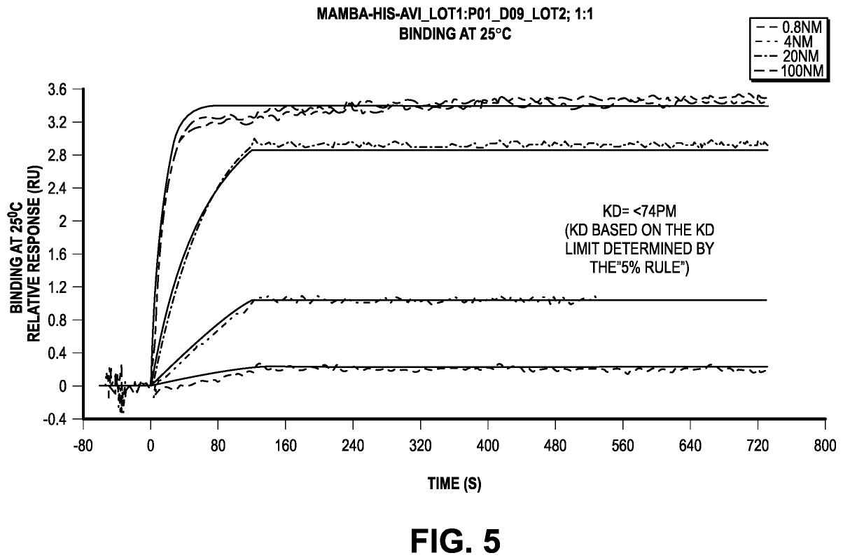

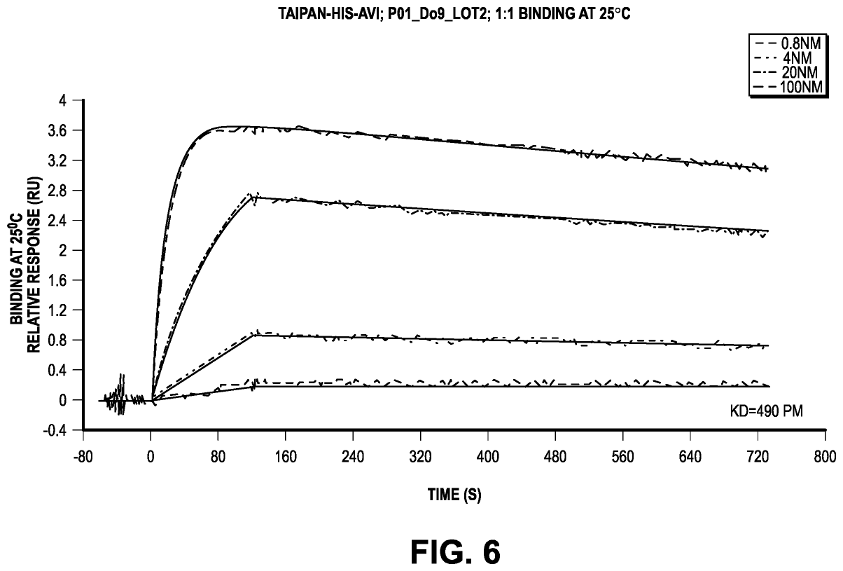

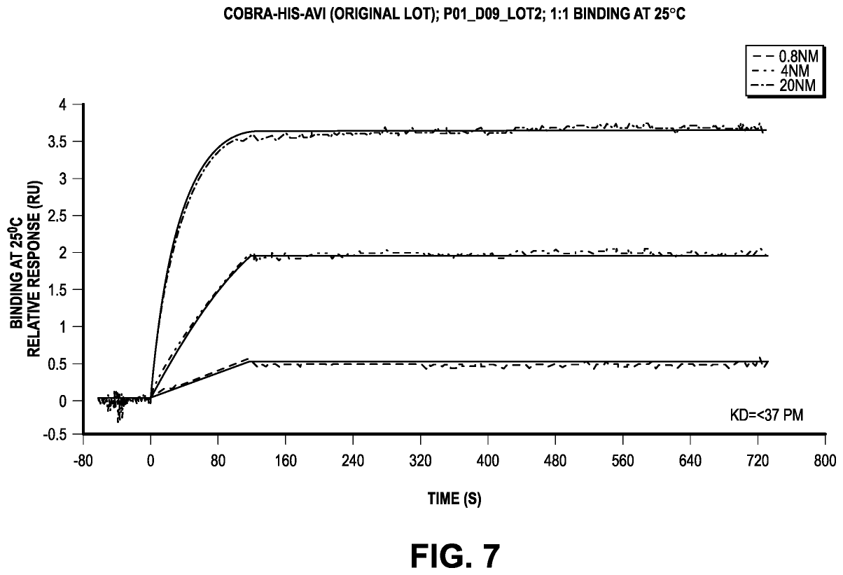

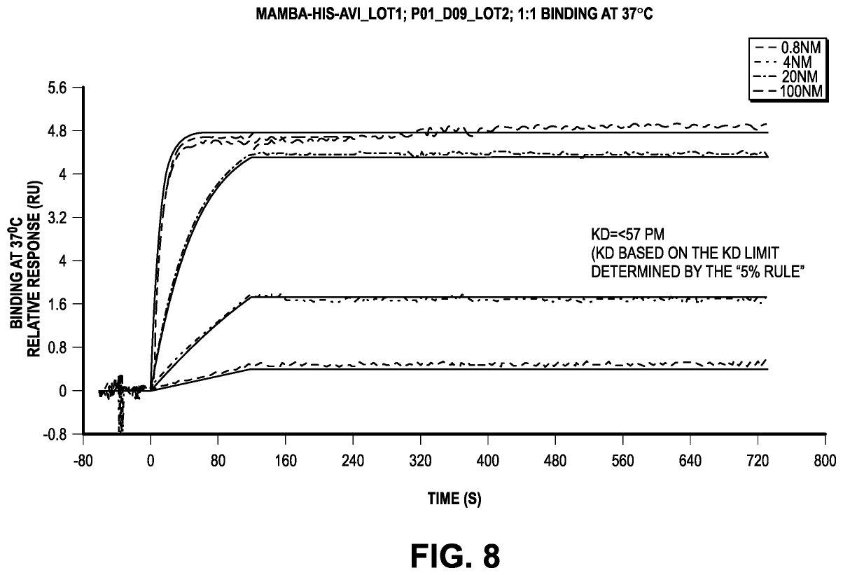

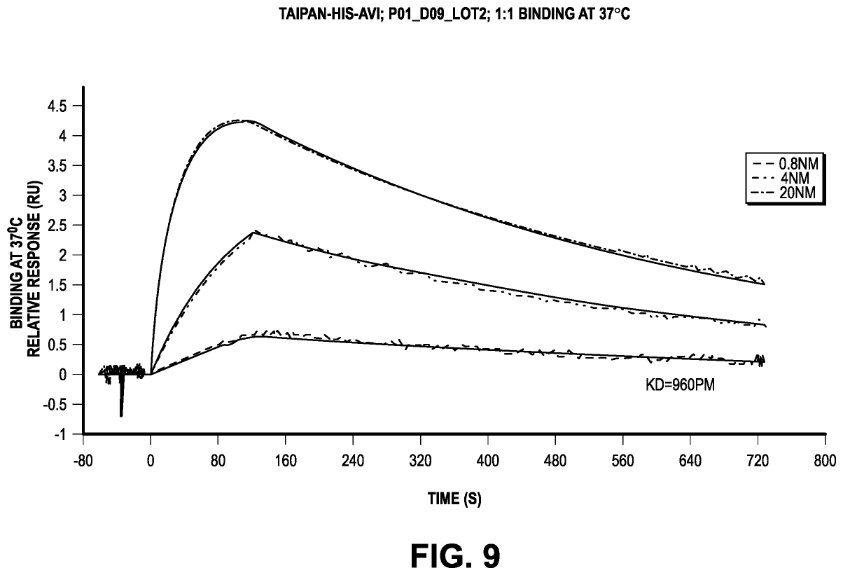

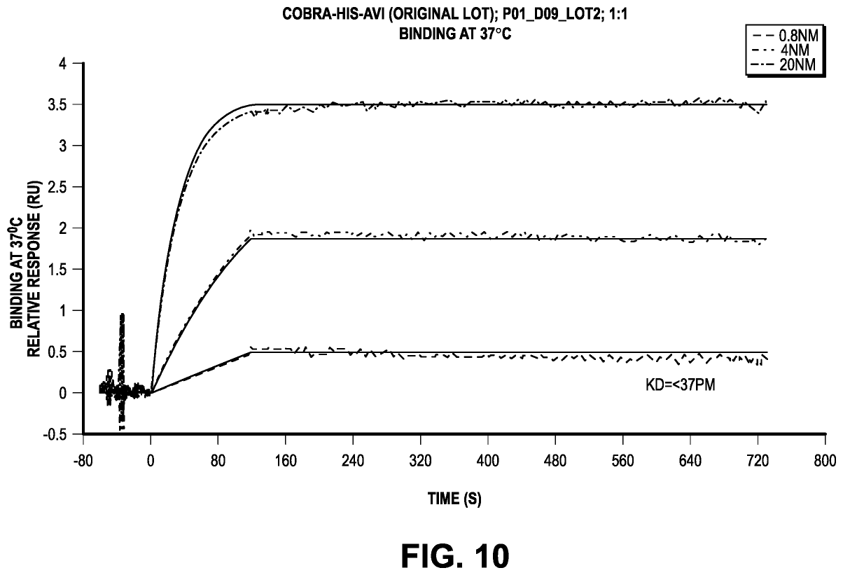

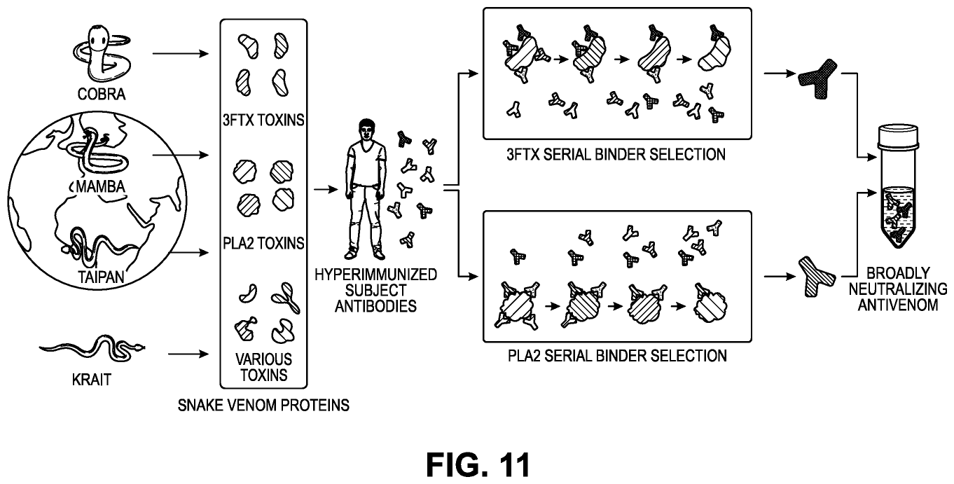

### DETAILED DESCRIPTION

Provided herein are universal anti-venom compositions and methods using the universal anti-venom compositions for treating venomous snake bites. The universal anti-venom compositions can comprise one or more antibodies or antigen-binding fragments. An antibody or antigen binding fragment selectively binds to a snake toxin described herein and comprises a VH CDR1, a VH CDR2, a VH CDR3, VL CDR1, a VL CDR2, and a VL CDR3.

Representative VH CDR3 sequences

An antibody or antigen-binding fragment can comprise a VH CDR3 having an amino acid (AA) sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 1.

TABLE 1
VH CDR3 sequences

| | | SEQ |
|---|---|---|
| | | ID |
| Clone Name | VH CDR3 AA Sequence | NO: |
| PLA2_P01_A04 | CTTDHRRRGLW | 1 |
| PLA2_P01_A08 | CTRRNPGGMDVW | 2 |
| PLA2_P01_B02 | CARLRVGRDYKGMDVR | 3 |
| PLA2_P01_B08 | CTRRNPGGMDVW | 2 |
| PLA2_P01_C02 | CTRRNPGGMDVW | 2 |
| PLA2_P01_C03 | CVTYTYCGGDCYSEDW | 4 |
| PLA2_P01_C04 | CAKVRRGYSYGTDYW | 5 |
| PLA2_P01_D06 | CATSRTFDYW | 6 |
| PLA2_P01_G05 | CALARSYDYYYGVDVW | 7 |
| PLA2_P01_G07 | CARGRRFDPW | 8 |
| PLA2_P01_G12 | CTTDHRRRGLW | 1 |
| KUNITZ_P01_A03 | CSRDYGYYFDFW | 9 |
| KUNITZ_P01_A07 | CATRSVLTGSPIW | 10 |
| KUNITZ_P01_D07 | CAREAVAGTHPQAGDFDLW | 11 |
| KUNITZ_PO1_H05 | CAREAVAGTHPQAGDFDLW | 11 |
| KUNITZ_P01_H07 | CAREAVAGTHPQAGDFDLW | 11 |
| SNEURO_P01_A04 | CVRGTLYHYTSGSYYSDAFDIW | 12 |
| SNEURO_P01_A08 | CVRGTLYHYTSGSYYSDAFDIW | 12 |
| SNEURO_P01_A12 | CARLRVGRDYKGMDVR | 3 |
| SNEURO_P01_B05 | CVRGTLYHYTSGSYYSDAFDIW | 12 |
| SNEURO_P01_B11 | CVRGTLYHYTSGSYYSDAFDIW | 12 |
| (Centi-B11) | | |
| SNEURO_P01_D09 | CVRGTLYHYTSGSYYSDAFDIW | 12 |
| (Centi-D09) | | |
| SNEURO_P01_E06 | CVRGTLYHYTSGSYYSDAFDIW | 12 |
| SNEURO_P01_E09 | CVRGTLYHYTSGSYYSDAFDIW | 12 |
| SNEURO_P01_F07 | CVRGTLYHYTSGSYYSDAFDIW | 12 |
| SNEURO_P01_F09 | CVRGTLYHYTSGSYYSDAFDIW | 12 |
| SNEURO_P01_H02 | CARGTLYHYTSGSYYSDAFDIW | 13 |
| VENM_M03_B12 | CAKAGSYDGSGYNYFDSW | 14 |
| VENM_M12_G05 | CVREGSLTGPGWENWFDPW | 15 |
| VENM_M03_A06 | CARGLRLGELKQW | 16 |
| SNEURO2_P01_H06 | CATRSVLTGSPIW | 10 |
| Centi-D09.0 | CVRGTLYHYTSGSYCSDAFDIW | 327 |
| Centi-D09.1 | CVRGTLYHYTSGSYYSDAFDIW | 328 |
| Centi-D09.2 | CARGTLYHYTSGSYYSDAFDIW | 329 |
| Centi-D09.3 | CVRGTLYHYTSGSYYSDAFDIW | 330 |
| Centi-D09.4 | CVRGTLYHYTSGSYYSDAFDIW | 331 |
| Centi-D09.5 | CVRGTLYHYTSGSYYSDAFDIW | 332 |
| Centi-D09.6 | CVRGTLYHYTSGSYYSDAFDIW | 333 |
| Centi-D09.7 | CVRGTLYHYTSGSYYSDAFDIW | 334 |
| Centi-D09.8 | CVRGTLYHYTSGSYYSDAFDIW | 335 |
| Centi-D09.9 | CVRGTLYHYTSGSYYSDAFDIW | 336 |
| Centi-D09.10 | CVRGTLYHYTSGSYYSDAFDIW | 337 |
| Centi-D09.11 | CVRGTLYHYTSGSYYSDAFDIW | 338 |
| Centi-D09.12 | CVRGTLYHYTSGSYYSDAFDIW | 339 |
| Centi-D09.13 | CVRGTLYHYTSGSYYSDAFDIW | 340 |
| Centi-D09.14 | CVRGTLYHYTSGSYYSDAFDIW | 341 |
| Centi-D09.15 | CVRGTLYHYTSGSYYSDAFDIW | 342 |
| Centi-D09.16 | CVRGTLYHYTSGSYYSDAFDIW | 343 |
| Centi-D09.17 | CVRGTLYHYTSGSYYSDAFDIW | 344 |
| Centi-D09.18 | CVRGTLYHYTSGSYYSDAFDIW | 345 |
| Centi-D09.19 | CVRGTLYHYTSGSYYSDAFDIW | 346 |
| Centi-D09.20 | CVRGTLYHYTSGSYYSDAFDIW | 347 |
| Centi-D09.21 | CVRGTLYHYTSGSYYSDAFDIW | 348 |

### Representative VH CDR1 Sequences

An antibody or antigen-binding fragment can comprise a VH CDR1 having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 2.

TABLE 2
VH CDR1 sequences

| | | SEQ |
|---|---|---|
| | VH CDR1 AA | ID |
| Clone Name | Sequence | NO: |
| PLA2_P01_A04 | FTFSSYAMS | 17 |
| PLA2_P01_A08 | FTLSDSTVH | 18 |
| PLA2_P01_B02 | FTFSSYAMS | 17 |
| PLA2_P01_B08 | FTLSDSTVH | 18 |
| PLA2_P01_C02 | FTLSDSTVH | 18 |
| PLA2_P01_C03 | FTFTNAWMG | 19 |
| PLA2_P01_C04 | FTFSSYAMS | 17 |
| PLA2_P01_D06 | FAFSSYAMS | 20 |
| PLA2_P01_G05 | FTFSGYGMH | 21 |
| PLA2_P01_G07 | FAFSSYAMN | 22 |
| PLA2_P01_G12 | FTFSSYAMS | 17 |
| KUNITZ_P01_A03 | GTFSSDAIT | 23 |
| KUNITZ_P01_A07 | GSINIGNWYN | 24 |
| KUNITZ_P01_D07 | HTFSDYYMH | 25 |
| KUNITZ_P01_H05 | YTFTSYHMH | 26 |
| KUNITZ_P01_H07 | YTFTDHYIH | 27 |
| SNEURO_P01_A04 | FTFSNFDMH | 28 |
| SNEURO_P01_A08 | FTFSNFDMH | 28 |
| SNEURO_P01_A12 | FTFSSYAMS | 17 |
| SNEURO_P01_B05 | FTFSNFDMH | 28 |
| SNEURO_P01_B11 | FTFSNFDMH | 28 |
| (Centi-B11) | | |
| SNEURO_P01_D09 | FTFSNFDMH | 28 |
| (Centi-D09) | | |
| SNEURO_P01_E06 | FTFSNFDMH | 28 |
| SNEURO_P01_E09 | FTFSNFDMH | 28 |
| SNEURO_P01_F07 | FTFSNFDMH | 28 |
| SNEURO_P01_F09 | FTFSNFDMH | 28 |
| SNEURO_P01_H02 | FTFSTFDMH | 29 |
| VENM_M03_B12 | FTFDDFAMH | 30 |
| VENM_M12_G05 | FIVSDYGMH | 31 |
| VENM_M03_A06 | GSFRDYAVS | 32 |
| SNEURO2_P01_H06 | GSINIGNWYN | 24 |
| Centi-D09.0 | FTFSNFDMH | 349 |
| Centi-D09.1 | FTFSNFDMH | 350 |
| Centi-D09.2 | FTFSTFDMH | 351 |
| Centi-D09.3 | FTFSNFDMH | 352 |
| Centi-D09.4 | FTFSNFDMH | 353 |
| Centi-D09.5 | FTFSNFDMH | 354 |
| Centi-D09.6 | FTLSNFDMH | 355 |
| Centi-D09.7 | FTFSNFDMH | 356 |
| Centi-D09.8 | FTFRNFDMH | 357 |
| Centi-D09.9 | FTFSNFDMH | 358 |
| Centi-D09.10 | FTFSNFDMH | 359 |
| Centi-D09.11 | FTFSNYDMH | 360 |
| Centi-D09.12 | FTFSNFDMH | 361 |
| Centi-D09.13 | FTFSNLDMH | 362 |
| Centi-D09.14 | FTFSNFDMH | 363 |
| Centi-D09.15 | FTFSNFDMH | 364 |
| Centi-D09.16 | FTFSNFDMH | 365 |
| Centi-D09.17 | FTFSNFDMH | 366 |
| Centi-D09.18 | FTFSNFDMH | 367 |
| Centi-D09.19 | FTFGNFDMH | 368 |
| Centi-D09.20 | FSFSNFDIH | 369 |
| Centi-D09.21 | FNFSNFDMH | 370 |

### Representative VH CDR2 Sequences

An antibody or antigen-binding fragment can comprise a VH CDR2 having an amino acid sequence that is at least 20%, 24%, 30%, 35%, 40%, 45%, 50%, 55%, 60%, 65%, 70%, 75%, 80%, 85%, 90%, 95%, 98%, 99%, or 100% identical to any one of the following sequences of Table 3.

TABLE 3
VH CDR2 sequences

| | | SEQ |
|---|---|---|
| | VH CDR2 AA | ID |
| Clone Name | Sequence | NO: |
| PLA2_P01_A04 | SAISGSGGSTYYA | 33 |
| PLA2_P01_A08 | AYIRSKVNSYATGYA | 34 |
| PLA2_P01_B02 | SAISGSGGSTYYA | 33 |
| PLA2_P01_B08 | AYIRSKVNSYATGYA | 34 |
| PLA2_P01_C02 | AYIRSKVNSYATGYA | 34 |
| PLA2_P01_C03 | ARIKSKSDGGKTDYA | 35 |
| PLA2_P01_C04 | SAISGSGGSTYYA | 33 |
| PLA2_P01_D06 | SAISGSGGSTYYA | 33 |
| PLA2_P01_G05 | ATISYDGSNKYYA | 36 |
| PLA2_P01_G07 | STVSGSGSTYYA | 37 |
| PLA2_P01_G12 | SAISGSGGSTYYA | 33 |
| KUNITZ_P01_A03 | GWINPNSGGTNYA | 38 |
| KUNITZ_P01_A07 | GEVYHSGSTNYN | 39 |
| KUNITZ_P01_D07 | GRINPDSGATDYA | 40 |
| KUNITZ_P01_H05 | GRINPDSGATDYA | 40 |
| KUNITZ_P01_H07 | GRINPDSGATDYA | 40 |
| SNEURO_P01_A04 | SGLDHSGGAHYA | 41 |
| SNEURO_P01_A08 | SGLDHSGGAHYA | 41 |
| SNEURO_P01_A12 | SAISGSGGSTYYA | 33 |
| SNEURO_P01_B05 | SGLDHSGGAHYA | 41 |
| SNEURO_P01_B11 | SGLDHSGGAHYA | 41 |
| (Centi-B11) | | |
| SNEURO_P01_D09 | SGLDHSGGAHYA | 41 |
| (Centi-D9) | | |
| SNEURO_P01_E06 | SGLDHSGGAHYA | 41 |
| SNEURO_P01_E09 | SGLDHSGGAHYA | 41 |
| SNEURO_P01_F07 | SGLDHSGGAHYA | 41 |
| SNEURO_P01_F09 | SGLDHSGGAHYA | 41 |
| SNEURO_P01_H02 | STFDHSGGAHYA | 42 |
| VENM_M03_B12 | SSISWNSVNIDYA | 43 |
| VENM_M12_G05 | AVMSYDGSQKFYS | 44 |
| VENM_M03_A06 | GGIIPMFGRGEYT | 45 |
| SNEURO2_P01_H06 | GEVYHSGSTNYN | 39 |
| Centi-D09.0 | SGLDHSGGAHYA | 371 |
| Centi-D09.1 | SGLDHSGGAHYA | 372 |
| Centi-D09.2 | STFDHSGGAHYA | 373 |
| Centi-D09.3 | SGVDHSGGAHYA | 374 |
| Centi-D09.4 | PGLDHSGGAHYA | 375 |
| Centi-D09.5 | SGLDHNGGAHYA | 376 |
| Centi-D09.6 | SGLDHSGGAHYA | 377 |
| Centi-D09.7 | SGLDHRGGAHYA | 378 |
| Centi-D09.8 | SVLDYSGGEHYA | 379 |
| Centi-D09.9 | SGLDLSGGAHYA | 380 |
| Centi-D09.10 | SGLAHSGGAHYA | 381 |
| Centi-D09.11 | SGLDHSGGAHYA | 382 |
| Centi-D09.12 | SGLDHSVGAHYA | 383 |
| Centi-D09.13 | SGLDHSGGAHYA | 384 |
| Centi-D09.14 | HGLDHSGGAHYA | 385 |
| Centi-D09.15 | SGLDHSGGAQYA | 386 |
| Centi-D09.16 | SGLDHSGGAHHA | 387 |
| Centi-D09.17 | SGIDHSGGAHYA | 388 |
| Centi-D09.18 | SGLDHSGGAHDA | 389 |
| Centi-D09.19 | SGLDHTGGAHSE | 390 |
| Centi-D09.20 | SGLGLCFGAHYA | 391 |
| Centi-D09.21 | SGLDHSGGAHYA | 392 |

### Representative VL CDR1 Sequences

An antibody or antigen-binding fragment can comprise a VL CDR1 having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 4.

TABLE 4
VL CDR1 sequences

| | | SEQ |
|---|---|---|
| | VL CDR1 AA | ID |
| Clone Name | Sequence | NO: |
| PLA2_P01_A04 | QASQDISNYLN | 46 |
| PLA2_P01_A08 | SGSSSNIGSNYVY | 47 |
| PLA2_P01_B02 | RASQNINNYLN | 48 |
| PLA2_P01_B08 | RASQGIRNDLG | 49 |
| PLA2_P01_C02 | RASRSIRNDLA | 50 |
| PLA2_P01_C03 | RASQSISSWLA | 51 |
| PLA2_P01_C04 | RASQDVSSWLA | 52 |
| PLA2_P01_D06.1 | GGDNIGDKRVH | 53 |
| and | | |
| PLA2_P01_D06.2 | | |
| PLA2_P01_G05 | RASQDIRNDLG | 54 |
| PLA2_P01_G07 | RASQSISSYLN | 55 |
| PLA2_P01_G12 | QASQDISNYLN | 46 |
| KUNITZ_P01_A03.1 | GGDRLHFKTVD | 56 |
| and | | |
| KUNITZ_P01_A03.2 | | |
| KUNITZ_P01_A07 | KSSQSVLDSS | 57 |
| | KNKNYLA | |
| KUNITZ_P01_D07 | RASQSINNWLA | 58 |
| KUNITZ_P01_H05.1 | GGNNIGSKSVH | 59 |
| and | | |
| KUNITZ_P01_H05.2 | | |
| KUNITZ_P01_H07.1 | TGSSSNIGA | 60 |
| and | GFDVH | |
| KUNITZ_P01_H07.2 | | |
| SNEURO_P01_A04 | RASQSISSYLN | 55 |
| SNEURO_P01_A08 | RASQSISSYLN | 55 |
| SNEURO_P01_A12 | RASQSISTYLN | 61 |
| SNEURO_P01_B05 | RASQSISSFLN | 62 |
| SNEURO_P01_B11 | RASQGISDNLN | 63 |
| (Centi-B11) | | |
| SNEURO_P01_D09 | QASQDISNYLN | 46 |
| (Centi-D9) | | |
| SNEURO_P01_E06 | RASETISIYLN | 64 |
| SNEURO_P01_E09 | RASQGISSWLA | 65 |
| SNEURO_P01_F07 | RASQDIRNDLG | 54 |
| SNEURO_P01_F09 | RASQSISSWLA | 51 |
| SNEURO_P01_H02 | RASQGISSWLA | 65 |
| VENM_M03_B12 | RASQSISSWLA | 51 |
| VENM_M12_G05 | SGGSSNIGDYDVS | 66 |
| VENM_M03_A06 | RASQGVSSWLA | 67 |
| SNEURO2_P01_H06 | KSSQSVLDSSKN | 57 |
| | KNYLA | |
| Centi-D09.0 | RASETISIYLN | 393 |
| Centi-D09.1 | RASQGISSWLA | 394 |
| Centi-D09.2 | QASQDISNYLN | 395 |
| Centi-D09.3 | RASQSISSYLN | 396 |
| Centi-D09.4 | RASQGISSWLA | 397 |
| Centi-D09.5 | RASQSISSYLN | 398 |
| Centi-D09.6 | RASQSISSFLN | 399 |
| Centi-D09.7 | RASQGISDNLN | 400 |
| Centi-D09.8 | RASQDIRNDLG | 401 |
| Centi-D09.9 | RASQSISSWLA | 402 |
| Centi-D09.10 | RASQSISSYLN | 403 |
| Centi-D09.11 | RASQGIRNDLN | 404 |

### Representative VL CDR2 Sequences

An antibody or antigen-binding fragment can comprise a VL CDR2 having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 5.

TABLE 5
VL CDR2 sequences

| | | SEQ |
|---|---|---|
| | VL CDR2 AA | ID |
| Clone Name | Sequence | NO: |
| PLA2_P01_A04 | GASNLHS | 68 |
| PLA2_P01_A08 | RNNQRPS | 69 |
| PLA2_P01_B02 | DASNLET | 70 |
| PLA2_P01_B08 | AASTLQS | 71 |
| PLA2_P01_C02 | DASNLET | 70 |
| PLA2_P01_C03 | KASSLES | 72 |
| PLA2_P01_C04 | AASTLQS | 71 |
| PLA2_P01_D06.1 | DDADRPT | 73 |
| and | | |
| PLA2_P01_D06.2 | | |
| PLA2_P01_G05 | AASTLQS | 71 |
| PLA2_P01_G07 | AASSLQS | 74 |
| PLA2_P01_G12 | GASNLHS | 68 |
| KUNITZ_P01_A03.1 | GDSDRPS | 75 |
| and | | |
| KUNITZ_P01_A03.2 | | |
| KUNITZ_P01_A07 | WASIRES | 76 |
| KUNITZ_P01_D07 | KASTLES | 77 |
| KUNITZ_P01_H05.1 | DDTNRPS | 78 |
| and | | |
| KUNITZ_P01_H05.2 | | |
| KUNITZ_P01_H07.1 | GNSNRPS | 79 |
| and | | |
| KUNITZ_P01_H07.2 | | |
| SNEURO_P01_A04 | AASSLQS | 74 |
| SNEURO_P01_A08 | AASSLQS | 74 |
| SNEURO_P01_A12 | AASSLQS | 74 |
| SNEURO_P01_B05 | AASSLQS | 74 |
| SNEURO_P01_B11 | AASTLQS | 71 |
| (Centi-B11) | | |
| SNEURO_P01_D09 | AASSLES | 80 |
| (Centi-D9) | | |
| SNEURO_P01_E06 | AASSLQS | 74 |
| SNEURO_P01_E09 | AASNLQM | 81 |
| SNEURO_P01_F07 | GASTLQS | 82 |
| SNEURO_P01_F09 | AASSLQS | 74 |
| SNEURO_P01_H02 | AASSLQS | 74 |
| VENM_M03_B12 | KASSLES | 72 |
| VENM_M12_G05 | RNNQRPS | 69 |
| VENM_M03_A06 | ATSRLQS | 83 |
| SNEURO2_P01_H06 | WASIRES | 76 |
| Centi-D09.0 | AASSLQS | 405 |
| Centi-D09.1 | AASSLQS | 406 |
| Centi-D09.2 | AASSLES | 407 |
| Centi-D09.3 | AASSLQS | 408 |
| Centi-D09.4 | AASNLQM | 409 |
| Centi-D09.5 | AASSLQS | 410 |
| Centi-D09.6 | AASSLQS | 411 |
| Centi-D09.7 | AASTLQS | 412 |
| Centi-D09.8 | GASTLQS | 413 |
| Centi-D09.9 | AASSLQS | 414 |
| Centi-D09.10 | AASTLQS | 415 |
| Centi-D09.11 | AASNSLS | 416 |

### Representative VL CDR3 Sequences

An antibody or antigen-binding fragment can comprise a VL CDR3 having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 6.

TABLE 6
VL CDR3 sequences

| | | SEQ |
|---|---|---|
| | VL CDR3 AA | ID |
| Clone Name | Sequence | NO: |
| PLA2 P01 A04 | CQQSYSTPLTF | 84 |
| PLA2 P01 A08 | CAAWDDRLAGYVF | 85 |
| PLA2 P01 B02 | CQQSHITPLTF | 86 |
| PLA2 P01 B08 | CQQANSYPVTF | 87 |
| PLA2 P01 C02 | CORSYTTPPTF | 88 |
| PLA2 P01 C03 | CQQAKSFPVSF | 89 |
| PLA2 P01 C04 | CQQHYTTPPTF | 90 |
| PLA2 P01 D06.1 | CQVYDRASNHVVF | 91 |
| and | | |
| PLA2 P01 D06.2 | | |
| PLA2 P01 G05 | CQQANSFPLTF | 92 |
| PLA2 P01 G07 | CQQSYSTPLTF | 84 |
| PLA2 P01 G12 | CQQSYSTPLTF | 84 |
| KUNITZ P01 A03.1 | CQVWDHNGDGRVF | 93 |
| and | | |
| KUNITZ P01 A03.2 | | |
| KUNITZ P01 A07 | CQQYYGTPYTF | 94 |
| KUNITZ P01 D07 | CQQYNNHSRTF | 95 |
| KUNITZ P01 H05.1 | CQLWDSSGEHWVF | 96 |
| and | | |
| KUNITZ P01 H05.2 | | |
| KUNITZ P01 H07.1 | CRSYDSSLSGSVF | 97 |
| and | | |
| KUNITZ P01 H07.2 | | |
| SNEURO P01 A04 | CQQSYSTPLTF | 84 |
| SNEURO P01 A08 | CQQSYSTHTF | 98 |
| SNEURO P01 A12 | CQQSYSTPYTF | 99 |
| SNEURO P01 B05 | CQQSYSPPLTF | 100 |
| SNEURO P01 B11 | CQQANSFPLTF | 92 |
| (Centi-B11) | | |
| SNEURO_P01 D09 | CQQANSFPYTF | 101 |
| (Centi-D9) | | |
| SNEURO P01 E06 | CQQSYSTPWTF | 102 |
| SNEURO P01 E09 | CQQSYTTPLTF | 103 |
| SNEURO P01 F07 | CQQSYITPLTF | 104 |
| SNEURO P01 F09 | CQQSYSTITF | 105 |
| SNEURO P01 H02 | CQQANSFPLTF | 92 |
| VENM M03 B12 | CQQYHSYPLTF | 106 |
| VENM M12 G05 | CAAWDDSLNGRVF | 107 |
| VENM M03 A06 | CQQSYFTPVTF | 108 |
| SNEURO2 P01 H06 | CQQYYGTPYTF | 94 |
| Centi-D09.0 | CQQSYSTPWTF | 417 |
| Centi-D09.1 | CQQANSFPLTF | 418 |
| Centi-D09.2 | CQQANSFPYTF | 419 |
| Centi-D09.3 | CQQSYSTPLTF | 420 |
| Centi-D09.4 | CQQSYTTPLTF | 421 |
| Centi-D09.5 | CQQSYSTHTF | 422 |
| Centi-D09.6 | CQQSYSPPLTF | 423 |
| Centi-D09.7 | CQQANSFPLTF | 424 |
| Centi-D09.8 | CQQSYITPLTF | 425 |
| Centi-D09.9 | CQQSYSTITF | 426 |
| Centi-D09.10 | CQQANSFPPTF | 427 |
| Centi-D09.11 | CQQANSFPLTF | 428 |

### Representative Combinations of CDRs

In one instance, an antibody or antigen binding fragment that selectively binds to a snake toxin or a combination of snake toxins comprises one of the following combinations of CDRs:

TABLE 7
Combinations of CDRS

| | VH | VH | VH | VL | VL | VL |
|---|---|---|---|---|---|---|
| | CDR1 | CDR2 | CDR3 | CDR1 | CDR2 | CDR3 |
| | SEQ ID | SEQ ID | SEQ ID | SEQ ID | SEQ ID | SEQ ID |
| Clone Name | NO: | NO: | NO: | NO: | NO: | NO: |
| PLA2_P01_A04 | 17 | 33 | 1 | 46 | 68 | 84 |
| PLA2_P01_A08 | 18 | 34 | 2 | 47 | 69 | 85 |
| PLA2_P01_B02 | 17 | 33 | 3 | 48 | 70 | 86 |
| PLA2_P01_B08 | 18 | 34 | 2 | 49 | 71 | 87 |
| PLA2_P01_C02 | 18 | 34 | 2 | 50 | 70 | 88 |
| PLA2_P01_C03 | 19 | 35 | 4 | 51 | 72 | 89 |
| PLA2_P01_C04 | 17 | 33 | 5 | 52 | 71 | 90 |
| PLA2_P01_D06.1 | 20 | 33 | 6 | 53 | 73 | 91 |
| and | | | | | | |
| PLA2_P01_D06.2 | | | | | | |
| PLA2_P01_G05 | 21 | 36 | 7 | 54 | 71 | 92 |
| PLA2_P01_G07 | 22 | 37 | 8 | 55 | 74 | 84 |
| PLA2_P01_G12 | 17 | 33 | 1 | 46 | 68 | 84 |
| KUNITZ_P01_A03.1 | 23 | 38 | 9 | 56 | 75 | 93 |
| and | | | | | | |
| KUNITZ_P01_A03.2 | | | | | | |
| KUNITZ_P01_A07 | 24 | 39 | 10 | 57 | 76 | 94 |
| KUNITZ_P01_D07 | 25 | 40 | 11 | 58 | 77 | 95 |
| KUNITZ_P01_H05.1 | 26 | 40 | 11 | 59 | 78 | 96 |
| and | | | | | | |
| KUNITZ_P01_H05.2 | | | | | | |
| KUNITZ_P01_H07.1 | 27 | 40 | 11 | 60 | 79 | 97 |
| and | | | | | | |
| KUNITZ_P01_H07.2 | | | | | | |
| SNEURO_P01_A04 | 28 | 41 | 12 | 55 | 74 | 84 |
| SNEURO_P01_A08 | 28 | 41 | 12 | 55 | 74 | 98 |
| SNEURO_P01_A12 | 17 | 33 | 3 | 61 | 74 | 99 |
| SNEURO_P01_B05 | 28 | 41 | 12 | 62 | 74 | 100 |
| SNEURO_P01_B11 | 28 | 41 | 12 | 63 | 71 | 92 |
| (Centi-B11) | | | | | | |
| SNEURO_P01_D09 | 28 | 41 | 12 | 46 | 80 | 101 |
| (Centi-D9) | | | | | | |
| SNEURO_P01_E06 | 28 | 41 | 12 | 64 | 74 | 102 |
| SNEURO_P01_E09 | 28 | 41 | 12 | 65 | 81 | 103 |
| SNEURO_P01_F07 | 28 | 41 | 12 | 54 | 82 | 104 |
| SNEURO_P01_F09 | 28 | 41 | 12 | 51 | 74 | 105 |
| SNEURO_P01_H02 | 29 | 42 | 13 | 65 | 74 | 92 |
| VENM_M03_B12 | 30 | 43 | 14 | 51 | 72 | 106 |
| VENM_M12_G05 | 31 | 44 | 15 | 66 | 69 | 107 |
| VENM_M03_A06 | 32 | 45 | 16 | 67 | 83 | 108 |
| SNEURO2_P01_H06 | 24 | 39 | 10 | 57 | 76 | 94 |

An antibody or antigen-binding fragment can be optimized to increase binding affinity. In one embodiment, such an antibody or antigen-binding fragment can have a combination of heavy chain CDRs as set forth in Table 7A:

TABLE 7A
Optimized VH CDR combinations

| | VH CDR1 | VH CDR2 | VH CDR3 |
|---|---|---|---|
| Clone | SEQ ID NO: | SEQ ID NO: | SEQ ID NO: |
| Centi-D09.0 | 349 | 371 | 327 |
| Centi-D09.1 | 350 | 372 | 328 |
| Centi-D09.2 | 351 | 373 | 329 |
| Centi-D09.3 | 352 | 374 | 330 |
| Centi-D09.4 | 353 | 375 | 331 |
| Centi-D09.5 | 354 | 376 | 332 |
| Centi-D09.6 | 355 | 377 | 333 |
| Centi-D09.7 | 356 | 378 | 334 |
| Centi-D09.8 | 357 | 379 | 335 |
| Centi-D09.9 | 358 | 380 | 336 |
| Centi-D09.10 | 359 | 381 | 337 |
| Centi-D09.11 | 360 | 382 | 338 |
| Centi-D09.12 | 361 | 383 | 339 |
| Centi-D09.13 | 362 | 384 | 340 |
| Centi-D09.14 | 363 | 385 | 341 |
| Centi-D09.15 | 364 | 386 | 342 |
| Centi-D09.16 | 365 | 387 | 343 |
| Centi-D09.17 | 366 | 388 | 344 |
| Centi-D09.18 | 367 | 389 | 345 |
| Centi-D09.19 | 368 | 390 | 346 |
| Centi-D09.20 | 369 | 391 | 347 |
| Centi-D09.21 | 370 | 392 | 348 |

An antibody or antigen-binding fragment can be optimized to increase binding affinity. In one embodiment, such an antibody or antigen-binding fragment can have a combination of light chain CDRs as set forth in Table 7B:

TABLE 7B
Optimized VL CDR combinations

| | VL CDR1 | VL CDR2 | VL CDR3 |
|---|---|---|---|
| Clone | SEQ ID NO: | SEQ ID NO: | SEQ ID NO: |
| Centi-D09.0 | 393 | 405 | 417 |
| Centi-D09.1 | 394 | 406 | 418 |
| Centi-D09.2 | 395 | 407 | 419 |
| Centi-D09.3 | 396 | 408 | 420 |
| Centi-D09.4 | 397 | 409 | 421 |
| Centi-D09.5 | 398 | 410 | 422 |
| Centi-D09.6 | 399 | 411 | 423 |
| Centi-D09.7 | 400 | 412 | 424 |
| Centi-D09.8 | 401 | 413 | 425 |
| Centi-D09.9 | 402 | 414 | 426 |
| Centi-D09.10 | 403 | 415 | 427 |
| Centi-D09.11 | 404 | 416 | 428 |

### Representative FW-H1 Sequences

An antibody or antigen-binding fragment can comprise a VH framework (FW) 1 (FW-H1) having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 8:

TABLE 8
FW-H1 sequences

| | | SEQ |
|---|---|---|
| | | ID |
| Clone Name | FW-H1 AA Sequence | NO |
| PLA2_P01_A04 | QVTLKESGGGVVQPGRSLRLSCAASG | 109 |
| PLA2_P01_A08 | EVQLVESGGGLVQPGGSLRLSCAASG | 110 |
| PLA2_P01_B02 | EVQLVESGGGVVQPGRSLRLSCAASG | 111 |
| PLA2_P01_B08 | EVQLVESGGDLVQPGGSLRLSCAASG | 112 |
| PLA2_P01_C02 | QVTLKESGGGLVQPGGSLRLSCAASG | 113 |
| PLA2_P01_C03 | QVTLKESGGGLVKPGGSLRLSCAASG | 114 |
| PLA2_P01_C04 | XVQLQESGGGVVQPGRSLRLSCAGSG | 115 |
| PLA2_P01_D06 | QVXLQESGGGLVQPGGSLRLSCAASG | 116 |
| PLA2_P01_G05 | HVTLKESGGGVVQPGRSLRLSCAASG | 117 |
| PLA2_P01_G07 | EVQLVESGGGLVQPGGSLRLSCAASG | 110 |
| PLA2_P01_G12 | QVTLKESGGGVVQPGRSLRLSCAASG | 109 |
| KUNITZ_P01_A03 | EVQLVESGAEVKKPGASVKVSCKASG | 118 |
| KUNITZ_P01_A07 | QVQLVESGPGLVAPSGTLSLTCTVSG | 119 |
| KUNITZ_P01_D07 | QVQLQQSGAEVKKPGASVKVSCKASG | 120 |
| KUNITZ_P01_H05 | QVQLVQSGAEVKEPGASVKVSCKASG | 121 |
| KUNITZ_P01_H07 | QVQLVQSGAEVKKPGASMKVSCKASG | 122 |
| SNEURO_P01_A04 | QVQLQESGGGLVQPGGSLRLSCAASG | 123 |
| SNEURO_P01_A08 | QVQLQQSGGGLVQPGGSLRLSCAASG | 124 |
| SNEURO_P01_A12 | EVQLVESGGGLVQPGGSLRLSCAASG | 110 |
| SNEURO_P01_B05 | QVQLQESGGGLVQPGGSLRLSCAASG | 123 |
| SNEURO_P01_B11 | QVQLQESGGGLVQPGGSLRLSCAASG | 123 |
| (Centi-B11) | | |
| SNEURO_P01_D09 | EVQLVESGGGFVQPGGSLRLSCAASG | 125 |
| (Centi-D9) | | |
| SNEURO_P01_E06 | QVTLKESGGGLVQPGGSLRLSCAASG | 113 |
| SNEURO_P01_E09 | QVTLKESGGGLVQPGGSLRLSCAASG | 113 |
| SNEURO_P01_F07 | QVQLVQSGGGLVQPGGSLRLSCAASG | 126 |
| SNEURO_P01_F09 | QVQLQESGGGLVQPGGSLRLSCAASG | 123 |
| SNEURO_P01_H02 | EVQLVESGGDLVQPGGSLRLSCAASG | 112 |
| VENM_M03_B12 | EVQLVESGGGLVQPGGSLRLSCAASG | 110 |
| VENM_M12_G05 | QVQLVQSGGGVVQPGSSLRLSCEASG | 127 |
| VENM_M03_A06 | EVQLLXSAAEVKRPGSSVRVSCKVSG | 128 |
| SNEURO2_P01_H06 | QVQLVESGPGLVAPSGTLSLTCTVSG | 119 |

### Representative FW-H2 Sequences

An antibody or antigen-binding fragment can comprise a VH framework (FW) 2 (FW-H2) having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 9:

TABLE 9
FW-H2 sequences

| | | SEQ |
|---|---|---|
| | FW-H2 AA | ID |
| Clone Name | Sequence | NO |
| PLA2_P01_A04 | WARXAPGKGLEWV | 129 |
| PLA2_P01_A08 | WVRQASGKGLERV | 130 |
| PLA2_P01_B02 | WVRQAPGKGLEWV | 131 |
| PLA2_P01_B08 | WVRQASGKGLERV | 130 |
| PLA2_P01_C02 | WVRQASGKGLERV | 130 |
| PLA2_P01_C03 | WVRQVPGTGLEWV | 132 |
| PLA2_P01_C04 | WVRQAPGKGLEWV | 131 |
| PLA2_P01_D06 | WVRQAPGKGLEWV | 131 |
| PLA2_P01_G05 | WVRQAPGKGLEWV | 131 |
| PLA2_P01_G07 | WVRQAPGKGLEWV | 131 |
| PLA2_P01_G12 | WARXAPGKGLEWV | 129 |
| KUNITZ_P01_A03 | WVRQAPGQGLEWM | 133 |
| KUNITZ_P01_A07 | WVRQSPGKGLEWI | 134 |
| KUNITZ_P01_D07 | WVRQAPGQGLEWM | 133 |
| KUNITZ_P01_H05 | WVRQAPGQGLEWM | 133 |
| KUNITZ_P01_H07 | WVRQAPGQGLEWM | 133 |
| SNEURO_P01_A04 | WVRQSPGKGLEWV | 135 |
| SNEURO_P01_A08 | WVRQSPGKGLEWV | 135 |
| SNEURO_P01_A12 | WVRQAPGKGLEWV | 131 |
| SNEURO_P01_B05 | WVRQSPGKGLEWV | 135 |
| SNEURO_P01_B11 | WVRQSPGKGLEWV | 135 |
| (Centi-B11) | | |
| SNEURO_P01_D09 | WVRQSPGKGLEWV | 135 |
| (Centi-D9) | | |
| SNEURO_P01_E06 | WVRQSPGKGLEWV | 135 |
| SNEURO_P01_E09 | WVRQSPGKGLEWV | 135 |
| SNEURO_P01_F07 | WVRQSPGKGLEWV | 135 |
| SNEURO_P01_F09 | WVRQSPGKGLEWV | 135 |
| SNEURO_P01_H02 | WVRQSPGKGLEWV | 135 |
| VENM_M03_B12 | WVRQAPGKSLEWV | 136 |
| VENM_M12_G05 | WVRQAPGKGLEWV | 131 |
| VENM_M03_A06 | WVRQAPGQGPEWM | 137 |
| SNEURO2_P01_H06 | WVRQSPGKGLEWI | 134 |

### Representative FW-H3 Sequences

An antibody or antigen-binding fragment can comprise a VH framework (FW) 3 (FW-H3) having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 10:

TABLE 10
FW-H3 sequences

| | | SEQ |
|---|---|---|
| | | ID |
| Clone Name | FW-H3 AA Sequence | NO |
| PLA2_P01_A04 | DSVRGRFTISRDNSKNTLYLQMNSLRAEDTAVYY | 138 |
| PLA2_P01_A08 | ASVKGRFTISRDDSKNTAYLQMNSLKIEDTAVYY | 139 |
| PLA2_P01_B02 | DSVKGRFTISRDNSKNMLYLQMNSLRAEDTALYY | 140 |
| PLA2_P01_B08 | ASVKGRFTISRDDSKNTAYLQMNSLKIEDTAVYY | 139 |
| PLA2_P01_C02 | ASVKGRFTISRDDSKNTAYLQMNSLKIEDTAVYY | 139 |
| PLA2_P01_C03 | APVKGRFTISRDDSQNTLYLQMNSLRTEDSAVYY | 141 |
| PLA2_P01_C04 | DSVKGRFTISRDNSKNTLYLQMNSLRAEDTAVYY | 142 |
| PLA2_P01_D06 | DSVKGRFTISRDNSKNTLYLQMNSLRAEDTAVYY | 142 |
| PLA2_P01_G05 | DSVKGRFTISRDNSKNTLYLQMNSLRAEDTAVYY | 142 |
| PLA2_P01_G07 | DSVKGRFTISRDNSKNTLSLQMNGLRAEDTAVYY | 143 |
| PLA2_P01_G12 | DSVRGRFTISRDNSKNTLYLQMNSLRAEDTAVYY | 138 |
| KUNITZ_P01_A03 | QKFQGRVTMTRDTSTSTVYMELSSLRSEDTAVYY | 144 |
| KUNITZ_P01_A07 | PSLESRVTISLDTSKNQFSLKLTSATAADTAVYY | 145 |
| KUNITZ_P01_D07 | QHFQGRVTMTRDTSTSTVYMELSSLRSEDTAVYY | 146 |
| KUNITZ_P01_H05 | QHFQGRVTMTRDTSTSTVYMELSSLRSEDTAVYY | 146 |
| KUNITZ_P01_H07 | QHFQGRVTMTRDTSTSTVYMELSSLRSEDTAVYY | 146 |
| SNEURO_P01_A04 | GSVKGRFTISREDAKNSLDLQMNNLRVDDTAVYF | 147 |
| SNEURO_P01_A08 | GSVKGRFTISREDAKNSLDLQMNNLR VDDTAVYF | 147 |
| SNEURO_P01_A12 | DSVKGRFTTSRDNAKNSLYLQMNSLRAEDTALYY | 148 |
| SNEURO_P01_B05 | GSVKGRFTISREDAKNSLDLQMNNLRVDDTAVYF | 147 |
| SNEURO_P01_B11 | GSVKGRFTISREDAKNSLDLQMNNLRVDDTAVYF | 147 |
| (Centi-B11) | | |
| SNEURO_P01_D09 | GSVKGRFTISREDAKNSLDLQMNNLR VDDTAVYF | 147 |
| (Centi-D9) | | |
| SNEURO_P01_E06 | GSVKGRFTISREDAKNSLDLQMNNLR VDDTAVYF | 147 |
| SNEURO_P01_E09 | GSVKGRFTISREDAKNSLDLQMNNLRVDDTAVYF | 147 |
| SNEURO_P01_F07 | GSVKGRFTISREDAKNSLDLQMNNLRVDDTAVYF | 147 |
| SNEURO_P01_F09 | GSVKGRFTISREDAKNSLDLQMNNLRVDDTAVYF | 147 |
| SNEURO_P01_H02 | ASVKGRFTISREAGKHSLDLQMNSLRVDDTAVYF | 149 |
| VENM_M03_B12 | DSVKGRFTISRDNAKSSLFLQMSSLRVEDTALYY | 150 |
| VENM_M12_G05 | GSVKGRFTISRDISNNTLFLQMNDLRPEDTAVYH | 151 |
| VENM_M03_A06 | AKFQDRVTISADDSTKTVFVDLISLASDDTAVYY | 152 |
| SNEURO2_P01_H06 | PSLESRVTISLDTSKNQFSLKLTSATAADTAVYY | 145 |

### Representative FW-H4 Sequences

An antibody or antigen-binding fragment can comprise a VH framework (FW) 4 (FW-H4) having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 11:

TABLE 11
FW-H4 sequences

| | | SEQ |
|---|---|---|
| | FW-H4 AA | ID |
| Clone Name | Sequence | NO |
| PLA2_P01_A04 | GRGTLVTVSS | 153 |
| PLA2_P01_A08 | GQGTTVTVSS | 154 |
| PLA2_P01_B02 | GQGTTVTVSS | 154 |
| PLA2_P01_B08 | GQGTLVTVSS | 155 |
| PLA2_P01_C02 | GQGTLVTVSS | 155 |
| PLA2_P01_C03 | GQGTLVTVSS | 155 |
| PLA2_P01_C04 | GQGTLVTVSS | 155 |
| PLA2_P01_D06 | GQGTLVTVSS | 155 |
| PLA2_P01_G05 | GRGTTVTVSS | 156 |
| PLA2_P01_G07 | GQGTLVTVSS | 155 |
| PLA2_P01_G12 | GRGTLVTVSS | 153 |
| KUNITZ_P01_A03 | GQGTLVTVSS | 155 |
| KUNITZ_P01_A07 | GQGTTVTVSS | 154 |
| KUNITZ_P01_D07 | GRGTLVTVSS | 153 |
| KUNITZ_P01_H05 | GRGTTVTVSS | 156 |
| KUNITZ_P01_H07 | GRGTTVTVSS | 156 |
| SNEURO_P01_A04 | GQGTLVTVSS | 155 |
| SNEURO_P01_A08 | GQGTTVTVSS | 154 |
| SNEURO_P01_A12 | GQGTTVTVSS | 154 |
| SNEURO_P01_B05 | GQGTLVTVSS | 155 |
| SNEURO_P01_B11 | GQGTLVTVSS | 155 |
| (Centi-B11) | | |
| SNEURO_P01_D09 | GQGTLVTVSS | 155 |
| (Centi-D9) | | |
| SNEURO_P01_E06 | GQGTLVTVSS | 155 |
| SNEURO_P01_E09 | GQGTLVTVSS | 155 |
| SNEURO_P01_F07 | GQGTLVTVSS | 155 |
| SNEURO_P01_F09 | GQGTLVTVSS | 155 |
| SNEURO_P01_H02 | GQGTLVTVSS | 155 |
| VENM_M03_B12 | GQGTLVTVSS | 155 |
| VENM_M12_G05 | GQGTLVTVSS | 155 |
| VENM_M03_A06 | GQGTLVTVSS | 155 |
| SNEURO2_P01_H06 | GQGTTVTVSS | 154 |

### Representative FW-L1 Sequences

An antibody or antigen-binding fragment can comprise a VL framework (FW) 1 (FW-L1) having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 12:

TABLE 12
FW-L1 sequences

| | | SEQ |
|---|---|---|
| | | ID |
| Clone Name | FW-L1 AA Sequence | NO |
| PLA2_P01_A04 | DVVMTQSPSSLSASVGDRVTITC | 157 |
| PLA2_P01_A08 | SNFMLTQPPSASGTPGQRVTISC | 158 |
| PLA2_P01_B02 | DVVMTQSPSSLSASVGDSVTITC | 159 |
| PLA2_P01_B08 | DIVMTQSPSTLSASVGDRVTITC | 160 |
| PLA2_P01_C02 | DVVMTQSPSSLSASVGDRVTITC | 157 |
| PLA2_P01_C03 | EIVLTQSPSTLSASVGDRVTITC | 161 |
| PLA2_P01_C04 | EIVLTQSPSSLSASVGDRVTITC | 162 |
| PLA2_P01_D06 | SSYELTQPPSVSVAPGQTARISC | 163 |
| PLA2_P01_G05 | EIVLTQSPSSLSASVGDRVTITC | 162 |
| PLA2_P01_G07 | DIQMTQSPSSLSASVGDRVTITC | 164 |
| PLA2_P01_G12 | DVVMTQSPSSLSASVGDRVTITC | 157 |
| KUNITZ_P01_A03 | SQSVVTQEPSLSVAPGKTAMITC | 165 |
| KUNITZ_P01_A07 | ETTLTQSPDSLAVSLGERATINC | 166 |
| KUNITZ_P01_D07 | DIVMTQSPSTLSASVGDR VIITC | 167 |
| KUNITZ_P01_H05 | SQPGLTQPPSVSMAPGQTASITC | 168 |
| KUNITZ_P01_H07 | SSYELTQPPSVSGAPGQRVTISC | 169 |
| SNEURO_P01_A04 | DIQMTQSPPSLSASVGDRVTITC | 170 |
| SNEURO_P01_A08 | DIQMTQSPSSLSASVGDRVTITC | 164 |
| SNEURO_P01_A12 | DIQMTQSPSSLSASVGDRVTITC | 164 |
| SNEURO_P01_B05 | EIVLTQSPSSLSASVGDRVTITC | 162 |
| SNEURO_P01_B11 | DIVMTQSPSSLSASVGDRVTITC | 171 |
| (Centi-B11) | | |
| SNEURO_P01_D09 | EIVLTQSPSSLSASVGDRVTITC | 162 |
| (Centi-D9) | | |
| SNEURO_P01_E06 | EIVLTQSPSSLSASVGDRVTITC | 162 |
| SNEURO_P01_E09 | DVVMTQSPSSVSASVGDRVTITC | 172 |
| SNEURO_P01_F07 | DVVMTQSPSSLFASVGDRVTITC | 173 |
| SNEURO_P01_F09 | DIVMTQSPSTLSASVGDRVTITC | 160 |
| SNEURO_P01_H02 | EIVLTQSPSSVSASVGDRVTITC | 174 |
| VENM_M03_B12 | ETTLTQSPSSVSASVGDRVTITC | 175 |
| VENM_M12_G05 | SNFMLTQPPSASGAPGHSVTISC | 176 |
| VENM_M03_A06 | EIVLTQSPSSLSASAGDRVTITC | 177 |
| SNEURO2_P01_H06 | ETTLTQSPDSLAVSLGERATINC | 166 |

### Representative FW-L2 Sequences

An antibody or antigen-binding fragment can comprise a VL framework (FW) 2 (FW-L2) having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 13:

TABLE 13
FW-L2 sequences

| Clone Name | FW-L2 AA Sequence | SEQ ID NO |
|---|---|---|
| PLA2_P01_A04 | WYQQKPGKAPKLLIY | 178 |
| PLA2_P01_A08 | WYHQLPGTAPKLLIQ | 179 |
| PLA2_P01_B02 | WYQQRPGKTPNLLIY | 180 |
| PLA2_P01_B08 | WYQQKPGKAPKLLIY | 178 |
| PLA2_P01_C02 | WYQQKPGKAPKLLIY | 178 |
| PLA2_P01_C03 | WYQQKPGKAPKLLIY | 178 |
| PLA2_P01_C04 | WYQQKPGKAPKLLIY | 178 |
| PLA2_P01_D06 | WYHQRPGQAPVLVVY | 181 |
| PLA2_P01_G05 | WYQQKPGKAPKLLIY | 178 |
| PLA2_P01_G07 | WYQQKPGKAPKLLIY | 178 |
| PLA2_P01_G12 | WYQQKPGKAPKLLIY | 178 |
| KUNITZ_P01_A03 | WFRQRPGQAPVLVVY | 182 |
| KUNITZ_P01_A07 | WYQQKPGQPPKLLIY | 183 |
| KUNITZ_P01_D07 | WYQQKSGKAPKLLIY | 184 |
| KUNITZ_P01_H05 | WFQQKSGQAPALVVY | 185 |
| KUNITZ_P01_H07 | WYQQLPGTAPKLLIY | 186 |
| SNEURO_P01_A04 | WYQQKPGKAPKLLIY | 178 |
| SNEURO_P01_A08 | WYQQKPGKAPKLLIY | 178 |
| SNEURO_P01_A12 | WYQQKPGKAPKLLIY | 178 |
| SNEURO_P01_B05 | WYQQKPGKAPKLLIY | 178 |
| SNEURO_P01_B11 | WYQQKPGKAPKLLIY | 178 |
| (Centi-B11) | | |
| SNEURO_P01_D09 | WYQQKPGKAPKLLIY | 178 |
| (Centi-D9) | | |
| SNEURO_P01_E06 | WYQQKPGEAPKFLIY | 187 |
| SNEURO_P01_E09 | WYQQKPGKAPKLLIY | 178 |
| SNEURO_P01_F07 | WYQQKPGKAPKLLIY | 178 |
| SNEURO_P01_F09 | WYQQKPGKAPKLLIY | 178 |
| SNEURO_P01_H02 | WYQQKPGKAPKLLIY | 178 |
| VENM_M03_B12 | WYQQKPGKAPKLLIN | 188 |
| VENM_M12_G05 | WYQQLPGTAPKVLIY | 189 |
| VENM_M03_A06 | WYQQKPGKAPRLLIY | 190 |
| SNEURO2_P01_H06 | WYQQKPGQPPKLLIY | 183 |

### Representative FW-L3 Sequences

An antibody or antigen-binding fragment can comprise a VL framework (FW) 3 (FW-L3) having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 14:

TABLE 14
FW-L3 sequences

| | | SEQ ID |
|---|---|---|
| Clone Name | FW-L3 AA Sequence | NO |
| PLA2_P01_A04 | GVPSRFSGSGSGTDFTLTISSLHPEDFATYY | 191 |
| PLA2_P01_A08 | GVPERFSGSKSGTSASLAISGLRSEDEADYF | 192 |
| PLA2_P01_B02 | GVPSRFSGSGSGTDFTLTISSLQPEDFATYY | 193 |
| PLA2_P01_B08 | GVPSRFSGSGSGTDFTLTISSLQPEDFATYY | 193 |
| PLA2_P01_C02 | GVPSRFSGSGSGTDFTLTISSLQPEDFATYY | 193 |
| PLA2_P01_C03 | GVPSRFSGSGSGTDFTLTISSLQPEDFATYY | 193 |
| PLA2_P01_C04 | GVPSRFSGSGSGTDFTLTISSLQPEDFATYY | 193 |
| PLA2_P01_D06 | GIPERFSGSNSGNTATLTISRVEAGDEADYY | 194 |
| PLA2_P01_G05 | GVPSRFSGSGSGTDFTLTIGSLQPEDFATYY | 195 |
| PLA2_P01_G07 | GVPSRFSGSGSGTDFTLTISSLQPEDFATYY | 193 |
| PLA2_P01_G12 | GVPSRFSGSGSGTDFTLTISSLHPEDFATYY | 191 |
| KUNITZ_P01_A03 | GIPERISGSKSGNTATLTISRVEDGDFADYY | 196 |
| KUNITZ_P01_A07 | GVPDRFSGSGSGTDFSLTISSLQAEDVAVYY | 197 |
| KUNITZ_P01_D07 | GVPSRFSGSGSGTEFTLTISSLQPDDIATYY | 198 |
| KUNITZ_P01_H05 | GIPERFSGSNSGNTATLTISRVEAGDEADYF | 199 |
| KUNITZ_P01_H07 | GVPDRFSGSKSGTSASLAITGLQAEDEADYY | 200 |
| SNEURO_P01_A04 | GVPSRFSGGGSGTDFTLTISSLQPEDFATYY | 201 |
| SNEURO_P01_A08 | GVPSRFSGSGSGTDFTLTISSLQPEDFATYY | 193 |
| SNEURO_P01_A12 | GVPSRFSGSGSGTDFTLTISSLQPEDFATYY | 193 |
| SNEURO_P01_B05 | GVPSRFSGRGSGTDFTLTISSLQPEDFTSYY | 202 |
| SNEURO_P01_B11 | GVPSRLSGSGSGTDFTLTISSLQPEDFATYY | 203 |
| (Centi-B11) | | |
| SNEURO_P01_D09 | GVPSRFSGSGSGTDFTLTISSQQPEDFATYY | 204 |
| (Centi-D9) | | |
| SNEURO_P01_E06 | GVPSRFSGSGSGTDFTLTISSPQPEDFATYY | 205 |
| SNEURO_P01_E09 | GVPSRFSGSGSGTDFTLTISSLQPEDSATYY | 206 |
| SNEURO_P01_F07 | GVPSRFSGSGSVADFTLTISSLQPEDFATYY | 207 |
| SNEURO_P01_F09 | GVPSRFSGSGSGTDFTLTISSLQPEDFATYY | 193 |
| SNEURO_P01_H02 | GVPSRFSGSGSGTDFTLTISSLQPEDFATYY | 193 |
| VENM_M03_B12 | GVPSRFSGSGSGTEFTLTISSLQPDDFATYY | 208 |
| VENM_M12_G05 | GVPDRFSASKSGTSASLAISGLRSEDEADYY | 209 |
| VENM_M03_A06 | GVPSRFSGSGSGTDFTLTISSLQPEDFATYY | 193 |
| SNEURO2_P01_H06 | GVPDRFSGSGSGTDFSLTISSLQAEDVAVYY | 197 |

### Representative FW-L4 Sequences

An antibody or antigen-binding fragment can comprise a VL framework (FW) 4 (FW-L4) having an amino acid sequence that is at least 20%, 25%, 30%, 35%, 40%, 45%, 50%, 55%, 60%, 65%, 70%, 75%, 80%, 85%, 90%, 95%, 98%, 99%, or 100% identical to any one of the following sequences of Table 15:

TABLE 15
FW-L4 sequences

| Clone Name | FW-L4 AA Sequence | SEQ ID NO |
|---|---|---|
| PLA2_P01_A04 | GGGTKLEIKR | 210 |
| PLA2_P01_A08 | GTGTKLTVLG | 211 |
| PLA2_P01_B02 | GGGTKVEIKR | 212 |
| PLA2_P01_B08 | GGGTKVEIKR | 212 |
| PLA2_P01_C02 | GQGTKVEIKR | 213 |
| PLA2_P01_C03 | GQGTKLEIKR | 214 |
| PLA2_P01_C04 | GQGTKVDIKR | 215 |
| PLA2_P01_D06.1 | GGGTKLTVLR | 216 |
| PLA2_P01_D06.2 | GGGTKLTVKR | 217 |
| PLA2_P01_G05 | GGGTKVDIKR | 218 |
| PLA2_P01_G07 | GGGTKVEIKR | 212 |
| PLA2_P01_G12 | GGGTKLEIKR | 210 |
| KUNITZ_P01_A03.1 | GTGTKLTVKR | 219 |
| KUNITZ_P01_A03.2 | GTGTKLTVLR | 220 |
| KUNITZ_P01_A07 | GQGTKLEIKR | 214 |
| KUNITZ_P01_D07 | GRGTKVEIKR | 221 |
| KUNITZ_P01_H05.1 | GGGTKLTVLR | 216 |
| KUNITZ_P01_H05.2 | GGGTKLTVKR | 217 |
| KUNITZ_P01_H07.1 | GGGTKLTVLR | 216 |
| KUNITZ_P01_H07.2 | GGGTKLTVKR | 217 |
| SNEURO_P01_A04 | GGGTKVEIKR | 212 |
| SNEURO_P01_A08 | GQGTKVEIKR | 213 |
| SNEURO_P01_A12 | GGGTNLEIKR | 222 |
| SNEURO_P01_B05 | GGGTKVDIKR | 218 |
| SNEURO_P01_B11 | GGGTKVEIKR | 212 |
| (Centi-B11) | | |
| SNEURO_P01_D09 | GQGTKVEIKR | 213 |
| (Centi-D9) | | |
| SNEURO_P01_E06 | GQGTKVDIKR | 215 |
| SNEURO_P01_E09 | GGGTKVEIKR | 212 |
| SNEURO_P01_F07 | GGGTKLEIKR | 210 |
| SNEURO_P01_F09 | GQGTKVDIKR | 215 |
| SNEURO_P01_H02 | GGGTKLEIKR | 210 |
| VENM_M03_B12 | GGGTKVDIKR | 218 |
| VENM_M12_G05 | GTGTKLTVLG | 211 |
| VENM_M03_A06 | GQGTRLEIKR | 223 |
| SNEURO2_P01_H06 | GQGTKLEIKR | 214 |

### Representative VH Sequences

An antibody or antigen-binding fragment can comprise an VH having an amino acid sequence that is at least 70%, 75%, 80%, 85%, 90%, 95%, 98%, 99%, or 100% identical to any one of the following sequences of Table 16:

TABLE 16
VH sequences

| | | SEQ |
|---|---|---|
| | | ID |
| Clone Name | VH AA Sequence | NO |
| PLA2_P01_ | QVTLKESGGGVVQPGRSLRLSCAASGFTFSSYAMSW | 224 |
| A04 | ARXAPGKGLEWVSAISGSGGSTYYADSVRGRFTISRD | |
| | NSKNTLYLQMNSLRAEDTAVYYCTTDHRRRGLWGR | |
| | GTLVTVSS | |
| PLA2_P01_ | EVQLVESGGGLVQPGGSLRLSCAASGFTLSDSTVHWV | 225 |
| A08 | RQASGKGLERVAYIRSKVNSYATGYAASVKGRFTISR | |
| | DDSKNTAYLQMNSLKIEDTAVYYCTRRNPGGMDVW | |
| | GQGTTVTVSS | |
| PLA2_P01_ | EVQLVESGGGVVQPGRSLRLSCAASGFTFSSYAMSW | 226 |
| B02 | VRQAPGKGLEWVSAISGSGGSTYYADSVKGRFTISRD | |
| | NSKNMLYLQMNSLRAEDTALYYCARLRVGRDYKGM | |
| | DVRGQGTTVTVSS | |
| PLA2_P01_ | EVQLVESGGDLVQPGGSLRLSCAASGFTLSDSTVHWV | 227 |
| B08 | RQASGKGLERVAYIRSKVNSYATGYAASVKGRFTISR | |
| | DDSKNTAYLQMNSLKIEDTAVYYCTRRNPGGMDVW | |
| | GQGTLVTVSS | |
| PLA2_P01_ | QVTLKESGGGLVQPGGSLRLSCAASGFTLSDSTVHWV | 228 |
| C02 | RQASGKGLERVAYIRSKVNSYATGYAASVKGRFTISR | |
| | DDSKNTAYLQMNSLKIEDTAVYYCTRRNPGGMDVW | |
| | GQGTLVTVSS | |
| PLA2_P01_ | QVTLKESGGGLVKPGGSLRLSCAASGFTFTNAWMGW | 229 |
| C03 | VRQVPGTGLEWVARIKSKSDGGKTDYAAPVKGRFTIS | |
| | RDDSQNTLYLQMNSLRTEDSAVYYCVTYTYCGGDCY | |
| | SEDWGQGTLVTVSS | |
| PLA2_P01_ | XVQLQESGGGVVQPGRSLRLSCAGSGFTFSSYAMSW | 230 |
| C04 | VRQAPGKGLEWVSAISGSGGSTYYADSVKGRFTISRD | |
| | NSKNTLYLQMNSLRAEDTAVYYCAKVRRGYSYGTD | |
| | YWGQGTLVTVSS | |
| PLA2_P01_ | QVXLQESGGGLVQPGGSLRLSCAASGFAFSSYAMSW | 231 |
| D06 | VRQAPGKGLEWVSAISGSGGSTYYADSVKGRFTISRD | |
| | NSKNTLYLQMNSLRAEDTAVYYCATSRTFDYWGQGT | |
| | LVTVSS | |
| PLA2_P01_ | HVILKESGGGVVQPGRSLRLSCAASGFTFSGYGMHW | 232 |
| G05 | VRQAPGKGLEWVATISYDGSNKYYADSVKGRFTISRD | |
| | NSKNTLYLQMNSLRAEDTAVYYCALARSYDYYYGV | |
| | DVWGRGTTVTVSS | |
| PLA2_P01_ | EVQLVESGGGLVQPGGSLRLSCAASGFAFSSYAMNW | 233 |
| G07 | VRQAPGKGLEWVSTVSGSGSTYYADSVKGRFTISRDN | |
| | SKNTLSLQMNGLRAEDTAVYYCARGRRFDPWGQGTL | |
| | VTVSS | |
| PLA2_P01_ | QVTLKESGGGVVQPGRSLRLSCAASGFTFSSYAMSW | 224 |
| G12 | ARXAPGKGLEWVSAISGSGGSTYYADSVRGRFTISRD | |
| | NSKNTLYLQMNSLRAEDTAVYYCTTDHRRRGLWGR | |
| | GTLVTVSS | |
| KUNITZ_P01_ | EVQLVESGAEVKKPGASVKVSCKASGGTFSSDAITWV | 234 |
| A03 | RQAPGQGLEWMGWINPNSGGTNYAQKFQGRVTMTR | |
| | DTSTSTVYMELSSLRSEDTAVYYCSRDYGYYFDFWG | |
| | QGTLVTVSS | |
| KUNITZ_P01_ | QVQLVESGPGLVAPSGTLSLTCTVSGGSINIGNWYNW | 235 |
| A07 | VRQSPGKGLEWIGEVYHSGSTNYNPSLESRVTISLDTS | |
| | KNQFSLKLTSATAADTAVYYCATRSVLTGSPIWGQGT | |
| | TVTVSS | |
| KUNITZ_P01_ | QVQLQQSGAEVKKPGASVKVSCKASGHTFSDXYMH | 236 |
| D07 | WVRQAPGQGLEWMGRINPDSGATDYAQHFQGRVTM | |
| | TRDTSTSTVYMELSSLRSEDTAVYYCAREAVAGTHPQ | |
| | AGDFDLWGRGTLVTVSS | |
| KUNITZ_P01_ | QVQLVQSGAEVKEPGASVKVSCKASGYTFTSYHMH | 237 |
| H05 | WVRQAPGQGLEWMGRINPDSGATDYAQHFQGRVTM | |
| | TRDTSTSTVYMELSSLRSEDTAVYYCAREAVAGTHPQ | |
| | AGDFDLWGRGTTVTVSS | |
| KUNITZ_P01_ | QVQLVQSGAEVKKPGASMKVSCKASGYTFTDHYIHW | 238 |
| H07 | VRQAPGQGLEWMGRINPDSGATDYAQHFQGRVTMT | |
| | RDTSTSTVYMELSSLRSEDTAVYYCAREAVAGTHPQA | |
| | GDFDLWGRGTTVTVSS | |
| SNEURO_P01_ | QVQLQESGGGLVQPGGSLRLSCAASGFTFSNFDMHW | 239 |
| A04 | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| SNEURO_P01_ | QVQLQQSGGGLVQPGGSLRLSCAASGFTFSNFDMHW | 240 |
| A08 | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTTVTVSS | |
| SNEURO_P01_ | EVQLVESGGGLVQPGGSLRLSCAASGFTFSSYAMSWV | 241 |
| A12 | RQAPGKGLEWVSAISGSGGSTYYADSVKGRFTTSRDN | |
| | AKNSLYLQMNSLRAEDTALYYCARLRVGRDYKGMD | |
| | VRGQGTTVTVSS | |
| SNEURO_P01_ | QVQLQESGGGLVQPGGSLRLSCAASGFTFSNFDMHW | 239 |
| B05 | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| SNEURO_P01_ | QVQLQESGGGLVQPGGSLRLSCAASGFTFSNFDMHW | 239 |
| B11 (Centi- | VRQSPGKGLEWVSGLDHSGGAHYAGSVKGRFTISRE | |
| B11) | DAKNSLDLQMNNLRVDDTAVYFCVRGTLYHYTSGSY | |
| | YSDAFDIWGQGTLVTVSS | |
| SNEURO_P01_ | EVQLVESGGGFVQPGGSLRLSCAASGFTFSNFDMHW | 242 |
| D09 (Ceti- | VRQSPGKGLEWVSGLDHSGGAHYAGSVKGRFTISRE | |
| D9) | DAKNSLDLQMNNLRVDDTAVYFCVRGTLYHYTSGSY | |
| | YSDAFDIWGQGTLVTVSS | |
| SNEURO_P01_ | QVTLKESGGGLVQPGGSLRLSCAASGFTFSNFDMHW | 243 |
| E06 | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| SNEURO_P01_ | QVTLKESGGGLVQPGGSLRLSCAASGFTFSNFDMHW | 243 |
| E09 | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| SNEURO_P01_ | QVQLVQSGGGLVQPGGSLRLSCAASGFTFSNFDMHW | 244 |
| F07 | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| SNEURO_P01_ | QVQLQESGGGLVQPGGSLRLSCAASGFTFSNFDMHW | 239 |
| F09 | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| SNEURO_P01_ | EVQLVESGGDLVQPGGSLRLSCAASGFTFSTFDMHW | 245 |
| H02 | VRQSPGKGLEWVSTFDHSGGAHYAASVKGRFTISREA | |
| | GKHSLDLQMNSLRVDDTAVYFCARGTLYHYTSGSYY | |
| | SDAFDIWGQGTLVTVSS | |
| VENM_M03_ | EVQLVESGGGLVQPGGSLRLSCAASGFTFDDFAMHW | 246 |
| B12 | VRQAPGKSLEWVSSISWNSVNIDYADSVKGRFTISRD | |
| | NAKSSLFLQMSSLRVEDTALYYCAKAGSYDGSGYNY | |
| | FDSWGQGTLVTVSS | |
| VENM_M12_ | QVQLVQSGGGVVQPGSSLRLSCEASGFIVSDYGMHW | 247 |
| G05 | VRQAPGKGLEWVAVMSYDGSQKFYSGSVKGRFTISR | |
| | DISNNTLFLQMNDLRPEDTAVYHCVREGSLTGPGWE | |
| | NWFDPWGQGTLVTVSS | |
| VENM_M03_ | EVQLLXSAAEVKRPGSSVRVSCKVSGGSFRDYAVSW | 248 |
| A06 | VRQAPGQGPEWMGGIIPMFGRGEYTAKFQDRVTISAD | |
| | DSTKTVFVDLISLASDDTAVYYCARGLRLGELKQWG | |
| | QGTLVTVSS | |
| SNEURO2 P | QVQLVESGPGLVAPSGTLSLTCTVSGGSINIGNWYNW | 235 |
| 01 H06 | VRQSPGKGLEWIGEVYHSGSTNYNPSLESRVTISLDTS | |
| | KNQFSLKLTSATAADTAVYYCATRSVLTGSPIWGQGT | |
| | TVTVSS | |
| Centi-D09.0 | EVQLVESGGGFVQPGGSLRLSCAASGFTFSNFDMHW | 429 |
| | VRQSPGKGLEWVSGLDHSGGAHYAGSVKGRFTISRE | |
| | DAKNSLDLQMNNLRVDDTAVYFCVRGTLYHYTSGSY | |
| | CSDAFDIWGQGTLVTVSS | |
| Centi-D09.1 | EVQLVESGGGFVQPGGSLRLSCAASGFTFSNFDMHW | 430 |
| | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| Centi-D09.2 | EVQLVESGGGFVQPGGSLRLSCAASGFTFSTFDMHWV | 431 |
| | RQSPGKGLEWVSTFDHSGGAHYAGSVKGRFTISREDA | |
| | KNSLDLQMNNLRVDDTAVYFCARGTLYHYTSGSYYS | |
| | DAFDIWGQGTLVTVSS | |
| Centi-D09.3 | EVQLVESGGGFVQPGGSLRLSCAASGFTFSNFDMHW | 432 |
| | VRQSPGKGLEWVSGVDHSGGAHYAGSVKGRFTISRE | |
| | DAKNSLDLQMNNLRVDDTAVYFCVRGTLYHYTSGSY | |
| | YSDAFDIWGQGTLVTVSS | |
| Centi-D09.4 | EVQLVESGGGFVQPGGSLRLSCAASGFTFSNFDMHW | 433 |
| | VRQSPGKGLEWVPGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| Centi-D09.5 | EVQLVESGGGFVQPGGSLRLSCAASGFTFSNFDMHW | 434 |
| | VRQSPGKGLEWVSGLDHNGGAHYAGSVKGRFTISRE | |
| | DAKNSLDLQMNNLRVDDTAVYFCVRGTLYHYTSGSY | |
| | YSDAFDIWGQGTLVTVSS | |
| Centi-D09.6 | EVQLVESGGGFVQPGGSLRLSCAASGFTLSNFDMHW | 435 |
| | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| Centi-D09.7 | EVQLVESGGGFVQPGGSLRLSCAASGFTFSNFDMHW | 436 |
| | VRQSPGKGLEWVSGLDHRGGAHYAGSVKGRFTISRE | |
| | DAKNSLDLQMNNLRVDDTAVYFCVRGTLYHYTSGSY | |
| | YSDAFDIWGQGTLVTVSS | |
| Centi-D09.8 | EVQLVESGGGFVQPGGSLRLSCAASGFTFRNFDMHW | 437 |
| | VRQSPGKGLEWVSVLDYSGGEHYAGSVKGRFTISRED | |
| | AKNSLDLQMNNLRVDDTAVYFCVRGTLYHYTSGSYY | |
| | SDAFDIWGQGTL VTVSS | |
| Centi-D09.9 | EVQLVESGGGFVQPGGSLRLSCAASGFTFSNFDMHW | 438 |
| | VRQSPGKGLEWVSGLDLSGGAHYAGSVKGRFTISRED | |
| | AKNSLDLQMNNLRVDDTAVYFCVRGTLYHYTSGSYY | |
| | SDAFDIWGQGTLVTVSS | |
| Centi-D09.10 | EVQLVESGGGFVQPGGSLRLSCAASGFTFSNFDMHW | 439 |
| | VRQSPGKGLEWVSGLAHSGGAHYAGSVKGRFTISRE | |
| | DAKNSLDLQMNNLRVDDTAVYFCVRGTLYHYTSGSY | |
| | YSDAFDIWGQGTLVTVSS | |
| Centi-D09.11 | EVQLVESGGGFVQPGGSLRLSCAASGFTFSNYDMHW | 440 |
| | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| Centi-D09.12 | EVQLVESGGGFVQPGGSLRLSCAASGFTFSNFDMHW | 441 |
| | VRQSPGKGLEWVSGLDHSVGAHYAGSVKGRFTISRE | |
| | DAKNSLDLQMNNLRVDDTAVYFCVRGTLYHYTSGSY | |
| | YSDAFDIWGQGTLVTVSS | |
| Centi-D09.13 | EVQLVESGGGFVQPGGSLRLSCAASGFTFSNLDMHW | 442 |
| | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| Centi-D09.14 | EVQLVESGGGFVQPGGSLRLSCAASGFTFSNFDMHW | 443 |
| | VRQSPGKGLEWVHGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| Centi-D09.15 | EVQLVESGGGFVQPGGSLRLSCAASGFTFSNFDMHW | 444 |
| | VRQSPGKGLEWVSGLDHSGGAQYAGSVKGRFTISRE | |
| | DAKNSLDLQMNNLRVDDTAVYFCVRGTLYHYTSGSY | |
| | YSDAFDIWGQGTLVTVSS | |
| Centi-D09.16 | EVQLVESGGGFVQPGGSLRLSCAASGFTFSNFDMHW | 445 |
| | VRQSPGKGLEWVSGLDHSGGAHHAGSVKGRFTISRE | |
| | DAKNSLDLQMNNLRVDDTAVYFCVRGTLYHYTSGSY | |
| | YSDAFDIWGQGTLVTVSS | |
| Centi-D09.17 | EVQLVESGGGFVQPGGSLRLSCAASGFTFSNFDMHW | 446 |
| | VRQSPGKGLEWVSGIDHSGGAHYAGSVKGRFTISRED | |
| | AKNSLDLQMNNLRVDDTAVYFCVRGTLYHYTSGSYY | |
| | SDAFDIWGQGTLVTVSS | |
| Centi-D09.18 | EVQLVESGGGFVQPGGSLRLSCAASGFTFSNFDMHW | 447 |
| | VRQSPGKGLEWVSGLDHSGGAHDAGSVKGRFTISRE | |
| | DAKNSLDLQMNNLRVDDTAVYFCVRGTLYHYTSGSY | |
| | YSDAFDIWGQGTLVTVSS | |
| Centi-D09.19 | EVQLVESGGGFVQPGGSLRLSCAASGFTFGNFDMHW | 448 |
| | VRQSPGKGLEWVSGLDHTGGAHSEGSVKGRFTISRED | |
| | AKNSLDLQMNNLRVDDTAVYFCVRGTLYHYTSGSYY | |
| | SDAFDIWGQGTL VTVSS | |
| Centi-D09.20 | EVQLVESGGGFVQPGGSLRLSCAASGFTFSNFDMHW | 449 |
| | VRQSPGKGLEWVSGLDHSGGAHYAGSVKGRFTISRE | |
| | DAKNSLDLQMNNLRVDDTAVYFCVRGTLYHYTSGSY | |
| | CSDAFDIWGQGTLVTVSS | |
| Centi-D09.21 | EVQLVESGGGFVQPGGSLRLSCAASGFTFSNFDMHW | 500 |
| | VRQSPGKGLEWVSGLDHSGGAHYAGSVKGRFTISRE | |
| | DAKNSLDLQMNNLRVDDTAVYFCVRGTLYHYTSGSY | |
| | YSDAFDIWGQGTLVTVSS | |
| SNEURO_P01_ | EVQLVESGGGLVQPGGSLRLSCAASGFTFSNFDMHW | 513 |
| D09 VH | VRQATGKGLEWVSGLDHSGGAHYAGSVKGRFTISRE | |
| germlined | NAKNSLYLQMNSLRAGDTAVYYCVRGTLYHYTSGSY | |
| | YSDAFDIWGQGTLVTVSS | |
| SNEURO_P01_ | EVQLVESGGGFVQPGGSLRLSCAASGFTFSNFDMHW | 514 |
| D09 VK | VRQSPGKGLEWVSGLDHSGGAHYAGSVKGRFTISRE | |
| germlined | DAKNSLDLQMNNLRVDDTAVYFCVRGTLYHYTSGSY | |
| | YSDAFDIWGQGTLVTVSS | |
| SNEURO_P01_ | EVQLVESGGGLVQPGGSLRLSCAASGFTFSNFDMHW | 515 |
| D09 FW | VRQATGKGLEWVSGLDHSGGAHYAGSVKGRFTISRE | |
| germlined | NAKNSLYLQMNSLRAGDTAVYYCVRGTLYHYTSGSY | |
| | YSDAFDIWGQGTLVTVSS | |
| SNEURO_P01_ | EVQLVESGGGFVQPGGSLRLSCAASGFTFSNFDMHW | 516 |
| D09 VK L3 | VRQSPGKGLEWVSGLDHSGGAHYAGSVKGRFTISRE | |
| NS→QS | DAKNSLDLQMNNLRVDDTAVYFCVRGTLYHYTSGSY | |
| | YSDAFDIWGQGTLVTVSS | |
| SNEURO_P01_ | EVQLVESGGGFVQPGGSLRLSCAASGFTFSNFDMHW | 517 |
| D09 VK L3 | VRQSPGKGLEWVSGLDHSGGAHYAGSVKGRFTISRE | |
| NS→NT | DAKNSLDLQMNNLRVDDTAVYFCVRGTLYHYTSGSY | |
| | YSDAFDIWGQGTLVTVSS | |
| Centi-DTX- | QVQLQESGPGLVKPSGTLSLTCAVSGDSFSSDGYSWS | 523 |
| B03 | WIRQAPGKGLEWIGEIYHIGSTNYNPSLKSRVSMSVD | |
| | KSKNQFSLRLTSVTAADTAVYYCAKHDYGKLWGGL | |
| | DVWGQGTLVTVSS | |

### Representative VL Sequences

An antibody or antigen-binding fragment can comprise an VL having an amino acid sequence that is at least 70%, 75%, 80%, 85%, 90%, 95%, 98%, 99%, or 100% identical to any one of the following sequences of Table 17:

TABLE 17
VL sequences

| | | SEQ |
|---|---|---|
| | | ID |
| Clone Name | VL AA Sequence | NO |
| PLA2_P01_A04 | DVVMTQSPSSLSASVGDRVTITCQASQDISNYLNWYQ | 249 |
| | QKPGKAPKLLIYGASNLHSGVPSRFSGSGSGTDFTLTIS | |
| | SLHPEDFATYYCQQSYSTPLTFGGGTKLEIKR | |
| PLA2_P01_A08 | SNFMLTQPPSASGTPGQRVTISCSGSSSNIGSNYVYWY | 250 |
| | HQLPGTAPKLLIQRNNQRPSGVPERFSGSKSGTSASLA | |
| | ISGLRSEDEADYFCAAWDDRLAGYVFGTGTKLTVLG | |
| PLA2_P01_B02 | DVVMTQSPSSLSASVGDSVTITCRASQNINNYLNWYQ | 251 |
| | QRPGKTPNLLIYDASNLETGVPSRFSGSGSGTDFTLTIS | |
| | SLQPEDFATYYCQQSHITPLTFGGGTKVEIKR | |
| PLA2_P01_B08 | DIVMTQSPSTLSASVGDRVTITCRASQGIRNDLGWYQ | 252 |
| | QKPGKAPKLLIYAASTLQSGVPSRFSGSGSGTDFTLTIS | |
| | SLQPEDFATYYCQQANSYPVTFGGGTKVEIKR | |
| PLA2_P01_C02 | DVVMTQSPSSLSASVGDRVTITCRASRSIRNDLAWYQ | 253 |
| | QKPGKAPKLLIYDASNLETGVPSRFSGSGSGTDFTLTIS | |
| | SLQPEDFATYYCQRSYTTPPTFGQGTKVEIKR | |
| PLA2_P01_C03 | EIVLTQSPSTLSASVGDRVTITCRASQSISSWLAWYQQ | 254 |
| | KPGKAPKLLIYKASSLESGVPSRFSGSGSGTDFTLTISS | |
| | LQPEDFATYYCQQAKSFPVSFGQGTKLEIKR | |
| PLA2_P01_C04 | EIVLTQSPSSLSASVGDRVTITCRASQDVSSWLAWYQ | 255 |
| | QKPGKAPKLLIYAASTLQSGVPSRFSGSGSGTDFTLTIS | |
| | SLQPEDFATYYCQQHYTTPPTFGQGTKVDIKR | |
| PLA2_P01_ | SSYELTQPPSVSVAPGQTARISCGGDNIGDKRVHWYH | 256 |
| D06.1 | QRPGQAPVLVVYDDADRPTGIPERFSGSNSGNTATLTI | |
| | SRVEAGDEADYYCQVYDRASNHVVFGGGTKLTVLR | |
| PLA2_P01_ | SSYELTQPPSVSVAPGQTARISCGGDNIGDKRVHWYH | 257 |
| D06.2 | QRPGQAPVLVVYDDADRPTGIPERFSGSNSGNTATLTI | |
| | SRVEAGDEADYYCQVYDRASNHVVFGGGTKLTVKR | |
| PLA2_P01_G05 | EIVLTQSPSSLSASVGDRVTITCRASQDIRNDLGWYQQ | 258 |
| | KPGKAPKLLIYAASTLQSGVPSRFSGSGSGTDFTLTIGS | |
| | LQPEDFATYYCQQANSFPLTFGGGTKVDIKR | |
| PLA2_P01_G07 | DIQMTQSPSSLSASVGDRVTITCRASQSISSYLNWYQQ | 259 |
| | KPGKAPKLLIYAASSLQSGVPSRFSGSGSGTDFTLTISS | |
| | LQPEDFATYYCQQSYSTPLTFGGGTKVEIKR | |
| PLA2_P01_G12 | DVVMTQSPSSLSASVGDRVTITCQASQDISNYLNWYQ | 249 |
| | QKPGKAPKLLIYGASNLHSGVPSRFSGSGSGTDFTLTIS | |
| | SLHPEDFATYYCQQSYSTPLTFGGGTKLEIKR | |
| KUNITZ_P01_ | SQSVVTQEPSLSVAPGKTAMITCGGDRLHFKTVDWFR | 260 |
| A03.1 | QRPGQAPVLVVYGDSDRPSGIPERISGSKSGNTATLTIS | |
| | RVEDGDFADYYCQVWDHNGDGRVFGTGTKLTVLR | |
| KUNITZ_P01_ | SQSVVTQEPSLSVAPGKTAMITCGGDRLHFKTVDWFR | 261 |
| A03.2 | QRPGQAPVLVVYGDSDRPSGIPERISGSKSGNTATLTIS | |
| | RVEDGDFADYYCQVWDHNGDGRVFGTGTKLTVKR | |
| KUNITZ_P01_ | ETTLTQSPDSLAVSLGERATINCKSSQSVLDSSKNKNY | 262 |
| A07 | LAWYQQKPGQPPKLLIYWASIRESGVPDRFSGSGSGT | |
| | DFSLTISSLQAEDVAVYYCQQYYGTPYTFGQGTKLEI | |
| | KR | |
| KUNITZ_P01_ | DIVMTQSPSTLSASVGDRVIITCRASQSINNWLAWYQ | 263 |
| D07 | QKSGKAPKLLIYKASTLESGVPSRFSGSGSGTEFTLTIS | |
| | SLQPDDIATYYCQQYNNHSRTFGRGTKVEIKR | |
| KUNITZ_P01_ | SQPGLTQPPSVSMAPGQTASITCGGNNIGSKSVHWFQ | 264 |
| H05.1 | QKSGQAPALVVYDDTNRPSGIPERFSGSNSGNTATLTI | |
| | SRVEAGDEADYFCQLWDSSGEHWVFGGGTKLTVLR | |
| KUNITZ_P01_ | SQPGLTQPPSVSMAPGQTASITCGGNNIGSKSVHWFQ | 265 |
| H05.2 | QKSGQAPALVVYDDTNRPSGIPERFSGSNSGNTATLTI | |
| | SRVEAGDEADYFCQLWDSSGEHWVFGGGTKLTVKR | |
| KUNITZ_P01_ | SSYELTQPPSVSGAPGQRVTISCTGSSSNIGAGFDVHW | 266 |
| H07.1 | YQQLPGTAPKLLIYGNSNRPSGVPDRFSGSKSGTSASL | |
| | AITGLQAEDEADYYCRSYDSSLSGSVFGGGTKLTVLR | |
| KUNITZ_P01_ | SSYELTQPPSVSGAPGQRVTISCTGSSSNIGAGFDVHW | 267 |
| H07.2 | YQQLPGTAPKLLIYGNSNRPSGVPDRFSGSKSGTSASL | |
| | AITGLQAEDEADYYCRSYDSSLSGSVFGGGTKLTVKR | |
| SNEURO_P01_ | DIQMTQSPPSLSASVGDRVTITCRASQSISSYLNWYQQ | 268 |
| A04 | KPGKAPKLLIYAASSLQSGVPSRFSGGGSGTDFTLTISS | |
| | LQPEDFATYYCQQSYSTPLTFGGGTKVEIKR | |
| SNEURO_P01_ | DIQMTQSPSSLSASVGDRVTITCRASQSISSYLNWYQQ | 269 |
| A08 | KPGKAPKLLIYAASSLQSGVPSRFSGSGSGTDFTLTISS | |
| | LQPEDFATYYCQQSYSTHTFGQGTKVEIKR | |
| SNEURO_P01_ | DIQMTQSPSSLSASVGDRVTITCRASQSISTYLNWYQQ | 270 |
| A12 | KPGKAPKLLIYAASSLQSGVPSRFSGSGSGTDFTLTISS | |
| | LQPEDFATYYCQQSYSTPYTFGGGTNLEIKR | |
| SNEURO_P01_ | EIVLTQSPSSLSASVGDRVTITCRASQSISSFLNWYQQK | 271 |
| B05 | PGKAPKLLIYAASSLQSGVPSRFSGRGSGTDFTLTISSL | |
| | QPEDFTSYYCQQSYSPPLTFGGGTKVDIKR | |
| SNEURO_P01_ | DIVMTQSPSSLSASVGDRVTITCRASQGISDNLNWYQ | 272 |
| B11 (Centi- | QKPGKAPKLLIYAASTLQSGVPSRLSGSGSGTDFTLTIS | |
| B11) | SLQPEDFATYYCQQANSFPLTFGGGTKVEIKR | |
| SNEURO_P01_ | EIVLTQSPSSLSASVGDRVTITCQASQDISNYLNWYQQ | 273 |
| D09 (Centi- | KPGKAPKLLIYAASSLESGVPSRFSGSGSGTDFTLTISS | |
| D9) | QQPEDFATYYCQQANSFPYTFGQGTKVEIKR | |
| SNEURO_P01_ | EIVLTQSPSSLSASVGDRVTITCRASETISIYLNWYQQK | 274 |
| E06 | PGEAPKFLIYAASSLQSGVPSRFSGSGSGTDFTLTISSP | |
| | QPEDFATYYCQQSYSTPWTFGQGTKVDIKR | |
| SNEURO_P01_ | DVVMTQSPSSVSASVGDRVTITCRASQGISSWLAWYQ | 275 |
| E09 | QKPGKAPKLLIYAASNLQMGVPSRFSGSGSGTDFTLTI | |
| | SSLQPEDSATYYCQQSYTTPLTFGGGTKVEIKR | |
| SNEURO_P01_ | DVVMTQSPSSLFASVGDRVTITCRASQDIRNDLGWYQ | 276 |
| F07 | QKPGKAPKLLIYGASTLQSGVPSRFSGSGSVADFTLTIS | |
| | SLQPEDFATYYCQQSYITPLTFGGGTKLEIKR | |
| SNEURO_P01_ | DIVMTQSPSTLSASVGDRVTITCRASQSISSWLAWYQQ | 277 |
| F09 | KPGKAPKLLIYAASSLQSGVPSRFSGSGSGTDFTLTISS | |
| | LQPEDFATYYCQQSYSTITFGQGTKVDIKR | |
| SNEURO_P01_ | EIVLTQSPSSVSASVGDRVTITCRASQGISSWLAWYQQ | 278 |
| H02 | KPGKAPKLLIYAASSLQSGVPSRFSGSGSGTDFTLTISS | |
| | LQPEDFATYYCQQANSFPLTFGGGTKLEIKR | |
| VENM_M03_ | ETTLTQSPSSVSASVGDRVTITCRASQSISSWLAWYQQ | 279 |
| B12 | KPGKAPKLLINKASSLESGVPSRFSGSGSGTEFTLTISS | |
| | LQPDDFATYYCQQYHSYPLTFGGGTKVDIKR | |
| VENM_M12_ | SNFMLTQPPSASGAPGHSVTISCSGGSSNIGDYDVSWY | 280 |
| G05 | QQLPGTAPKVLIYRNNQRPSGVPDRFSASKSGTSASLA | |
| | ISGLRSEDEADYYCAAWDDSLNGRVFGTGTKLTVLG | |
| VENM_M03_ | EIVLTQSPSSLSASAGDRVTITCRASQGVSSWLAWYQ | 281 |
| A06 | QKPGKAPRLLIYATSRLQSGVPSRFSGSGSGTDFTLTIS | |
| | SLQPEDFATYYCQQSYFTPVTFGQGTRLEIKR | |
| SNEURO2_P01_ | ETTLTQSPDSLAVSLGERATINCKSSQSVLDSSKNKNY | 282 |
| H06 | LAWYQQKPGQPPKLLIYWASIRESGVPDRFSGSGSGT | |
| | DFSLTISSLQAEDVAVYYCQQYYGTPYTFGQGTKLEI | |
| | KR | |
| Centi-D09.0 | EIVLTQSPSSLSASVGDRVTITCRASETISIYLNWYQQK | 501 |
| | PGKAPKLLIYAASSLQSGVPSRFSGSGSGTDFTLTISSQ | |
| | QPEDFATYYCQQSYSTPWTFGQGTKVEIKR | |
| Centi-D09.1 | EIVLTQSPSSLSASVGDRVTITCRASQGISSWLAWYQQ | 502 |
| | KPGKAPKLLIYAASSLQSGVPSRFSGSGSGTDFTLTISS | |
| | QQPEDFATYYCQQANSFPLTFGQGTKVEIKR | |
| Centi-D09.2 | EIVLTQSPSSLSASVGDRVTITCQASQDISNYLNWYQQ | 503 |
| | KPGKAPKLLIYAASSLESGVPSRFSGSGSGTDFTLTISS | |
| | QQPEDFATYYCQQANSFPYTFGQGTKVEIKR | |
| Centi-D09.3 | EIVLTQSPSSLSASVGDRVTITCRASQSISSYLNWYQQ | 504 |
| | KPGKAPKLLIYAASSLQSGVPSRFSGSGSGTDFTLTISS | |
| | QQPEDFATYYCQQSYSTPLTFGQGTKVEIKR | |
| Centi-D09.4 | EIVLTQSPSSLSASVGDRVTITCRASQGISSWLAWYQQ | 505 |
| | KPGKAPKLLIYAASNLQMGVPSRFSGSGSGTDFTLTIS | |
| | SQQPEDFATYYCQQSYTTPLTFGQGTKVEIKR | |
| Centi-D09.5 | EIVLTQSPSSLSASVGDRVTITCRASQSISSYLNWYQQ | 506 |
| | KPGKAPKLLIYAASSLQSGVPSRFSGSGSGTDFTLTISS | |
| | QQPEDFATYYCQQSYSTHTFGQGTKVEIKR | |
| Centi-D09.6 | EIVLTQSPSSLSASVGDRVTITCRASQSISSFLNWYQQK | 507 |
| | PGKAPKLLIYAASSLQSGVPSRFSGSGSGTDFTLTISSQ | |
| | QPEDFATYYCQQSYSPPLTFGQGTKVEIKR | |
| Centi-D09.7 | EIVLTQSPSSLSASVGDRVTITCRASQGISDNLNWYQQ | 508 |
| | KPGKAPKLLIYAASTLQSGVPSRFSGSGSGTDFTLTISS | |
| | QQPEDFATYYCQQANSFPLTFGQGTKVEIKR | |
| Centi-D09.8 | EIVLTQSPSSLSASVGDRVTITCRASQDIRNDLGWYQQ | 509 |
| | KPGKAPKLLIYGASTLQSGVPSRFSGSGSGTDFTLTISS | |
| | QQPEDFATYYCQQSYITPLTFGQGTKVEIKR | |
| Centi-D09.9 | EIVLTQSPSSLSASVGDRVTITCRASQSISSWLAWYQQ | 510 |
| | KPGKAPKLLIYAASSLQSGVPSRFSGSGSGTDFTLTISS | |
| | QQPEDFATYYCQQSYSTITFGQGTKVEIKR | |
| Centi-D09.10 | EIVLTQSPSSLSASVGDRVTITCRASQSISSYLNWYQQ | 511 |
| | KPGKAPKLLIYAASTLQSGVPSRFSGSGSGTDFTLTISS | |
| | QQPEDFATYYCQQANSFPPTFGQGTKVEIKR | |
| Centi-D09.11 | EIVLTQSPSSLSASVGDRVTITCRASQGIRNDLNWYQQ | 512 |
| | KPGKAPKLLIYAASNSLSGVPSRFSGSGSGTDFTLTISS | |
| | QQPEDFATYYCQQANSFPLTFGQGTKVEIKR | |
| SNEURO_P01_ | EIVLTQSPSSLSASVGDRVTITCQASQDISNYLNWYQQ | 518 |
| D09 VH | KPGKAPKLLIYAASSLESGVPSRFSGSGSGTDFTLTISS | |
| germlined | QQPEDFATYYCQQANSFPYTFGQGTKVEIK | |
| SNEURO_P01_ | DIQMTQSPSSLSASVGDRVTITCQASQDISNYLNWYQ | 519 |
| D09 VK | QKPGKAPKLLIYAASSLESGVPSRFSGSGSGTDFTLTIS | |
| germlined | SLQPEDFATYYCQQANSFPYTFGQGTKVEIK | |
| SNEURO_P01_ | DIQMTQSPSSLSASVGDRVTITCQASQDISNYLNWYQ | 520 |
| D09 FW | QKPGKAPKLLIYAASSLESGVPSRFSGSGSGTDFTLTIS | |
| germlined | SLQPEDFATYYCQQANSFPYTFGQGTKVEIK | |
| SNEURO_P01_ | EIVLTQSPSSLSASVGDRVTITCQASQDISNYLNWYQQ | 521 |
| D09 VK L3 | KPGKAPKLLIYAASSLESGVPSRFSGSGSGTDFTLTISS | |
| NS→QS | QQPEDFATYYCQQAQSFPYTFGQGTKVEIKR | |
| SNEURO_P01_ | EIVLTQSPSSLSASVGDRVTITCQASQDISNYLNWYQQ | 522 |
| D09 VK L3 | KPGKAPKLLIYAASSLESGVPSRFSGSGSGTDFTLTISS | |
| NS→NT | QQPEDFATYYCQQANTFPYTFGQGTKVEIKR | |
| Centi-DTX- | QPGLTQPPSVSVAPGKTAMITCGGNNIGTKSVHWYQQ | 524 |
| B03 | KPGQAPVLVVFDDSDRPSGIPERFSGSNSGNTATLAIT | |
| | GLQAEDQADYYCQSYDSSLSGVIFGGGTKLTVL | |

### Exemplary Antibodies

An antibody or antigen-binding fragment herein can comprise a variable heavy chain (VH) amino acid sequence and a variable light chain (VL) amino acid sequence as described herein.

An antibody or antigen-binding fragment herein can comprise a VH having an amino acid sequence of SEQ ID NO: 224 and a VL having an amino acid sequence of SEQ ID NO: 249 (PLA2_P01_A04).

An antibody or antigen-binding fragment herein can comprise a VH having an amino acid sequence of SEQ ID NO: 225 and a VL having an amino acid sequence of SEQ ID NO: 250 (PLA2_P01_A08).

An antibody or antigen-binding fragment herein can comprise a VH having an amino acid sequence of SEQ ID NO: 226 and a VL having an amino acid sequence of SEQ ID NO: 251 (PLA2_P01_B02).

An antibody or antigen-binding fragment herein can comprise a VH having an amino acid sequence of SEQ ID NO: 227 and a VL having an amino acid sequence of SEQ ID NO: 252 (PLA2_P01_B08).

An antibody or antigen-binding fragment herein can comprise a VH having an amino acid sequence of SEQ ID NO: 228 and a VL having an amino acid sequence of SEQ ID NO: 253 (PLA2_P01_C02).

An antibody or antigen-binding fragment herein can comprise a VH having an amino acid sequence of SEQ ID NO: 229 and a VL having an amino acid sequence of SEQ ID NO: 254 (PLA2_P01_C03).

An antibody or antigen-binding fragment herein can comprise a VH having an amino acid sequence of SEQ ID NO: 230 and a VL having an amino acid sequence of SEQ ID NO: 255 (PLA2_P01_C04).

An antibody or antigen-binding fragment herein can comprise a VH having an amino acid sequence of SEQ ID NO: 231 and a VL having an amino acid sequence of SEQ ID NO: 256 (PLA2_P01_D06.1).

An antibody or antigen-binding fragment herein can comprise a VH having an amino acid sequence of SEQ ID NO: 231 and a VL having an amino acid sequence of SEQ ID NO: 257 (PLA2_P01_D06.2).

An antibody or antigen-binding fragment herein can comprise a VH having an amino acid sequence of SEQ ID NO: 232 and a VL having an amino acid sequence of SEQ ID NO: 258 (PLA2_P01_G05).

An antibody or antigen-binding fragment herein can comprise a VH having an amino acid sequence of SEQ ID NO: 233 and a VL having an amino acid sequence of SEQ ID NO: 259 (PLA2_P01_G07).

An antibody or antigen-binding fragment herein can comprise a VH having an amino acid sequence of SEQ ID NO: 224 and a VL having an amino acid sequence of e SEQ ID NO: 249 (PLA2_P01_G12).

An antibody or antigen-binding fragment herein can comprise a VH having an amino acid sequence of SEQ ID NO: 234 and a VL having an amino acid sequence of SEQ ID NO: 260 (KUNITZ_P01_A03.1).

An antibody or antigen-binding fragment herein can comprise a VH having an amino acid sequence of SEQ ID NO: 234 and a VL having an amino acid sequence of SEQ ID NO: 261 (KUNITZ_P01_A03.2).

An antibody or antigen-binding fragment herein can comprise a VH having an amino acid sequence of SEQ ID NO: 235 and a VL having an amino acid sequence of SEQ ID NO: 262 (KUNITZ_P01_A07).

An antibody or antigen-binding fragment herein can comprise a VH having an amino acid sequence of SEQ ID NO: 236 and a VL having an amino acid sequence of SEQ ID NO: 263 (KUNITZ_P01_D07).

An antibody or antigen-binding fragment herein can comprise a VH having an amino acid sequence of SEQ ID NO: 237 and a VL having an amino acid sequence of SEQ ID NO: 264 (KUNITZ_P01_H05.1).

An antibody or antigen-binding fragment herein can comprise a VH having an amino acid sequence of SEQ ID NO: 237 and a VL having an amino acid sequence of SEQ ID NO: 265 (KUNITZ_P01_H05.2).

An antibody or antigen-binding fragment herein can comprise a VH having an amino acid sequence of SEQ ID NO: 238 and a VL having an amino acid sequence of SEQ ID NO: 266 (KUNITZ_P01_H07.1).

An antibody or antigen-binding fragment herein can comprise a VH having an amino acid sequence of SEQ ID NO: 238 and a VL having an amino acid sequence of SEQ ID NO: 267 (KUNITZ_P01_H07.2).

An antibody or antigen-binding fragment herein can comprise a VH having an amino acid sequence of SEQ ID NO: 239 and a VL having an amino acid sequence of SEQ ID NO: 268 (SNEURO_P01_A04).

An antibody or antigen-binding fragment herein can comprise a VH having an amino acid sequence of SEQ ID NO: 240 and a VL having an amino acid sequence of SEQ ID NO: 269 (SNEURO_P01_A08).

An antibody or antigen-binding fragment herein can comprise a VH having an amino acid sequence of SEQ ID NO: 241 and a VL having an amino acid sequence of SEQ ID NO: 270 (SNEURO_P01_A12).

An antibody or antigen-binding fragment herein can comprise a VH having an amino acid sequence of SEQ ID NO: 239 and a VL having an amino acid sequence of SEQ ID NO: 271 (SNEURO_P01_B05).

An antibody or antigen-binding fragment herein can comprise a VH having an amino acid sequence of SEQ ID NO: 239 and a VL having an amino acid sequence of SEQ ID NO: 272 (SNEURO_P01_B11).

An antibody or antigen-binding fragment herein can comprise a VH having an amino acid sequence of SEQ ID NO: 242 and a VL having an amino acid sequence of SEQ ID NO: 273 (SNEURO_P01_D09).

An antibody or antigen-binding fragment herein can comprise a VH having an amino acid sequence of SEQ ID NO: 243 and a VL having an amino acid sequence of SEQ ID NO: 274 (SNEURO_P01_E06).

An antibody or antigen-binding fragment herein can comprise a VH having an amino acid sequence of SEQ ID NO: 243 and a VL having an amino acid sequence of SEQ ID NO: 275 (SNEURO_P01_E09).

An antibody or antigen-binding fragment herein can comprise a VH having an amino acid sequence of SEQ ID NO: 244 and a VL having an amino acid sequence of SEQ ID NO: 276 (SNEURO_P01_F07).

An antibody or antigen-binding fragment herein can comprise a VH having an amino acid sequence of SEQ ID NO: 239 and a VL having an amino acid sequence of SEQ ID NO: 277 (SNEURO_P01_F09).

An antibody or antigen-binding fragment herein can comprise a VH having an amino acid sequence of SEQ ID NO: 245 and a VL having an amino acid sequence of SEQ ID NO: 278 (SNEURO_P01_H02).

An antibody or antigen-binding fragment herein can comprise a VH having an amino acid sequence of SEQ ID NO: 246 and a VL having an amino acid sequence of SEQ ID NO: 279 (VENM_M03_B12).

An antibody or antigen-binding fragment herein can comprise a VH having an amino acid sequence of SEQ ID NO: 247 and a VL having an amino acid sequence of SEQ ID NO: 280 (VENM_M12_G05).

An antibody or antigen-binding fragment herein can comprise a VH having an amino acid sequence of SEQ ID NO: 248 and a VL having an amino acid sequence of SEQ ID NO: 281 (VENM_M03_A06).

An antibody or antigen-binding fragment herein can comprise a VH having an amino acid sequence of SEQ ID NO: 235 and a VL having an amino acid sequence of SEQ ID NO: 282 (SNEURO_P01_H06).

An antibody or antigen-binding fragment herein can comprise a VH of SEQ ID NO: 429 and a VL of any one of SEQ ID NOS: 501-512.

An antibody or antigen-binding fragment herein can comprise a VH of SEQ ID NO: 430 and a VL of any one of SEQ ID NOS: 501-512.

An antibody or antigen-binding fragment herein can comprise a VH of SEQ ID NO: 431 and a VL of any one of SEQ ID NOS: 501-512.

An antibody or antigen-binding fragment herein can comprise a VH of SEQ ID NO: 432 and a VL of any one of SEQ ID NOS: 501-512.

An antibody or antigen-binding fragment herein can comprise a VH of SEQ ID NO: 433 and a VL of any one of SEQ ID NOS: 501-512.

An antibody or antigen-binding fragment herein can comprise a VH of SEQ ID NO: 434 and a VL of any one of SEQ ID NOS: 501-512.

An antibody or antigen-binding fragment herein can comprise a VH of SEQ ID NO: 435 and a VL of any one of SEQ ID NOS: 501-512.

An antibody or antigen-binding fragment herein can comprise a VH of SEQ ID NO: 436 and a VL of any one of SEQ ID NOS: 501-512.

An antibody or antigen-binding fragment herein can comprise a VH of SEQ ID NO: 437 and a VL of any one of SEQ ID NOS: 501-512.

An antibody or antigen-binding fragment herein can comprise a VH of SEQ ID NO: 438 and a VL of any one of SEQ ID NOS: 501-512.

An antibody or antigen-binding fragment herein can comprise a VH of SEQ ID NO: 439 and a VL of any one of SEQ ID NOS: 501-512.

An antibody or antigen-binding fragment herein can comprise a VH of SEQ ID NO: 440 and a VL of any one of SEQ ID NOS: 501-512.

An antibody or antigen-binding fragment herein can comprise a VH of SEQ ID NO: 441 and a VL of any one of SEQ ID NOS: 501-512.

An antibody or antigen-binding fragment herein can comprise a VH of SEQ ID NO: 442 and a VL of any one of SEQ ID NOS: 501-512.

An antibody or antigen-binding fragment herein can comprise a VH of SEQ ID NO: 443 and a VL of any one of SEQ ID NOS: 501-512.

An antibody or antigen-binding fragment herein can comprise a VH of SEQ ID NO: 444 and a VL of any one of SEQ ID NOS: 501-512.

An antibody or antigen-binding fragment herein can comprise a VH of SEQ ID NO: 445 and a VL of any one of SEQ ID NOS: 501-512.

An antibody or antigen-binding fragment herein can comprise a VH of SEQ ID NO: 446 and a VL of any one of SEQ ID NOS: 501-512.

An antibody or antigen-binding fragment herein can comprise a VH of SEQ ID NO: 447 and a VL of any one of SEQ ID NOS: 501-512.

An antibody or antigen-binding fragment herein can comprise a VH of SEQ ID NO: 448 and a VL of any one of SEQ ID NOS: 501-512.

An antibody or antigen-binding fragment herein can comprise a VH of SEQ ID NO: 449 and a VL of any one of SEQ ID NOS: 501-512.

An antibody or antigen-binding fragment herein can comprise a VH of SEQ ID NO: 450 and a VL of any one of SEQ ID NOS: 501-512.

An antibody or antigen-binding fragment herein can comprise a VH of SEQ ID NO: 513 and a VL of SEQ ID NO: 518. (SNEURO_P01_D09 VH germlined)

An antibody or antigen-binding fragment herein can comprise a VH of SEQ ID NO: 514 and a VL of SEQ ID NO: 519. (SNEURO_P01_D09 VK germlined)

An antibody or antigen-binding fragment herein can comprise a VH of SEQ ID NO: 515 and a VL of SEQ ID NO: 520. (SNEURO_P01_D09 FW germlined)

An antibody or antigen-binding fragment herein can comprise a VH of SEQ ID NO: 516 and a VL of SEQ ID NO: 521. (SNEURO_P01_D09 VK L3 NS->QS)

An antibody or antigen-binding fragment herein can comprise a VH of SEQ ID NO: 517 and a VL of SEQ ID NO: 522. (SNEURO_P01_D09 VK L3 NS->NT)

An antibody or antigen-binding fragment herein can comprise a VH of SEQ ID NO: 523 and a VL of SEQ ID NO: 524. (Centi-DTX-B03)

In one embodiment, the antibody or antigen-binding fragment herein comprises a VH CDR1, a VH CDR2, a VH CDR3, a VL CDR1, a VL CDR2, and a VL CDR3, wherein: (i) the VH CDR1 comprises the amino acid sequence FX₂X₃X₄X₅X₆DX₈H, wherein X₂ is selected from N, T, and S; X₃ is selected from F, L; X₄ is selected from R, G, and S; X₅ is selected from N and T; X₆ is selected from Y, F, L; and Xx is selected from M, I; (ii) the VH CDR2 comprises the amino acid sequence X₁X₂X₃X₄X₅X₆X₇GX₉X₁₀X₁₁X₁₂, wherein X₁ is selected from P, S, and H; X₂ is selected from V, G, and T; X₃ is selected from V, F, L, and I; X₄ is selected from D, G, and A; X₅ is selected from Y, L, and H; X₆ is selected from N, C, R, and T, and S; X₇ is selected from V, F, and G; X₉ is selected from A, E; X₁₀ is selected from Q, H; and X₁₁ is selected from S, D, Y, and H; and X₁₂ is selected from A and E; (iii) the VH CDR3 comprises the amino acid sequence CX₂RGTLYHYTSGSYX₁₅SDAFDIW, wherein X₂ is selected from V and A; and X₁₅ is selected from Y and C (SEQ ID NO: 527); (iv) the VL CDR1 comprises the amino acid sequence X₁ASX₄X₅IX₇X₈X₉LX₁₁, wherein X₁ is selected from Q and R; X₄ is selected from Q and E; X₅ is selected from D, G, T, and S; X₇ is selected from R and S; X₈ is selected from S, D, N, and I; X₉ is selected from N, F, Y, W, and D; and X₁₁ is selected from N, G, and A (SEQ ID NO: 528); (v) the VL CDR2 comprises the amino acid sequence X₁ASX₄X₅X₆X₇, wherein X₁ is selected from G and A; X₄ is selected from N, T, and S; X₅ is selected from L and S; X₆ is selected from Q, L, and E; and X₇ is selected from M and S; and (vi) the VL CDR3 comprises the amino acid sequence CQQSYSTX&TF, wherein X₈ is selected from I and H (SEQ ID NO: 530); or the VL CDR3 comprises the amino acid sequence CQQX₄X₅X₆X₇PX₉TF, wherein X₄ is selected from A and S; X₅ is selected from N and Y; X₆ is selected from I, T, and S; X₇ is selected from P, F, and T; and X₉ is selected from P, Y, L, and W (SEQ ID NO: 531).

In some embodiments, the antibody or antigen-binding fragment of the invention specifically binds multiple homologous members of the three-fingered-toxin (3FTX) family found in Elapidae. The antibody or antigen-binding fragment may specifically bind to one or more homologous members of the 3FTX family that are alpha-neurotoxins. The antibody or antigen-binding fragment may specifically bind to one or more homologous members of the 3FTX family that are long neurotoxins. In a certain embodiment, the antibody or antigen-binding fragment neutralizes the effect of the long neurotoxin. In a certain embodiment, the antibody or antigen-binding fragment binds one or more three-fingered toxins in such a manner that prevents binding of the toxin to human nicotinic acetylcholine receptors (nAChRs). In some embodiments, the antibody or antigen-binding fragment binds at least 30%, 40%, 50%, 60%, 70%, 80%, or 90% of the toxin accessible surface area that is buried upon the toxin binding nAChR.

In a certain embodiment, the antibody or antigen-binding fragment binds two or more long-neurotoxin members of the 3FTX family using the same poly-specific paratope (i.e., a single set of CDRs that can bind to two or more long-neurotoxin members of the 3FTX family).

In some embodiments, the antibody or antigen-binding fragment specifically binds members of the long neurotoxin 3FTX family that the hyperimmune subject from which the antibody was derived had not been exposed to.

The antibody or antigen-binding fragment may specifically bind multiple members of the long-neurotoxin 3FTX family. The antibody or antigen-binding fragment may specifically bind two three-fingered toxins that share less than 90%, 80%, 70%, 60%, or 50% sequence identity. The antibody or antigen-binding fragment may specifically bind three three-fingered toxins, no pair of which shares more than 90%, 80%, 70%, 60%, or 50% sequence identity. The antibody or antigen-binding fragment may specifically bind four, five, or six three-fingered toxins, no pair of which shares more than 90%, 80%, 70%, 60%, or 50% sequence identity.

In one embodiment, the antibody or antigen-binding fragment binds alpha-bungarotoxin found in common krait, alpha-elapitoxin found in black mamba, and alpha-neurotoxin found in monocled cobra. In one embodiment, the antibody or antigen-binding fragment may bind long-neurotoxin and short-neurotoxin members of the 3FTX family.

In one embodiment, the antibody or antigen-binding fragment binds long-neurotoxin three-fingered toxins found in two or three or more of the following: in a mamba species (in the genus dendroaspis), in a cobra species (in the genus naja), in a taipan species (in the genus oxyuranus), and in a krait species (in the genus bungarus). In one embodiment, the antibody or antigen-binding fragment binds long-neurotoxin three-fingered toxins found in at least one mamba species, in at least one cobra species, in at least one taipan species, and in at least one krait species. In one embodiment, the antibody or antigen-binding fragment binds long-neurotoxin three-fingered toxins from two or three or more of the following species: black mamba (dendroaspis polylepis), monocled cobra (naja kaouthia), coastal taipan (oxyuranus scutellatus), and common krait (bungarus caeruleus). In a certain embodiment, the antibody or antigen-binding fragment binds long-neurotoxin three-fingered toxins from black mamba (dendroaspis polylepis), monocled cobra (naja kaouthia), coastal taipan (oxyuranus scutellatus), and common krait (bungarus caeruleus).

In one embodiment, the antibody or antigen-binding fragment binds long-neurotoxin from two or more species of cobra (in the genus naja). In one embodiment, the antibody or antigen-binding fragment binds long-neurotoxin from two or more species of cobra that are found on different continents. The antibody or antigen-binding fragment may bind 3FTX found in an Asian snake species and 3FTX found in an African snake species. For example, the antibody or antigen-binding fragment may bind Indian cobra (naja naja) and Egyptian cobra (naja haje). The antibody or antigen-binding fragment may bind 3FTX found in a cobra found in India, a cobra found in Africa, and a cobra found in Indonesia.

In one embodiment, the antibody or antigen-binding fragment binds long-neurotoxin from one or more species in the family Elapidae found in Australia, long-neurotoxin from one or more species in the family Elapidae found in India, and long-neurotoxin from one or more species in the family Elapidae found in Africa. For example, the antibody or antigen-binding fragment may bind to long-neurotoxins found in coastal taipan (Australia), Indian cobra (India), and black mamba (Africa).

The antibody or antigen-binding fragment may bind at least one long neurotoxin found in a cobra of the genus naja and at least one long neurotoxin found in a cobra of the genus ophiophagus.

In one embodiment, the antibody or antigen-binding fragment binds at least one long-neurotoxin found in the venom of a snake in the family Elapidae not in the subfamily Hydrophiinde and at least one long-neurotoxin found in the venom of a snake in the subfamily Hydrophiinde. In one embodiment, the antibody or antigen-binding fragment binds long-neurotoxin from banded sea krait (laticauda colubrina) and long-neurotoxin from at least one species in the family Elapidae. In one embodiment, the antibody or antigen-binding fragment binds long-neurotoxin found in two or more of the following: in a aipysurus species, in a hydrophis species, and in a laticauda species.

A combination or a population of antibodies or antigen-binding fragments herein can comprise one, two, three, four, five, six, seven, eight, nine, ten, or more different antibodies. In one instance, a population of antibodies or antigen-binding fragments herein comprises one antibody or antigen-binding fragments herein. In one instance, a combination or a population of antibodies or antigen-binding fragments herein comprises two different antibodies or antigen-binding fragments. In one instance, a combination or a population of antibodies or antigen-binding fragments herein comprises three different antibodies or antigen-binding fragments. In one instance, a combination or a population of antibodies or antigen-binding fragments herein comprises four different antibodies or antigen-binding fragments. In one instance, a combination or a population of antibodies or antigen-binding fragments herein comprises five different antibodies or antigen-binding fragments. In one instance, a combination or a population of antibodies or antigen-binding fragments herein comprises six different antibodies or antigen-binding fragments. In one instance, a combination or a population of antibodies or antigen-binding fragments herein comprises seven different antibodies or antigen-binding fragments. In one instance, a combination or a population of antibodies or antigen-binding fragments herein comprises eight different antibodies or antigen-binding fragments. In one instance, a combination or a population of antibodies or antigen-binding fragments herein comprises nine different antibodies or antigen-binding fragments. In one instance, a combination or a population of antibodies or antigen-binding fragments herein comprises ten different antibodies or antigen-binding fragments. In one instance, a combination or a population of antibodies or antigen-binding fragments herein comprises more than ten different antibodies or antigen-binding fragments. A combination or a population of antibodies or antigen-binding fragments herein can comprise antibodies that bind to snakes from a continent, country, or region. For example, a combination or a population of antibodies or antigen-binding fragments herein can be useful for treating snake bites in north, central America, south America, the middle east, India, China, Africa, Asia, Australia, etc. Alternatively, a combination or a population of antibodies or antigen-binding fragments herein can be useful for treating snake bites in a region, for example, northeast United States, southeast United States, southwest United States, or northwest United States. Such combinations or populations of antibodies or antigen-binding fragments herein are useful when a subject who has been bitten cannot identify a particular snake.

This application also discloses methods for the identification of broadly-neutralizing antibodies against a family of homologous antigens (e.g., snake venom toxins). Families of homologous antigens found in snake venom include the three-finger toxins (3FTXs), long neurotoxins, short neurotoxins, snake venom metalloproteinases (SVMPs), snake venom serine proteases (SVSPs), phospholipase A2s (PLA2s). The family of homologous antigens may be another toxin or family of toxins that is present in multiple species of snakes.

The method may comprise immunizations of a human or another animal with two or more homologous antigens (the “hyperimmune subject”). In some embodiments, the method may comprise immunizations with 2, 3, 4, 5, 6, 7, 8, 9, or 10 homologous antigens. The method may comprise immunizations with between 10 and 20 homologous antigens. The method may comprise immunizations with between 20 and 30 homologous antigens. The method may comprise immunizations with between 30 and 40 homologous antigens. In some embodiments, the immunizations may comprise recombinant homologous antigens. These antigens may be selected to sample a diverse subset of the homologous antigens. In some embodiments, the immunizations may comprise whole venom from different species that contain homologous toxin antigens. These species may be selected to be phylogenetically diverse.

Each of these immunizations may be repeated two or more times. In some embodiments, the method may comprise immunizations that are repeated 2, 3, 4, 5, 6, 7, 8, 9, or 10 times. The method may comprise immunizations that are repeated between 10 and 20 times. The method may comprise immunizations that are repeated between 20 and 30 times. The method may comprise immunizations that are repeated between 30 and 40 times. The method may comprise immunizations that are repeated more than 40 times. For example, an immunization schedule comprising three homologous antigens that repeats twice may be as follows: immunization with antigen A1, then A2, then A3; then A1, A2, and A3 again.

The method may further comprise identification of monoclonal antibodies from the subject with cross-reactivity to two or more homologous antigens. In a certain embodiment, the method comprises iterative rounds of selection to identify such cross-reactive monoclonal antibodies. Examples of such methods for selection include immunoprecipitation of antibodies from serum; fluorescence-activated cell sorting (FACS) of B cells; or panning of a library displayed in phage or yeast.

Independent of the specific selection technology employed, the method of the invention comprises iterative selection using multiple homologous antigens to specifically enrich for antibodies derived from the hyperimmune subject that are cross-reactive and broadly-neutralizing against multiple members of the homologous antigen family. The iterative rounds of selection may utilize a sequence of different homologous antigens to preferentially select for antibodies that are cross-reactive. The iterative rounds of selection may utilize members of the homologous antigen family that the hyperimmune subject was not immunized against to preferentially select for antibodies that are cross-reactive. The selection process may employ use of 2, 3, 4, 5, 6, 7, 8, 9, or 10 different members of the homologous antigen family. Clones identified using the iterative down-selection are guaranteed to be cross-reactive for all members of the homologous antigen family included in the iterative down-selection method.

An exemplary method for identification of cross-reactive antibodies using iterative selection using the phage display is given here. The method may comprise isolation of PBMCs or B cells from said hyperimmune subject. The method may further comprise extraction of RNA. The method may further comprise reverse transcription of cDNA from the extracted RNA. The method may further comprise amplification of antibody variable domains (e.g., by PCR). The method may further comprise construction of a DNA library of said antibody variable domains. The method may further comprise digestion and cloning of the DNA library into a phage display vector. The method may further comprise panning against one or more toxins. The method may comprise panning against 2, 3, 4, 5, 6, 7, 8, 9, or 10 different members of the homologous antigen family; such that retained clones must be cross-reactive to all members utilized in the iterative panning process. The toxins may comprise toxins the hyperimmune subject had prior exposure to, in addition to homologous toxins the hyperimmune subject did not have prior exposure to. The method may further comprise screening antibody clones for binding, for example by ELISA or kinetics (e.g., Octet HTX or Biacore).

### Modified Antibodies

The present disclosure provides for modified antibodies. Modified antibodies can comprise antibodies which have one or more modifications which can enhance their activity, binding, specificity, selectivity, or another feature. In one aspect, the present disclosure provides for modified antibodies (which can be heteromultimers) that comprise an antibody as described herein. Reference to a modified antibody herein also refers to a modified antigen-binding fragment. A modified antibody can comprise a bispecific modified antibody or antigen-binding fragment, a trispecific modified antibody or antigen-binding fragment, or a tetraspecific modified antibody or antigen-binding fragment.

A modified antibody can comprise a human modified antibody. Also included herein are amino acid sequence variants of the modified antibody which can be prepared by introducing appropriate nucleotide changes into the modified antibody DNA, or by synthesis of the desired modified antibody polypeptide. Such variants include, for example, deletions from, or insertions or substitutions of, residues within the amino acid sequences of the first and second polypeptides forming the modified antibody. Any combination of deletion, insertion, and substitution is made to arrive at the final construct, provided that the final construct possesses the desired antigen-binding characteristics. The amino acid changes also may alter post-translational processes of the modified antibody, such as changing the number or position of glycosylation sites.

“Alanine scanning mutagenesis” can be a useful method for identification of certain residues or regions of the modified antibody polypeptides that might be preferred locations for mutagenesis. Here, a residue or group of target residues are identified (e.g., charged residues such as Arg, Asp, His, Lys, and Glu) and replaced by a neutral or negatively charged amino acid (for example, alanine or polyalanine) to affect the interaction of the amino acids with the surrounding aqueous environment in or outside the cell. Those domains demonstrating functional sensitivity to the substitutions then are refined by introducing further or other variants at or for the sites of substitution. Thus, while the site for introducing an amino acid sequence variation is predetermined, the nature of the mutation per se need not be predetermined.

Normally the substitutions can involve conservative amino acid replacements in non-functional regions of the modified antibody. Exemplary substitutions are shown below.

TABLE 18
Exemplary substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Covalent modifications of antibody, antigen-binding fragment, or modified antibody polypeptides are included within the scope of this disclosure. Covalent modifications of the modified antibody can be introduced into the molecule by reacting targeted amino acid residues of the modified antibody or fragments thereof with an organic derivatizing agent that can be capable of reacting with selected side chains or the N- or C-terminal residues. Another type of covalent modification of the modified antibody polypeptide can comprise altering the native glycosylation pattern of the polypeptide. Herein, “altering” can mean deleting one or more carbohydrate moieties found in the original modified antibody, and/or adding one or more glycosylation sites that are not present in the original modified antibody. Addition of glycosylation sites to the modified antibody polypeptide can be accomplished by altering the amino acid sequence such that it contains one or more N-linked glycosylation sites. The alteration may also be made by the addition of, or substitution by, one or more serine or threonine residues to the original modified antibody sequence (for O-linked glycosylation sites). For ease, the modified antibody amino acid sequence can be altered through changes at the DNA level, particularly by mutating the DNA encoding the modified antibody polypeptide at preselected bases such that codons are generated that will translate into the desired amino acids. Another means of increasing the number of carbohydrate moieties on the modified antibody polypeptide is by chemical or enzymatic coupling of glycosides to the polypeptide. Removal of carbohydrate moieties present on the modified antibody can be accomplished chemically or enzymatically.

Another type of covalent modification of modified antibody comprises linking the modified antibody polypeptide to one of a variety of non-proteinaceous polymers, e.g., polyethylene glycol, polypropylene glycol, or polyoxyalkylenes.

Methods for complexing binding agents or the antibody or antigen-binding fragments thereof herein with another agent are known in the art. Such methods may utilize one of several available heterobifunctional reagents used for coupling or linking molecules.

In one instance, Fc portions of antibodies can be modified to increase half-life of the molecule in the circulation in blood when administered to a subject.

Additionally, antibodies may be produced or expressed so that they do not contain fucose on their complex N-glycoside-linked sugar chains to increase effector functions. Similarly, antibodies can be attached at their C-terminal end to all, or part, of an immunoglobulin heavy chain derived from any antibody isotype, e.g., IgG, IgA, IgE, IgD, and IgM, and any of the isotype sub-classes, e.g., IgG1, IgG2b, IgG2a, IgG3, and IgG4.

Glycosylation of immunoglobulins has been shown to have significant effects on their effector functions, structural stability, and rate of secretion from antibody-producing cells. Antibodies and antigen-binding fragments herein may be glycosylated. Glycosylation at a variable domain framework residue can alter the binding interaction of the antibody with antigen. The present disclosure includes criteria by which a limited number of amino acids in the framework or CDRs of an immunoglobulin chain can be chosen to be mutated (e.g., by substitution, deletion, and/or addition of residues) in order to increase the affinity of an antibody.

Linkers for conjugating antibodies to other moieties are within the scope of the present disclosure. Associations (binding) between antibodies and labels include, but are not limited to, covalent and non-covalent interactions, chemical conjugation, as well as recombinant techniques.

Antibodies, or antigen-binding fragments thereof, can be modified for various purposes such as, for example, by addition of polyethylene glycol (PEG). PEG modification (PEGylation) can lead to one or more of improved circulation time, improved solubility, improved resistance to proteolysis, reduced antigenicity and immunogenicity, improved bioavailability, reduced toxicity, improved stability, and easier formulation.

An antibody or antigen-binding fragment can be conjugated to, or recombinantly engineered with, an affinity tag (e.g., a purification tag). Affinity tags such as, for example, His6 tags (His-His-His-His-His-His) (SEQ ID NO: 283) have been described.

Since it is often difficult to predict in advance the characteristics of a variant modified antibody, it will be appreciated that some screening of the recovered variants may be needed to select an optimal variant. Exemplary methods of screening the recovered variants are described below in the Examples.

### Methods of Expressing Antibodies

Also provided herein are methods of making any of these antibodies or polypeptides. The polypeptides can be produced by proteolytic or other degradation of the antibodies, by recombinant methods (i.e., single or fusion polypeptides) as described above, or by chemical synthesis. Polypeptides of the antibodies, especially shorter polypeptides up to about 50 amino acids, can be made by chemical synthesis. Methods of chemical synthesis are commercially available. For example, an antibody could be produced by an automated polypeptide synthesizer employing a solid phase method.

Antibodies may be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. Methods for making derivatives of antibodies, e.g., single chain, etc. are also within the scope of the present disclosure.

As used herein, “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected with a polynucleotide(s) of this disclosure.

DNA encoding an antibody may be readily isolated and sequenced using conventional procedures (e.g., by using oligonucleotide probes that are capable of binding specifically to genes encoding the heavy and light chains of the monoclonal antibodies). Hybridoma cells may serve as a source of such DNA. Once isolated, the DNA may be placed into one or more expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, or by covalently joining to the immunoglobulin coding sequence all or part of the coding sequence for a non-immunoglobulin polypeptide. In that manner, “chimeric” or “hybrid” antibodies are prepared that have the binding specificity of an antibody herein.

Contemplated herein are vectors that encode the one or more antibodies or antigen-binding fragments herein. As used herein, “vector” means a construct, which is capable of delivering, and possibly expressing, one or more gene(s) or sequence(s) of interest in a host cell. Examples of vectors include, but are not limited to, viral vectors; naked DNA or RNA expression vectors; plasmid, cosmid, or phage vectors; DNA or RNA expression vectors associated with cationic condensing agents; DNA or RNA expression vectors encapsulated in liposomes; and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed. An expression vector can be used to direct expression of an antibody. Expression vectors can be administered to obtain expression of an exogenous protein in vivo.

For high level production, a widely used mammalian expression system is one which utilizes Lonza's GS Gene Expression System™. This system uses a viral promoter and selection via glutamine metabolism to provide development of high-yielding and stable mammalian cell lines.

For alternative high-level production, a widely used mammalian expression system is one which utilizes gene amplification by dihydrofolate reductase deficient (“dhfr”) Chinese hamster ovary cells. The system is based upon the dihydrofolate reductase “dhfr” gene, which encodes the DHFR enzyme, which catalyzes conversion of dihydrofolate to tetrahydrofolate. In order to achieve high production, dhfr-CHO cells are transfected with an expression vector containing a functional DHFR gene, together with a gene that encodes a desired protein. In this case, the desired protein is recombinant antibody heavy chain and/or light chain.

By increasing the amount of the competitive DHFR inhibitor methotrexate (MTX), the recombinant cells develop resistance by amplifying the dhfr gene. In standard cases, the amplification unit employed is much larger than the size of the dhfr gene, and as a result the antibody heavy chain is co-amplified.

When large scale production of the protein, such as the antibody chain, is desired, both the expression level and the stability of the cells being employed are taken into account.

The present application provides one or more isolated polynucleotides (nucleic acids) encoding an antibody or an antigen-binding fragment herein, vectors containing such polynucleotides, and host cells and expression systems for transcribing and translating such polynucleotides into polypeptides.

The present application also provides constructs in the form of plasmids, vectors, transcription or expression cassettes which comprise at least one polynucleotide as above.

The present application also provides a recombinant host cell which comprises one or more constructs as above. A nucleic acid encoding any antibody or antigen-binding fragment herein forms an aspect of the present application, as does a method of production of the antibody, which method comprises expression from encoding nucleic acid therefrom. Expression can be achieved by culturing under appropriate conditions recombinant host cells containing the nucleic acid. Following production by expression, an antibody or a portion thereof can be isolated and/or purified using any suitable technique, then used as appropriate.

Systems for cloning and expression of a polypeptide in a variety of different host cells are contemplated for use herein.

A further aspect provides a host cell containing nucleic acid herein using any suitable method. A still further aspect provides a method comprising introducing such nucleic acid into a host cell. The introduction can be followed by causing or allowing expression from the nucleic acid, e.g., by culturing host cells under conditions for expression of the gene.

One or more polynucleotides encoding an antibody or an antigen-binding fragment can be prepared recombinantly/synthetically in addition to, or rather than, cloned. In a further embodiment, the full DNA sequence of the recombinant DNA molecule or cloned gene(s) of an antibody or antigen-binding fragment herein can be operatively linked to an expression control sequence which can be introduced into an appropriate host using any suitable method.

Nucleic acid sequences can be expressed by operatively linking them to an expression control sequence in an appropriate expression vector and employing that expression vector to transform an appropriate host cell. Any of a wide variety of expression control sequences—sequences that control the expression of a nucleic acid sequence operatively linked to it—can be used in these vectors to express the nucleic acid sequences.

A wide variety of host/expression vector combinations can be employed in expressing the nucleic acid sequences of this disclosure. It will be understood that not all vectors, expression control sequences, and hosts will function equally well to express the nucleic acid sequences. Neither will all hosts function equally well with the same expression system. In some embodiments, in selecting a vector, the host is considered such that the vector can function in it. The vector's copy number, the ability to control that copy number, and the expression of any other proteins encoded by the vector, such as antibiotic markers, may also be considered. In certain embodiments, in selecting a vector, the host is considered such that the vector functions in it. The vector's copy number, the ability to control that copy number, and the expression of any other proteins encoded by the vector, such as antibiotic markers, can also be considered.

The present application also provides a method which comprises using a construct as stated above in an expression system in order to express the antibodies (or portions thereof) as above. Considering these and other factors, a variety of vector/expression control sequence/host combinations can be constructed that can express the nucleic acid sequences on fermentation or in large scale animal culture.

Simultaneous incorporation of the antibody (or portion thereof)-encoding nucleic acids and the selected amino acid position changes can be accomplished by a variety of suitable methods including, for example, recombinant and chemical synthesis.

Provided herein are methods of expressing an antibody or antigen-binding fragment (e.g., an antibody or antigen-binding fragment) in a subject comprising administering to the subject a composition comprising a polynucleotide (e.g., mRNA) encoding the antibody or antigen-binding fragment.

In some cases, administering the polynucleotide to the subject can comprise enteral, gastroenteral, oral, transdermal, epicutaneous, intradermal, subcutaneous, nasal administration, intravenous, intraperitoneal, intraarterial, intramuscular, intraosseous infusion, transmucosal, insufflation, or sublingual administration. In some cases, a polynucleotide can be administered via more than one route.

Antibodies or antigen-binding fragments can be synthesized in the subject based at least in part on the polynucleotide encoding the antibody or antigen-binding fragment. For example, a polynucleotide can enter a cell of the subject, and the antibody or antigen-binding fragment can be synthesized at least in part by using the subject's cellular transcription and/or translation machinery. In some cases, for example, where the polynucleotide is an mRNA molecule, the antibody or antigen-binding fragment can be synthesized at least in part by using the subject's cellular translation machinery (e.g., ribosomes, tRNA, etc.). In some cases, antibody or antigen-binding fragments can be transported from a cell to the plasma of the subject after translation.

### Compositions

Compositions comprising an antibody or antigen-binding fragment herein may be prepared for storage by mixing an antibody or antigen-binding fragment having the desired degree of purity with optional pharmaceutically acceptable carriers, excipients or stabilizers (Remington, The Science and Practice of Pharmacy 20th Ed. Mack Publishing (2000)), in the form of lyophilized formulations or aqueous solutions.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, Ed., Mack Publishing Co., Easton, Pa., 1990; and Remington, The Science and Practice of Pharmacy 20th Ed. Mack Publishing, 2000).

Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

The compositions to be used for in vivo administration may be sterilized. This may be accomplished by, for example, filtration through sterile filtration membranes, or any other art-recognized method for sterilization. Antibody compositions are generally placed into a container having a sterile access port, for example, an intravenous solution bag or vial having a stopper pierceable by a hypodermic injection needle. Other methods for sterilization and filtration are known in the art and are contemplated herein.

In some embodiments of the present disclosure, the compositions are formulated to be free of pyrogens such that they are acceptable for administration to a subject.

The compositions according to the present disclosure may be in unit dosage forms such as solutions or suspensions, tablets, pills, capsules, powders, granules, or suppositories, etc., for intravenous, oral, parenteral or rectal administration, or administration by inhalation or insufflation.

The phrase “pharmaceutically acceptable” refers to molecular entities and compositions that are physiologically tolerable and do not typically produce an allergic or similar untoward reaction, such as gastric upset, dizziness and the like, when administered to a subject.

In some instances, an antibody or antigen-binding fragment can be bound to one or more carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the disclosure. Those skilled in the art will know of other suitable carriers for binding antibodies, or will be able to ascertain such, using routine experimentation.

Various embodiments contemplate the use of the antibodies and antigen-binding fragments to manufacture a medicament for treating a condition, disease or disorder described herein. Medicaments can be formulated based on the physical characteristics of the subject needing treatment and can be formulated in single or multiple formulations based on the stage of the condition, disease or disorder. Medicaments can be packaged in a suitable package with appropriate labels for the distribution to hospitals and clinics wherein the label is for the indication of treating a subject having a disease described herein. Medicaments can be packaged as a single unit or multiple units. Instructions for the dosage and administration of the compositions can be included with the packages as described below. The disclosure is further directed to medicaments of an antibody or antigen-binding fragment and a pharmaceutically acceptable carrier.

### Kits

Provided herein are kits that comprise one or more antibodies or antigen-binding fragments herein. Provided herein is a container means comprising one or more antibodies or antigen-binding fragments herein. The container means may be any suitable container which may house a liquid or lyophilized composition including, but not limited to, a vial, a syringe, a bottle, an intravenous (IV) bag, an ampoule, or any other suitable container. A syringe may be able to hold any volume of liquid suitable for injection into a subject including, but not limited to, 0.5 cc, 1 cc, 2 cc, 5 cc, 10 cc or more. In some embodiments, the antibody or antigen-binding fragment is lyophilized, and the kit comprises one or more suitable buffers for reconstitution prior to injection.

The kit may comprise one or more instruction sheets describing the use of the one or more antibodies or antigen-binding fragments. The kit may include one or more labels describing the contents and use of the one or more antibodies.

### Methods of Treatment

The present disclosure provides methods of preventing or treating a subject who suffers from an envenomation, comprising administering to the subject an antibody or antigen-binding fragment herein. In one instance, the subject to be treated is symptomatic prior to administration of the antibody. In another instance, the subject to be treated is asymptomatic prior to administration of the antibody. A “subject” as described herein, includes, but is not limited to, a human, a rodent, a primate, etc. Treatment can result in partial or complete treatment of one or more symptoms of envenomation.

A subject can be administered an antibody or antigen-binding fragment herein in an amount that achieves at least a partial or complete reduction of one or more symptoms. Reduction can be, for example, a decrease of one or more symptoms by about 5% or more compared to prior to treatment. For the administration to human patients, the compositions can be formulated by methodology known by one in the art. The amount of an antibody necessary to bring about therapeutic treatment of a snake bite is not fixed per se. The amount of antibody administered may vary with the extensiveness of the disease, and size of the human suffering from a snake bite. Treatment, in one instance, lowers infection rates in a population of subjects. Treatment may also result in a shortened recovery time, in fewer symptoms, or in less severe symptoms, or a combination thereof compared to an untreated subject who has a snake bite.

“Administering” is referred to herein as providing one or more compositions to a patient in a manner that results in the composition being inside the patient's body. Such an administration can be by any route including, without limitation, locally, regionally, or systemically, by subcutaneous, intradermal, intravenous, intra-arterial, intraperitoneal, or intramuscular administration (e.g., injection). In one instance, administration is via intradermal injection. In another instance, administration is via subcutaneous injection. In one embodiment, a subject is administered one of the antibodies or antigen-binding fragments herein one or more times. In another embodiment, a subject is administered two of the antibodies or antigen-binding fragments herein one or more times. In another embodiment, a subject is administered three of the antibodies or antigen-binding fragments herein one or more times. In another embodiment, a subject is administered four of the antibodies or antigen-binding fragments herein one or more times. Administered can be any route available, including, but not limited to, enteral, gastroenteral, oral, transdermal, epicutaneous, intradermal, subcutaneous, nasal administration, intravenous, intraperitoneal, intraarterial, intramuscular, intraosseous infusion, transmucosal, insufflation, or sublingual administration.

Actual dosage levels of antibody can be varied so as to obtain an amount of the active ingredient that is effective to achieve the desired therapeutic response without being toxic to the patient. The selected dosage level will depend upon a variety of factors including the activity of the particular antibody employed, the route of administration, the time of administration, the rate of excretion of the particular antibody being employed, the duration of the treatment, other drugs, compounds and/or materials used in combination with the particular composition employed, the age, sex, weight, condition, general health, and prior medical history of the patient being treated, and like factors.

The antibodies and antigen-binding fragments herein can be administered to a subject in various dosing amounts and over various time frames.

A physician or veterinarian can readily determine and prescribe the effective amount (ED50) of the antibody required. For example, the physician or veterinarian could start doses of the antibody employed in the composition at levels lower than that required in order to achieve the desired therapeutic effect and gradually increase the dosage until the desired effect is achieved. Alternatively, a dose can remain constant.

The antibody can be administered to a patient by any convenient route such as described above. Regardless of the route of administration selected, the antibodies of the present disclosure, which can be used in a suitable hydrated form, and/or the compositions, are formulated into acceptable dosage forms.

Toxicity and therapeutic efficacy of compounds can be determined by standard procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD50/ED50. While compounds that exhibit toxic side effects may be used, care should be taken to design a delivery system that targets such compounds to the site of affected tissue in order to minimize potential damage to healthy cells and, thereby, reduce side effects.

Data obtained from cell culture assays and/or animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound, a therapeutically effective dose can be estimated initially from cell culture assays. A dose can be formulated in animal models to achieve a circulating plasma concentration that includes the IC50 (i.e., the concentration of the test compound which achieves a half-maximal inhibition) as determined in cell culture. Levels in plasma can be measured, for example, by high performance liquid chromatography. Such information can be used to more accurately determine useful doses in humans.

It will be understood that administration of one or more of the antibodies or antigen-binding fragments herein can be supplemented by one or more additional therapies or drugs such as, for example, a non-steroidal anti-inflammatory drug (NSAID). The most prominent NSAIDs are aspirin, acetaminophen, ibuprofen, and naproxen, all available over the counter (OTC) in most countries. Additional non-limiting examples of NSAIDS include, but are not limited to, diflunisal, dexibuprofen, fenoprofen, ketoprofen, dexketoprofen, flurbiprofen, oxaprozin, loxoprofen, indomethacin, tolmetin, sulindac, etodolac, ketorolac, diclofenac, aceclofenac, bromfenac, nabumetone, piroxicam, meloxicam, tenoxicam, droxicam, lornoxicam, naproxen sodium, acetyl salicylic acid, mefenamic acid, celecoxib, and phenylbutazone. The one or more additional therapies or drugs may comprise, for example, a PLA2 inhibitor. PLA2 inhibitors include, but are not limited to, comprises varespladib, methylvarespladib, or a combination thereof.

### EXEMPLARY DEFINITIONS

The term “about” as used herein, generally refers to a range that is 2%, 5%, 10%, 15% greater than or less than (±) a stated numerical value within the context of the particular usage. For example, “about 10” would include a range from 8.5 to 11.5. As used herein, the terms “about” and “approximately,” when used to modify a numeric value or numeric range, indicate that deviations of up to about 0.2%, about 0.5%, about 1%, about 2%, about 5%, about 7.5%, or about 10% (or any integer between about 1% and 10%) above or below the value or range remain within the intended meaning of the recited value or range.

As used in this specification and the appended claims, the singular forms “a,” “an,” and “the” include plural references unless the context clearly dictates otherwise. Thus, for example, references to “a method” include one or more methods, and/or steps of the type herein and/or which will become apparent to those persons skilled in the art upon reading this disclosure.

An antigen can be a molecule that can be recognized by the immune system, which can include venom, for example, of a snake. For example, an antigen can be a foreign substance that can induce an immune response. In some cases, the induced immune response can comprise the production of antibodies. In some cases, an antibody can recognize, attach to, or bind to an antigen.

A “snake” as referenced herein, refers to any venomous snake including, but not limited to, Boiga irregularis (Brown Tree Snake), Boiga cyanea (Green Cat Snake), Boiga dendrophila (Mangrove Snake), Dispholidus typus (Boomslang), Salvadora grahamiae (Mountain Patchnose Snake), Spalerosophis diadema (Diadem Snake), Tantilla nigriceps (Plains Blackhead Snake), Thelotornis capensis (Southern Twig Snake), Thelotornis kirtlandii (Northern Twig Snake), Toxicodryas blandingii (Blandings Tree Snake), Trimorphodon lambda (Sonoran Lyre Snake), Amphiesma stolatum (Buff Striped Keelback), Natrix tessellate (Dice Snake), Rhabdophis subminiatus (Red Necked Keelback), Rhabdophis tigrinus (Tiger Keelback), Thamnophis elegans (Western Terrestrial Garter Snake), Thamnophis sirtalis (Common Garter Snake), Ahaetulla nasuta (Long Nosed Whip Snake), Atractaspis bibronii (Bibron's Burrowing Asp), Atractaspis dahomeyensis (Dahomey Burrowing Asp), Atractaspis engaddensis (Palestinian Mole Viper), Atractaspis microlepidota (Small Scaled Burrowing Asp), Malpolon monspessulanus (Montpellier Snake), Acanthophis antarcticus (Common Death Adder), Aipysurus laevis (Olive Brown Sea Snake), Aipysurus duboisii (Dubois' Sea Snake), Austrelaps superbus (Lowland Copperhead), Cryptophis nigrescens (Small Eyed Snake), Demansia olivacea (Olive Whip Snake), Emydocephalus annulatus (Annulated Turtle Headed Sea Snake), Furina tristis (Stephen's Banded Snake), Hydrophis melanocephalus (Black Headed Slender Necked Sea Snake), Hydrophis curtus (Short Sea Snake), Hydrophis gracilis (Slender Sea Snake), Hydrophis elegans (Elegant Sea Snake), Hydrophis jerdonii (Cone Nosed Sea Snake), Hydrophis klossi (Selangor Sea Snake), Hydrophis peronii (Horned Sea Snake), Hydrophis belcheri (Belcher's Sea Snake), Hydrophis stricticollis (Bengal Sea Snake), Hydrophis major (Olive Headed Sea Snake), Hydrophis stokesii (Large Headed Sea Snake), Hydrophis melanosoma (Black Banded Robust Sea Snake), Hydrophis hardwickii (Spine Bellied Sea Snake), Hydrophis cyanocinctus (Annulated Sea Snake), Hydrophis spiralis (Narrow Banded Sea Snake), Hydrophis nigrocinctus (Black Banded Sea Snake), Hydrophis platurus (Yellowbelly Sea Snake), Hydrophis ornatus (Ornate Reef Sea Snake), Hydrophis viperinus (Viperine Sea Snake), Hydrophis schistosus (Beaked Sea Snake), Notechis scutatus (Mainland Tiger Snake), Oxyuranus scutellatus (Coastal Taipan), Oxyuranus temporalis (Central Ranges Taipan), Pseudechis australis (Mulga Snake), Pseudechis butleri (Butler's Black Snake), Pseudechis colletti (Collett's Black Snake), Pseudechis guttatus (Blue Bellied Black Snake), Pseudechis papuanus (Papuan Black Snake), Pseudechis porphyriacus (Red Bellied Black Snake), Pseudonaja affinis (Dugite), Pseudonaja guttata (Speckled Brown Snake), Pseudonaja inframacula (Peninsula Brown Snake), Pseudonaja nuchalis (Western Brown Snake), Pseudonaja textilis (Eastern Brown Snake), Tropidechis carinatus (Clarence River Snake), Aspidelaps lubricus (Cape Coral Snake), Aspidelaps scutatus (Shield Nose Snake), Bungarus fasciatus (Banded Krait), Bungarus caeruleus (Indian Krait), Bungarus candidus (Blue Krait), Bungarus flaviceps (Red Headed Krait), Bungarus multicinctus (Many Banded Krait), Dendroaspis viridis (Western Green Mamba), Dendroaspis angusticeps (Eastern Green Mamba), Dendroaspis jamesoni (Jameson's Mamba), Dendroaspis polylepis (Black Mamba), Elapsoidea sundevallii (Sundevall's African Garter Snake), Hemachatus haemachatus (Rinkhals), Laticauda colubrina (Common Yellow Lipped Sea Krait), Laticauda laticaudata (Blue Lipped Sea Krait), Laticauda semifasciata (Black Banded Sea Krait), Micrurus obscurus (Bolivian Coral Snake), Micrurus frontalis (Southern Coral Snake), Micrurus alleni (Allen's Coral Snake), Micrurus altirostris (Uruguayan Coral Snake), Micrurus clarki (Clark's Coral Snake), Micrurus corallinus (Painted Coral Snake), Micrurus distans (West Mexican Coral Snake), Micrurus dumerilii (Dumeril's Coral Snake), Micrurus fulvius (Eastern Coral Snake), Micrurus hemprichii (Hemprich's Coral Snake), Micrurus ibiboboca (Caatinga Coral Snake), Micrurus lemniscatus (South American Coral Snake), Micrurus mipartitus (Redtail Coral Snake), Micrurus mosquitensis (Costa Rican Coral Snake), Micrurus multifasciatus (Many Banded Coral Snake), Micrurus nigrocinctus (Central American Coral Snake), Micrurus pyrrhocryptus (Argentinean Coral Snake), Micrurus spixii (Amazon Coral Snake), Micrurus surinamensis (Aquatic Coral Snake), Micrurus tener (Texas Coral Snake), Micrurus tschudii (Desert Coral Snake), Naja siamensis (Indochinese Spitting Cobra), Naja annulata (Ringed Water Cobra), Naja annulifera (Banded Cobra), Naja ashei (Giant Spitting Cobra), Naja atra (Chinese Cobra), Naja christyi (Congo Water Cobra), Naja haje (Egyptian Cobra), Naja kaouthia (Monocled Cobra), Naja katiensis (Mali Cobra), Naja melanoleuca (Forest Cobra), Naja mossambica (Mozambique Spitting Cobra), Naja naja (Spectacled Cobra), Naja nigricollis (Black Necked Spitting Cobra), Naja nivea (Cape Cobra), Naja nubiae (Nubian Spitting Cobra), Naja oxiana (Caspian Cobra), Naja pallida (Red Spitting Cobra), Naja philippinensis (Northern Philippine Cobra), Naja samarensis (Samar Cobra), Naja sputatrix (Javan Spitting Cobra), Naja sumatrana (Sumatran Spitting Cobra), Ophiophagus hannah (King Cobra), Walterinnesia aegyptia (Western Black Desert Cobra), Homalopsis buccata (Linne's Water Snake), Myrrophis chinensis (Chinese Mud Snake), Subsessor bocourti (Bocourt's Water Snake), Azemiops fede (Fea's Viper), Agkistrodon bilineatus (Mexican Cantil), Agkistrodon contortrix (Copperhead), Agkistrodon piscivorus (Cottonmouth), Agkistrodon taylori (Castellana), Agkistrodon laticinctus (Broad Banded Copperhead), Atropoides picadoi (Picado's Jumping Pitviper), Bothriechis lateralis (Side Striped Palm Viper), Bothriechis nigroviridis (Black Speckled Palm Pitviper), Bothriechis schlegelii (Eyelash Palm Pitviper), Bothrops diporus (Chaco Lancehead), Bothrops erythromelas (Caatinga Lancehead), Bothrops insularis (Golden Lancehead Viper), Bothrops jararaca (Jararaca), Bothrops neuwiedi (Neuwied's Lancehead), Bothrops pauloensis (Black Faced Lancehead), Bothrops asper (Terciopelo), Bothrops atrox Common Lancehead), Bothrops ayerbei Ayerbe's Lancehead), Bothrops caribbaeus Saint Lucia Lancehead), Bothrops jararacussu Jararacussu), Bothrops lanceolatus (Martinique Lancehead), Bothrops leucurus (Whitetail Lancehead), Bothrops moojeni (Brazilian Lancehead), Bothrops alternatus (Urutú), Bothrops cotiara (Cotiara), Bothrops fonsecai (Fonseca's Lancehead), Bothrops itapetiningae (São Paulo Lancehead), Bothrops taeniatus (Speckled Forest Pitviper), Bothrops mattogrossensis (Mato Grosso Lanzenotter), Calloselasma rhodostoma (Malayan Pitviper), Cerrophidion godmani (Godman's Montane Pitviper), Cerrophidion sasai (Costa Rica Montane Pitviper), Crotalus viridis (Prairie Rattlesnake), Crotalus atrox (Western Diamondback Rattlesnake), Crotalus adamanteus (Eastern Diamondback Rattlesnake), Crotalus basiliscus (Mexican West Coast Rattlesnake), Crotalus catalinensis (Santa Catalina Island Rattlesnake), Crotalus cerastes (Sidewinder Rattlesnake), Crotalus Cerberus (Arizona Black Rattlesnake), Crotalus durissus (South American Rattlesnake), Crotalus enyo (Baja California Rattlesnake), Crotalus horridus (Timber Rattlesnake), Crotalus lepidus (Mottled Rock Rattlesnake), Crotalus mitchellii (San Lucan Speckled Rattlesnake), Crotalus molossus (Northern Black Tailed Rattlesnake), Crotalus oreganus (Northern Pacific Rattlesnake), Crotalus pricei (Western Twin Spotted Rattlesnake), Crotalus pusillus (Tancitaran Dusky Rattlesnake), Crotalus ravus (Mexican Pygmy Rattlesnake), Crotalus ruber (Red Diamond Rattlesnake), Crotalus scutulatus (Mohave Rattlesnake), Crotalus simus (Central American Rattlesnake), Crotalus tigris (Tiger Rattlesnake), Crotalus totonacus (Totonacan Rattlesnake), Crotalus tzabcan (Yucatán Neotropical Rattlesnake), Crotalus willardi (Arizona Ridgenose Rattlesnake), Crotalus Pyrrhus (Southwestern Speckled Rattlesnake), Crotalus vegrandis (Uracoan Rattlesnake), Deinagkistrodon acutus (Chinese Sharp Nosed Pitviper), Gloydius intermedius (Intermediate Mamushi), Gloydius blomhoffii (Mamushi), Gloydius brevicaudus (Short Tailed Mamushi), Gloydius halys (Siberian Pitviper), Gloydius shedaoensis (Shedao Island Pitviper), Gloydius ussuriensis (Ussuri Mamushi), Hypnale hypnale (Hump Nosed Pitviper), Lachesis melanocephala (Black Headed Bushmaster), Lachesis muta (Atlantic Bushmaster), Ovophis okinavensis (Hime Habu), Porthidium nasutum (Rainforest Hog Nosed Pitviper), Porthidium ophryomegas (Slender Hognosed Pitviper), Protobothrops elegans (Sakishima Habu), Protobothrops flavoviridis (Okinawa Habu), Protobothrops mangshanensis (Mangshan Pitviper), Protobothrops mucrosquamatus (Taiwan Habu), Protobothrops tokarensis (Tokara Island Pit Viper), Sistrurus catenatus (Massassauga), Sistrurus miliarius (Pygmy Rattlesnake), Trimeresurus stejnegeri (Chinese Green Tree Viper), Trimeresurus albolabris (White Lipped Pitviper), Trimeresurus erythrurus (Red Tailed Bamboo Pitviper), Trimeresurus gramineus (Bamboo Pitviper), Trimeresurus labialis (Nicobar Bamboo Pitviper), Trimeresurus macrops (Large Eyed Pitviper), Trimeresurus malabaricus (Malabar Pitviper), Trimeresurus popeiorum (Pope's Bamboo Pitviper), Trimeresurus purpureomaculatus (Mangrove Pitviper), Trimeresurus sumatranus (Sumatran Pitviper), Tropidolaemus wagleri (Wagler's Pitviper), Metlapilcoatlus mexicanus (Mexican Jumping Pitviper), Metlapilcoatlus nummifer (Central American Jumping Pitviper), Atheris squamigera (African Bush Viper), Bitis arietans (Puff Adder), Bitis Atropos (Cape Mountain Adder), Bitis caudalis (Horned Puff Adder), Bitis cornuta (Western Many Horned Adder), Bitis gabonica (Central African Gaboon Viper), Bitis nasicornis (Rhinoceros Viper), Bitis parviocula (Ethiopian Mountain Adder), Bitis rhinoceros (West African Gaboon Viper), Causus rhombeatus (Common Night Adder), Cerastes cerastes (Horned Viper), Cerastes gasperettii (Arabian Horned Viper), Cerastes vipera (Sahara Sand Viper), Daboia palaestinae (Palestine Viper), Daboia russelii (Russell's Viper), Daboia siamensis (Eastern Russell's Viper), Daboia mauritanica (Moorish Viper), Echis ocellatus (West African Carpet Viper), Echis carinatus (Saw Scaled Viper), Echis coloratus (Painted Saw Scaled Viper), Echis pyramidum (Egyptian Saw Scaled Viper), Macrovipera schweizeri (Milos Viper), Macrovipera lebetinus (Levantine Viper), Montivipera bornmuelleri (Lebanon Viper), Montivipera latifii (Latifi's Viper), Montivipera raddei (Armenian Mountain Viper), Montivipera xanthine (Ottoman Viper), Pseudocerastes fieldi (Field's Horned Viper), Pseudocerastes persicus (Persian Horned Viper), Vipera ammodytes (European Nose Horned Viper), Vipera aspis (Aspic Viper), Vipera berus (Common European Adder), Vipera latastei (Lataste's Viper), Vipera lotievi (Caucasian Meadow Viper), Vipera seoanei (Baskian Viper), Vipera ursinii (Ursini's Viper), Diadophis punctatus (Ringneck Snake), Hydrodynastes gigas (False Water Cobra), Hypsiglena torquata (Night Snake), Hypsiglena jani (San Luis Potosi Night Snake), Leptodeira ashmeadii, or Philodryas olfersii (Lichtenstein's Green Racer).

Venom (e.g., snake venom) can be a component of a modified saliva that can comprise a zootoxin that can facilitate immobilization and/or digestion of prey or defense against threats. Venom can be introduced to a subject, for example, via biting or spitting.

Venom can comprise proteins or polypeptides. In some embodiments, venom can comprise a mixture of proteins, enzymes, or other substances with toxic or lethal properties which can affect a subject. Effects of venom on a subject can include effects on biological functions such as blood coagulation, blood pressure regulation, or transmission of nervous or muscular impulses.

Proteins can constitute 90-95% of venom's dry weight and can be responsible for its biological effects. Venom can comprise toxins (e.g., neurotoxins), nontoxic proteins (which can also have pharmacological properties), and/or enzymes, especially hydrolytic ones. Enzymes can make up 80-90% of viperid and 25-70% of elapid venoms, and can include digestive hydrolases, L-amino acid oxidase, phospholipases, thrombin-like pro-coagulant, and kallikrein-like serine proteases or metalloproteinases (hemorrhagins), which can damage vascular endothelia. Polypeptide toxins can include cytotoxins, cardiotoxins, and postsynaptic neurotoxins (such as α-bungarotoxin and α-Cobratoxin), which can bind to acetylcholine receptors, for example, at neuromuscular junctions. Compounds with low molecular weight (e.g., up to 1.5 KDa) include metals, peptides, lipids, nucleosides, carbohydrates, amines, or oligopeptides, which inhibit angiotensin converting enzyme (ACE) or potentiate bradykinin (BPP). Inter-species and intra-species variation in venom chemical composition can be geographical and/or ontogenic. Phosphodiesterases can interfere with a cardiac system, for example, to lower the blood pressure. Phospholipase A2 can cause hemolysis by lysing the phospholipid cell membranes of red blood cells. Amino acid oxidases and proteases can be used for digestion. Amino acid oxidase can trigger other enzymes and can be responsible for the yellow color of the venom of some species. Hyaluronidase can increase tissue permeability to accelerate absorption of other enzymes into tissues. Some snake venoms can carry fasciculins, for example, the mambas (Dendroaspis), which can inhibit cholinesterase to make the prey lose muscle control. Examples of enzymes of snake venom are provided in Table 19.

TABLE 19
Snake venom enzymes

| Type | Name | Origin |
|---|---|---|
| Oxidoreductases | dehydrogenase lactate | Elapidae |
| | L-amino-acid oxidase | All species |
| | Catalase | All species |
| Transferases | Alanine amino transferase (ALT) | |
| | Aspartate aminotransferase (AST) | |
| Hydrolases | Phospholipase A₂ | All species |
| | Lysophospholipase | Elapidae, |
| | | Viperidae |
| | Acetylcholinesterase | Elapidae |
| | Alkaline phosphatase | Bothrops atrox |
| | Acid phosphatase | Deinagkistrodon |
| | | acutus |
| | 5′-Nucleotidase | All species |
| | Phosphodiesterase | All species |
| | Deoxyribonuclease | All species |
| | Ribonuclease 1 | All species |
| | Adenosine triphosphatase | All species |
| | Amylase | All species |
| | Hyaluronidase | All species |
| | NAD-Nucleotidase | All species |
| | Kininogenase | Viperidae |
| | Factor-X activator | Viperidae, |
| | | Crotalinae |
| | Heparinase | Crotalinae |
| | α-Fibrinogenase | Viperidae, |
| | | Crotalinae |
| | β-Fibrinogenase | Viperidae, |
| | | Crotalinae |
| | α-β-Fibrinogenase | Bitis gabonica |
| | Fibrinolytic enzyme | Crotalinae |
| | Prothrombin activator | Crotalinae |
| | Collagenase | Viperidae |
| | Elastase | Viperidae |
| Lyases | Glucosamine ammonium lyase | |

Toxins in venom such as snake venom can vary in function. Classes of toxins that can be found in snake venom can comprise neurotoxins, hemotoxins, cytotoxins, myotoxins, dendrotoxins, cardiotoxins, fasciculins, neutrotoxins, sarafotoxins, hemorrhagins, etc. Non-limiting examples of types of toxins that can be in venom such as snake venom can be found in Table 20.

TABLE 20

| Toxin | Examples |
|---|---|
| α-neurotoxins | α-Bungarotoxin, α-toxin, erabutoxin, |
| | cobratoxin |
| β-neurotoxins (PLA2) | β-Bungarotoxin, Notexin, ammodytoxin, |
| | crotoxin, taipoxin |
| κ-neurotoxins | Kappa-bungarotoxin |
| Dendrotoxins (Kunitz) | Dendrotoxin, toxins I and K; possibly |
| | β-Bungarotoxin chain B |
| Cardiotoxins | Naja nigricollis y-toxin, cardiotoxin |
| | III (aka cytotoxins) |
| Myotoxins | Myotoxin-a, crotamine |
| Sarafotoxins | Sarafotoxins a, b, and c |
| Hemorrhagins | Mucrolysin, Atrolysins, Acutolysins, etc. |
| (metalloprotease) | |
| Hemotoxins (serine | Venombin A |
| protease) | |

### Antibodies

As used herein, the term “antibody” refers to an immunoglobulin (Ig) refers to a polypeptide or a protein having a binding domain which is, or is homologous to, an antigen-binding domain. The term further includes “antigen-binding fragments” and other interchangeable terms for similar binding fragments such as described below. Native antibodies and native immunoglobulins (Igs) are usually heterotetrameric glycoproteins of about 150,000 Daltons, composed of two identical light chains and two identical heavy chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}”) followed by a number of constant domains (“C_{H}”). Each light chain has a variable domain at one end (“V_{L}”) and a constant domain (“C_{L}”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

In some instances, an antibody or an antigen-binding fragment thereof comprises an isolated antibody or antigen-binding fragment thereof, a purified antibody or antigen-binding fragment thereof, a recombinant antibody or antigen-binding fragment thereof, a modified antibody or antigen-binding fragment thereof, or a synthetic antibody or antigen-binding fragment thereof. It would be understood that the antibodies described herein can be modified as described below or as known in the art.

Antibodies and antigen-binding fragments herein can be partly or wholly synthetically produced. An antibody or antigen-binding fragment can be a polypeptide or protein having a binding domain which can be or can be homologous to an antigen binding domain. In one instance, an antibody or an antigen-binding fragment thereof can be produced in an appropriate in vivo animal model and then isolated and/or purified.

Depending on the amino acid sequence of the constant domain of its heavy chains, immunoglobulins (Igs) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The subunit structures and three-dimensional configurations of different classes of immunoglobulins are well known in the art. An Ig or portion thereof can, in some cases, be a human Ig. In some instances, a C_{H}3 domain can be from an immunoglobulin. In some cases, a chain or a part of an antibody or antigen binding fragment thereof, a modified antibody or antigen-binding fragment thereof, or a binding agent can be from an Ig. In such cases, an Ig can be IgG, an IgA, an IgD, an IgE, or an IgM. In cases where the Ig is an IgG, it can be a subtype of IgG, wherein subtypes of IgG can include IgG1, an IgG2a, an IgG2b, an IgG3, and an IgG4. In some cases, a C_{H}3 domain can be from an immunoglobulin selected from the group consisting of an IgG, an IgA, an IgD, an IgE, and an IgM.

The “light chains” of antibodies (immunoglobulins) from any vertebrate species can be assigned to one of two clearly distinct types, called kappa (“κ” or “K”) or lambda (“λ”), based on the amino acid sequences of their constant domains.

In the present disclosure, the following abbreviations (in the parentheses) are used in accordance with the customs, as necessary: Heavy chain (H chain), light chain (L chain), heavy chain variable region (VH), light chain variable region (VL), complementarity determining region (CDR), first complementarity determining region (CDR1), second complementarity determining region (CDR2), third complementarity determining region (CDR3), heavy chain first complementarity determining region (VH CDR1), heavy chain second complementarity determining region (VH CDR2), heavy chain third complementarity determining region (VH CDR3), light chain first complementarity determining region (VL CDR1), light chain second complementarity determining region (VL CDR2), light chain third complementarity determining region (VL CDR3), first heavy chain frame work region (FW-H1), second heavy chain frame work region (FW-H2), third heavy chain frame work region (FW-H3), fourth heavy chain frame work region (FW-H4), first light chain frame work region (FW-L1), second light chain frame work region (FW-L2), third light chain frame work region (FW-L3), and fourth light chain frame work region (FW-L4).

A “variable region” of an antibody refers to the variable region of the antibody light chain or the variable region of the antibody heavy chain, either alone or in combination. The variable regions of the heavy and light chain each consist of four framework regions (FW) connected by three CDRs also known as hypervariable regions. The CDRs in each chain are held together in close proximity by the FWs and, with the CDRs from the other chain, contribute to the formation of the antigen-binding site of antibodies.

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FWs.” The variable domains of unmodified heavy and light chains each contain four FRs (FW1, FW2, FW3 and FW4), largely adopting a β-sheet configuration interspersed with three CDRs which form loops connecting and, in some cases, part of the β-sheet structure. The CDRs in each chain are held together in close proximity by the FWs and, with the CDRs from the other chain, contribute to the formation of the antigen-binding site of antibodies.

The terms “hypervariable region” and “CDR” when used herein, refer to the amino acid residues of an antibody which are responsible for antigen-binding. The CDRs comprise amino acid residues from three sequence regions which bind in a complementary manner to an antigen and are known as CDR1, CDR2, and CDR3 for each of the V_{H} and V_{L} chains. It is understood that the CDRs of different antibodies may contain insertions, thus the amino acid numbering may differ. CDR sequences of the antibodies and antigen-binding fragments thereof have been provided herein below.

As used herein, “framework region,” “FW,” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat et al., (Sequences of Proteins of Immunological Interest, 5th Ed. Public Health Service, National Institutes of Health, Bethesda, Md., 1991). The Fc region of an immunoglobulin generally comprises two constant domains, C_{H}2 and C_{H}3.

In one instance, an antibody or antigen binding fragment can comprise variable regions. A variable region can be the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies.

In one instance, an antibody or antigen binding fragment can comprise light chain regions, heavy chain regions, or light chain and heavy chain regions that confer specific binding to, for example, a toxin. In another instance, an antibody or antigen binding fragment can comprise constant regions. A constant region can include the constant region of the antibody light chain either alone or in combination with the constant region of the antibody heavy chain.

“Antibodies” useful in the present disclosure encompass, but are not limited to, monoclonal antibodies, polyclonal antibodies, chimeric antibodies, bispecific antibodies, multispecific antibodies, heteroconjugate antibodies, humanized antibodies, human antibodies, deimmunized antibodies, mutants thereof, fusions thereof, immunoconjugates thereof, antigen-binding fragments thereof, and/or any other modified configuration of the immunoglobulin molecule that comprises an antigen recognition site of the required specificity, including glycosylation variants of antibodies, amino acid sequence variants of antibodies, and covalently modified antibodies.

As used herein, a “monoclonal antibody” refers to an antibody obtained from a population of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), each monoclonal antibody is directed against a single determinant on the antigen (epitope). The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example. Other methods are known in the art and are contemplated for use herein.

As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in, for example, WO 99/58572. Other forms of humanized antibodies have one or more CDRs (one, two, three, four, five or six) which are altered with respect to the original antibody, which are also termed one or more CDRs “derived from” one or more CDRs from the original antibody.

If needed, an antibody or an antigen binding fragment thereof described herein can be assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized. Analysis of immunogenicity and T-cell epitopes present in the antibodies and antigen-binding fragments described herein can be carried out via the use of software and specific databases known in the art. Exemplary software and databases include iTope™ developed by Antitope of Cambridge, England. iTope™, is an in silico technology for analysis of peptide binding to human MHC class II alleles. The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7 and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

An antibody can be a human antibody. As used herein, a “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS USA, 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an subject or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

Bispecific antibodies are antibodies that have binding specificities for at least two different antigens, can be prepared using the antibodies disclosed herein. Methods for making bispecific antibodies are known in the art (see, e.g., Suresh et al., 1986, Methods in Enzymology 121:210). Traditionally, the recombinant production of bispecific antibodies was based on the co-expression of two immunoglobulin heavy chain-light chain pairs, with the two heavy chains having different specificities (Millstein and Cuello, 1983, Nature, 305, 537-539). Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690.

According to one approach to making bispecific antibodies, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Heteroconjugate antibodies, comprising two covalently joined antibodies, are also within the scope of the disclosure. Such antibodies have been used to target immune system cells to unwanted cells (U.S. Pat. No. 4,676,980). Heteroconjugate antibodies may be made using any convenient cross-linking methods. Suitable cross-linking agents and techniques are known in the art and are described, for example, in U.S. Pat. No. 4,676,980.

“Chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin is inserted in place of the murine Fc.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Provided herein are antibodies and antigen-binding fragments thereof, modified antibodies and antigen-binding fragments thereof, and binding agents that specifically bind to one or more epitopes on one or more target antigens. In one instance, a binding agent specifically binds to an epitope on a single antigen. In another instance, a binding agent is bivalent and either specifically binds to two distinct epitopes on a single antigen or binds to two distinct epitopes on two distinct antigens. In another instance, a binding agent is multivalent (i.e., trivalent, quatravalent, etc.) and the binding agent binds to three or more distinct epitopes on a single antigen or binds to three or more distinct epitopes on two or more (multiple) antigens.

Functional fragments of any of the antibodies herein are also contemplated. The terms “antigen-binding portion” of an antibody, “antigen-binding fragment,” “antigen-binding domain,” “antibody fragment,” or a “functional fragment of an antibody” are used interchangeably herein to refer to one or more fragments of an antibody that retain the ability to specifically bind to an antigen. Representative antigen-binding fragments include, but are not limited to, a Fab, a Fab′, a F(ab′)₂, a Fv, a scFv, a dsFv, a variable heavy domain, a variable light domain, a variable NAR domain, bi-specific scFv, a bi-specific Fab₂, a tri-specific Fab₃, an AVIMER®, a minibody, a diabody, a maxibody, a camelid, a VHH, an intrabody, fusion proteins comprising an antibody portion (e.g., a domain antibody), a single chain binding polypeptide, a scFv-Fc, or a Fab-Fc.

“F(ab′)₂” and “Fab” moieties can be produced by treating an Ig with a protease such as pepsin and papain and include antigen-binding portions generated by digesting immunoglobulin near the disulfide bonds existing between the hinge regions in each of the two heavy chains. For example, papain cleaves IgG upstream of the disulfide bonds existing between the hinge regions in each of the two heavy chains to generate two homologous antibody fragments in which an light chain composed of V_{L} and C_{L} (light chain constant region), and a heavy chain fragment composed of V_{H} and C_{Hγ1} (γ1) region in the constant region of the heavy chain) are connected at their C terminal regions through a disulfide bond. Each of these two homologous antibody fragments is called Fab′. Pepsin also cleaves IgG downstream of the disulfide bonds existing between the hinge regions in each of the two heavy chains to generate an antibody fragment slightly larger than the fragment in which the two above-mentioned Fab′ are connected at the hinge region. This antibody fragment is called F(ab′)₂.

The Fab fragment also contains the constant domain of the light chain and the first constant domain (C_{H}1) of the heavy chain. Fab′ fragments differ from Fab fragments by the addition of a few residues at the carboxyl terminus of the heavy chain C_{H}1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

A “Fv” as used herein refers to an antibody fragment which contains a complete antigen-recognition and antigen-binding site. This region consists of a dimer of one heavy chain and one light chain variable domain in tight, non-covalent or covalent association (disulfide linked Fvs have been described in the art, Reiter et al. (1996) Nature Biotechnology 14:1239-1245). It is in this configuration that the three CDRs of each variable domain interact to define an antigen-binding site on the surface of the V_{H}-V_{L} dimer. Collectively, a combination of one or more of the CDRs from each of the V_{H} and V_{L} chains confer antigen-binding specificity to the antibody. For example, it would be understood that, for example, the CDRH3 and CDRL3 could be sufficient to confer antigen-binding specificity to an antibody when transferred to V_{H} and V_{L} chains of a recipient antibody or antigen-binding fragment thereof and this combination of CDRs can be tested for binding, specificity, affinity, etc. using any of the techniques described herein. Even a single variable domain (or half of an Fv comprising only three CDRs specific for an antigen) has the ability to recognize and bind antigen, although likely at a lower specificity or affinity than when combined with a second variable domain. Furthermore, although the two domains of a Fv fragment (V_{L} and V_{H}), are coded for by separate genes, they can be joined using recombinant methods by a synthetic linker that enables them to be made as a single protein chain in which the V_{L} and V_{H} regions pair to form monovalent molecules (known as single chain Fv (scFv); Bird et al. (1988) Science 242:423-426; Huston et al. (1988) Proc. Natl. Acad. Sci. USA 85:5879-5883; and Osbourn et al. (1998) Nat. Biotechnol. 16:778). Such scFvs are also intended to be encompassed within the term “antigen-binding portion” of an antibody. Any V_{H} and V_{L} sequences of specific scFv can be linked to an Fc region cDNA or genomic sequences, in order to generate expression vectors encoding complete Ig (e.g., IgG) molecules or other isotypes. V_{H} and V_{L} can also be used in the generation of Fab, Fv or other fragments of Igs using either protein chemistry or recombinant DNA technology.

“Single-chain Fv” or “sFv” antibody fragments comprise the V_{H} and V_{L} domains of an antibody, wherein these domains are present in a single polypeptide chain. In some embodiments, the Fv polypeptide further comprises a polypeptide linker between the V_{H} and V_{L} domains which enables the sFv to form the desired structure for antigen binding. For a review of sFvs, see, e.g., Pluckthun in The Pharmacology of Monoclonal Antibodies, Vol. 113, Rosenburg and Moore eds. Springer-Verlag, New York, pp. 269-315 (1994).

The term “AVIMER®” refers to a class of therapeutic proteins of human origin, which are unrelated to antibodies and antibody fragments, and are composed of several modular and reusable binding domains, referred to as A-domains (also referred to as class A module, complement type repeat, or LDL-receptor class A domain). They were developed from human extracellular receptor domains by in vitro exon shuffling and phage display (Silverman et al., 2005, Nat. Biotechnol. 23:1493-1494; Silverman et al., 2006, Nat. Biotechnol. 24:220). The resulting proteins can contain multiple independent binding domains that can exhibit improved affinity and/or specificity compared with single-epitope binding proteins. Each of the known 217 human A-domains comprises ˜35 amino acids (˜4 kDa); and these domains are separated by linkers that average five amino acids in length. Native A-domains fold quickly and efficiently to a uniform, stable structure mediated primarily by calcium binding and disulfide formation. A conserved scaffold motif of only 12 amino acids is required for this common structure. The end result is a single protein chain containing multiple domains, each of which represents a separate function. Each domain of the proteins binds independently, and the energetic contributions of each domain are additive.

Antigen-binding polypeptides also include heavy chain dimers such as, for example, antibodies from camelids and sharks. Camelid and shark antibodies comprise a homodimeric pair of two chains of V-like and C-like domains (neither has a light chain). Since the V_{H} region of a heavy chain dimer IgG in a camelid does not have to make hydrophobic interactions with a light chain, the region in the heavy chain that normally contacts a light chain is changed to hydrophilic amino acid residues in a camelid. V_{H} domains of heavy-chain dimer IgGs are called V_{HH} domains. Shark Ig-NARs comprise a homodimer of one variable domain (termed a V-NAR domain) and five C-like constant domains (C-NAR domains). In camelids, the diversity of antibody repertoire is determined by the CDRs 1, 2, and 3 in the V_{H} or V_{HH} regions. The CDR3 in the camel V_{HH} region is characterized by its relatively long length, averaging 16 amino acids (Muyldermans et al., 1994, Protein Engineering, 7(9): 1129). This is in contrast to CDR3 regions of antibodies of many other species. For example, the CDR3 of mouse VII has an average of 9 amino acids. Libraries of camelid-derived antibody variable regions, which maintain the in vivo diversity of the variable regions of a camelid, can be made by, for example, the methods disclosed in U.S. patent application Ser. No. 20/050,037421.

As used herein, a “maxibody” refers to a bivalent scFv covalently attached to the Fc region of an immunoglobulin, see, for example, Fredericks et al., Protein Engineering, Design & Selection, 17:95-106 (2004) and Powers et al., Journal of Immunological Methods, 251:123-135 (2001).

As used herein, a “dsFv” can be a Fv fragment obtained by introducing a Cys residue into a suitable site in each of a heavy chain variable region and a light chain variable region, and then stabilizing the heavy chain variable region and the light chain variable region by a disulfide bond. The site in each chain, into which the Cys residue is to be introduced, can be determined based on a conformation predicted by molecular modeling. In the present disclosure, for example, a conformation is predicted from the amino acid sequences of the heavy chain variable region and light chain variable region of the above-described antibody, and DNA encoding each of the heavy chain variable region and the light chain variable region, into which a mutation has been introduced based on such prediction, is then constructed. The DNA construct is incorporated then into a suitable vector and prepared from a transformant obtained by transformation with the aforementioned vector.

Single chain variable region fragments (“scFv”) of antibodies are described herein. Single chain variable region fragments may be made by linking light and/or heavy chain variable regions by using a short linking peptide. Bird et al. (1988) Science 242:423-426. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

Diabodies can be single chain antibodies. Diabodies can be bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al., Proc. Natl. Acad. Sci. USA, 90:6444-6448 (1993); and Poljak, R. J., et al., Structure, 2:1121-1123 (1994)).

As used herein, a “minibody” refers to a scFv fused to CH3 via a peptide linker (hingeless) or via an IgG hinge has been described in Olafsen, et al., Protein Eng Des Sel., April 2004; 17(4):315-23.

As used herein, an “intrabody” refers to a single chain antibody which demonstrates intracellular expression and can manipulate intracellular protein function (Biocca, et al., EMBO J. 9:101-108, 1990; Colby et al., Proc Natl Acad. Sci. USA. 101:17616-21, 2004). Intrabodies, which comprise cell signal sequences which retain the antibody construct in intracellular regions, may be produced as described in Mhashilkar et al., (EMBO J., 14:1542-51, 1995) and Wheeler et al. (FASEB J. 17:1733-5. 2003). Transbodies are cell-permeable antibodies in which a protein transduction domains (PTD) is fused with single chain variable fragment (scFv) antibodies Heng et al., (Med Hypotheses. 64:1105-8, 2005).

Suitable linkers known in the art may be used to multimerize binding agents. A non-limiting example of a linking peptide is (GGGGS)₃ (SEQ ID NO: 283), which bridges approximately 3.5 nm between the carboxy terminus of one variable region and the amino terminus of the other variable region. Linkers of other sequences have been designed and used. Bird et al. (Id.) Linkers can in turn be modified for additional functions, such as attachment of drugs or attachment to solid supports.

“Epitope” refers to that portion of an antigen or other macromolecule capable of forming a binding interaction with the variable region binding pocket of an antibody. Such binding interactions can be manifested as an intermolecular contact with one or more amino acid residues of one or more CDRs. Antigen-binding can involve, for example, a CDR3 or a CDR3 pair or, in some cases, interactions of up to all six CDRs of the V_{H} and V_{L} chains. An epitope can be a linear peptide sequence (“continuous”) or can be composed of noncontiguous amino acid sequences (“conformational” or “discontinuous”). An antibody can recognize one or more amino acid sequences; therefore, an epitope can define more than one distinct amino acid sequence. Epitopes recognized by antibodies can be determined by peptide mapping and sequence analysis techniques well known to one of skill in the art. Binding interactions are manifested as intermolecular contacts between an epitope on an antigen and one or more amino acid residues of a CDR.

An antibody selectively binds to a target if it binds with greater affinity, avidity, more readily, and/or with greater duration than it binds to other substances. For example, an antibody or antigen-binding fragment that selectively binds to, for example, a snake venom toxin is an antibody or antigen-binding fragment that binds this epitope with greater affinity, avidity, more readily, and/or with greater duration than it binds to a protein that is not a snake venom toxin.

The term “kon”, as used herein, is intended to refer to the rate constant for association of an antibody to an antigen.

The term “Koff”, as used herein, is intended to refer to the rate constant for dissociation of an antibody from the antibody/antigen complex.

As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique.

As used herein, the term “affinity” refers to the equilibrium constant for the reversible binding of two agents and is expressed as binding affinity (K_{D}). In some cases, K_{D} can be represented as a ratio of k_{off}, which can refer to the rate constant for dissociation of an antibody from the antibody or antigen-binding fragment/antigen complex, to kₒₙ, which can refer to the rate constant for association of an antibody or antigen binding fragment to an antigen. Binding affinity may be determined using methods known in the art including, for example, surface plasmon resonance (SPR; Biacore), Kinexa Biocensor, scintillation proximity assays, enzyme linked immunosorbent assay (ELISA), ORIGEN immunoassay (IGEN), fluorescence quenching, fluorescence transfer, yeast display, or any combination thereof. Binding affinity may also be screened using a suitable bioassay. The binding affinity (K_{D}) of an antibody or antigen-binding fragment herein can be less than 600 nM, 590 nM, 580 nM, 570 nM, 560 nM, 550 nM, 540 nM, 530 nM, 520 nM, 510 nM, 500 nM, 490 nM, 480 nM, 470 nM, 460 nM, 450 nM, 440 nM, 430 nM, 420 nM, 410 nM, 400 nM, 390 nM, 380 nM, 370 nM, 360 nM, 350 nM, 340 nM, 330 nM, 320 nM, 310 nM, 300 nM, 290 nM, 280 nM, 270 nM, 260 nM, 250 nM, 240 nM, 230 nM, 220 nM, 210 nM, 200 nM, 190 nM, 180 nM, 170 nM, 160 nM, 150 nM, 140 nM, 130 nM, 120 nM, 110 nM, 100 nM, 90 nM, 80 nM, 70 nM, 50 nM, 50 nM, 49 nM, 48 nM, 47 nM, 46 nM, 45 nM, 44 nM, 43 nM, 42 nM, 41 nM, 40 nM, 39 nM, 38 nM, 37 nM, 36 nM, 35 nM, 34 nM, 33 nM, 32 nM, 31 nM, 30 nM, 29 nM, 28 nM, 27 nM, 26 nM, 25 nM, 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, or any integer therebetween.

The terms “polypeptide,” “oligopeptide,” “peptide,” and “protein” are used interchangeably herein and generally refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other suitable modifications. It is understood that, because the polypeptides of this disclosure are based upon an antibody, the polypeptides can occur as single chains or associated chains.

“Polynucleotide” or “nucleic acid,” as used interchangeably herein, generally refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s).

Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses, or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO, or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl, or araldyl. Not all linkages in a polynucleotide need be identical. The preceding description applies to all polynucleotides referred to herein, including RNA and DNA.

As used herein, “identity” can mean the percentage of identical nucleotide or amino acid residues at corresponding positions in two or more sequences when the sequences are aligned to maximize sequence matching (taking into account gaps and insertions). Identity can be readily calculated by known methods including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well-known Smith Waterman algorithm may also be used to determine identity.

Ranges of desired degrees of sequence identity are from about 80% to about 100% and integer values therebetween. In general, this disclosure encompasses sequences with about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with any sequence provided herein. The letter “X” or “Xaa” as used in amino acid sequences herein is intended to indicate that any of the twenty standard amino acids may be placed at this position unless specifically noted otherwise.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1: Kinetics of Anti-Venom Antibodies

Kinetics measurements were made to calculate binding affinities of two antibodies to recombinant alpha neurotoxins of Mamba, Taipan, and Cobra snakes. The alpha-neurotoxins were expressed and purified by affinity-chromatography. Antibodies isolated from a subject having previous exposure to snake bites were incubated with the alpha neurotoxins, and the kinetics of the binding of the alpha-neurotoxins to the antibodies were measured. The kinetic data are provided in Table 21A and 21B.

TABLE 21A
Kinetics of anti-venom antibodies

| Antibody | Protein | kₐ (M⁻¹s⁻¹) | k_{d} (s⁻¹) | K_{D} (nM) |
|---|---|---|---|---|
| P01_B11 (hlg | Mamba-Avi-His | 1.8E+05 | 3.8E−04 | 2.1 |
| G1_2dA) | Taipan-Avi-His | 1.5E+05 | 5.2E−03 | 35 |
| | Cobra-Avi-His | 2.2E+05 | 9.0E−05 | 0.41 |
| P01_D09 (hlg | Mamba-Avi-His | 2.2E+05 | 1.0E−04 | 0.46 |
| G1_2dA) | Taipan-Avi-His | 2.1E+05 | 3.2E−04 | 1.5 |
| | Cobra-Avi-His | 2.4E+05 | 2.4E−05 | 0.10 |

TABLE 21B
Kinetics of anti-venom antibodies

| Antibody | Protein | k_{d} (1/s) |
|---|---|---|
| Aneuro_CX01_A04; D09 VH | Taipan LNX | 4.88E−04 |
| germlined | Mamba LNX | 8.54E−05 |
| Aneuro_CX01_A05; D09 VL | Taipan LNX | 3.92E−04 |
| germlined | Krait LNX | 5.59E−05 |
| | Mamba LNX | 6.95E−05 |
| Aneuro_CX01_A06; D09 FW | Taipan LNX | 3.66E−04 |
| germlined | Mamba LNX | 3.04E04 |
| Aneuro_CX01_A07; D09 VK | Taipan LNX | 3.04E−04 |
| L3 NS→QS germlined | | |
| Aneuro_CX01_A08; D09 VK | Taipan LNX | 7.69E−04 |
| L3 NS→NT germlined | | |

### Example 2: Toxicity of Venom Toxins in Mice

Toxicity of differing doses of neurotoxins in mice were determined. Recombinant alpha neurotoxins from various snake species were expressed and purified by affinity chromatography. An intraperitoneal dose greater than the LD50 (dose at which 50 percent of mice are dead after compound) of each toxin was administered to mice (5 mice per toxin group). Body weight and rectal body temperature for each mouse was measured prior to toxin administration and again every 20 minutes post-dose, up to 2 hours, and subsequently 24 hours after administration, along with general observations. Mice that either recovered or were not affected by a given dose of toxin showed full recovery between about 2 hours and 4 hours post-dose and remained healthy beyond 24 hours post-dose. If the dose of a given toxin was lethal, for the given doses described, mice were dead or euthanized by 60 minutes post-dose. Survival data are presented in Table 22:

TABLE 22
Toxicity of toxins in mice

| | | | Time to | |
|---|---|---|---|---|
| Toxin species | Dose | Survival | euthanasia | |
| short-neurotoxin-mamba-4LFT | 1 mg/kg | 0/5 | ~40 minutes | |
| | | | (min) | |
| short-neurotoxin-mamba-4LFT | 2 mg/kg | 0/5 | ~30 | min |
| short-neurotoxin-cobra (1CTX-like) | 1 mg/kg | 0/5 | ~55 | min |
| short-neurotoxin-taipan-1TXA | 1 mg/kg | 3/5 | ~40 | min |
| short-neurotoxin-taipan-1TXA | 2 mg/kg | 3/5 | ~20 | min |

### Example 3: Protective Effects of Antibodies Against Venom Toxins in Mice

Individual antibody clones of the antibodies tested in Example 1 were tested for their ability to neutralize the toxins tested in Example 2 by premixing the neurotoxin and antibody in an Eppendorf tube and incubating at room temperature for 1 hour. The mix was subsequently administered to a group of mice (n=5) to observe lethality as described above. Neutralization of lethal snake neurotoxins by antibody clones is provided in Table 23.

TABLE 23
Neutralization data of lethal snake neurotoxins by antibodies

| | Toxin | | Ab | | Molar |
|---|---|---|---|---|---|
| | Dose | Ab | Dose | | ratio of |
| Toxin species | mg/kg | clone | mg/kg | Survival | Toxin:Ab |
| short-neurotoxin- | 1 | P01-D09 | 17.2 | 5/5 | 1:0.8 |
| mamba-4LFT- | | | | | |
| His-Avitag | | | | | |
| short-neurotoxin- | 2 | P01-D09 | 17.2 | 5/5 | 1:0.4 |
| taipan-1TXA- | | | | | |
| His-Avitag | | | | | |
| short-neurotoxin- | 1 | P01-B11 | 14.8 | 5/5 | 1:0.35 |
| cobra (1CTX-like)- | | | | | |
| His-Avitag | | | | | |

Mice did not display any signs of morbidity. Molar ratio of toxin to antibody (accounting for bivalency) is shown.

### Example 4: Information Regarding Snakes, Toxins, and Sequences

TABLE 24
Exemplary Snakes

| Geographic | | | | Sub- | | |
|---|---|---|---|---|---|---|
| Region | Name | Suborder | Family | Family | Genus | Species |
| Asia | common | Serpentes | Elapidae | elapid | Bungarus | B. caeruleus |
| | krait | | | | | |
| Africa | black | Serpentes | Elapidae | elapid | Dendroaspis | D. polylepsis |
| | mamba | | | | | |
| Asia | Indian | Serpentes | Elapidae | elapid | Naja | N. naja |
| | cobra | | | | | |
| Australia | tiger snake | Serpentes | Elapidae | elapid | Notechis | N. scutatus |
| Australia | inland | Serpentes | Elapidae | elapid | Oxyuranus | O. microlepidotus |
| | taipan | | | | | |
| Australia | eastern | Serpentes | Elapidae | elapid | Pseudonaja | P. textilis |
| | brown | | | | | |
| | snake | | | | | |
| America | water | Serpentes | Viperidae | viperine | Agkistrodon | A. piscivorus |
| | moccasin | | | | | |
| America | lancehead | Serpentes | Viperidae | crotaline | Bothrops | atrox |
| America | rattle | Serpentes | Viperidae | crotaline | Crotalus | C. adamanteus |
| | snake | | | | | |
| Asia | Russell's | Serpentes | Viperidae | viperine | Daboia | D. russelii |
| | viper | | | | | |
| Africa | saw-scaled | Serpentes | Viperidae | viperine | Echis | E. ocellatus |
| | or carpet | | | | | |
| | viper | | | | | |
| Asia | saw-scaled | Serpentes | Viperidae | viperine | Echis | E. carinatus |
| | viper | | | | | |
| Africa | puff adder | Sepentes | Viperidae | viperine | Bitis | Arietans |
| Australia | death | Serpentes | Elapidae | elapid | Acanthophis | laevis |
| | adder | | | | | |

TABLE 25
Lethality, Subcutaneous LD50, Primary Toxins, Symptoms, and Effects

| Name | Lethality | Subcutaneous LD50 | | Primary Toxin(s) | Symptoms and Effects |
|---|---|---|---|---|---|
| common krait | 70-80% | 0.325 | mg/kg | neurotoxin | minimal local swelling & pain |
| black mamba | 100% | 0.28 | mg/kg | dendrotoxin, cardiotoxin, | collapse 45 min, death 7 hours |
| | | | | fasciculins | |
| Indian cobra | 20-30% | 0.45 | mg/kg | neutrotoxin, cardiotoxin | 15 min-2 hours |
| tiger snake | 40-60% | 0.2 | mg/kg | neurotoxins, coagulants, | local pain, numbness, tingling, sweating, |
| | | | | haemolysins, myotoxins | breathing difficulties paralysis. |
| inland taipan | 80% | 0.025 | mg/kg | neutrotoxin, blood clotting | reports are in contrast with Tim's |
| | | | | | presentation - novel biology here |
| eastern brown snake | 10-20% | 0.0365 | ug/kg | neurotoxins, blood | diarrhea, dizziness, convulsions, renal |
| | | | | coagulants | failure, paralysis, cardiac arrest |
| Cottonmouth | | 2 | mg/kg | hemotoxins | severe, immediate pain with rapid swelling; |
| (water moccasin) | | | | | discoloration of the skin; difficult or |
| | | | | | rapid breathing; changes in heart rate |
| | | | | | or rhythm; metallic, rubbery, or minty |
| | | | | | taste in the mouth; numbness or tingling |
| | | | | | around the mouth, tongue, scalp, feet, |
| | | | | | or the bite area; swelling in lymph |
| | | | | | nodes near the bite injury; and shock |
| lancehead | | 4-22 | mg/kg | hemotoxins | local pain and swelling, nausea and |
| | | | | | vomiting, blood blisters, bruising, blood |
| | | | | | in the vomit and urine, intestinal bleeding, |
| | | | | | kidney failure, brain hemorrhage and severe |
| | | | | | necrosis of muscular tissue |
| rattle snake | 10-20% | 14.5-10 | mg/kg | neurotoxins, hemotoxins | burning pain and edema if envenomation |
| | | | | | occurred. Pain, edema, anxiety, tingling, |
| | | | | | weakness, nausea, vomiting, hemorrhaging, |
| | | | | | perspiration, heart failure |
| Russell's viper | | 0.75 | mg/kg | hemotoxins | pain may last weeks, pituitary gland damage |
| carpet viper | 1-10% | | | neurotoxins, cardiotoxins, | local pain, swelling, blistering, necrosis and |
| | | | | hemotoxins, cytotoxins | coagulopathy, bleeding, renal failure, pain, shock |
| saw-scaled viper | 20% | 5.1-5.5 | mg/kg | hemorrhage, anticoagulation | pain, hemorrhage, shock, oliguria/anuria |
| | | | | | within 6 days, kidney dialysis |
| puff adder | 15-52% | 1.0-7.75 | mg/kg | cytotoxin | bleeding, swelling, severe pain, tenderness, |
| | | | | | superficial or deep necrosis, compartment |
| | | | | | syndrome, inflexibility, hemorrhage or |
| | | | | | coagulation in the affected muscles, residual |
| | | | | | induration, edema, shock, watery blood oozing |
| | | | | | from the puncture wounds, nausea, vomiting, |
| | | | | | subcutaneous bruising, blood blisters, painful |
| | | | | | swelling of the regional lymph nodes, hypotension, |
| | | | | | weakness, dizziness, periods of semi-consciousness, |
| | | | | | unconsciousness |
| death adder | | 0.38 | mg/kg | Neurotoxins, myotoxins, | Nausea, vomiting, paralysis of extra ocular |
| | | | | pro-coagulants, | muscles, abdominal pain, headache, drowsiness, |
| | | | | anticoagulants and | and enlargement of regional lymph nodes |
| | | | | toxins which interfere | |
| | | | | with platelet aggregation | |

TABLE 26
Exemplary Toxin Amino Acid Sequences

| | | SEQ ID |
|---|---|---|
| Toxin-Snake | Toxin Amino Acid Sequence | NO |
| dendrotoxin1- | QPLRKLCILHRNPGRCYQKIPAFYYNQKKKQCEG | 284 |
| mamba | FTWSGCGGNSNRFKTIEECRRTCIRK | |
| long-neurotoxin- | YTLLCHTTSTSPISTVTCPSGENLCYTKMWCDAF | 285 |
| krait (bungarus) | CSSRGKVIELGCVATCPQPKPYEEVTCCSTDKCN | |
| (alpha- | PHPKQRPG | |
| bungarotoxin) | | |
| long-neurotoxin- | RTCNKTFSDQSKICPPGENICYTKTWCDAFCSQR | 286 |
| mamba | GKRVELGCAATCPKVKAGVEIKCCSTDNCNKFQ | |
| (dendroapsis) | FGKPR | |
| (alpha-elapitoxin- | | |
| dpp2d) | | |
| long-neurotoxin- | IRCFITPDVTSQACPDGHVCYTKMWCDNFCGMR | 287 |
| cobra (naja) | GKRVDLGCAATCPKVKPGVNIKCCSRDNCNPFP | |
| (alpha-neurotoxin) | TRKRS | |
| long-neurotoxin- | RRCFTTPSVRSERCPPGQEVCYTKTWTDGHGGS | 288 |
| taipan | RGKRVDLGCAATCPTPKKKDIKIICCSTDNCNTFP | |
| | KWP | |
| long-neutroxin- | LICYMGPKTPRTCPRGQNLCYTKTWCDAFCSSR | 289 |
| tigersnake- | GKVVELGCAATCPIAKSYEDVTCCSTDNCNPFPV | |
| 3L21_NOTSC | RPRPHP | |
| Alpha-elapitoxin- | | |
| Nss2a | | |
| long-neurotoxin- | RTCFITPDVKSKPCPPGQEVCYTKTWCDGFCGIR | 290 |
| easternbrown-snake- | GKRVDLGCAATCPTPKKTGIDIICCSTDDCNTFPL | |
| 3L2P_PSETE | RPRGRLSSIKDHP | |
| pseudonajatoxin b | | |
| long-neurotoxin- | LICYLDFSVPHTCAPGEKLCYTRTWNDGRGTRIE | 291 |
| easternbrown-snake- | RGCAATCPIPKKPEIHVTCCSTDRCNPHPKQKPH | |
| 3L21_PSETE | | |
| long neurotoxin 1 | | |
| short-neutroxin- | MTCCNQQSSQPKTTTTCAESSCYKKTWRDHRGT | 292 |
| tigersnake- | ITERGCGCPNVKPGVQINCCKTDECNN | |
| 3S11_NOTSC | | |
| short neurotoxin 1 | | |
| short-neurotoxin2- | MTCYNQQSSEAKTTTTCSGGVSSCYKKTWSDGR | 293 |
| taipan-3NDS | GTIIERGCGCPSVKKGIERICCRTDKCNN | |
| short-neurotoxin2- | RICYNHQSTTRATTKSCEENSCYKKYWRDHRGTI | 294 |
| mamba-1NTX | IERGCGCPKVKPGVGIHCCQSDKCNY | |
| short-neurotoxin2- | MICHNQQSSQRPTIKTCPGETNCYKKRWRDHRG | 295 |
| cobra-1NEA | TIIERGCGCPSVKKGVGIYCCKTDKCNR | |
| pla2-1.mocassin- | SVLELGKMILQETGKNAITSYGSYGCNCGWGHR | 296 |
| 1PPA | GQPKDATDRCCFVHKCCYKKLTDCNHKTDRYS | |
| | YSWKNKAIICEEKNPCLKEMCECDKAVAICLREN | |
| | LDTYNKKYKAYFKLKCKKPDTC | |
| pla2-1.rattler | SLVELGKMILQETGKNPITSYGIYGCNCGVGSRH | 297 |
| | KPKDGTDRCCFVHKCCYKKLTDCDPKMDGYTY | |
| | SFKDKTIICDVNNPCLKEMCECDKAVAICLRENL | |
| | DTYNKKYKIYPKFLCKKPDTC | |
| pla2-1.sawscaled- | SVVELGKMIIQETGKSPFPSYTSYGCFCGGGERGP | 298 |
| 2QHD | PLDATDRCCLAHSCCYDTLPDCSPKTDRYKYKR | |
| | ENGEIICENSTSCKKRICECDKAVAVCLRKNLNT | |
| | YNKKYTYYPNFWCKGDIEKC | |
| pla2-mocassin- | NLFQFEKLIKKMTGKSGMLWYSAYGCYCGWGG | 299 |
| 1VAP | QGRPKDATDRCCFVHDCCYGKVTGCNPKMDIY | |
| | TYSVDNGNIVCGGTNPCKKQICECDRAAAICFRD | |
| | NLKTYDSKTYWKYPKKNCKEESEPC | |
| pla2-rattler-1PP2 | SLVQFETLIMKIAGRSGLLWYSAYGCYCGWGGH | 300 |
| | GLPQDATDRCCFVHDCCYGKATDCNPKTVSYTY | |
| | SEENGEIICGGDDPCGTQICECDKAAAICFRDNIPS | |
| | YDNKYWLFPPKNCREEPEPC | |
| pla2-sawscaled- | NLYQFGKMIKNKTGKPAMFSYSAYGCYCGWGG | 301 |
| 1OZ6 | QGKPQDPSDRCCFLHDCCYTRVNNCSPKMTLYS | |
| | YRFENGDIICGDNDPCRKAVCECDREAAICLGEN | |
| | VNTYDEKYRFYSSSYCTEEESEKC | |
| pla2-krait-1PO8 | NLYQLMNMIQCANTRTWPSYTNYGCYCGKGGS | 302 |
| | GTPVDDLDRCCYTHDHCYNDAKNIDGCNPVTKT | |
| | YSYTCTEPTITCNDSKDKCARFVCDCDRTAAICF | |
| | AKAPYNTSNVMIRSTNSCQ | |
| plat2-taipan-1AE7 | NLLQFGFMIRCANRRSRPVWHYMDYGCYCGKG | 303 |
| | GSGTPVDDLDRCCQVHDECYGEAVRRFGCAPY | |
| | WTLYSWKCYGKAPTCNTKTRCQRFVCRCDAKA | |
| | AECFARSPYQNSNWNINTKARCR | |
| plat2-taipan- | NLVQFGKMIECAIRNRRPALDFMNYGCYCGKGG | 304 |
| taipoxin-3 VC0 | SGTPVDDLDRCCQVHDECYAEAEKHGCYPSLTT | |
| | YTWECRQVGPYCNSKTQCEVFVCACDFAAAKC | |
| | FAQEDYNPAHSNINTGERCK | |
| pla2-russles- | NFFQFAEMIVKMTGKEAVHSYAIYGCYCGWGG | 305 |
| Daboiatoxin-2H4C | QGKPQDATDRCCFVHDCCYGTVNDCNPKMATY | |
| | SYSFENGDIVCGDNNLCLKTVCECDRAAAICLGQ | |
| | NVNTYDKNYENYAISHCTEESEQC | |
| pla2-cobra-1A3D | NLYQFKNMIKCTVPSRSWWDFADYGCYCGRGG | 306 |
| | SGTPVDDLDRCCQVHDNCYNEAEKISGCWPYFK | |
| | TYSYECSQGTLTCKGDNNACAASVCDCDRLAAI | |
| | CFAGAPYNDNNYNIDLKARCQ | |
| pla2-tigersnake- | NLVQFSYLIQCANHGKRPTWHYMDYGCYCGAG | 307 |
| totexin-1AE7 | GSGTPVDELDRCCKIHDDCYDEAGKKGCFPKMS | |
| | AYDYYCGENGPYCRNIKKKCLRFVCDCDVEAAF | |
| | CFAKAPYNNANWNIDTKKRCQ | |
| pla2-kingbrown- | NLIQFGNMIQCANKGSRPSLDYADYGCYCGWGG | 308 |
| ACII4-3V9M | SGTPVDELDRCCQVHDNCYEQAGKKGCFPKLTL | |
| | YSWKCTGNVPTCNSKPGCKSFVCACDAAAAKCF | |
| | AKAPYKKENYNIDTKKRCK | |
| pla2-krait-1G2X | NLQQFKNMIQCAGTRTWTAYINYGCYCGKGGS | 309 |
| | GTPVDKLDRCCYTHDHCYNQADSIPGCNPNIKT | |
| | YSYTCTQPNITCTRTADACAKFLCDCDRTAAICF | |
| | ASAPYNINNIMISASNSCQ | |
| pla2- | SLIQFETLIMKVVKKSGMFWYSAYGCYCGWGG | 310 |
| Deinagkistrodon- | HGRPQDATDRCCFVHDCCYGKVTGCDPKMDSY | |
| 1IJL | TYSEENGDIVCGGDDPCKREICECDRVAADCFRD | |
| | NLDTYNSDTYWRYPRQDCEESPEPC | |
| pla2- | SLVQFETLIMKVAKKSGMQWYSNYGCYCGWGG | 311 |
| agkistrodon.halys- | QGRPQDATDRCCFVHDCCYGKVTGCDPKMDVY | |
| 1M8R | SFSEENGDIVCGGDDPCKKEICECDRAAAICFRD | |
| | NLNTYNDKKYWAFGAKNCPQEESEPC | |
| pla2-sawscaled2- | NLYQFGRMIWNRTGKLPILSYGSYGCYCGWGGQ | 312 |
| 1OZ6 | GPPKDATDRCCLVHDCCYTRVGDCSPKMTLYSY | |
| | RFENGDIICDNKDPCKRAVCECDREAAICLGENV | |
| | NTYDKKYKSYEDCTEEVQEC | |
| snaclec-mocassin- | NNCPHDWLPMNGLCYKIFDELKAWEDAERFCR | 313 |
| 1JZN | KYKPGCHLASFHTYGESLEIAEYISDYHKGQAEV | |
| | WIGLWDKKKDFSWEWTDRSCTDYLTWDKNQP | |
| | DVYQNKEFCVELVSLTGYRLWNDQVCESKNAFL | |
| | CQCKF | |
| snaclec-rattler- | NNCPLDWLPMNGLCYKIFNQLKTWEDAEMFCR | 314 |
| 1JZN | KYKPGCHLASFHRYGESLEIAEYISDYHKGQENV | |
| | WIGLRDKKKDFSWEWTDRSCTDYLTWDKNQPD | |
| | HYQNKEFCVELVSLTGYRLWNDQVCESKDAFLC | |
| | QCKF | |
| snaclec-sawscale- | VCCPLGWSGYDQNCYKAFEELMNWADAEKFCT | 315 |
| 1OZ7 | QQHKGSHLVSLHNIAEADFVVKKIVSVLKDGVI | |
| | WMGLNDVWNECNWGWTDGAQLDYKAWNVES | |
| | NCFIFKTAENHWSRTDCSGTHSFVCKSPA | |
| snaclec-taipan | SCCCPQDWLPKNGFCYKVFNDHKNWNDAEMFC | 316 |
| | RKFKPGCHLASIHSNADSADLAEYVSDYLKSDG | |
| | NVWIGLNDPRKQRTWVWSDRSPTNYYSWNRGE | |
| | PNNSKNNEYCVHLWALSGYLKWNDTPCKALYS | |
| | FICQCRF | |
| kunitz-taipan-6A5I | RPKFCHLPPKPGPCRAAIPRFYYNPHSKQCEKFIY | 317 |
| | GGCHGNANSFKTPDECNYTCLGVSLPK | |
| kunitz-cobra-1DTK | RPRFCELPAETGLCKARIRSFHYNRAAQQCLEFIY | 318 |
| | GGCGGNANRFKTIDECHRTCVG | |
| kunitz-mamba- | LQHRTFCKLPAEPGPCKASIPAFYYNWAAKKCQ | 319 |
| 1DTK | LFHYGGCKGNANRFSTIEKCRHACVG | |
| cysrich-mocassin- | SVDFDSESPRKPEIQNQIVDLHNSLRRSVNPTASN | 320 |
| 6IMF | MLKMEWYPEAAANAERWAYRCIESHSPRNSRV | |
| | LGGIKCGENIYMSSIPIKWTEIIHAWHGENKNFKY | |
| | GIGADPPNAVIGHFTQIVWYKSYLVGCAAAYCPS | |
| | SEYSYFYVCQYCPAGNIIGKIATPYKSGPPCGDCP | |
| | SACVNGLCTNPCTKEDKYTNCKSLVQQYGCQD | |
| | KQMQSECSAICFCQNKII | |
| cysrich-rattler- | SVDFDSESPRKPEIQNKIVDLHNFLRRSVNPTASN | 321 |
| 6IMF | MLKMEWYPEAAANAERWAYRCIESHSPRDSRV | |
| | LGGIKCGENIYMSPVPIKWTEIIHAWHGENKNFK | |
| | YGIGAVPPNAVTGHFSQVVWYKSYRIGCAAAYC | |
| | PSSKYSYFYVCQYCPAGNIIGKTATPYKSGPPCG | |
| | DCPSACDNGLCTNPCTKEDKYTNCKSLVQQAGC | |
| | QDKQMQSDCPAICFCQNKII | |
| cysrich-taipan- | TVDFASESSNKKDYRKEIVDKHNDLRRSVKPTA | 322 |
| 2DDB | RNMLQMKWNSRAAQNAKRWANRCTFAHSPPY | |
| | TRTVGKLRCGENIFMSSQPFAWSGVVQAWYDEV | |
| | KKFVYGIGAKPPSSVIGHYTQVVWYKSHLLGCA | |
| | SAKCSSTKYLYVCQYCPAGNIIGSIATPYKSGPPC | |
| | GDCPSACDNGLCTNPCKHNNDFSNCKALAKKSK | |
| | CQTEWIKSKCPATCFCRTEII | |
| PA2-sawscaled- | SIVELGKMIIQETGKSPFPSYTSYGCFCGGGERGP | 323 |
| viper-2QHE | PLDATDRCCLAHSCCYDTLPDCSPKTDRYKYKR | |
| | ENGEIICENSTSCKKRICECDKAMAVCLRKNLNT | |
| | YNKKYTYYPNFWCKGDIEKC | |
| PA2-water- | SVLELGKMILQETGKNAITSYGSYGCNCGWGHR | 296 |
| moccassin-1PPA | GQPKDATDRCCFVHKCCYKKLTDCNHKTDRYS | |
| | YSWKNKAIICEEKNPCLKEMCECDKAVAICLREN | |
| | LDTYNKKYKAYFKLKCKKPDTC | |
| PA2-diamondback- | SLVELGKMILQETGKNPITSYGIYGCNCGVGSRH | 297 |
| rattler-1PPA | KPKDGTDRCCFVHKCCYKKLTDCDPKMDGYTY | |
| | SFKDKTIICDVNNPCLKEMCECDKAVAICLRENL | |
| | DTYNKKYKIYPKFLCKKPDTC | |
| PA2-water- | NLFQFEKLIKKMTGKSGMLWYSAYGCYCGWGG | 299 |
| moccassin2-1VAP | QGRPKDATDRCCFVHDCCYGKVTGCNPKMDIY | |
| | TYSVDNGNIVCGGTNPCKKQICECDRAAAICFRD | |
| | NLKTYDSKTYWKYPKKNCKEESEPC | |
| PA2-diamondback- | SLVQFETLIMKIAGRSGLLWYSAYGCYCGWGGH | 300 |
| rattler2-1PP2 | GLPQDATDRCCFVHDCCYGKATDCNPKTVSYTY | |
| | SEENGEIICGGDDPCGTQICECDKAAAICFRDNIPS | |
| | YDNKYWLFPPKNCREEPEPC | |
| PA2-sawscaled- | NLYQFGRMIWNRTGKLPILSYGSYGCYCGWGGQ | 312 |
| viper-10Z6 | GPPKDATDRCCLVHDCCYTRVGDCSPKMTLYSY | |
| | RFENGDIICDNKDPCKRAVCECDREAAICLGENV | |
| | NTYDKKYKSYEDCTEEVQEC | |
| PA2-krait-1BUN | NLINFMNMIRYTIPCEKTWGEYTNYGCYCGAGG | 324 |
| | SGTPIDALDRCCYVHDNCYGDAEKIHKCSPKTQS | |
| | YSYKLTRRTIICYGAAGTCARIVCDCDRTAALCF | |
| | GDSEYIEGHKRIDTKRYCQ | |
| PA2-taipan-1P7O | SLLNFANLIECANHGTRSALAYADYGCYCGKGG | 325 |
| | RGTPLDDLDRCCHVHDDCYGEAEKLPACNYLM | |
| | SSPYFNSYSYKCNEGKVTCTDDNDECKAFICNCD | |
| | RTAAICFAGATYNDENFMISKKRNDICQ | |
| PA2-taipan2-1AE7 | NLLQFGFMIRCANRRSRPVWHYMDYGCYCGKG | 303 |
| | GSGTPVDDLDRCCQVHDECYGEAVRRFGCAPY | |
| | WTLYSWKCYGKAPTCNTKTRCQRFVCRCDAKA | |
| | AECFARSPYQNSNWNINTKARCR | |

### Example 5: Exemplary Treatment Method

A randomized double-blind controlled trial is conducted to compare the effect of low dose versus high dose. Patients presenting within 24 hours of snake bite with hematological or neurological evidence of systemic envenomation are included in the study. Patients are randomized either to receive high dose or low dose treatment with a composition that comprises one or more antibodies or antigen-binding fragments herein.

Primary outcome measures include, but are not limited to, (1) composite endpoint: number of patients who die, need assisted ventilation, or show a worsening of neurotoxicity, which is defined as the appearance of 2 new neurotoxic signs or the appearance of a severe neurotoxic sign (e.g., loss of gag reflex or paradoxical breathing); and/or number of patients with a serious adverse events.

### Example 6: Exemplary Mouse Model Establishing MTD to Demonstrate Toxins Were Active

Toxicity/tolerability for toxins from each species was determined in mice by delivering an intraperitoneal dose, sufficient to achieve greater than the lethal dose 50 (LD50: 50% of mice dead after compound administration).

Body weight and rectal body temperature for each mouse was measured prior to toxin administration. Toxin was administered intraperitoneally to each mouse (5 mice per group). Rectal body temperature measurements and general observations were made every 20 minutes post-dose, up to 2 hours, and subsequently 24 hours after administration. It was found that, for the given toxins tested, mice that either recovered or were not affected by a given dose of toxin showed full recovery 2-4 hours post-dose and remained healthy beyond 24 hours. If the dose of a given toxin was lethal due to rapid rectal temperature drop (>2 degrees from baseline) for the given doses described, mice were dead or euthanized by 60 minutes post-dose. Table 27 provides a description of the treatment groups.

TABLE 27
Treatment Groups

| | | | Time to | |
|---|---|---|---|---|
| Toxin Species | Dose | Survival | Euthanasia | |
| Short-neurotoxin-mamba-4LFT | 1 mg/kg | 0/5 | ~40 minutes | |
| | | | (min) | |
| Short-neurotoxin-mamba-4LFT | 2 mg/kg | 0/5 | ~30 | min |
| Short-neurotoxin-cobra (1CTX-like) | 1 mg/kg | 0/5 | ~55 | min |
| Short-neurotoxin-taipan-1TXA | 1 mg/kg | 3/5 | ~40 | min |
| Short-neurotoxin-taipan-1TXA | 2 mg/kg | 3/5 | ~20 | min |

### Example 7: In Vivo Effect of Anti-Venom Antibodies

An experiment was conducted to determine >LD50 for purified recombinant snake alpha neurotoxin when treated with (1) S-Neurotoxin+SNEURO_P01_B11, (2) S-Neurotoxin+SNEURO_P01_D9, or (3) no antibody control (S-Neurotoxin). Survival is shown as mice that either did not have any significant body temperature change or the body temperature returned to normal by 2 hours and beyond (Table 28).

TABLE 28
In vivo number of mice dying after alpha-neurotoxin injection

| Control or Antibody | Batch 1 | | | Batch 2 | | |
|---|---|---|---|---|---|---|
| Clone | Cobra | Mamba | Taipan | Cobra | Mamba | Taipan |
| S-Neurotoxin | 5/5 | 5/5 | 3/5 | 5/5 | 5/5 | 3/5 |
| S-Neurotoxin + | 0/5 | | | | 5/5 | 4/5 |
| SNEURO_P01_B11 | | | | | | |
| (Centi-B11) | | | | | | |
| S-Neurotoxin + | | 0/5 | 0/5 | 1/5 | | |
| SNEURO_P01_D9 | | | | | | |
| (Centi-D09) | | | | | | |

### Example 8: Antibody Libraries

This example demonstrates production of an antibody library from a hyperimmune subject. Hyperimmune subject (HiS). The inventors have collected B-cell samples from a human subject with a 17-year immunization history against snake venom, by self-administering dilute amounts of venom from taipans, cobras, rattlesnakes, coral snakes, kraits, tiger snakes, eastern brown snakes, and mambas. This subject gradually increased the concentration of venom and has achieved sufficient antibody titers to tolerate full snake bites without succumbing to death or amputation. The HiS has documented administration of over 700 escalating doses of venom and over 200 live bites.

Antivenom antibody library construction. In a passive study design, 20 ml of peripheral blood were taken at day 0 prior to, and 28 days post, the routine immunization schedule utilized by the hyperimmune subject that they had established over a 17-year period to maintain hyperimmunity. PBMCs were isolated by Ficoll separation and cDNA was extracted. Antibody variable domain VH, Vκ and Vλ diversity was extracted using primer sets designed for simultaneous amplification of human repertoires for both high-throughput sequencing and antibody display library construction. The bar-coded library samples were sequenced by MiSeq 2×300 paired end reads to a depth of 500,000 reads per library. The same material was digested and cloned into a scFv-pIII fusion display vector and transformed to a final library size of 2e9. The post-transformed library was also sequence-confirmed by NGS to have a distribution of >1e8 heavy chains and >1e5 light chains. Although the heavy chains and light chains were cloned separately, the size of the library is such that any starting clone with a frequency greater than 5e-4 will have the native pair present in the resulting library. The library was confirmed to contain all human native V-genes. 2×10⁹ transformants were obtained during library transformation.

A set of 25 homologs were selected for toxin-Fc-AviTag fusions and toxin-10 His-AviTag fusions (“10 His” disclosed as SEQ ID NO: 532). Fc-tag and 6xHis tags (His-His-His-His-His-His; SEQ ID NO: 326) were provided to assist in toxin purification, and AviTags were provided to enable site-specific biotinylation to magnetic beads for automated soluble phase panning. When induced for expression in HEK293 cells, 21 of the 25 toxins produced measurable titers. All recombinant toxins were then exposed to hyperimmune donor's serum, and 15 of 21 toxins had strong reactivity to the serum, including multiple species that the donor had never immunized against directly. The results validated that (1) the hyperimmune subject had reactivity against these specific toxins and (2) that toxins were produced in a form that was recognizable to the donor's antibodies.

Isolation of broadly neutralizing anti-alpha-neurotoxin antibody. The inventors demonstrated that the approach technologies were able to successfully isolate the key proof-of-principle for the effort: a fully human, broadly neutralizing antibody against alpha-neurotoxin, one of the “big four” toxins in venom (Centi-D09). With in vivo protection models in multiple snake species, crystallography of the bnAb in complex with alpha-neurotoxin from multiple snake species, kinetics and biophysical characterization of the bnAb, the molecular basis of broadly neutralizing antivenom antibody has been established.

In certain aspects, innovation of embodiments described herein lies in three features of the next generation antivenom: 1) sourcing of our antibodies; 2) technological development; and 3) storage of the final product.

The technology described in this application utilizes a novel, diverse immune library harvested from a middle-aged male who has been administering dose-escalating self-immunizations for the past 19 years (over 700 boosts and 200 live bites) from 17 diverse snake species including: Western Diamondback, Mojave, coral snake, black mamba, western green mamba, eastern green mamba, Jameson mamba, monocled cobra, Egyptian cobra, forest cobra, water cobra, Cape cobra, Common Krait, Banded Krait, Coastal Taipan, PNG Taipan, tiger snake, and eastern brown snake. These antibodies are safe and effective in our middle-aged male, and are an advantage compared to humanizing antibodies from animal sources or naïve human libraries. Moreover, their single-domain nature can result in a reduced half-life in comparison to fully human IgGs. If not properly neutralized with a single dose, repeated injections are warranted due to their small size that allows for renal elimination.

Roboticized cross-species phage panning. The technology described herein enabled the generation of an immune library using unique Next Generation Sequencing (NGS) to guide amplification and tracking of antibody variable domain repertoires from blood draws before and 28 days post venom immunization of the male subject, followed by deep sequencing and antibody phage display, enabling downstream venom binders to be traced back to the source blood draw. The antibody library is enriched for highly affinity-matured antivenom antibodies that were selected for breadth across snakes and have demonstrated capacity to protect from high doses of lethal snake venom in vivo. In addition, our roboticized homolog cross-panning protocols enable the rapid isolation of antivenom broadly neutralizing antibodies. We have already demonstrated the value of this combination of both library and robotic cross-panning in the recovery of the in vivo protecting anti-long-neuro-toxin bnAb Centi-D9.

Thermostabilized lyophilized antibodies Standard antivenom requires continuous refrigeration at 2-8° C. to preserve stability. This poses a significant challenge in rural communities, often in arid regions with endemic venomous species, that may not have the infrastructure to properly store antivenom. In certain instances, antibodies described herein are assessed with respect to the stability of lyophilized or high-concentration formulation antivenoms at room temperature. This application describes thermostabilized antibodies with more lenient storage requirements with longer-shelf life, greatly expanding the types of health centers that can store and access it and enable field-deployment of lifesaving medicine at the site of envenomation.

The experiments described herein identified and characterized a pool of cross-reactive high affinity antivenom antibody candidates; (2) explored the mechanism of action by epitope characterization from whole venom pools; and (3) determined a unique pattern of protection by in vivo challenge in mice.

The inventors have demonstrated that broadly neutralizing antibodies (bnAbs) against snake venom exist. Centi-D9, a broadly neutralizing, ultra-high affinity antibody against long-neurotoxin was able to neutralize and provide in vivo protection against long-neurotoxins from cobras, taipans, mambas and cobras.

The inventors have also demonstrated that biophysical properties of lead clone Centi-D9 can be optimized by mutation of residues in Centi-D9 to the germline residues at particular positions; and removal of biochemical modification sites such as N-linked glycosylation motif NS. Thermostability and aggregation for these variants was tested for modified clones based on parental Centi-D9 in Table 29. Table 21B demonstrates that these modified clones retain affinity for multiple long neurotoxins derived from snake venoms in the family Elapidae.

TABLE 29

| Antibody | Tm | Tagg 266 |
|---|---|---|
| Parental (SNEURO_P01_D09) | 76 | 76.5 |
| SNEURO_P01_D09 VH germlined | 75.1 | 74.6 |
| SNEURO_P01_D09 VK germlined | 76 | 76.5 |
| SNEURO_P01_D09 FW germlined | 75.5 | 74.7 |
| SNEURO_P01_D09 VK L3 NS−>QS | 71.7 | 75.4 |
| SNEURO_P01_D09 VK L3 NS−>NT | 76.5 | 69.6 |

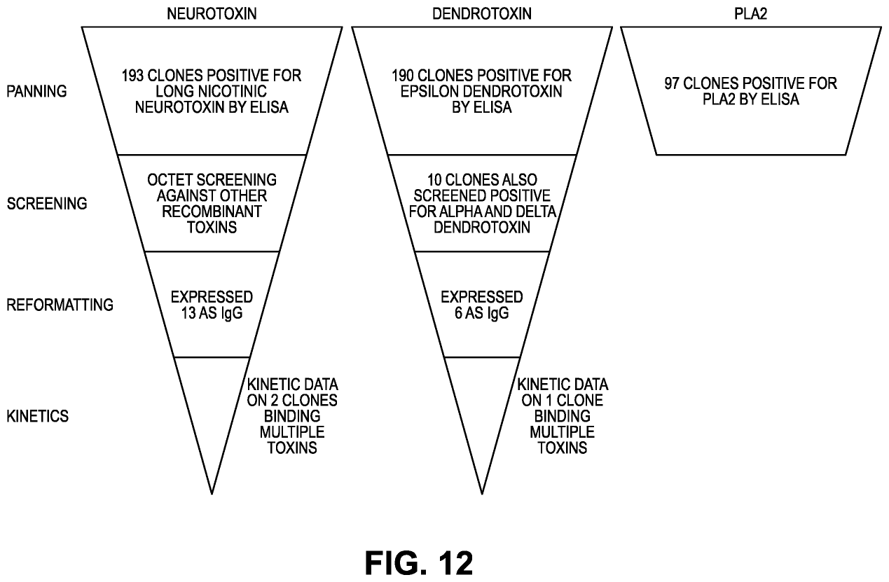

Identification of antibody candidates for three toxins: We identified by ELISA screening 193, 97, and 190 antibody clones against neurotoxin, phospholipase A2 (PLA2), and E-dendrotoxin, respectively (FIG. 12). For long-neurotoxin, screening was conducted by ELISA; Thirteen clones were selected by ranked off-rate of periplasmic extra (PPE) using Octet HTX and then reformatted and expressed as IgG. High resolution kinetics data were generated for two candidates, Centi-D09 and Centi-B11, against three four toxins (long-neurotoxin from Taipan, Cobra, Mamba, Krait) with K_{D}s against various neurotoxins ranging from 2.75e-09 to 2.63e-10 and 3.3e08 to 1.75e09, respectively. As assessed by Octet HTX and BiaCORE 8K at both 25° C. and 37° C., Centi-D9 is an ultra-high affinity (picomolar) bnAb against the three snake species of taipan, cobra, and mamba (490 pM, 37 pM, 74 pM, respectively). For dendrotoxin, ten of the initial 190 epsilon (E) dendrotoxin-positive clones were also ELISA positive against A and D-dendrotoxins. Six of these 10 with the most breadth against three different dendrotoxins were reformatted to IgG. The binding kinetics of the reformatted dendrotoxin antibody candidates were characterized, with one clone (Centi-DTX-B03) which showed strong binding activity against dendrotoxin from 4 different species, demonstrating the neutralizing breadth of that antibody. Crystallographic evidence of complexes has been observed for one clone. Anti-PLA2 and anti-Kunitz clones were identified and characterized for breadth by ELISA.

The following was achieved: 1) generation of kinetics data for each antibody tested against each venom; 2) reformatting antibody candidates into fully human IgG; and 3) characterization of the binding affinity of the reformatted IgG antibodies. The PLA2 enzyme was found to be toxic to mammalian cells expressing the recombinant protein, so we adapted our protocol to use PLA2 isolated from whole snake venom, confirmed to be active through phospholipase enzymatic assay, and biotinylated for panning. This product was then used successfully in panning and screening.

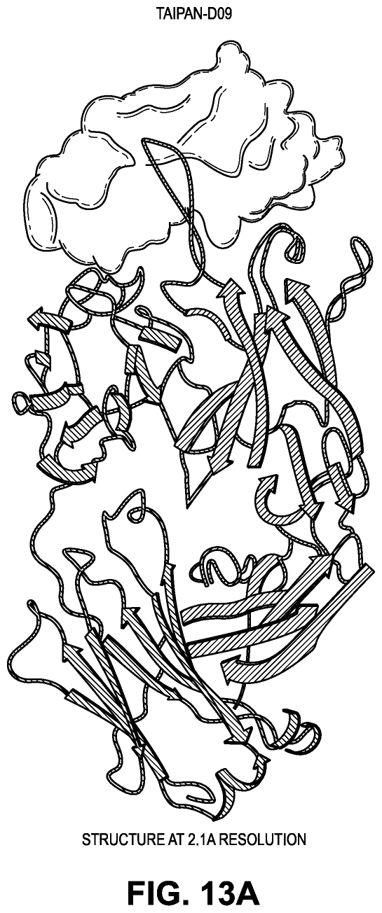

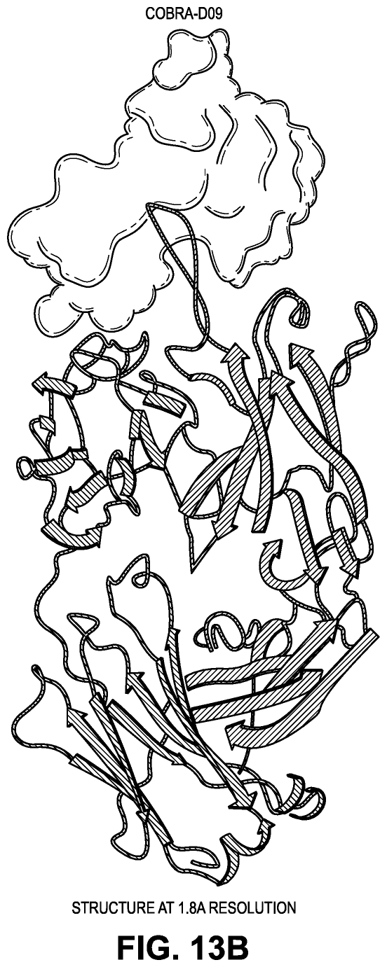

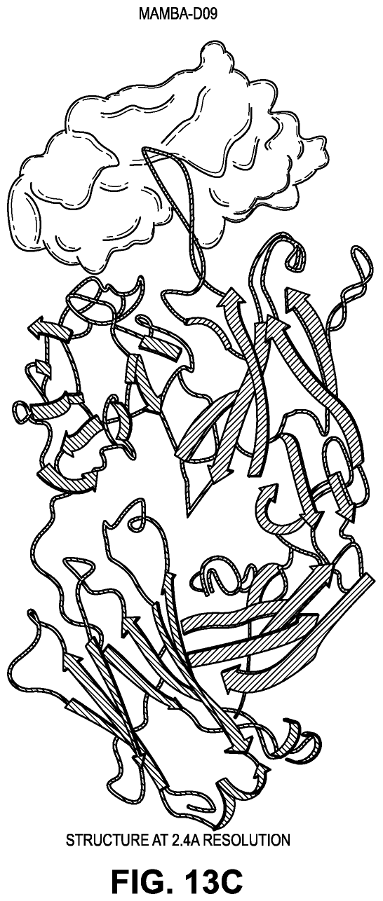

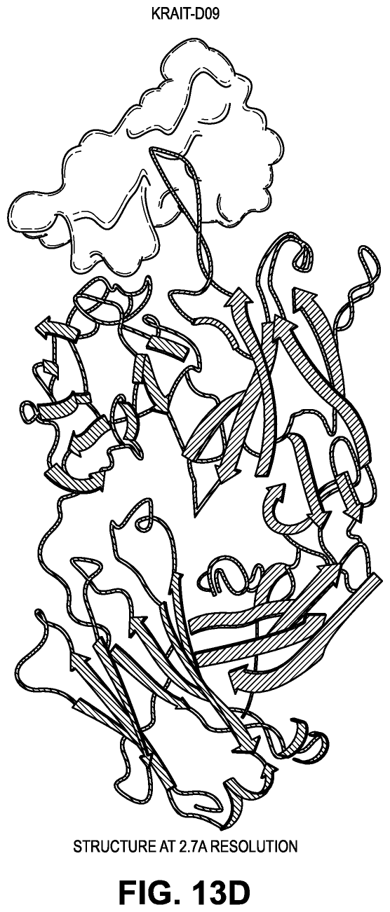

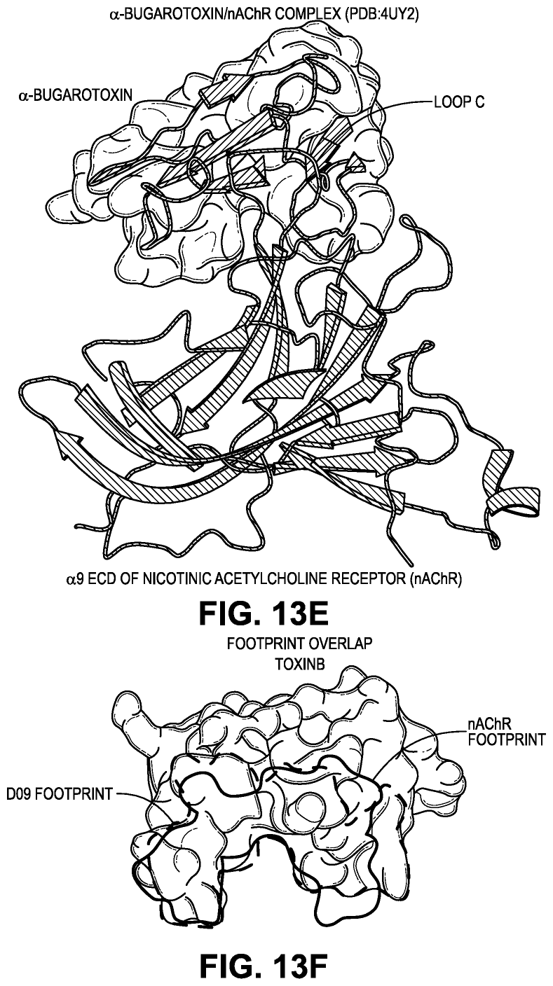

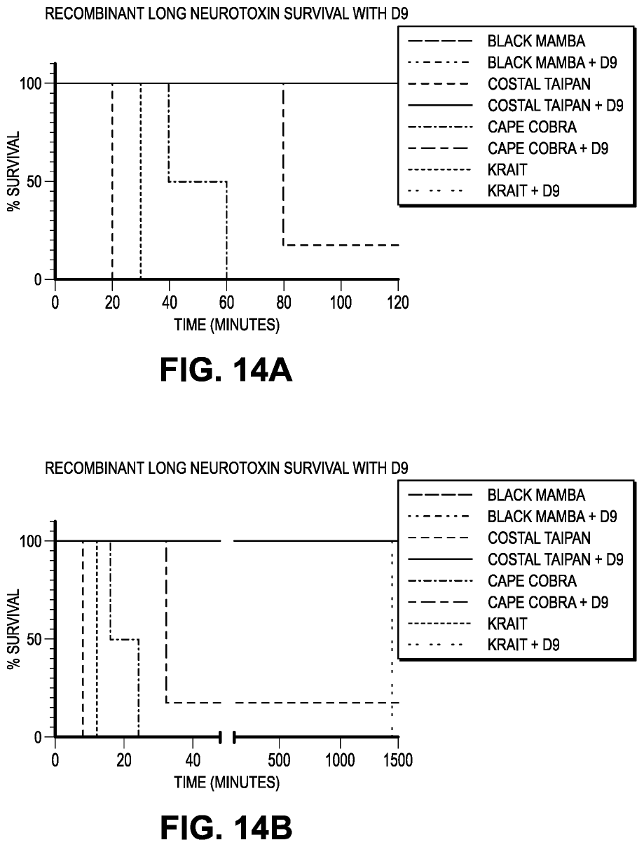

Kinetics and binding mode of antibody Centi-D9 (SNEURO_P01_D09). Crystal structures were generated to define its binding orientation and parameters (FIG. 13). The 4 crystals mapped Centi-D09 in complex with long-neurotoxin from krait (Bungarus caeruleus, referred to as alpha bungarotoxin in this species), black mamba (Dendroaspis polylepsis), coastal taipan (Oxyuranus scutatellus), and cape cobra (Naja nivea). The crystal structures demonstrated Centi-D9 binds long-neurotoxin from all four species in a similar binding mode, which prevents the long-neurotoxin from binding the nicotinic acetylcholine receptor (nAChR). This is demonstrated by the binding footprint for Centi-D9 and nAChR being almost completely overlapping on the surface of long-neurotoxin (FIG. 13F). Both Centi-D9 and Centi-B11 (SNEURO_P01_B11) bind toxins from these four elapid species, with Centi-D9 showing favorable kinetics relative to Centi-B11 on full Biacore800 and Octet HTX kinetics assays. The crystal structure of Centi-D9 in complex with these long neurotoxins clearly demonstrates a conserved binding mode/motif across these diverse toxins (no pair having greater than 65% sequence identity between them): the VH CDR3 of Centi-D9 comes in contact with at least one residue in ‘finger’ loop I and at least one residue in ‘finger’ loop II.

Centi-D9 in live challenge studies in mouse models. Live-challenge Centi-D9 mouse models of envenomation were utilized to establish the lethal dose range for four elapid species (Table 30) and determine the functional activity and broad-spectrum efficacy of Centi-D9 against recombinant toxins and whole snake venoms in vivo.

TABLE 30

| Species | LD100¹ | |
|---|---|---|
| Oxyuranus scatatellus | 0.05 | mg/kg |
| Naja kauthia | 0.5 | mg/kg |
| Naja nivea | 1 | mg/kg |
| Dendroapsis polylepsis | 0.5 | mg/kg |

¹Empirically derived _{LD100} for 4 whole snake venoms tested during dose-range testing to establish _{LD50} to LD100.

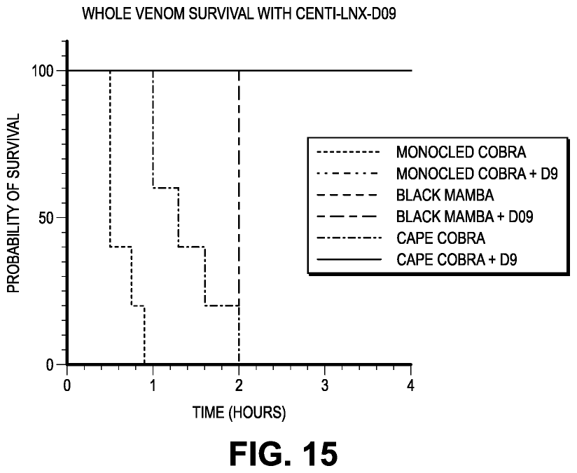

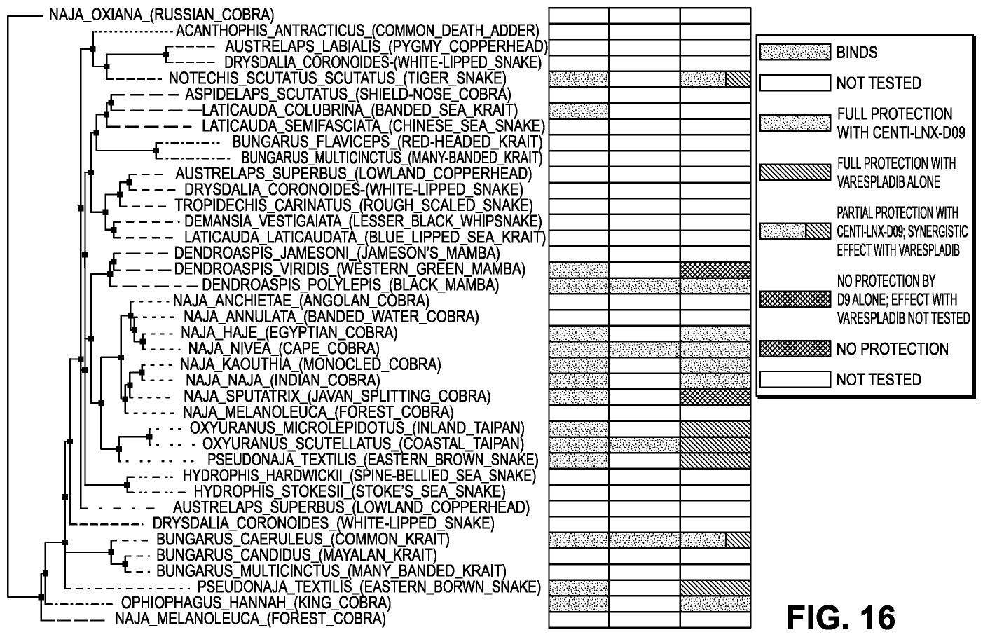

Mice exposed to lethal doses of monocled cobra, black mamba, and cape cobra venoms were completely protected from death by pre-treatment with Centi-D9 through broadly neutralizing activity, significantly exceeding expectations in protection from whole venom challenges as a single antibody (as opposed to a cocktail) (FIG. 15). We choose to focus on the elapid family based on abundant evidence pointing towards long-neurotoxin being the principal driver of lethality in the majority of elapids, and therefore critical basis of a universal antivenom cocktail for elapids. Centi-D09 was demonstrated to be a broadly neutralizing antibody, protective in vivo against whole venom of six elapid species (FIG. 16).

The existence of Centi-D09 provided evidence that broadly neutralizing antibodies against homologous snake toxins exist and can be isolated as a basis for a universal antivenom. Furthermore, the identification of Centi-D09 demonstrates that broadly-neutralizing antibodies against snake venom toxins from a variety of species can be successfully isolated from a hyper-immune subject who has a history of repeatedly mounting humoral immune responses against a variety of such toxins.

A broadly-neutralizing antivenom may require combinations of agents that neutralize separate components found in the two major classes of venomous snakes: elapids and viperids (Table 31). For the neurotoxic elapids, venom's virulence is driven by long-neurotoxin (3 FT) and PLA2. An anti-PLA2 bnAb from the panel of candidate binders can be combined with Centi-D9, the anti-long-neurotoxin bnAb to create a 2-member cocktail that provides protection against elapids.

TABLE 31
Venoms to Target

Target

| Venom | Elapids | Viperids |
|---|---|---|
| Phospholipase A1 (PLA2) | ′1′1′1 | ′1′1′1 |
| Three-finger toxin (3FT) | ′1′1′1′1 | |
| Kunitz Peptide (KUN) | ′1′1 | ′1 |
| Snake venom serine protease (SVSP) | | ′1′1′1 |
| Snake venom metalloprotease (SVMP) | | ′1′1′1 |

1Toxins listed can vary widely in abundance within specific venoms of individual snake species from both families, but these are the main toxins of consequence, as well as a general representation of relevant abundance, notated by the number of checkmarks.

Centi-D09, a single 3 FT bnAb, was shown to work against black mamba (Dendroaspis polylepsis), Cape cobra (Naja nivea), and Monocled cobra (Naja kaouthia). Centi-D09 was further shown to work against additional elapid members Egyptian cobra (Naja haje), Indian cobra (Naja naja), and king cobra (Ophiphagus hannah). Centi-D09 was further shown to bind 3FTX long-neurotoxins in venom obtained from tiger snake (notechis scutatus scutatus), banded sea krait (laticauda colubrina), western green mamba (dendoapsis viridis), javan spitting cobra (naja sputatrix), inland taipan (oxyuranus microlepidotus), and eastern brown snake (pseudonaja textilis), by Gator BLI assay (FIG. 16).

In some instances, Centi-D09 can be combined with an anti-PLA2 inhibitor. For example, Centi-D09 can be combined with PLA2 small molecule inhibitor varespladib for coastal taipan. Alternatively, or in addition, the small molecule inhibitor can be replaced with a PLA2 bnAb. In other instances, Centi-D09 can be combined with an antibody against dendrotoxin (kunitz-like peptide) specific to dendroaspis genus.

Production of recombinant toxins. The EXPi-293 mammalian expression system is used for expression of venom toxin antigens. Toxin proteins are purified by protein G DYNABEADS™, biotinylated, and quality-controlled for biotinylation, and binding to control antibodies. Some toxins are difficult to produce using this method, as the toxins are lethal to the protein-producing cells. In these cases (such as PLA2), whole snake venom is obtained and fractionated using HPLC to obtain purified toxin for biotinylation.

Panning of phage libraries against venom antigens. The immune library (2×10⁹) is heated for 10 min at 72° C. and de-selected against Protein G DYNABEADS™ (INVITROGEN®), M-280 Streptavidin DYNABEADS™ (INVITROGEN®), Histone from Calf Thymus (Sigma), Human IgG (Sigma) ssDNA-Biotin NNK (Integrated DNA Technologies), and DNA-Biotin NNK (Integrated DNA Technologies). Next, the library is serially panned against the venom antigens, captured by M-280 Streptavidin DYNABEADS™ using an automated protocol on Kingfisher FLEX (THERMO FISHER SCIENTIFIC®). Selected phages are acid eluted from the beads and neutralized using Tris-HCl PH 7.9 (TEKNOVA®). ER2738 cells are infected with the neutralized phage pools at OD600=0.5 at a 1:10 ratio and after 40 min incubation at 37° C. and 100 rpm, the phage pools are centrifuged and incubated on agar with antibiotic selection overnight at 30° C. The rescued phages are precipitated by PEG and subjected to three additional rounds of soluble-phase automated panning. The resulting antibody clones are screened by ELISA for binding activity against recombinant proteins or whole snake venom.

Kinetic characterization of preliminary ELISA positives by SPR. The kinetic properties of a pool of ELISA positive antibodies are characterized against whole snake venoms and fractionated toxins using CARTERRA® LSA (CSLA), a high-throughput SPR platform. Each antibody is tested against all venoms or fractionated toxins for kinetic characterization and cross-reactive binding. Antibodies are further characterized for thermostability and aggregation by fluorescence and light scattering technologies using Unchained Labs (UNcle). Selecting clones with the fastest on-rate (ka), and slowest off-rate (kd) minimizes trauma to the victim as venom must be neutralized quickly and not released.

Binding kinetics of our ELISA positive antibody candidates are tested using CLSA. Data suggests these candidates bind to snake venoms they were panned against; their binding affinity is characterized to determine potential functionality to neutralize target venom/toxin. It is determined if any of our candidates can bind to ≥2 venom species or toxins, as cross-reactive molecules are utilized in achieving a broad spectrum antivenom treatment. SPR technologies allow for accurate determination of kinetic data (ka [association rate], kd [dissociation rate], K_{D}) [equilibrium dissociation constant]) without purifying the bacterial supernatants expressing our antibody candidates, currently expressed using a proprietary scFv phagemid, allowing for phage display. K_{D} is determined using CARTERRA® software, which utilizes the antigen's maximum resonance ([A]Rmax) at a certain concentration binding to the scFv on the chip divided by the resonance equilibrium (KD=[A]Rmax/Req). The CLSA Platform advantageously tests ≥4 unique antigens on 384 antibody candidates in <18 hours (h). This high-throughput technology determines the relative affinities of all candidates to the original venom they were panned against and ≥20 different whole venoms/fractionated peptide toxins in <7 days. Candidates with the greatest affinities to venoms/toxins (<10 nM) are reformatted as IgG in our mammalian expression system. IgGs are purified and tested for thermostability and aggregation propensity. ScFvs expressed by the bacterial cell line ER2738 are captured using an anti-V5 antibody coupled to the CLSA chip. Centivax's phagemid vector inserts a C-terminal V5 tag allowing for downstream scFv capture. Whole snake venoms and toxins are flowed over every captured clone using an 8-point dilution scheme with a maximum concentration of 3 μM and a minimum concentration of 450 pM to determine binding kinetics and presence of cross-reactivity. Anti-Her2 scFv are captured as a negative control against all venoms.

The CLSA Platform is a versatile, high throughput monoclonal antibody characterization method combining patented continuous flow microfluidics with array SPR detection, delivering massive data generation in reduced time and consumable costs. The inventors are the first to use this technology to determine binding or cross-reactivity of antibodies to snake species; most snakebite laboratories do not have access to such an SPR instrument.

In the case of low-affinity binders, a proprietary affinity maturation POLISH technology may be utilized to create millions of variants of these clones by CDRH3 mutagenesis and light-chain shuffling. Sets of these clones are screened by CLSA for higher affinity (<10 nM). If poor cross-reactivity arises, NGS data from panning rounds can be utilized to express certain clones that were enriched across pannings but did not show up during the initial ELISA screening and test them on ELISA and CARTERRA®, in addition to the initial positives.

Kinetics data is generated for each antibody tested against each venom. The accepted affinity, <10 nM K_{D}, is determined by an on-rate [ka] of 104 1/Ms to 105 1/Ms and an off-rate [kd] of 10-4 1/s to 10-6 1/s. Clones with the highest-affinity to ≥1 whole venom or toxin (<10 nM) and/or cross-reactive (as indicated by binding ≥1 species) antibodies are selected for IgG reformatting.

Reformatting antibody candidates to human IgG. Antibody candidates identified are reformatted from scFvs into fully human IgGs by PCR. An IgG backbone with minimal effector function (e.g., LALA mutations) is used, as the goal is to bind and neutralize the venom rather than cause a downstream inflammatory immune response. Designed primers add adapted ends suitable for cloning into a mammalian expression vector, pTT5. Restriction sites suitable to the pTT5 vector are added to each scFv candidate via PCR for the heavy chain V-gene and light chain V-gene (previously characterized by SS). Resulting products are digested and cloned into DH5-alpha bacterial cells. Proper cloning confirmation is performed by Sanger Sequencing (Eton Bioscience). Clonal DNA is midi-prepped using a commercial kit (ZYMO RESEARCH®) and transiently transfected into HEK293 cells for mammalian expression. Supernatants from each are harvested 5 days post-transfection. IgGs are purified and quantified with Protein A using Octet QK with a minimum expression of 50 μg/mL; expression purity is analyzed with SDS-PAGE. The pTT5 vector is utilized for reformatting and expressing antibody candidates.

Stability and kinetic characterization of IgG antibody candidates. The binding affinity of the reformatted antibody candidates in IgG format on the CLSA is determined against the previously tested venoms/peptides. While scFvs contain the antibody domains determining antigen binding, affinities may alter when in complex with the constant region of a full IgG. Binding efficiency can increase due to avidity, as a full IgG contains two binding sites compared to one on scFv or Fab. IgG candidates' stability is characterized using UNchained Labs (UNcle). UNcle uses dynamic light scattering, static light scattering, and fluorescence to determine a protein's melting temperature, aggregation propensity, and isothermal stability crucial for determining overall stability of the IgG candidates and quality of the downstream antivenom.

IgGs may be thermally unstable or aggregation prone. To rectify this, the IgG complementary-determining regions (CDRs) can be mutated and can be grafted into more thermostable human germline frameworks. As the driving force between antigen binding, the scaffold for which the CDRs are loaded into can be changed and retain binding to create a more thermostable molecule. This can be done by having known thermostable human germline scaffolds with the CDRs of our chosen antibody candidates synthesized by IDT or Twist and cloned into our pTT5 vector. Binding kinetics is retested to determine binding retainment.

IgGs may bind with the same affinity or greater as they did in scFv format (<10 nM) with improved thermostability and aggregation propensity by UNcle. Only IgGs that are 95% monomeric with a melting temperature (Tm) of 68° C. or greater and a KD of <10 nM will be used in subsequent Aims.

The antibodies/antibody cocktails of the invention may be lyophilized. The formulation for lyophilization may comprise histidine, methionine, and polysorbate 80; for example: 50 mM histidine, 20 mM methionine, 0.05% polysorbate 80 (w/v), pH 6.8. The lyophilization formulation may further comprise combinations of bulking agents/lyophilization stabilizers (including, but not limited to: sorbitol, mannitol, sucrose, dextrose, and glycine). The lyophilized dosage formulations are stored at 50° C. and analyzed at regular timepoints for 28 days by the following methods: visual appearance, SEC-HPLC, optical density, pH, dynamic light scattering, Tmelting, Taggregation, particulates, moisture content, or any combination thereof.

In certain instances, multiple antibodies can be lyophilized in a single cocktail when a common set of lyophilization parameters allows for stable processing of all antibodies together. Alternatively, the antibodies can be lyophilized separately, the freeze-dried powders mixed by weight, and the combined powder reconstituted for downstream analysis.

In one aspect, described in this application are a series of monoclonal antibodies against lethal toxins in snake venoms; that is, cocktails of antibodies that are broadly protective against the viper and elapid families of snakes. Each of these antibodies are individually optimized to broadly neutralize whole families of polymorphic toxins (e.g., a single antibody capable of neutralizing dendrotoxins from elapids around the globe). By combining several broad-spectrum anti-toxin antibodies, we can eliminate snakebite lethality by neutralizing the most potent toxins present in the venoms of the deadliest snakes. The unique combination of a cocktail strategy with antibodies of high potency and breadth allows us to create snakebite medicines that will not require identification of the envenoming species and allow for significantly more rapid treatment. Furthermore, through lyophilization of the antibody cocktails, medicines are created that are ultra-stable and can be stored at ambient temperature for long periods of time. Also described is a dual-chambered autoinjector for use by untrained individuals or in a field care setting which can be carried and used easily by travelers, health care workers or armer forces members in regions where lethal snakebite is of serious concern.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating a subject who suffers from an envenomation, comprising administering to the subject a composition comprising an effective amount of a broadly-neutralizing anti-venom that comprises one or more antibodies or one or more antigen-binding fragments.

2. The method according to claim 1, wherein the envenomation occurs from one or more snake(s).

3. (canceled)

4. The method of claim 1, wherein the envenomation is from (a) saw-scaled viper, water moccasin, lancehead, rattlesnake, Russell's viper, puff adder, or a combination thereof; or (b) krait (Bungarus caeruleus), black mamba (Dendroaspis polylepsis), coastal taipan (Oxyuranus scutatellus), cape cobra (Naja nivea), or a combination thereof.

5.-8. (canceled)

9. The method of claim 1, that further comprises administering an additional therapy or drug to the subject.

10. The method of claim 9, wherein the additional therapy or drug comprises a PLA2 inhibitor, wherein the PLA2 inhibitor comprises varespladib, methylvarespladib, or a combination thereof.

11. (canceled)

12. (canceled)

13. The method of claim 1, wherein the one or more antibodies or antigen-binding fragments comprise a variable heavy chain (VH) complementarity determining region 3 (CDR3) having an amino acid sequence that is at least about 90% identical to any one of SEQ ID NOS: 12 or 327-348.

14. The method of claim 1, wherein the one or more antibodies or antigen-binding fragments comprise a VH CDR2 having an amino acid sequence that is at least about 90% identical to any one of SEQ ID NOS: 41 or 371-385.

15. The method of claim 1, wherein the one or more antibodies or antigen-binding fragments comprise a VH CDR1 having an amino acid sequence that is at least about 80% identical to any one of SEQ ID NOS: 28 or 349-370.

16. The method of claim 1, wherein the one or more antibodies or antigen-binding fragments comprise a variable light chain (VL) CDR1 having an amino acid sequence that is at least about 90% identical to any one of SEQ ID NOS: 46 or 393-404.

17. The method of claim 1, wherein the one or more antibodies or antigen-binding fragments comprise a VL CDR2 having an amino acid sequence that is at least about 80% identical to any one of SEQ ID NOS: 80 or 405-416.

18. The method of claim 1, wherein the one or more antibodies or antigen-binding fragments comprise a VL CDR3 having an amino acid sequence that is at least about 90% identical to any one of SEQ ID NOS: 101 or 417-428.

19.-26. (canceled)

27. The method of claim 1, wherein the one or more antibodies or antigen-binding fragments comprise a VH having an amino acid sequence that is at least about 90% identical to any one of SEQ ID NOS: 242 or 429-500.

28. The method of claim 1, wherein the one or more antibodies or antigen-binding fragments comprise a VL having an amino acid sequence that is at least about 90% identical to any one of SEQ ID NOS: 273 or 501-512.

29.-74. (canceled)

75. The method of claim 1, wherein the one or more antibodies or antigen-binding fragments comprises a VH CDR3 of SEQ ID NO: 12, a VH CDR1 of SEQ ID NO: 28, a VH CDR2 of SEQ ID NO: 41, a VL CDR1 of SEQ ID NO: 46, a VL CDR2 of SEQ ID NO: 80, and a VL CDR3 of SEQ ID NO: 101.

76. The method of claim 1, wherein the one or more antibodies or antigen-binding fragments comprises a VH of SEQ ID NO: 242 and a VL of SEQ ID NO: 273.

77.-156. (canceled)

157. A broadly-neutralizing anti-venom composition comprising one or more an anti-venom antibody(ies) or antigen-binding fragment(s) that selectively bind(s) to one or more toxin(s) from one or more species of snake(s).

158.-229. (canceled)

230. The broadly-neutralizing anti-venom composition of claim 157, wherein the one or more antibodies or antigen-binding fragments comprises a VH CDR3 of SEQ ID NO: 12, a VH CDR1 of SEQ ID NO: 28, a VH CDR2 of SEQ ID NO: 41, a VL CDR1 of SEQ ID NO: 46, a VL CDR2 of SEQ ID NO: 80, and a VL CDR3 of SEQ ID NO: 101.

231. The broadly-neutralizing anti-venom composition of claim 157, wherein the one or more antibodies or antigen-binding fragments comprises a VH of SEQ ID NO: 242 and a VL of SEQ ID NO: 273.

232.-311. (canceled)

312. The method of claim 1, wherein the one or more antibodies or one or more antigen-binding fragments are broadly neutralizing antibodies against two or more members of the family of three-fingered toxins (3FTx).

313.-318. (canceled)

319. A method of identifying a broadly-neutralizing antibody or antigen-binding fragment that selectively binds to 2 or more snake venom toxins belonging to a family of homologous antigens, the method comprising
(a) immunizing a subject with two or more homologous antigens; and
(b) conducting an iterative selection process to specifically identify cross-reactive antibodies or antigen-binding fragments from the B cell repertoire of the subject, wherein the iterative selection process down-selects possible candidates using 2 or more homologous antigens.

320.-330. (canceled)
